(19) United States
(12) Patent Application Publication
(10) Pub. No.: US 2023/0053028 A1
(43) Pub. Date: Feb. 16, 2023

(54) ENGINEERED CELLS FOR THERAPY

(71) Applicant: Editas Medicine, Inc., Cambridge, MA (US)

(72) Inventors: G. Grant Welstead, Newton, MA (US); Jung Il Moon, Lexington, MA (US)

(21) Appl. No.: 17/786,753

(22) PCT Filed: Dec. 18, 2020
(86) PCT No.: PCT/US2020/066256

§ 371 Date: Jun. 17, 2022

Related U.S. Application Data
(60) Provisional application No. 63/115,592, filed on Nov. 18, 2020.
(60) Provisional application No. 63/025,735, filed on May 15, 2020.
(60) Provisional application No. 62/950,063, filed on Dec. 18, 2019.

(51) Int. Cl. C12N 5/0783; A61K 35/17; C07K 14/725

(52) U.S. Cl. CPC C12N 5/0646; A61K 35/17; C07K 14/7051; C12N 2506/45; C12N 2510/00; C12N 2501/15; C12N 2501/604; C12N 2501/603; C12N 2501/602; C12N 2501/605; C12N 2501/606; C12N 2501/608; C12N 2501/65

(57) ABSTRACT

Methods of culturing embryonic stem cells, induced pluripotent stem cells and/or differentiated cells in culture medium comprising activin are described. In one aspect, the disclosure features a pluripotent human stem cell, wherein the stem cell comprises: (i) a genomic edit that results in loss of function of Cytokine Inducible SH2 Containing Protein (CISH) and (ii) a genomic edit that results in a loss of function of an agonist of the TGF beta signaling pathway, or a genomic edit that results in a loss of function of adenosine Ata receptor.

### CROSS REFERENCE TO RELATED APPLICATIONS

This application claims the benefit of U.S. Provisional Application No. 62/950,063, filed Dec. 18, 2019, U.S. Provisional Application No. 63/025,735, filed May 15, 2020, and U.S. Provisional Application No. 63/115,592, filed Nov. 18, 2020, the contents of all of which are hereby incorporated herein in their entirety.

### BACKGROUND

There remains a need for engineered cells for therapeutic interventions, as well as for methods of culturing stem cells, such as embryonic stem cells and induced pluripotent cells, such that pluripotency is maintained.

### SUMMARY

In one aspect, the disclosure features a pluripotent human stem cell, wherein the stem cell comprises: (i) a genomic edit that results in loss of function of Cytokine Inducible SH2 Containing Protein (CISH) and (ii) a genomic edit that results in a loss of function of an agonist of the TGF beta signaling pathway, or a genomic edit that results in a loss of function of adenosine A2a receptor (ADORA2A). In some embodiments, the stem cell comprises a genomic edit that results in a loss of function of an agonist of the TGF beta signaling pathway and a genomic edit that results in a loss of function of ADORA2A.

In some embodiments, the stem cell comprises a genomic edit that results in a loss of function of a TGF beta receptor or a dominant-negative variant of a TGF beta receptor. In some embodiments, the TGF beta receptor is a TGF beta receptor II (TGFβRII).

In some embodiments, the stem cell expresses one or more pluripotency markers selected from the group consisting of SSEA-3, SSEA-4, TRA-1-60, TRA-1-81, TRA-2-49/6E, ALP, Sox2, E-cadherin, UTF-1, Oct4, Rex1, and Nanog.

In some embodiments, the disclosure features a differentiated cell, wherein the differentiated cell is a daughter cell of a pluripotent human stem cell described herein. In some embodiments, the differentiated cell is an immune cell. In some embodiments, the differentiated cell is a lymphocyte. In some embodiments, the differentiated cell is a natural killer cell. In some embodiments, the stem cell is a human induced pluripotent stem cell (iPSC), and wherein the differentiated daughter cell is an iNK cell. In some embodiments, the cell: (a) does not express endogenous CD3, CD4, and/or CD8; and (b) expresses at least one endogenous gene encoding: (i) CD56 (NCAM), CD49, CD43, and/or CD45, or any combination thereof (ii) NK cell receptor immunoglobulin gamma Fc region receptor III (FcγRIII, cluster of differentiation 16 (CD16)); (iii) natural killer group-2 member D (NKG2D); (iv) CD69; (v) a natural cytotoxicity receptor; or any combination of two or more thereof.

In some embodiments, any of the cells described herein comprises one or more additional genomic edits. In some embodiments, the cell (1) comprises at least one genomic edit characterized by an exogenous nucleic acid expression construct that comprises a nucleic acid sequence encoding: (i) a chimeric antigen receptor (CAR); (ii) a FcγRIII (CD16) or a variant (e.g., non-naturally occurring variant) of FcγRIII (CD16) (iii) interleukin 15 (IL-15); (iv) an IL-15 receptor (IL-15R) agonist, or a constitutively active variant of an IL-15 receptor; (v) an IL-12 receptor (IL-12R) agonist, or a constitutively active variant of an IL-12 receptor; (vi) an IL-12 receptor (IL-12R) agonist, or a constitutively active variant of an IL-12 receptor; (vii) human leukocyte antigen G (HLA-G); (viii) human leukocyte antigen E (HLA-E); (ix) leukocyte surface antigen cluster of differentiation CD47 (CD47); or any combination of two or more thereof and/or (2) comprises at least one genomic edit that results in a loss of function of at least one of: (i) ADORA2A; (ii) T cell immunoreceptor with Ig and ITIM domains (TIGIT); (iii) β-2 microglobulin (B2M); (iv) programmed cell death protein 1 (PD-1); (v) class II, major histocompatibility complex, transactivator (CIITA); (vi) natural killer cell receptor NKG2A (natural killer group 2A); (vii) two or more HLA class II histocompatibility antigen alpha chain genes, and/or two or more HLA class II histocompatibility antigen beta chain genes; (viii) cluster of differentiation 32B (CD32B, FCGR2B); (ix) T cell receptor alpha constant (TRAC); or any combination of two or more thereof.

In another aspect, the disclosure features a human induced pluripotent stem cell (iPSC), wherein the iPSC comprises a genomic edit that results in a loss of function of adenosine A2a receptor (ADORA2A). In some embodiments, the iPSC comprises a genomic edit that results in a loss of function of an agonist of the TGF beta signaling pathway or a genomic edit that results in loss of function of Cytokine Inducible SH2 Containing Protein (CISH). In some embodiments, the iPSC comprises a genomic edit that results in a loss of function of an agonist of the TGF beta signaling pathway and a genomic edit that results in loss of function of CISH.

In some embodiments, the iPSC comprises a genomic edit that results in a loss of function of a TGF beta receptor or a dominant-negative variant of a TGF beta receptor. In some embodiments, TGF beta receptor is a TGF beta receptor II (TGFβRII).

In some embodiments, the iPSC expresses one or more pluripotency markers selected from the group consisting of SSEA-3, SSEA-4, TRA-1-60, TRA-1-81, TRA-2-49/6E, ALP, Sox2, E-cadherin, UTF-1, Oct4, Rex1, and Nanog.

In some embodiments, the disclosure features a differentiated cell, wherein the differentiated cell is a daughter cell of a human iPSC described herein. In some embodiments, the differentiated cell is an immune cell. In some embodiments, the differentiated cell is a lymphocyte. In some embodiments, the differentiated cell is a natural killer cell. In some embodiments, the differentiated daughter cell is an iNK cell. In some embodiments, the cell: (a) does not express endogenous CD3, CD4, and/or CD8; and (b) expresses at least one endogenous gene encoding: (i) CD56 (NCAM), CD49, CD43, and/or CD45, or any combination thereof (ii) NK cell receptor immunoglobulin gamma Fc region receptor III (FcγRIII, cluster of differentiation 16 (CD16)); (iii) natural killer group-2 member D (NKG2D); (iv) CD69; (v) a natural cytotoxicity receptor; or any combination of two or more thereof.

In some embodiments, any of the cells described herein comprises one or more additional genomic edits. In some embodiments, the cell: (1) comprises at least one genomic edit characterized by an exogenous nucleic acid expression construct that comprises a nucleic acid sequence encoding: (i) a chimeric antigen receptor (CAR); (ii) a FcγRIII (CD16) or a variant (e.g., non-naturally occurring variant) of FcγRIII (CD16); (iii) interleukin 15 (IL-15); (iv) an IL-15 receptor (IL-15R) agonist, or a constitutively active variant of an IL-15 receptor; (v) an IL-12 receptor (IL-12R) agonist, or a constitutively active variant of an IL-12 receptor; (vi) an IL-12 receptor (IL-12R) agonist, or a constitutively active variant of an IL-12 receptor; (vii) human leukocyte antigen G (HLA-G); (viii) human leukocyte antigen E (HLA-E); (ix) leukocyte surface antigen cluster of differentiation CD47 (CD47); or any combination of two or more thereof and/or (2) comprises at least one genomic edit that results in a loss of function of at least one of: (i) cytokine inducible SH2 containing protein (CISH); (ii) T cell immunoreceptor with Ig and ITIM domains (TIGIT); (iii) β-2 microglobulin (B2M); (iv) programmed cell death protein 1 (PD-1); (v) class II, major histocompatibility complex, transactivator (CIITA); (vi) natural killer cell receptor NKG2A (natural killer group 2A); (vii) two or more HLA class II histocompatibility antigen alpha chain genes, and/or two or more HLA class II histocompatibility antigen beta chain genes; (viii) cluster of differentiation 32B (CD32B, FCGR2B); (ix) T cell receptor alpha constant (TRAC); or any combination of two or more thereof.

In some embodiments, a genomic edit resulting in loss of function of CISH in any of the cells described herein was produced using a guide RNA comprising a targeting domain sequence comprising or consisting of the nucleotide sequence according to any one of SEQ ID NO: 258-364, 1155, and 1162. In some embodiments, a genomic edit resulting in loss of function of CISH in any of the cells described herein was produced using a guide RNA comprising a targeting domain sequence comprising or consisting of a nucleotide sequence that is identical to, or differs by no more than 1, 2, or 3 nucleotides from, any one of SEQ ID NO: 258-364, 1155, and 1162. In some embodiments, a genomic edit resulting in loss of function of CISH in any of the cells described herein was produced using a guide RNA comprising (i) a targeting domain sequence comprising or consisting of the nucleotide sequence of SEQ ID NO:1155 or 1162, and (ii) a 5′ extension sequence depicted in Table 3. In some embodiments, a genomic edit resulting in loss of function of CISH in any of the cells described herein was produced using a guide RNA comprising (i) a targeting domain sequence comprising or consisting of the nucleotide sequence of SEQ ID NO:1155 or 1162, (ii) a scaffold sequence comprising or consisting of the nucleotide sequence of SEQ ID NO: 1153 located 5′ of the targeting domain sequence, and (iii) the nucleotide sequence of SEQ ID NO:1154 at the 5′ of the scaffold sequence.

In some embodiments, a genomic edit resulting in loss of function of TGFβRII in any of the cells described herein was produced using a guide RNA comprising a targeting domain sequence comprising or consisting of the nucleotide sequence according to any one of SEQ ID NO: 29-257, 1157, and 1161. In some embodiments, a genomic edit resulting in loss of function of TGFβRII in any of the cells described herein was produced using a guide RNA comprising a targeting domain sequence comprising or consisting of a nucleotide sequence that is identical to, or differs by no more than 1, 2, or 3 nucleotides from, any one of SEQ ID NO: 29-257, 1157, and 1161. In some embodiments, a genomic edit resulting in loss of function of TGFβRII in any of the cells described herein was produced using a guide RNA comprising (i) a targeting domain sequence comprising or consisting of the nucleotide sequence of SEQ ID NO: 1157 or 1161, and (ii) a 5′ extension sequence depicted in Table 3. In some embodiments, a genomic edit resulting in loss of function of TGFβRII in any of the cells described herein was produced using a guide RNA comprising (i) a targeting domain sequence comprising or consisting of the nucleotide sequence of SEQ ID NO: 1157 or 1161, (ii) a scaffold sequence comprising or consisting of the nucleotide sequence of SEQ ID NO: 1153 located 5′ of the targeting domain sequence, and (iii) the nucleotide sequence of SEQ ID NO:1154 at the 5′ of the scaffold sequence.

In some embodiments, a genomic edit resulting in loss of function of ADORA2A in any of the cells described herein was produced using a guide RNA comprising a targeting domain sequence comprising or consisting of the nucleotide sequence according to any one of SEQ ID NO: 827-1143, 1159, and 1163. In some embodiments, a genomic edit resulting in loss of function of ADORA2A in any of the cells described herein was produced using a guide RNA comprising a targeting domain sequence comprising or consisting of a nucleotide sequence that is identical to, or differs by no more than 1, 2, or 3 nucleotides from, any one of SEQ ID NO: 827-1143, 1159, and 1163. In some embodiments, a genomic edit resulting in loss of function of ADORA2A in any of the cells described herein was produced using a guide RNA comprising (i) a targeting domain sequence comprising or consisting of the nucleotide sequence of SEQ ID NO: 1159 or 1163, and (ii) a 5′ extension sequence depicted in Table 3. In some embodiments, a genomic edit resulting in loss of function of ADORA2A in any of the cells described herein was produced using a guide RNA comprising (i) a targeting domain sequence comprising or consisting of the nucleotide sequence of SEQ ID NO: 1159 or 1163, (ii) a scaffold sequence comprising or consisting of the nucleotide sequence of SEQ ID NO: 1153 located 5′ of the targeting domain sequence, and (iii) the nucleotide sequence of SEQ ID NO:1154 at the 5′ of the scaffold sequence.

In some embodiments, a genomic edit resulting in loss of function of CISH in any of the cells described herein was produced using a ribonucleoprotein (RNP) complex comprising (i) an RNA-guided nuclease (e.g., a Cas12a variant, e.g., a Cas12a variant comprising 1, 2, or 3 of the amino acid substitutions selected from M537R, F870L, and H800A, e.g., a Cas12a variant comprising an amino acid sequence having 90%, 95%, or 100% identity to SEQ ID NO:1148) and (ii) a guide RNA comprising a targeting domain sequence comprising or consisting of the nucleotide sequence according to any one of SEQ ID NO: 258-364, 1155, and 1162. In some embodiments, a genomic edit resulting in loss of function of CISH in any of the cells described herein was produced using a ribonucleoprotein (RNP) complex comprising (i) an RNA-guided nuclease (e.g., a Cas12a variant, e.g., a Cas12a variant comprising 1, 2, or 3 of the amino acid substitutions selected from M537R, F870L, and H800A, e.g., a Cas12a variant comprising an amino acid sequence having 90%, 95%, or 100% identity to SEQ ID NO:1148) and (ii) a guide RNA comprising a targeting domain sequence comprising or consisting of a nucleotide sequence that is identical to, or differs by no more than 1, 2, or 3 nucleotides from, any one of SEQ ID NO: 258-364, 1155, and 1162. In some embodiments, a genomic edit resulting in loss of function of CISH in any of the cells described herein was produced using a ribonucleoprotein (RNP) complex comprising (i) an RNA-guided nuclease (e.g., a Cas12a variant, e.g., a Cas12a variant comprising 1, 2, or 3 of the amino acid substitutions selected from M537R, F870L, and H800A, e.g., a Cas12a variant comprising an amino acid sequence having 90%, 95%, or 100% identity to SEQ ID NO:1148) and (ii) a guide RNA comprising (i) a targeting domain sequence comprising or consisting of the nucleotide sequence of SEQ ID NO:1155 or 1162, and (ii) a 5′ extension sequence depicted in Table 3. In some embodiments, a genomic edit resulting in loss of function of CISH in any of the cells described herein was produced using a ribonucleoprotein (RNP) complex comprising (i) an RNA-guided nuclease (e.g., a Cas12a variant, e.g., a Cas12a variant comprising 1, 2, or 3 of the amino acid substitutions selected from M537R, F870L, and H800A, e.g., a Cas12a variant comprising an amino acid sequence having 90%, 95%, or 100% identity to SEQ ID NO:1148) and (ii) a guide RNA comprising (i) a targeting domain sequence comprising or consisting of the nucleotide sequence of SEQ ID NO:1155 or 1162, (ii) a scaffold sequence comprising or consisting of the nucleotide sequence of SEQ ID NO: 1153 located 5′ of the targeting domain sequence, and (iii) the nucleotide sequence of SEQ ID NO:1154 at the 5′ of the scaffold sequence.

In some embodiments, a genomic edit resulting in loss of function of TGFβRII in any of the cells described herein was produced using a ribonucleoprotein (RNP) complex comprising (i) an RNA-guided nuclease (e.g., a Cas12a variant, e.g., a Cas12a variant comprising 1, 2, or 3 of the amino acid substitutions selected from M537R, F870L, and H800A, e.g., a Cas12a variant comprising an amino acid sequence having 90%, 95%, or 100% identity to SEQ ID NO:1148), and (ii) a guide RNA comprising a targeting domain sequence comprising or consisting of the nucleotide sequence according to any one of SEQ ID NO: 29-257, 1157, and 1161. In some embodiments, a genomic edit resulting in loss of function of TGFβRII in any of the cells described herein was produced using a ribonucleoprotein (RNP) complex comprising (i) an RNA-guided nuclease (e.g., a Cas12a variant, e.g., a Cas12a variant comprising 1, 2, or 3 of the amino acid substitutions selected from M537R, F870L, and H800A, e.g., a Cas12a variant comprising an amino acid sequence having 90%, 95%, or 100% identity to SEQ ID NO:1148) and (ii) a guide RNA comprising a targeting domain sequence comprising or consisting of a nucleotide sequence that is identical to, or differs by no more than 1, 2, or 3 nucleotides from, any one of SEQ ID NO: 29-257, 1157, and 1161. In some embodiments, a genomic edit resulting in loss of function of TGFβRII in any of the cells described herein was produced using a ribonucleoprotein (RNP) complex comprising (i) an RNA-guided nuclease (e.g., a Cas12a variant, e.g., a Cas12a variant comprising 1, 2, or 3 of the amino acid substitutions selected from M537R, F870L, and H800A, e.g., a Cas12a variant comprising an amino acid sequence having 90%, 95%, or 100% identity to SEQ ID NO:1148), and (ii) a guide RNA comprising (i) a targeting domain sequence comprising or consisting of the nucleotide sequence of SEQ ID NO: 1157 or 1161, and (ii) a 5′ extension sequence depicted in Table 3. In some embodiments, a genomic edit resulting in loss of function of TGFβRII in any of the cells described herein was produced using a ribonucleoprotein (RNP) complex comprising (i) an RNA-guided nuclease (e.g., a Cas12a variant, e.g., a Cas12a variant comprising 1, 2, or 3 of the amino acid substitutions selected from M537R, F870L, and H800A, e.g., a Cas12a variant comprising an amino acid sequence having 90%, 95%, or 100% identity to SEQ ID NO:1148), and (ii) a guide RNA comprising (i) a targeting domain sequence comprising or consisting of the nucleotide sequence of SEQ ID NO: 1157 or 1161, (ii) a scaffold sequence comprising or consisting of the nucleotide sequence of SEQ ID NO: 1153 located 5′ of the targeting domain sequence, and (iii) the nucleotide sequence of SEQ ID NO:1154 at the 5′ of the scaffold sequence.

In some embodiments, a genomic edit resulting in loss of function of ADORA2A in any of the cells described herein was produced using a ribonucleoprotein (RNP) complex comprising (i) an RNA-guided nuclease (e.g., a Cas12a variant, e.g., a Cas12a variant comprising 1, 2, or 3 of the amino acid substitutions selected from M537R, F870L, and H800A, e.g., a Cas12a variant comprising an amino acid sequence having 90%, 95%, or 100% identity to SEQ ID NO:1148) and (ii) a guide RNA comprising a targeting domain sequence comprising or consisting of the nucleotide sequence according to any one of SEQ ID NO: 827-1143, 1159, and 1163. In some embodiments, a genomic edit resulting in loss of function of ADORA2A in any of the cells described herein was produced using a ribonucleoprotein (RNP) complex comprising (i) an RNA-guided nuclease (e.g., a Cas12a variant, e.g., a Cas12a variant comprising 1, 2, or 3 of the amino acid substitutions selected from M537R, F870L, and H800A, e.g., a Cas12a variant comprising an amino acid sequence having 90%, 95%, or 100% identity to SEQ ID NO:1148) and (ii) a guide RNA comprising a targeting domain sequence comprising or consisting of a nucleotide sequence that is identical to, or differs by no more than 1, 2, or 3 nucleotides from, any one of SEQ ID NO: 827-1143, 1159, and 1163. In some embodiments, a genomic edit resulting in loss of function of ADORA2A in any of the cells described herein was produced using a ribonucleoprotein (RNP) complex comprising (i) an RNA-guided nuclease (e.g., a Cas12a variant, e.g., a Cas12a variant comprising 1, 2, or 3 of the amino acid substitutions selected from M537R, F870L, and H800A, e.g., a Cas12a variant comprising an amino acid sequence having 90%, 95%, or 100% identity to SEQ ID NO:1148) and (ii) a guide RNA comprising (i) a targeting domain sequence comprising or consisting of the nucleotide sequence of SEQ ID NO: 1159 or 1163, and (ii) a 5′ extension sequence depicted in Table 3. In some embodiments, a genomic edit resulting in loss of function of ADORA2A in any of the cells described herein was produced using a ribonucleoprotein (RNP) complex comprising (i) an RNA-guided nuclease (e.g., a Cas12a variant, e.g., a Cas12a variant comprising 1, 2, or 3 of the amino acid substitutions selected from M537R, F870L, and H800A, e.g., a Cas12a variant comprising an amino acid sequence having 90%, 95%, or 100% identity to SEQ ID NO:1148) and (ii) guide RNA comprising (i) a targeting domain sequence comprising or consisting of the nucleotide sequence of SEQ ID NO: 1159 or 1163, (ii) a scaffold sequence comprising or consisting of the nucleotide sequence of SEQ ID NO: 1153 located 5′ of the targeting domain sequence, and (iii) the nucleotide sequence of SEQ ID NO:1154 at the 5′ of the scaffold sequence.

In another aspect, the disclosure features a method of making a cell, e.g., a cell described herein, the method comprising contacting a cell (e.g., a pluripotent human stem cell or human induced pluripotent stem cell) with one or more of: an RNA-guided nuclease and a guide RNA comprising a targeting domain sequence comprising or consisting of a nucleotide sequence that is identical to, or differs by no more than 1, 2, or 3 nucleotides from, any one of SEQ ID NO: 258-364, 1155, and 1162; an RNA-guided nuclease and a guide RNA comprising a targeting domain sequence comprising or consisting of a nucleotide sequence that is identical to, or differs by no more than 1, 2, or 3 nucleotides from, any one of SEQ ID NO: 29-257, 1157, and 1161; and/or an RNA-guided nuclease and a guide RNA comprising a targeting domain sequence comprising or consisting of a nucleotide sequence that is identical to, or differs by no more than 1, 2, or 3 nucleotides from, any one of SEQ ID NO: 827-1143, 1159, and 1163.

In some embodiments, the method comprises contacting the cell with one or more of: (1) a guide RNA comprising (i) a targeting domain sequence comprising or consisting of the nucleotide sequence of SEQ ID NO:1155 or 1162, and (ii) a 5′ extension sequence depicted in Table 3; (2) a guide RNA comprising (i) a targeting domain sequence comprising or consisting of the nucleotide sequence of SEQ ID NO: 1157 or 1161, and (ii) a 5′ extension sequence depicted in Table 3; and (3) a guide RNA comprising (i) a targeting domain sequence comprising or consisting of the nucleotide sequence of SEQ ID NO: 1159 or 1163, and (ii) a 5′ extension sequence depicted in Table 3.

In some embodiments, the method comprises contacting the cell with one or more of: (1) a guide RNA comprising (i) a targeting domain sequence comprising or consisting of the nucleotide sequence of SEQ ID NO:1155 or 1162, (ii) a scaffold sequence comprising or consisting of the nucleotide sequence of SEQ ID NO: 1153 located 5′ of the targeting domain sequence, and (iii) the nucleotide sequence of SEQ ID NO:1154 at the 5′ of the scaffold sequence; (2) a guide RNA comprising (i) a targeting domain sequence comprising or consisting of the nucleotide sequence of SEQ ID NO: 1157 or 1161, (ii) a scaffold sequence comprising or consisting of the nucleotide sequence of SEQ ID NO: 1153 located 5′ of the targeting domain sequence, and (iii) the nucleotide sequence of SEQ ID NO:1154 at the 5′ of the scaffold sequence; and (3) a guide RNA comprising (i) a targeting domain sequence comprising or consisting of the nucleotide sequence of SEQ ID NO: 1159 or 1163, (ii) a scaffold sequence comprising or consisting of the nucleotide sequence of SEQ ID NO: 1153 located 5′ of the targeting domain sequence, and (iii) the nucleotide sequence of SEQ ID NO:1154 at the 5′ of the scaffold sequence.

In some embodiments, the RNA-guided nuclease is a Cas12a variant. In some embodiments, the Cas12a variant comprises one or more amino acid substitutions selected from M537R, F870L, and H800A. In some embodiments, the Cas12a variant comprises amino acid substitutions M537R, F870L, and H800A. In some embodiments, the Cas12a variant comprises an amino acid sequence having 90%, 95%, or 100% identity to SEQ ID NO:1148.

In another aspect, the disclosure features a method of making a cell, e.g., a cell described herein, the method comprising contacting a cell (e.g., a pluripotent human stem cell or a human induced pluripotent stem cell) with one or more of: a ribonucleoprotein (RNP) complex comprising (i) an RNA-guided nuclease (e.g., a Cas12a variant, e.g., a Cas12a variant comprising 1, 2, or 3 of the amino acid substitutions selected from M537R, F870L, and H800A, e.g., a Cas12a variant comprising an amino acid sequence having 90%, 95%, or 100% identity to SEQ ID NO:1148) and (ii) a guide RNA comprising a targeting domain sequence comprising or consisting of a nucleotide sequence that is identical to, or differs by no more than 1, 2, or 3 nucleotides from, any one of SEQ ID NO: 258-364, 1155, and 1162; a ribonucleoprotein (RNP) complex comprising (i) an RNA-guided nuclease (e.g., a Cas12a variant, e.g., a Cas12a variant comprising 1, 2, or 3 of the amino acid substitutions selected from M537R, F870L, and H800A, e.g., a Cas12a variant comprising an amino acid sequence having 90%, 95%, or 100% identity to SEQ ID NO:1148) and (ii) a guide RNA comprising a targeting domain sequence comprising or consisting of a nucleotide sequence that is identical to, or differs by no more than 1, 2, or 3 nucleotides from, any one of SEQ ID NO: 29-257, 1157, and 1161; and/or a ribonucleoprotein (RNP) complex comprising (i) an RNA-guided nuclease (e.g., a Cas12a variant, e.g., a Cas12a variant comprising 1, 2, or 3 of the amino acid substitutions selected from M537R, F870L, and H800A, e.g., a Cas12a variant comprising an amino acid sequence having 90%, 95%, or 100% identity to SEQ ID NO:1148) and (ii) a guide RNA comprising a targeting domain sequence comprising or consisting of a nucleotide sequence that is identical to, or differs by no more than 1, 2, or 3 nucleotides from, any one of SEQ ID NO: 827-1143, 1159, and 1163.

In some embodiments, the method comprises contacting the cell with one or more of: (1) an RNP comprising a guide RNA comprising (i) a targeting domain sequence comprising or consisting of the nucleotide sequence of SEQ ID NO:1155 or 1162, and (ii) a 5′ extension sequence depicted in Table 3; (2) an RNP comprising a guide RNA comprising (i) a targeting domain sequence comprising or consisting of the nucleotide sequence of SEQ ID NO: 1157 or 1161, and (ii) a 5′ extension sequence depicted in Table 3; and (3) an RNP comprising a guide RNA comprising (i) a targeting domain sequence comprising or consisting of the nucleotide sequence of SEQ ID NO: 1159 or 1163, and (ii) a 5′ extension sequence depicted in Table 3.

In some embodiments, the method comprises contacting the cell with one or more of: (1) an RNP comprising a guide RNA comprising (i) a targeting domain sequence comprising or consisting of the nucleotide sequence of SEQ ID NO:1155 or 1162, (ii) a scaffold sequence comprising or consisting of the nucleotide sequence of SEQ ID NO: 1153 located 5′ of the targeting domain sequence, and (iii) the nucleotide sequence of SEQ ID NO:1154 at the 5′ of the scaffold sequence; (2) an RNP comprising a guide RNA comprising (i) a targeting domain sequence comprising or consisting of the nucleotide sequence of SEQ ID NO: 1157 or 1161, (ii) a scaffold sequence comprising or consisting of the nucleotide sequence of SEQ ID NO: 1153 located 5′ of the targeting domain sequence, and (iii) the nucleotide sequence of SEQ ID NO:1154 at the 5′ of the scaffold sequence; and (3) an RNP comprising a guide RNA comprising (i) a targeting domain sequence comprising or consisting of the nucleotide sequence of SEQ ID NO: 1159 or 1163, (ii) a scaffold sequence comprising or consisting of the nucleotide sequence of SEQ ID NO: 1153 located 5′ of the targeting domain sequence, and (iii) the nucleotide sequence of SEQ ID NO:1154 at the 5′ of the scaffold sequence.

In some embodiments, the RNA-guided nuclease is a Cas12a variant. In some embodiments, the Cas12a variant comprises one or more amino acid substitutions selected from M537R, F870L, and H800A. In some embodiments, the Cas12a variant comprises amino acid substitutions M537R, F870L, and H800A. In some embodiments, the Cas12a variant comprises an amino acid sequence having 90%, 95%, or 100% identity to SEQ ID NO:1148.

In another aspect, the disclosure features a method of making a cell, e.g., a cell described herein, the method comprising contacting a cell (e.g., a pluripotent human stem cell or a human induced pluripotent stem cell) with (i) a guide RNA comprising a targeting domain sequence comprising or consisting of the nucleotide sequence of SEQ ID NO: 1155 or 1162; a guide RNA comprises a targeting domain sequence comprising or consisting of the nucleotide sequence of SEQ ID NO: 1157 or 1161; and a guide RNA comprises a targeting domain sequence comprising or consisting of the nucleotide sequence of SEQ ID NO: 1159 or 1163; and (ii) an RNA-guided nuclease comprising an amino acid sequence having 90%, 95%, or 100% identity to one of SEQ ID NO:1144-1151 (or a portion thereof).

In another aspect, the disclosure features a method of making a cell, e.g., a cell described herein, the method comprising contacting a cell (e.g., a pluripotent human stem cell or a human induced pluripotent stem cell) with (1) an RNP comprising (i) a guide RNA comprising a targeting domain sequence comprising or consisting of the nucleotide sequence of SEQ ID NO: 1155 or 1162; and (ii) an RNA-guided nuclease comprising an amino acid sequence having 90%, 95%, or 100% identity to one of SEQ ID NO:1144-1151 (or a portion thereof); (2) an RNP comprising (i) a guide RNA comprises a targeting domain sequence comprising or consisting of the nucleotide sequence of SEQ ID NO: 1157 or 1161, and (ii) an RNA-guided nuclease comprising an amino acid sequence having 90%, 95%, or 100% identity to one of SEQ ID NO:1144-1151 (or a portion thereof); and (3) an RNP comprising (i) a guide RNA comprises a targeting domain sequence comprising or consisting of the nucleotide sequence of SEQ ID NO: 1159 or 1163, and (ii) an RNA-guided nuclease comprising an amino acid sequence having 90%, 95%, or 100% identity to one of SEQ ID NO:1144-1151 (or a portion thereof).

In another aspect, the disclosure features a method of making a cell, e.g., a cell described herein, the method comprising contacting a cell (e.g., a pluripotent human stem cell or a human induced pluripotent stem cell) with (1) a guide RNA comprising (i) a targeting domain sequence comprising or consisting of the nucleotide sequence of SEQ ID NO:1155 or 1162, and (ii) a 5′ extension sequence depicted in Table 3; (2) a guide RNA comprising (i) a targeting domain sequence comprising or consisting of the nucleotide sequence of SEQ ID NO: 1157 or 1161, and (ii) a 5′ extension sequence depicted in Table 3; (3) a guide RNA comprising (i) a targeting domain sequence comprising or consisting of the nucleotide sequence of SEQ ID NO: 1159 or 1163, and (ii) a 5′ extension sequence depicted in Table 3; and (4) an RNA-guided nuclease comprising an amino acid sequence having 90%, 95%, or 100% identity to one of SEQ ID NO:1144-1151 (or a portion thereof).

In another aspect, the disclosure features a method of making a cell, e.g., a cell described herein, the method comprising contacting a cell (e.g., a pluripotent human stem cell or a human induced pluripotent stem cell) with (1) an RNP comprising (a) a guide RNA comprising (i) a targeting domain sequence comprising or consisting of the nucleotide sequence of SEQ ID NO:1155 or 1162, and (ii) a 5′ extension sequence depicted in Table 3; and (b) an RNA-guided nuclease comprising an amino acid sequence having 90%, 95%, or 100% identity to one of SEQ ID NO:1144-1151 (or a portion thereof); (2) an RNP comprising (a) a guide RNA comprising (i) a targeting domain sequence comprising or consisting of the nucleotide sequence of SEQ ID NO: 1157 or 1161, and (ii) a 5′ extension sequence depicted in Table 3; and (b) an RNA-guided nuclease comprising an amino acid sequence having 90%, 95%, or 100% identity to one of SEQ ID NO:1144-1151 (or a portion thereof); and (3) an RNP comprising (a) a guide RNA comprising (i) a targeting domain sequence comprising or consisting of the nucleotide sequence of SEQ ID NO: 1159 or 1163, and (ii) a 5′ extension sequence depicted in Table 3; and (b) an RNA-guided nuclease comprising an amino acid sequence having 90%, 95%, or 100% identity to one of SEQ ID NO:1144-1151 (or a portion thereof).

In another aspect, the disclosure features a method of making a cell, e.g., a cell described herein, the method comprising contacting a cell (e.g., a pluripotent human stem cell or a human induced pluripotent stem cell) with (1) a guide RNA comprising (i) a targeting domain sequence comprising or consisting of the nucleotide sequence of SEQ ID NO:1155 or 1162, (ii) a scaffold sequence comprising or consisting of the nucleotide sequence of SEQ ID NO: 1153 located 5′ of the targeting domain sequence, and (iii) the nucleotide sequence of SEQ ID NO:1154 at the 5′ of the scaffold sequence; (2) a guide RNA comprising (i) a targeting domain sequence comprising or consisting of the nucleotide sequence of SEQ ID NO: 1157 or 1161, (ii) a scaffold sequence comprising or consisting of the nucleotide sequence of SEQ ID NO: 1153 located 5′ of the targeting domain sequence, and (iii) the nucleotide sequence of SEQ ID NO:1154 at the 5′ of the scaffold sequence (3) a guide RNA comprising (i) a targeting domain sequence comprising or consisting of the nucleotide sequence of SEQ ID NO: 1159 or 1163, (ii) a scaffold sequence comprising or consisting of the nucleotide sequence of SEQ ID NO: 1153 located 5′ of the targeting domain sequence, and (iii) the nucleotide sequence of SEQ ID NO:1154 at the 5′ of the scaffold sequence; and (4) an RNA-guided nuclease comprising an amino acid sequence having 90%, 95%, or 100% identity to one of SEQ ID NO:1144-1151 (or a portion thereof).

In another aspect, the disclosure features a method of making a cell, e.g., a cell described herein, the method comprising contacting a cell (e.g., a pluripotent human stem cell or a human induced pluripotent stem cell) with (1) an RNP comprising (a) a guide RNA comprising (i) a targeting domain sequence comprising or consisting of the nucleotide sequence of SEQ ID NO:1155 or 1162, (ii) a scaffold sequence comprising or consisting of the nucleotide sequence of SEQ ID NO: 1153 located 5′ of the targeting domain sequence, and (iii) the nucleotide sequence of SEQ ID NO:1154 at the 5′ of the scaffold sequence; and (b) an RNA-guided nuclease comprising an amino acid sequence having 90%, 95%, or 100% identity to one of SEQ ID NO:1144-1151 (or a portion thereof); (2) an RNP comprising (a) a guide RNA comprising (i) a targeting domain sequence comprising or consisting of the nucleotide sequence of SEQ ID NO: 1157 or 1161, (ii) a scaffold sequence comprising or consisting of the nucleotide sequence of SEQ ID NO: 1153 located 5′ of the targeting domain sequence, and (iii) the nucleotide sequence of SEQ ID NO:1154 at the 5′ of the scaffold sequence; and (b) an RNA-guided nuclease comprising an amino acid sequence having 90%, 95%, or 100% identity to one of SEQ ID NO:1144-1151 (or a portion thereof); and (3) an RNP comprising (a) a guide RNA comprising (i) a targeting domain sequence comprising or consisting of the nucleotide sequence of SEQ ID NO: 1159 or 1163, (ii) a scaffold sequence comprising or consisting of the nucleotide sequence of SEQ ID NO: 1153 located 5′ of the targeting domain sequence, and (iii) the nucleotide sequence of SEQ ID NO:1154 at the 5′ of the scaffold sequence; and (b) an RNA-guided nuclease comprising an amino acid sequence having 90%, 95%, or 100% identity to one of SEQ ID NO:1144-1151 (or a portion thereof).

In another aspect, the disclosure features a pluripotent human stem cell, wherein the stem cell comprises a disruption in the transforming growth factor beta (TGF beta) signaling pathway. In some embodiments, the stem cell comprises a genetic modification that results in a loss of function of an agonist of the TGF beta signaling pathway. In some embodiments, the genetic modification is a genomic edit. In some embodiments, the stem cell comprises a loss of function of a TGF beta receptor or a dominant-negative variant of a TGF beta receptor. In some embodiments, the TGF beta receptor is a TGF beta receptor II (TGFβRII).

In some embodiments, the stem cell further comprises a loss of function of an antagonist of interleukin signaling. In some embodiments, the stem cell further comprises a genomic modification that results in the loss of function of an antagonist of interleukin signaling. In some embodiments, the antagonist of interleukin signaling is an antagonist of the IL-15 signaling pathway and/or of the IL-2 signaling pathway.

In some embodiments, the stem cell comprises a loss of function of Cytokine Inducible SH2 Containing Protein (CISH). In some embodiments, the stem cell comprises a genomic modification that results in the loss of function of CISH.

In some embodiments, the stem cell expresses one or more pluripotency markers selected from the group consisting of SSEA-3, SSEA-4, TRA-1-60, TRA-1-81, TRA-2-49/6E, ALP, Sox2, E-cadherin, UTF-1, Oct4, Rex1, and Nanog.

In some embodiments, the stem cell comprises one or more additional genetic modifications. In some embodiments, the stem cell: (1) comprises at least one genetic modification characterized by an exogenous nucleic acid expression construct that comprises a nucleic acid sequence encoding: (i) a chimeric antigen receptor (CAR); (ii) a FcγRIII (CD16) or a variant (e.g., non-naturally occurring variant) of FcγRIII (CD16); (iii) interleukin 15 (IL-15); (iv) an IL-15 receptor (IL-15R) agonist, or a constitutively active variant of an IL-15 receptor; (v) an IL-12 receptor (IL-12R) agonist, or a constitutively active variant of an IL-12 receptor; (vi) an IL-12 receptor (IL-12R) agonist, or a constitutively active variant of an IL-12 receptor; (vii) human leukocyte antigen G (HLA-G); (viii) human leukocyte antigen E (HLA-E); (ix) leukocyte surface antigen cluster of differentiation CD47 (CD47); or any combination of two or more thereof; and/or (2) comprises at least one genetic modification that results in a loss of function of at least one of: (i) cytokine inducible SH2 containing protein (CISH); (ii) adenosine A2a receptor (ADORA2A); (iii) T cell immunoreceptor with Ig and ITIM domains (TIGIT); (iv) β-2 microglobulin (B2M); (v) programmed cell death protein 1 (PD-1); (vi) class II, major histocompatibility complex, transactivator (CIITA); (vii) natural killer cell receptor NKG2A (natural killer group 2A); (viii) two or more HLA class II histocompatibility antigen alpha chain genes, and/or two or more HLA class II histocompatibility antigen beta chain genes; (ix) cluster of differentiation 32B (CD32B, FCGR2B); (x) T cell receptor alpha constant (TRAC); or any combination of two or more thereof.

In some embodiments, the stem cell comprises a genetic modification in a TGFβRII gene made using an RNA-guided nuclease and a gRNA molecule comprising a targeting domain sequence that is the same as, or differs by no more than 3 nucleotides from, any one of SEQ ID NOs:29-257, 1157, and 1161. In some embodiments, the stem cell comprises a genetic modification in a CISH gene made using an RNA-guided nuclease and a gRNA molecule comprising a targeting domain sequence that is the same as, or differs by no more than 3 nucleotides from, any one of SEQ ID NOs:258-364, 1155, and 1162. In some embodiments, the stem cell comprises a genetic modification in a ADORA2A gene made using an RNA-guided nuclease and a gRNA molecule comprising a targeting domain sequence that is the same as, or differs by no more than 3 nucleotides from, any one of SEQ ID NOs:827-1143, 1159, and 1163. In some embodiments, the stem cell comprises a genetic modification in a TIGIT gene made using an RNA-guided nuclease and a gRNA molecule comprising a targeting domain sequence that is the same as, or differs by no more than 3 nucleotides from, any one of SEQ ID NOs:631-826. In some embodiments, the stem cell comprises a genetic modification in a B2M gene made using an RNA-guided nuclease and a gRNA molecule comprising a targeting domain sequence that is the same as, or differs by no more than 3 nucleotides from, any one of SEQ ID NOs:365-576. In some embodiments, the stem cell comprises a genetic modification in a NKG2A gene made using an RNA-guided nuclease and a gRNA molecule comprising a targeting domain sequence that is the same as, or differs by no more than 3 nucleotides from, any one of SEQ ID NOs:577-630.

In another aspect, the disclosure features a differentiated cell, wherein the differentiated cell is a daughter cell of a pluripotent human stem cell described herein. In some embodiments, the differentiated cell is an immune cell. In some embodiments, the differentiated cell is a lymphocyte. In some embodiments, the differentiated cell is a natural killer cell. In some embodiments, the stem cell is a human induced pluripotent stem cell (iPSC), and wherein the differentiated daughter cell is an induced Natural Killer (iNK) cell.

In some embodiments, the differentiated cell: (a) does not express endogenous CD3, CD4, and/or CD8; and (b) expresses at least one endogenous gene encoding: (i) CD56 (NCAM), CD49, CD43, and/or CD45, or any combination thereof (ii) NK cell receptor immunoglobulin gamma Fc region receptor III (FcγRIII, cluster of differentiation 16 (CD16)); (iii) natural killer group-2 member D (NKG2D); (iv) CD69; (v) a natural cytotoxicity receptor; or any combination of two or more thereof.

In some embodiments, the differentiated stem cell comprises one or more additional genetic modifications. In some embodiments, the differentiated stem cell: (1) comprises at least one genetic modification characterized by an exogenous nucleic acid expression construct that comprises a nucleic acid sequence encoding: (i) a chimeric antigen receptor (CAR); (ii) a FcγRIII (CD16) or a variant (e.g., non-naturally occurring variant) of FcγRIII (CD16); (iii) interleukin 15 (IL-15); (iv) an IL-15 receptor (IL-15R) agonist, or a constitutively active variant of an IL-15 receptor; (v) an IL-12 receptor (IL-12R) agonist, or a constitutively active variant of an IL-12 receptor; (vi) an IL-12 receptor (IL-12R) agonist, or a constitutively active variant of an IL-12 receptor; (vii) human leukocyte antigen G (HLA-G); (viii) human leukocyte antigen E (HLA-E); (ix) leukocyte surface antigen cluster of differentiation CD47 (CD47); or any combination of two or more thereof and/or (2) comprises at least one genetic modification that results in a loss of function of at least one of: (i) cytokine inducible SH2 containing protein (CISH); (ii) adenosine A2a receptor (ADORA2A); (iii) T cell immunoreceptor with Ig and ITIM domains (TIGIT); (iv) β-2 microglobulin (B2M); (v) programmed cell death protein 1 (PD-1); (vi) class II, major histocompatibility complex, transactivator (CIITA); (vii) natural killer cell receptor NKG2A (natural killer group 2A); (viii) two or more HLA class II histocompatibility antigen alpha chain genes, and/or two or more HLA class II histocompatibility antigen beta chain genes; (ix) cluster of differentiation 32B (CD32B, FCGR2B); (x) T cell receptor alpha constant (TRAC); or any combination of two or more thereof.

In some embodiments, the differentiated stem cell comprises a genetic modification in a TGFβRII gene made using an RNA-guided nuclease and a gRNA molecule comprising a targeting domain sequence that is the same as, or differs by no more than 3 nucleotides from, any one of SEQ ID NOs:29-257, 1157, and 1161. In some embodiments, the differentiated stem cell comprises a genetic modification in a CISH gene made using an RNA-guided nuclease and a gRNA molecule comprising a targeting domain sequence that is the same as, or differs by no more than 3 nucleotides from, any one of SEQ ID NOs:258-364, 1155, and 1162. In some embodiments, the differentiated stem cell comprises a genetic modification in a ADORA2A gene made using an RNA-guided nuclease and a gRNA molecule comprising a targeting domain sequence that is the same as, or differs by no more than 3 nucleotides from, any one of SEQ ID NOs:827-1143, 1159, and 1163. In some embodiments, the differentiated stem cell comprises a genetic modification in a TIGIT gene made using an RNA-guided nuclease and a gRNA molecule comprising a targeting domain sequence that is the same as, or differs by no more than 3 nucleotides from, any one of SEQ ID NOs:631-826. In some embodiments, the differentiated stem cell comprises a genetic modification in a B2M gene made using an RNA-guided nuclease and a gRNA molecule comprising a targeting domain sequence that is the same as, or differs by no more than 3 nucleotides from, any one of SEQ ID NOs:365-576. In some embodiments, the differentiated stem cell comprises a genetic modification in a NKG2A gene made using an RNA-guided nuclease and a gRNA molecule comprising a targeting domain sequence that is the same as, or differs by no more than 3 nucleotides from, any one of SEQ ID NOs:577-630.

In another aspect, the disclosure features a method of culturing a pluripotent human stem cell, comprising culturing the stem cell in a medium comprising activin. In some embodiments, the pluripotent human stem cell is an embryonic stem cell or an induced pluripotent stem cell. In some embodiments, the pluripotent human stem cell does not express TGFβRII. In some embodiments, the pluripotent human stem cell is genetically engineered not to express TGFβRII. In some embodiments, the pluripotent human stem cell is genetically engineered to knock out a gene encoding TGFβRII.

In some embodiments, the activin is activin A. In some embodiments, the medium does not comprise TGFβ.

In some embodiments, the culturing is performed for a defined period of time (e.g., 1, 2, 3, 4, 5, 6, 7, 8, 9, 10, 11, 12 days, or more). In some embodiments, at one or more times during or following the culturing step, the pluripotent human stem cell maintains pluripotency (e.g., exhibits one or more pluripotency markers). In some embodiments, at one or more times during or following the culturing step, the pluripotent human stem cell expresses a detectable level of one or more of SSEA-3, SSEA-4, TRA-1-60, TRA-1-81, TRA-2-49/6E, ALP, Sox2, E-cadherin, UTF-1, Oct4, Rex1, and Nanog. In some embodiments, at a time during or following the culturing step, the pluripotent human stem cell is differentiated into cells of endoderm, mesoderm, and/or ectoderm lineage. In some embodiments, the pluripotent human stem cell, or its progeny, is further differentiated into a natural killer (NK) cell.

In some embodiments, the pluripotent human stem cell is differentiated into an NK cell in a medium comprising human serum. In some embodiments, the medium comprises NKMACS+human serum (e.g., 5%, 10%, 15%, 20% or more human serum). In some embodiments, the NK cells exhibit improved cellular expansion, increased NK maturity (as exhibited by increased marker expression (e.g., CD45, CD56, CD16, and/or KIR)), and/or increased cytotoxicity, relative to an NK cell differentiated in a media without serum.

In some embodiments, the pluripotent human stem cell (1) comprises at least one genetic modification characterized by an exogenous nucleic acid expression construct that comprises a nucleic acid sequence encoding: (i) a chimeric antigen receptor (CAR); (ii) a FcγRIII (CD16) or a variant (e.g., non-naturally occurring variant) of FcγRIII (CD16); (iii) interleukin 15 (IL-15); (iv) an IL-15 receptor (IL-15R) agonist, or a constitutively active variant of an IL-15 receptor; (v) an IL-12 receptor (IL-12R) agonist, or a constitutively active variant of an IL-12 receptor; (vi) an IL-12 receptor (IL-12R) agonist, or a constitutively active variant of an IL-12 receptor; (vii) human leukocyte antigen G (HLA-G); (viii) human leukocyte antigen E (HLA-E); (ix) leukocyte surface antigen cluster of differentiation CD47 (CD47); or any combination of two or more thereof; and/or (2) comprises at least one genetic modification that results in a loss of function of at least one of: (i) cytokine inducible SH2 containing protein (CISH); (ii) adenosine A2a receptor (ADORA2A); (iii) T cell immunoreceptor with Ig and ITIM domains (TIGIT); (iv) β-2 microglobulin (B2M); (v) programmed cell death protein 1 (PD-1); (vi) class II, major histocompatibility complex, transactivator (CIITA); (vii) natural killer cell receptor NKG2A (natural killer group 2A); (viii) two or more HLA class II histocompatibility antigen alpha chain genes, and/or two or more HLA class II histocompatibility antigen beta chain genes; (ix) cluster of differentiation 32B (CD32B, FCGR2B); (x) T cell receptor alpha constant (TRAC); or any combination of two or more thereof.

In some embodiments, the pluripotent human stem cell comprises a genetic modification in a TGFβRII gene made using an RNA-guided nuclease and a gRNA molecule comprising a targeting domain sequence that is the same as, or differs by no more than 3 nucleotides from, any one of SEQ ID NOs:29-257, 1157, and 1161. In some embodiments, the pluripotent human stem cell comprises a genetic modification in a CISH gene made using an RNA-guided nuclease and a gRNA molecule comprising a targeting domain sequence that is the same as, or differs by no more than 3 nucleotides from, any one of SEQ ID NOs:258-364, 1155, and 1162. In some embodiments, the pluripotent human stem cell comprises a genetic modification in a ADORA2A gene made using an RNA-guided nuclease and a gRNA molecule comprising a targeting domain sequence that is the same as, or differs by no more than 3 nucleotides from, any one of SEQ ID NOs:827-1143, 1159, and 1163. In some embodiments, the pluripotent human stem cell comprises a genetic modification in a TIGIT gene made using an RNA-guided nuclease and a gRNA molecule comprising a targeting domain sequence that is the same as, or differs by no more than 3 nucleotides from, any one of SEQ ID NOs:631-826. In some embodiments, the pluripotent human stem cell comprises a genetic modification in a B2M gene made using an RNA-guided nuclease and a gRNA molecule comprising a targeting domain sequence that is the same as, or differs by no more than 3 nucleotides from, any one of SEQ ID NOs:365-576. In some embodiments, the pluripotent human stem cell comprises a genetic modification in a NKG2A gene made using an RNA-guided nuclease and a gRNA molecule comprising a targeting domain sequence that is the same as, or differs by no more than 3 nucleotides from, any one of SEQ ID NOs:577-630.

In some embodiments, the method further comprises (1) genetically modifying the pluripotent human stem cell such that the pluripotent human stem cell expresses a nucleic acid sequence encoding: (i) a chimeric antigen receptor (CAR); (ii) a non-naturally occurring variant of FcγRIII (CD16); (iii) interleukin 15 (IL-15); (iv) an IL-15 receptor (IL-15R) agonist, or a constitutively active variant of an IL-15 receptor; (v) an IL-12 receptor (IL-12R) agonist, or a constitutively active variant of an IL-12 receptor; (vi) an IL-12 receptor (IL-12R) agonist, or a constitutively active variant of an IL-12 receptor; (vii) human leukocyte antigen G (HLA-G); (viii) human leukocyte antigen E (HLA-E); (ix) leukocyte surface antigen cluster of differentiation CD47 (CD47); or any combination of two or more thereof; and/or (2) genetically modifying the pluripotent human stem cell to lose function of at least one of: (i) cytokine inducible SH2 containing protein (CISH); (ii) adenosine A2a receptor (ADORA2A); (iii) T cell immunoreceptor with Ig and ITIM domains (TIGIT); (iv) β-2 microglobulin (B2M); (v) programmed cell death protein 1 (PD-1); (vi) class II, major histocompatibility complex, transactivator (CIITA); (vii) natural killer cell receptor NKG2A (natural killer group 2A); (viii) two or more HLA class II histocompatibility antigen alpha chain genes, and/or two or more HLA class II histocompatibility antigen beta chain genes; (ix) cluster of differentiation 32B (CD32B, FCGR2B); (x) T cell receptor alpha constant (TRAC); or any combination of two or more thereof.

In some embodiments, the method further comprises genetically modifying a TGFβRII gene using an RNA-guided nuclease and a gRNA molecule comprising a targeting domain sequence that is the same as, or differs by no more than 3 nucleotides from, any one of SEQ ID NOs:29-257, 1157, and 1161. In some embodiments, the method further comprises genetically modifying a CISH gene using an RNA-guided nuclease and a gRNA molecule comprising a targeting domain sequence that is the same as, or differs by no more than 3 nucleotides from, any one of SEQ ID NOs:258-364, 1155, and 11162. In some embodiments, the method further comprises genetically modifying a ADORA2A gene using an RNA-guided nuclease and a gRNA molecule comprising a targeting domain sequence that is the same as, or differs by no more than 3 nucleotides from, any one of SEQ ID NOs:827-1143, 1159, and 1163. In some embodiments, the method further comprises genetically modifying a TIGIT gene using an RNA-guided nuclease and a gRNA molecule comprising a targeting domain sequence that is the same as, or differs by no more than 3 nucleotides from, any one of SEQ ID NOs:631-826. In some embodiments, the method further comprises genetically modifying a B2M gene using an RNA-guided nuclease and a gRNA molecule comprising a targeting domain sequence that is the same as, or differs by no more than 3 nucleotides from, any one of SEQ ID NOs:365-576. In some embodiments, the method further comprises genetically modifying a NKG2A gene using an RNA-guided nuclease and a gRNA molecule comprising a targeting domain sequence that is the same as, or differs by no more than 3 nucleotides from, any one of SEQ ID NOs:577-630.

In another aspect, the disclosure features a cell culture comprising (i) a pluripotent human stem cell and (ii) a cell culture medium comprising activin, wherein the pluripotent human stem cell comprises a disruption in the transforming growth factor beta (TGF beta) signaling pathway. In some embodiments, the stem cell comprises a genetic modification that results in a loss of function of an agonist of the TGF beta signaling pathway. In some embodiments, the genetic modification is a genomic edit. In some embodiments, the stem cell comprises a loss of function of a TGF beta receptor or a dominant-negative variant of a TGF beta receptor. In some embodiments, the TGF beta receptor is a TGF beta receptor II (TGFβRII).

In some embodiments, the pluripotent human stem cell: (1) comprises at least one genetic modification characterized by an exogenous nucleic acid expression construct that comprises a nucleic acid sequence encoding: (i) a chimeric antigen receptor (CAR); (ii) a FcγRIII (CD16) or a variant (e.g., non-naturally occurring variant) of FcγRIII (CD16); (iii) interleukin 15 (IL-15); (iv) an IL-15 receptor (IL-15R) agonist, or a constitutively active variant of an IL-15 receptor; (v) an IL-12 receptor (IL-12R) agonist, or a constitutively active variant of an IL-12 receptor; (vi) an IL-12 receptor (IL-12R) agonist, or a constitutively active variant of an IL-12 receptor; (vii) human leukocyte antigen G (HLA-G); (viii) human leukocyte antigen E (HLA-E); (ix) leukocyte surface antigen cluster of differentiation CD47 (CD47); or any combination of two or more thereof; and/or (2) comprises at least one genetic modification that results in a loss of function of at least one of: (i) cytokine inducible SH2 containing protein (CISH); (ii) adenosine A2a receptor (ADORA2A); (iii) T cell immunoreceptor with Ig and ITIM domains (TIGIT); (iv) β-2 microglobulin (B2M); (v) programmed cell death protein 1 (PD-1); (vi) class II, major histocompatibility complex, transactivator (CIITA); (vii) natural killer cell receptor NKG2A (natural killer group 2A); (viii) two or more HLA class II histocompatibility antigen alpha chain genes, and/or two or more HLA class II histocompatibility antigen beta chain genes; (ix) cluster of differentiation 32B (CD32B, FCGR2B); (x) T cell receptor alpha constant (TRAC); or any combination of two or more thereof.

In some embodiments, the pluripotent human stem cell comprises a genetic modification in a TGFβRII gene made using an RNA-guided nuclease and a gRNA molecule comprising a targeting domain sequence that is the same as, or differs by no more than 3 nucleotides from, any one of SEQ ID NOs:29-257, 1157, and 1161. In some embodiments, the pluripotent human stem cell comprises a genetic modification in a CISH gene made using an RNA-guided nuclease and a gRNA molecule comprising a targeting domain sequence that is the same as, or differs by no more than 3 nucleotides from, any one of SEQ ID NOs:258-364, 1155, and 1162. In some embodiments, the pluripotent human stem cell comprises a genetic modification in a ADORA2A gene made using an RNA-guided nuclease and a gRNA molecule comprising a targeting domain sequence that is the same as, or differs by no more than 3 nucleotides from, any one of SEQ ID NOs:827-1143, 1159, and 1163. In some embodiments, the pluripotent human stem cell comprises a genetic modification in a TIGIT gene made using an RNA-guided nuclease and a gRNA molecule comprising a targeting domain sequence that is the same as, or differs by no more than 3 nucleotides from, any one of SEQ ID NOs:631-826. In some embodiments, the pluripotent human stem cell comprises a genetic modification in a B2M gene made using an RNA-guided nuclease and a gRNA molecule comprising a targeting domain sequence that is the same as, or differs by no more than 3 nucleotides from, any one of SEQ ID NOs:365-576. In some embodiments, the pluripotent human stem cell comprises a genetic modification in a NKG2A gene made using an RNA-guided nuclease and a gRNA molecule comprising a targeting domain sequence that is the same as, or differs by no more than 3 nucleotides from, any one of SEQ ID NOs:577-630.

In another aspect, the method comprises a method of increasing a level of iNK cell activity comprising: (i) providing a pluripotent human stem cell comprising a disruption in the transforming growth factor beta (TGF beta) signaling pathway; and (ii) differentiating the pluripotent human stem cell into an iNK cell, wherein the iNK cell has a higher level of cell activity as compared to an iNK cell not comprising a disruption of the TGF beta signaling pathway.

In some embodiments, the iNK is differentiated from a pluripotent human stem cell cultured in a medium comprising activin. In some embodiments, the method further comprises culturing the pluripotent human stem cell in a medium comprising activin before and/or during the differentiating step.

In some embodiments, the pluripotent human stem cell is differentiated into an NK cell in a medium comprising human serum. In some embodiments, the medium comprises NKMACS+human serum (e.g., 5%, 10%, 15%, 20% or more human serum). In some embodiments, the NK cells exhibit improved cellular expansion, increased NK maturity (as exhibited by increased marker expression (e.g., CD45, CD56, CD16, and/or KIR)), and/or increased cytotoxicity, relative to an NK cell differentiated in a media without serum.

In some embodiments, the method further comprises disrupting the transforming growth factor beta (TGF beta) signaling pathway in the pluripotent human stem cell. In some embodiments, the stem cell comprises a genetic modification that results in a loss of function of an agonist of the TGF beta signaling pathway. In some embodiments, the genetic modification is a genomic edit. In some embodiments, the stem cell comprises a loss of function of a TGF beta receptor or a dominant-negative variant of a TGF beta receptor. In some embodiments, the TGF beta receptor is a TGF beta receptor II (TGFβRID.

In some embodiments, the pluripotent human stem cell: (1) comprises at least one genetic modification characterized by an exogenous nucleic acid expression construct that comprises a nucleic acid sequence encoding: (i) a chimeric antigen receptor (CAR); (ii) a FcγRIII (CD16) or a variant (e.g., non-naturally occurring variant) of FcγRIII (CD16); (iii) interleukin 15 (IL-15); (iv) an IL-15 receptor (IL-15R) agonist, or a constitutively active variant of an IL-15 receptor; (v) an IL-12 receptor (IL-12R) agonist, or a constitutively active variant of an IL-12 receptor; (vi) an IL-12 receptor (IL-12R) agonist, or a constitutively active variant of an IL-12 receptor; (vii) human leukocyte antigen G (HLA-G); (viii) human leukocyte antigen E (HLA-E); (ix) leukocyte surface antigen cluster of differentiation CD47 (CD47); or any combination of two or more thereof; and/or (2) comprises at least one genetic modification that results in a loss of function of at least one of: (i) cytokine inducible SH2 containing protein (CISH); (ii) adenosine A2a receptor (ADORA2A); (iii) T cell immunoreceptor with Ig and ITIM domains (TIGIT); (iv) β-2 microglobulin (B2M); (v) programmed cell death protein 1 (PD-1); (vi) class II, major histocompatibility complex, transactivator (CIITA); (vii) natural killer cell receptor NKG2A (natural killer group 2A); (viii) two or more HLA class II histocompatibility antigen alpha chain genes, and/or two or more HLA class II histocompatibility antigen beta chain genes; (ix) cluster of differentiation 32B (CD32B, FCGR2B); (x) T cell receptor alpha constant (TRAC); or any combination of two or more thereof.

In some embodiments, the pluripotent human stem cell comprises a genetic modification in a TGFβRII gene made using an RNA-guided nuclease and a gRNA molecule comprising a targeting domain sequence that is the same as, or differs by no more than 3 nucleotides from, any one of SEQ ID NOs:29-257, 1157, and 1161. In some embodiments, the pluripotent human stem cell comprises a genetic modification in a CISH gene made using an RNA-guided nuclease and a gRNA molecule comprising a targeting domain sequence that is the same as, or differs by no more than 3 nucleotides from, any one of SEQ ID NOs:258-364, 1155, and 1162. In some embodiments, the pluripotent human stem cell comprises a genetic modification in a ADORA2A gene made using an RNA-guided nuclease and a gRNA molecule comprising a targeting domain sequence that is the same as, or differs by no more than 3 nucleotides from, any one of SEQ ID NOs:827-1143, 1159, and 1163. In some embodiments, the pluripotent human stem cell comprises a genetic modification in a TIGIT gene made using an RNA-guided nuclease and a gRNA molecule comprising a targeting domain sequence that is the same as, or differs by no more than 3 nucleotides from, any one of SEQ ID NOs:631-826. In some embodiments, the pluripotent human stem cell comprises a genetic modification in a B2M gene made using an RNA-guided nuclease and a gRNA molecule comprising a targeting domain sequence that is the same as, or differs by no more than 3 nucleotides from, any one of SEQ ID NOs:365-576. In some embodiments, the pluripotent human stem cell comprises a genetic modification in a NKG2A gene made using an RNA-guided nuclease and a gRNA molecule comprising a targeting domain sequence that is the same as, or differs by no more than 3 nucleotides from, any one of SEQ ID NOs:577-630.

In some embodiments, the method further comprises (1) genetically modifying the pluripotent human stem cell such that the pluripotent human stem cell expresses a nucleic acid sequence encoding: (i) a chimeric antigen receptor (CAR); (ii) a FcγRIII (CD16) or a variant (e.g., non-naturally occurring variant) of FcγRIII (CD16); (iii) interleukin 15 (IL-15); (iv) an IL-15 receptor (IL-15R) agonist, or a constitutively active variant of an IL-15 receptor; (v) an IL-12 receptor (IL-12R) agonist, or a constitutively active variant of an IL-12 receptor; (vi) an IL-12 receptor (IL-12R) agonist, or a constitutively active variant of an IL-12 receptor; (vii) human leukocyte antigen G (HLA-G); (viii) human leukocyte antigen E (HLA-E); (ix) leukocyte surface antigen cluster of differentiation CD47 (CD47); or any combination of two or more thereof; and/or (2) genetically modifying the pluripotent human stem cell to lose function of at least one of: (i) cytokine inducible SH2 containing protein (CISH); (ii) adenosine A2a receptor (ADORA2A); (iii) T cell immunoreceptor with Ig and ITIM domains (TIGIT); (iv) β-2 microglobulin (B2M); (v) programmed cell death protein 1 (PD-1); (vi) class II, major histocompatibility complex, transactivator (CIITA); (vii) natural killer cell receptor NKG2A (natural killer group 2A); (viii) two or more HLA class II histocompatibility antigen alpha chain genes, and/or two or more HLA class II histocompatibility antigen beta chain genes; (ix) cluster of differentiation 32B (CD32B, FCGR2B); (x) T cell receptor alpha constant (TRAC); or any combination of two or more thereof.

In some embodiments, the method further comprises genetically modifying a TGFβRII gene using an RNA-guided nuclease and a gRNA molecule comprising a targeting domain sequence that is the same as, or differs by no more than 3 nucleotides from, any one of SEQ ID NOs:29-257, 1157, and 1161. In some embodiments, the method further comprises genetically modifying a CISH gene using an RNA-guided nuclease and a gRNA molecule comprising a targeting domain sequence that is the same as, or differs by no more than 3 nucleotides from, any one of SEQ ID NOs:258-364, 1155, and 1162. In some embodiments, the method further comprises genetically modifying a ADORA2A gene using an RNA-guided nuclease and a gRNA molecule comprising a targeting domain sequence that is the same as, or differs by no more than 3 nucleotides from, any one of SEQ ID NOs:827-1143, 1159, and 1163. In some embodiments, the method further comprises genetically modifying a TIGIT gene using an RNA-guided nuclease and a gRNA molecule comprising a targeting domain sequence that is the same as, or differs by no more than 3 nucleotides from, any one of SEQ ID NOs:631-826. In some embodiments, the method further comprises genetically modifying a B2M gene using an RNA-guided nuclease and a gRNA molecule comprising a targeting domain sequence that is the same as, or differs by no more than 3 nucleotides from, any one of SEQ ID NOs:365-576. In some embodiments, the method further comprises genetically modifying a NKG2A gene using an RNA-guided nuclease and a gRNA molecule comprising a targeting domain sequence that is the same as, or differs by no more than 3 nucleotides from, any one of SEQ ID NOs:577-630.

In another aspect, the disclosure features a method of culturing a stem cell, for example, a human stem cell, such as, e.g., a human embryonic stem cell, a human induced pluripotent stem cell, or a human pluripotent stem cell, comprising culturing the stem cell in a medium that comprises activin, e.g., activin A. In some embodiments, the stem cell is an embryonic stem cell or an induced pluripotent stem cell. In some embodiments, the stem cell comprises a modification, e.g., a genetic modification, that disrupts a TGF (transforming growth factor) signaling pathway in the stem cell. In some embodiments, the genetic modification is a modification that disrupts (e.g., reduces or abolishes) TGF beta signaling in the stem cell. For example, in some embodiments, the modification is a modification of a gene encoding a protein of the TGF beta signaling pathway, such as a TGF beta receptor. In some embodiments, the modification results in a loss of function and/or a loss of expression of the protein of the TGF beta signaling pathway. In some embodiments, the modification results in a knockout of the protein of the TGF beta signaling pathway. In some embodiments, the stem cell does not express a functional TGFβ receptor protein, e.g., the stem cell does not express a TGFβRII protein or does not express a functional TGFβRII protein. In some embodiments, the stem cell expresses a dominant negative variant of an agonist of a protein of the TGF beta signaling pathway, e.g., a dominant negative variant of TGFβRII. In some embodiments, the stem cell over-expresses an antagonist of the TGF beta signaling pathway. In some embodiments, the stem cell does not express TGFβRII. In some embodiments, the stem cell is genetically engineered not to express TGFβRII. In some embodiments, the stem cell is genetically engineered to knock out a gene encoding TGFβRII. In some embodiments, the genetic modification is a modification that enhances (e.g., maintains or increases) IL-15 signaling in the stem cell. For example, in some embodiments, the modification is a modification of a gene encoding a protein that acts on the IL-15 signaling pathway, such as Cytokine Inducible SH2 Containing Protein (CISH), a negative regulator of IL-15 signaling. In some embodiments, the modification results in a loss of function and/or a loss of expression of the protein that acts on the IL-15 signaling pathway. In some embodiments, the modification results in a knockout of the protein that acts on the IL-15 signaling. In some embodiments, the stem cell does not express a functional CISH gene, e.g., the stem cell does not express a CISH protein or does not express a functional CISH protein. In some embodiments, the stem cell does not express CISH. In some embodiments, the stem cell is genetically engineered not to express CISH. In some embodiments, the stem cell is genetically engineered to knock out a gene encoding CISH (i.e., CISH, cytokine-inducible SH2-containing protein). In some embodiments, the stem cell does not express TGFβRII or CISH. In some embodiments, the stem cell is genetically engineered not to express each of TGFβRII or CISH. In some embodiments, the stem cell is genetically engineered to knock out a gene encoding TGFβRII and a gene encoding CISH in the same cell (double KO). In some embodiments, the stem cell has been edited, e.g., via CRISPR/Cas editing or other suitable technology, to disrupt a gene encoding a gene product involved in TGF signaling, e.g., in TGF beta signaling, such as, for example, a gene encoding a TGF beta RII protein, or e.g., IL-15 signaling, such as, for example, a gene encoding a CIS protein, within the genome of the cell. In some embodiments, e.g., in embodiments, where two copies or alleles of the gene encoding a gene product involved in TGF signaling and/or IL-15 signaling is present in the cell, the cell is modified (e.g., edited), so that both copies or alleles are modified, e.g., in that expression of the gene, or of a functional gene product encoded by the gene, is disrupted, decreased, or abolished from both alleles.

In some embodiments, the activin is activin A. In some embodiments, the medium does not comprise TGFβ.

In some embodiments, the culturing is performed for a defined period of time (e.g., 1, 2, 3, 4, 5, 6, 7, 8, 9, 10, 11, 12 days, or more). In some embodiments, at one or more times during or following the culturing step, the human stem cell maintains pluripotency (e.g., exhibits one or more measure of pluripotency). In some embodiments, at one or more times during or following the culturing step, the human stem cell expresses a detectable level of one or more of SSEA-3, SSEA-4, TRA-1-60, TRA-1-81, TRA-2-49/6E, ALP, Sox2, E-cadherin, UTF-1, Oct4, Rex1, and Nanog. In some embodiments, at a time during or following the culturing step, the human stem cell retains the capacity to differentiate into cells of endoderm, mesoderm, and ectoderm germ layers.

In another aspect, the disclosure features a cell culture comprising (i) an embryonic stem cell or an induced pluripotent stem cell and (ii) a cell culture medium comprising activin, wherein the embryonic stem cell or an induced pluripotent stem cell is genetically engineered not to express TGFβRII and/or CISH.

In some embodiments, an RNA-guided nuclease is a Cas12a variant. In some embodiments, the Cas12a variant comprises amino acid substitutions selected from M537R, F870L, and H800A. In some embodiments, the Cas12a variant comprises amino acid substitutions M537R, F870L, and H800A. In some embodiments, the Cas12a variant comprises an amino acid sequence according to SEQ ID NO: 1148.

### BRIEF DESCRIPTION OF THE DRAWING

The present teachings described herein will be more fully understood from the following description of various illustrative embodiments, when read together with the accompanying drawings. It should be understood that the drawings described below are for illustration purposes only and are not intended to limit the scope of the present teachings in any way.

FIG. 1 shows microscopy of cell morphology and flow cytometry of pluripotency markers of human induced pluripotent stem cells (hiPSCs) grown in various media in the absence or presence of Activin A (1 ng/ml or 4 ng/ml ActA).

FIG. 2 shows morphology of TGFβRII knockout hiPSCs (clone 7) or CISH/TGFβRII DKO hiPSCs (clone 7) cultured in media with or without Activin A (1 ng/mL, 2 ng/mL, 4 ng/mL, or 10 ng/mL).

FIG. 3 shows morphology of TGFβRII knockout hiPSCs (clone 9) cultured in media with our without Activin A (1 ng/mL, 2 ng/mL, 4 ng/mL, or 10 ng/mL).

FIG. 4A shows the bulk editing rates at the CISH and TGFβRII loci for single knockout and double knockout hiPSCs.

FIG. 4B shows expression of Oct4 and SSEA4 in TGFβRII knockout hiPSCs, CISH knockout hiPSCs, and double knockout hiPSCs cultured in Activin A.

FIG. 5 shows expression of Nanog and Tra-1-60 in TGFβRII knockout hiPSCs, CISH knockout hiPSCs, and double knockout hiPSCs cultured in Activin A.

FIG. 6 is a schematic of the procedure related to the STEMdiff™ Trilineage Differentiation Kit (STEMCELL Technologies Inc.).

FIG. 7A shows expression of differentiation markers of TGFβRII knockout hiPSCs, CISH knockout hiPSCs, and double knockout hiPSCs cultured in Activin A.

FIG. 7B shows karyotypes of TGFβRII/CISH double knockout hiPSCs cultured in Activin A.

FIG. 7C shows an expanded Activin A concentration curve performed on an unedited parental PSC line, an edited TGFβRII KO clone (C7), and an additional representative (unedited) cell line designated RUCDR. The minimum concentration of Activin A required to maintain each line varied slightly with the TGFβRII KO clone requiring a higher baseline amount of Activin A as compared to the parental control (0.5 ng/ml vs 0.1 ng/ml).

FIG. 7D shows the stemness marker expression in an unedited parental PSC line, an edited TGFβRII KO clone (C7), and an unedited RUCDR cell line, when cultured with the base medias alone (no supplemental Activin A). The TGFβRII KO iPSCs did not maintain stemness marker expression while the two unedited lines were able to maintain stemness marker expression in E8.

FIG. 8A is a schematic representation of an exemplary method for creating edited iPSC clones, followed by the differentiation to and characterization of enhanced CD56+ iNK cells.

FIG. 8B is a schematic of an iNK cell differentiation process utilizing STEMDiff APEL2 during the second stage of the differentiation process.

FIG. 8C is a schematic of an iNK cell differentiation process utilizing NK-MACS with 15% serum during the second stage of the differentiation process.

FIG. 8D shows the fold-expansion of unedited PCS-derived iNK cells and the percentage of iNK cells expressing CD45 and CD56 at day 39 of differentiation when differentiated using NK-MACS or Apel2 methods as depicted in FIG. 8C and FIG. 8B respectively.

FIG. 8E shows in the upper panel a heat map of the surface expression phenotypes (measured as a percentage of the population) of differentiated iNK cells derived from unedited PCS iPSCs when differentiated using NK-MACS or APEL2 methods as depicted in FIG. 8C and FIG. 8B respectively. The bottom panel displays representative histogram plots to illustrate the differences in the iNKs generated by the two methods.

FIG. 8F shows a heat map of the surface expression phenotypes (measured as a percentage of the population) of differentiated edited iNKs (TGFβRII knockout, CISH knockout, and double knockout (DKO)) and unedited parental iPSCs (WT) when differentiated using NK-MACS or APEL2 methods as depicted in FIG. 8C and FIG. 8B respectively.

FIG. 8G shows unedited iNK cell effector function when differentiated using NK-MACS or APEL2 methods as depicted in FIG. 8C and FIG. 8B respectively.

FIG. 9 shows differentiation phenotypes of edited clones (TGFβRII knockout, CISH knockout, and double knockout) as compared to parental wild type clones.

FIG. 10 shows surface expression phenotype of edited iNKs (TGFβRII knockout, CISH knockout, and double knockout) as compared to parental clone iNKs and wild type cells.

FIG. 11A shows surface expression phenotype of edited iNKs (TGFβRII knockout, CISH knockout, and double knockout) as compared to parental clone iNKs (“WT”) and peripheral blood-derived natural killer cells.

FIG. 11B is a flow cytometry histogram plot that shows the surface expression phenotype of edited iNK cells (TGFβRII/CISH double knockout) as compared to parental clone iNK cells (“unedited iNK cells”).

FIG. 11C shows surface expression phenotypes (measured as a percentage of the population) of edited iNK cells (TGFβRII/CISH double knockout) as compared to parental clone iNK cells (“unedited iNK cells”) at day 25, day 32, and day 39 post-hiPSC differentiation (average values from at least 5 separate differentiations).

FIG. 11D shows pSTAT3 expression phenotypes (measured as a percentage of the population) of edited CD56+ iNK cells (“CISH KO iNKs”) as compared to parental clone CD56+ iNK cells (“unedited iNKs”) at 10 minutes and 120 minutes following IL-15 induced activation. Briefly, the day 39 or day 40 iNKs are plated the day before in a cytokine starve condition. The next day the cells are stimulated with 10 ng/ml of IL15 for the length of time indicated. The cells are fixed immediately at the end of the time point, stained for CD56 followed by an intracellular stain. The cells were processed on a NovoCyte Quanteon and the data was analyzed in FlowJo. Data shown is a representative experiment of >3 experiments performed.

FIG. 11E shows pSMAD2/3 expression phenotypes (measured as a percentage of the population) of edited CD56+ iNK cells (TGFβRII/CISH double knockout, “DKO iNKs”) as compared to parental clone CD56+ iNK cells (“unedited iNK cells”) at 10 minutes and 120 minutes following IL-15 and TGF-β induced activation Briefly, the day 39 or day 40 iNKs were plated the day before in a cytokine starve condition. The next day the cells were stimulated with 10 ng/ml of IL-15 and 50 ng/ml of TGF-β for the length of time indicated. The cells were fixed immediately at the end of the time point, stained for CD56 followed by an intracellular stain. The cells were processed on a NovoCyte Quanteon and the data was analyzed in FlowJo. Data shown is a representative experiment of >3 experiments performed.

FIG. 11F shows IFN-γ expression phenotypes (measured as a percentage of the population) of edited CD56+ iNK cells (TGFβRII/CISH double knockout, “DKO IFNg”) as compared to parental clone CD56+ iNK cells (unedited iNKs, “WT IFNg”) with or without phorbol myristate acetate (PMA) and ionomycin (IMN) stimulation. The data is representative. It is generated from a single differentiation and each condition in the assay is run with 2 technical replicates. **p<0.05 vs unedited iNK cells (paired t test).

FIG. 11G shows TNF-α expression phenotypes (measured as a percentage of the population) of edited CD56+ iNK cells (TGFβRII/CISH double knockout, “DKO TNF a”) as compared to parental clone CD56+ iNK cells (unedited iNK cells, “WT TNFa”) with or without Phorbol myristate acetate (PMA) and Ionomycin (IMN) stimulation. The data is representative. It is generated from a single differentiation and each condition in the assay is run with 2 technical replicates. **p<0.05 vs unedited iNK cells (paired t test).

FIG. 12A is a schematic representation of an exemplary solid tumor cell killing assay, depicting the use of edited iNK cells (TGFβRII/CISH double knockout) to kill SK-OV-3 ovarian cells in the presence or absence of IL-15 and TGF-0.

FIG. 12B shows the results of a solid tumor killing assay as described in FIG. 12A. iNK cells function to reduce tumor cell spheroid size. Certain edited iNK cells (CISH single knockout, “CISH_2, 4, 5, and 8”) were not significantly different from the parental clone iNK cells (“WT_2”), while certain edited iNK cells (TGFβRII single knockout, “TGFβRII_7”, and TGFβRII/CISH double knockout “DKO”) functioned significantly better at effector-target (E:T) ratios of 1 or greater when measured in the presence of TGF-β as compared to parental clone iNK cells (“WT_2”). ****p<0.0001 vs unedited iNK cells (two-way ANOVA, Sidak's multiple comparisons test).

FIG. 12C shows edited iNK cell effector function as compared to unedited iNK cells.

FIG. 13 shows the results of an in-vitro serial killing assay, where iNK cells are serially challenged with hematological cancer cells (e.g., Nalm6 cells) in the presence of 10 ng/ml of IL-15 and 10 ng/ml of TGF-β; the X axis represents time, with tumor cells being added every 48 hours, while the Y axis represents killing efficacy as measured by normalized total red object area (e.g., presence of tumor cells). The data shows that edited iNK cells (TGFβRII/CISH double knockout) continue to kill hematological cancer cells while unedited iNK cells lose this function at equivalent time points.

FIG. 14 shows surface expression phenotypes (measured as a percentage of the population) of certain edited iNK clonal cells (CISH single knockout “CISH_C2, C4, C5, and C8”, TGFβRII single knockout “TGFβRII-C7”, and TGFβRII/CISH double knockout “DKO-C1”) as compared to parental clone iNK cells (“WT”) at day 25, day 32, and day 39 post-hiPSC differentiation when cultured in the presence of 1 ng/mL or 10 ng/mL IL-15.

FIG. 15A is a schematic of an in-vivo tumor killing assay. Mice were intraperitoneally inoculated with 1×10⁶ SKOV3-luc cells, mice are randomized, and 4 days later, 20×10⁶ iNK cells were introduced intraperitoneally. Mice were followed for up to 60 days post-tumor implantation. The X axis represents time since implantation, while the Y axis represents killing efficacy as measured by total bioluminescence (p/s).

FIG. 15B shows the results of an in-vivo tumor killing assay as described in FIG. 15A. An individual mouse is represented by each horizontal line. The data show that both unedited iNK cells (“unedited iNK”) and DKO edited iNK cells (TGFβRII/CISH double knockout) prevent tumor growth better than vehicle, while edited iNK cells kill tumor cells significantly better than vehicle in-vivo. Each experimental group had 9 animals each. ***p<0.001, ****p<0.0001 by a 2-way ANOVA analysis.

FIG. 15C shows the averaged results with standard error of the mean of the in-vivo tumor killing assay described in FIG. 15B. Populations of mice are represented by each horizontal line. The data show that DKO edited iNK cells (TGFβRII/CISH double knockout) prevent tumor growth and kill tumor cells significantly better than vehicle or unedited iNK cells in-vivo. ***p<0.001, ****p<0.0001 by a 2-way ANOVA analysis.

FIG. 16A shows surface expression phenotypes (measured as a percentage of the population) of bulk edited iNK cells (left panel—ADORA2A single knockout) or certain edited iNK clonal cells (right panel—ADORA2A single knockout) as compared to parental clone iNK cells (“PCS WT”) at day 25, day 32, and day 39 or at day 28, day 36, and day 39 post-hiPSC differentiation. Representative data from multiple differentiations.

FIG. 16B shows cyclic AMP (cAMP) concentration phenotypes following 5′-(N-Ethylcarboxamido)adenosine (“NECA”, adenosine agonist) activation for edited iNK clonal cells (ADORA2A single knockout) as compared to parental clone iNK cells (“unedited iNKs”). The Y axis represents average cAMP concentration in nM (a proxy for ADORA2A activation), while the X axis represents NECA concentration in nM.

FIG. 16C shows the results of an in-vitro serial killing assay, where iNK cells are serially challenged with hematological cancer cells (e.g., Nalm6 cells) in the presence of 100 μM NECA, and 10 ng/ml of IL-15; the X axis represents time, with tumor cells being added every 48 hours, while the Y axis represents killing efficacy as measured by total red object area (e.g., presence of tumor cells). The data shows that edited iNK cells (“ADORA2A KO iNK”) kill hematological cancer cells more effectively than unedited iNK cells (“Ctrl iNK”) under conditions that mimic adenosine suppression.

FIG. 17A shows surface expression phenotypes (measured as a percentage of the population) of certain edited iNK clonal cells (TGFβRII/CISH/ADORA2A triple knockout, “CRA_6” and “CR+A_8”) as compared to parental clone iNK cells (“WT_2”) at day 25, day 32, and day 39 post-hiPSC differentiation. Data is representative of multiple differentiations.

FIG. 17B shows cyclic AMP (cAMP) concentration phenotypes following NECA (adenosine agonist) activation for edited iNK clonal cells (TGFβRII/CISH/ADORA2A triple knockout, “TKO iNKs”) as compared to parental clone iNK cells (“unedited iNKs”). The Y axis represents average cAMP concentration in nM (a proxy for ADORA2A activation), while the X axis represents NECA concentration in nM.

FIG. 17C shows the results of a solid tumor killing assay as described in FIG. 12A without IL-15. iNK cells function to reduce tumor cell spheroid size. The Y axis measures total integrated red object (e.g., presence of tumor cells), while the X axis represents the effector to target (E:T) cell ratio. The edited iNK cells (ADORA2A single knockout “ADORA2A”, TGFβRII/CISH double knockout “DKO”, or TGFβRII/CISH/ADORA2A triple knockout “TKO”) had lower EC50 rates when measured in the presence of TGF-β as compared to parental clone iNK cells (“Control”) (average values from at least 3 separate differentiations).

FIG. 18 shows the results of guide RNA selection assays for the loci TGFβRII, CISH, ADORA2A, TIGIT, and NKG2A utilizing in-vitro editing in iPSCs.

### DETAILED DESCRIPTION

Some aspects of the disclosure are based, at least in part, on the recognition that, surprisingly, stem cells, e.g., embryonic stem cells or induced pluripotent stem cells, can be cultured in a culture medium that includes activin A, and that the presence of activin in the culture media abrogates a requirement for the presence of a TGF signaling agonist, e.g., of TGF beta, in the culture medium. Some aspects of the present disclosure relate to the recognition that, surprisingly, stem cells, including human stem cells, such as, for example, human embryonic stem cells or human induced pluripotent stem cells, retain their pluripotency when cultured in media comprising activin, e.g., activin A, even in the absence of a TGF beta signaling agonist, such as, for example, TGF beta, in the culture medium. Additionally, the disclosure is based, in part, on the recognition that, surprisingly, iPSCs lacking TGFβIIR (e.g., genetically knocked out, for example, via gene editing) can be cultured in a culture medium that includes activin, and that such cells not only grow but maintain their pluripotency. The present disclosure additionally encompasses cell cultures comprising embryonic stem cells and a culture medium comprising activin, as well as methods of culturing such stem cells and/or progeny thereof.

### Definitions and Abbreviations

Unless otherwise specified, each of the following terms have the meaning set forth in this section.

The indefinite articles “a” and “an” refer to at least one of the associated noun, and are used interchangeably with the terms “at least one” and “one or more.” The conjunctions “or” and “and/or” are used interchangeably as non-exclusive disjunctions.

The term “cancer” (also used interchangeably with the terms, “hyperproliferative” and “neoplastic”), as used herein, refers to cells having the capacity for autonomous growth, i.e., an abnormal state or condition characterized by rapidly proliferating cell growth. Cancerous disease states may be categorized as pathologic, i.e., characterizing or constituting a disease state, e.g., malignant tumor growth, or may be categorized as non-pathologic, i.e., a deviation from normal but not associated with a disease state, e.g., cell proliferation associated with wound repair. The term is meant to include all types of cancerous growths or oncogenic processes, metastatic tissues or malignantly transformed cells, tissues, or organs, irrespective of histopathologic type or stage of invasiveness. In some embodiments, “cancer” includes malignancies of or affecting various organ systems, such as lung, breast, thyroid, lymphoid, gastrointestinal, and genito-urinary tract. In some embodiments, “cancer” includes adenocarcinomas which include malignancies such as most colon cancers, renal-cell carcinoma, prostate cancer and/or testicular tumors, non-small cell carcinoma of the lung, cancer of the small intestine and/or cancer of the esophagus.

As used herein, the term “carcinoma” is refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. The term carcinoma, as used herein, is well-recognized in the art. Exemplary carcinomas include those forming from tissue of the cervix, lung, prostate, breast, head and neck, colon and ovary. In some embodiments, carcinoma also includes carcinosarcomas, e.g., which include malignant tumors composed of carcinomatous and sarcomatous tissues. In some embodiments, an “adenocarcinoma” is a carcinoma derived from glandular tissue or in which the tumor cells form recognizable glandular structures. In some embodiments, a “sarcoma” is art recognized and refers to malignant tumors of mesenchymal derivation.

The term “differentiation” as used herein is the process by which an unspecialized (“uncommitted”) or less specialized cell acquires the features of a specialized cell such as, for example, a blood cell or a muscle cell. In some embodiments, a differentiated or differentiation-induced cell is one that has taken on a more specialized (“committed”) position within the lineage of a cell. For example, an iPSC can be differentiated into various more differentiated cell types, for example, a neural or a hematopoietic stem cell, a lymphocyte, a cardiomyocyte, and other cell types, upon treatment with suitable differentiation factors in the cell culture medium. In some embodiments, suitable methods, differentiation factors, and cell culture media for the differentiation of pluri- and multipotent cell types into more differentiated cell types are well known to those of skill in the art. In some embodiments, the term “committed”, is applied to the process of differentiation to refer to a cell that has proceeded through a differentiation pathway to a point where, under normal circumstances, it would or will continue to differentiate into a specific cell type or subset of cell types, and cannot, under normal circumstances, differentiate into a different cell type (other than a specific cell type or subset of cell types) nor revert to a less differentiated cell type.

The terms “differentiation marker,” “differentiation marker gene,” or “differentiation gene,” as used herein refers to genes or proteins whose expression are indicative of cell differentiation occurring within a cell, such as a pluripotent cell. In some embodiments, differentiation marker genes include, but are not limited to, the following genes: CD34, CD4, CD8, CD3, CD56 (NCAM), CD49, CD45; NK cell receptor (cluster of differentiation 16 (CD16)), natural killer group-2 member D (NKG2D), CD69, NKp30, NKp44, NKp46, CD158b, FOXA2, FGF5, SOX17, XIST, NODAL, COL3A1, OTX2, DUSP6, EOMES, NR2F2, NROB1, CXCR4, CYP2B6, GAT A3, GATA4, ERBB4, GATA6, HOXC6, INHA, SMAD6, RORA, NIPBL, TNFSF11, CDH11, ZIC4, GAL, SOX3, PITX2, APOA2, CXCL5, CER1, FOXQ1, MLL5, DPP10, GSC, PCDH10, CTCFL, PCDH20, TSHZ1, MEGF10, MYC, DKK1, BMP2, LEFTY2, HES1, CDX2, GNAS, EGR1, COL3A1, TCF4, HEPH, KDR, TOX, FOXA1, LCK, PCDH7, CD1D FOXG1, LEFTY1, TUJ1, T gene (Brachyury), ZIC1, GATA1, GATA2, HDAC4, HDAC5, HDAC7, HDAC9, NOTCH1, NOTCH2, NOTCH4, PAX5, RBPJ, RUNX1, STAT1 and STAT3.

The terms “differentiation marker gene profile,” or “differentiation gene profile,” “differentiation gene expression profile,” “differentiation gene expression signature,” “differentiation gene expression panel,” “differentiation gene panel,” or “differentiation gene signature” as used herein refer to expression or levels of expression of a plurality of differentiation marker genes.

The term “edited iNK cell” as used herein refers to a natural killer cell which has been modified to change at least one expression product of at least one gene at some point in the development of the cell. In some embodiments, a modification can be introduced using, e.g., gene editing techniques such as CRISPR-Cas or, e.g., dominant-negative constructs. In some embodiments, an iNK cell is edited at a time point before it has differentiated into an iNK cell, e.g., at a precursor stage, at a stem cell stage, etc. In some embodiments, an edited iNK cell is compared to a non-edited iNK cell (an NK cell produced by differentiating an iPSC cell, which iPSC cell and/or iNK cell do not have modifications, e.g., genetic modifications).

The term “embryonic stem cell” as used herein refers to pluripotent stem cells derived from the inner cell mass of the embryonic blastocyst. In some embodiments, embryonic stem cells are pluripotent and give rise during development to all derivatives of the three primary germ layers: ectoderm, endoderm and mesoderm. In some such embodiments, embryonic stem cells do not contribute to the extra-embryonic membranes or the placenta, i.e., are not totipotent.

The term “endogenous,” as used herein in the context of nucleic acids (e.g., genes, protein-encoding genomic regions, promoters), refers to a native nucleic acid or protein in its natural location, e.g., within the genome of a cell.

The term “exogenous,” as used herein in the context of nucleic acids, e.g., expression constructs, cDNAs, indels, and nucleic acid vectors, refers to nucleic acids that have artificially been introduced into the genome of a cell using, for example, gene-editing or genetic engineering techniques, e.g., CRISPR-based editing techniques.

The term “genome editing system” refers to any system having RNA-guided DNA editing activity.

The terms “guide RNA” and “gRNA” refer to any nucleic acid that promotes the specific association (or “targeting”) of an RNA-guided nuclease such as a Cas9 or a Cpf1 (Cas12a) to a target sequence such as a genomic or episomal sequence in a cell.

The terms “hematopoietic stem cell,” or “definitive hematopoietic stem cell” as used herein, refer to CD34-positive stem cells. In some embodiments, CD34-positive stem cells are capable of giving rise to mature myeloid and/or lymphoid cell types. In some embodiments, the myeloid and/or lymphoid cell types include, for example, T cells, natural killer cells and/or B cells.

The terms “induced pluripotent stem cell” or “iPSC” as used herein to refer to a stem cell obtained from a differentiated somatic (e.g., adult, neonatal, or fetal) cell by a process referred to as reprogramming (e.g., dedifferentiation). In some embodiments, reprogrammed cells are capable of differentiating into tissues of all three germ or dermal layers: mesoderm, endoderm, and ectoderm. iPSCs are not found in nature.

The term “multipotent stem cell” as used herein refers to a cell that has the developmental potential to differentiate into cells of one or more germ layers (ectoderm, mesoderm and endoderm), but not all three germ layers. Thus, in some embodiments, a multipotent cell may also be termed a “partially differentiated cell.” Multipotent cells are well-known in the art, and examples of multipotent cells include adult stem cells, such as for example, hematopoietic stem cells and neural stem cells. In some embodiments, “multipotent” indicates that a cell may form many types of cells in a given lineage, but not cells of other lineages. For example, a multipotent hematopoietic cell can form the many different types of blood cells (red, white, platelets, etc.), but it cannot form neurons. Accordingly, in some embodiments, “multipotency” refers to a state of a cell with a degree of developmental potential that is less than totipotent and pluripotent.

The term “pluripotent” as used herein refers to ability of a cell to form all lineages of the body or soma (i.e., the embryo proper) or a given organism (e.g., human). For example, embryonic stem cells are a type of pluripotent stem cells that are able to form cells from each of the three germs layers, the ectoderm, the mesoderm, and the endoderm. Generally, pluripotency may be described as a continuum of developmental potencies ranging from an incompletely or partially pluripotent cell (e.g., an epiblast stem cell or EpiSC), which is unable to give rise to a complete organism to the more primitive, more pluripotent cell, which is able to give rise to a complete organism (e.g., an embryonic stem cell or an induced pluripotent stem cell).

The term “pluripotency” as used herein refers to a cell that has the developmental potential to differentiate into cells of all three germ layers (Ectoderm, mesoderm, and endoderm). In some embodiments, pluripotency can be determined, in part, by assessing pluripotency characteristics of the cells. In some embodiments, pluripotency characteristics include, but are not limited to: (i) pluripotent stem cell morphology; (ii) the potential for unlimited self-renewal; (iii) expression of pluripotent stem cell markers including, but not limited to SSEA1 (mouse only), SSEA3/4, SSEA5, TRA1- 60/81, TRA1-85, TRA2-54, GCTM-2, TG343, TG30, CD9, CD29, CD133/prominin, CD140a, CD56, CD73, CD90, CD105, OCT4, NANOG, SOX2, CD30 and/or CD50; (iv) ability to differentiate to all three somatic lineages (ectoderm, mesoderm and endoderm); (v) teratoma formation consisting of the three somatic lineages; and (vi) formation of embryoid bodies consisting of cells from the three somatic lineages.

The term “pluripotent stem cell morphology” as used herein refers to the classical morphological features of an embryonic stem cell. In some embodiments, normal embryonic stem cell morphology is characterized as small and round in shape, with a high nucleus-to-cytoplasm ratio, the notable presence of nucleoli, and typical intercell spacing.

The term “polynucleotide” (including, but not limited to “nucleotide sequence”, “nucleic acid”, “nucleic acid molecule”, “nucleic acid sequence”, and “oligonucleotide”) as used herein refer to a series of nucleotide bases (also called “nucleotides”) in DNA and RNA, and mean any chain of two or more nucleotides. In some embodiments, polynucleotides, nucleotide sequences, nucleic acids etc. can be chimeric mixtures or derivatives or modified versions thereof, single-stranded or double-stranded. In some such embodiments, modifications can occur at the base moiety, sugar moiety, or phosphate backbone, for example, to improve stability of the molecule, its hybridization parameters, etc. In general, a nucleotide sequence typically carries genetic information, including, but not limited to, the information used by cellular machinery to make proteins and enzymes. In some embodiments, a nucleotide sequence and/or genetic information comprises double- or single-stranded genomic DNA, RNA, any synthetic and genetically manipulated polynucleotide, and/or sense and/or antisense polynucleotides. In some embodiments, nucleic acids containing modified bases.

Conventional IUPAC notation is used in nucleotide sequences presented herein, as shown in Table 1, below (see also Cornish-Bowden A, Nucleic Acids Res. 1985 May 10; 13(9):3021-30, incorporated by reference herein). It should be noted, however, that “T” denotes “Thymine or Uracil” in those instances where a sequence may be encoded by either DNA or RNA, for example in gRNA targeting domains.

TABLE 1
IUPAC nucleic acid notation

| Character | Base |
|---|---|
| A | Adenine |
| T | Thymine or Uracil |
| G | Guanine |
| C | Cytosine |
| U | Uracil |
| K | G or T/U |
| M | A or C |
| R | A or G |
| Y | C or T/U |
| S | C or G |
| W | A or T/U |
| B | C, G or T/U |
| V | A, C or G |
| H | A, C or T/U |
| D | A, G or T/U |
| N | A, C, G or T/U |

The terms “potency” or “developmental potency” as used herein refers to the sum of all developmental options accessible to the cell (i.e., the developmental potency), particularly, for example in the context of cellular developmental potential, In some embodiments, the continuum of cell potency includes, but is not limited to, totipotent cells, pluripotent cells, multipotent cells, oligopotent cells, unipotent cells, and terminally differentiated cells.

The terms “prevent,” “preventing,” and “prevention” as used herein refer to the prevention of a disease in a mammal, e.g., in a human, including (a) avoiding or precluding the disease; (b) affecting the predisposition toward the disease; or (c) preventing or delaying the onset of at least one symptom of the disease.

The terms “protein,” “peptide” and “polypeptide” as used herein are used interchangeably to refer to a sequential chain of amino acids linked together via peptide bonds. The terms include individual proteins, groups or complexes of proteins that associate together, as well as fragments or portions, variants, derivatives and analogs of such proteins. Unless otherwise specified, peptide sequences are presented herein using conventional notation, beginning with the amino or N-terminus on the left, and proceeding to the carboxyl or C-terminus on the right. Standard one-letter or three-letter abbreviations can be used.

The terms “reprogramming” or “dedifferentiation” or “increasing cell potency” or “increasing developmental potency” as used herein refer to a method of increasing potency of a cell or dedifferentiating a cell to a less differentiated state. For example, in some embodiments, a cell that has an increased cell potency has more developmental plasticity (i.e., can differentiate into more cell types) compared to the same cell in the non-reprogrammed state. That is, in some embodiments, a reprogrammed cell is one that is in a less differentiated state than the same cell in a non-reprogrammed state. In some embodiments, “reprogramming” refers to de-differentiating a somatic cell, or a multipotent stem cell, into a pluripotent stem cell, also referred to as an induced pluripotent stem cell, or iPSC. Suitable methods for the generation of iPSCs from somatic or multipotent stem cells are well known to those of skill in the art.

The terms “RNA-guided nuclease” and “RNA-guided nuclease molecule” are used interchangeably herein. In some embodiments, the RNA-guided nuclease is a RNA-guided DNA endonuclease enzyme. In some embodiments, the RNA-guided nuclease is a CRISPR nuclease. Non-limiting examples of RNA-guided nucleases are listed in Table 2 below, and the methods and compositions disclosed herein can use any combination of RNA-guided nucleases disclosed herein, or known to those of ordinary skill in the art. Those of ordinary skill in the art will be aware of additional nucleases and nuclease variants suitable for use in the context of the present disclosure, and it will be understood that the present disclosure is not limited in this respect.

TABLE 2
RNA-Guided Nucleases

| | Length | | |
|---|---|---|---|
| Nuclease | (a.a.) | PAM | Reference |
| SpCas9 | 1368 | NGG | Cong et al., Science. 2013; 339 (6121): 819-23 |
| SaCas9 | 1053 | NNGRRT | Ran et al., Nature. 2015; 520 (7546): 186-91. |
| (KKH) | 1067 | NNNRRT | Kleinstiver et al., Nat Biotechnol. |
| SaCas9 | | | 2015; 33 (12): 1293-1298 |
| AsCpf1 | 1353 | TTTV | Zetsche et al., Nat Biotechnol. 2017; 35 (1): 31-34. |
| (AsCas12a) | | | |
| LbCpf1 | 1274 | TTTV | Zetsche et al., Cell. 2015; 163 (3): 759-71. |
| (LbCas12a) | | | |
| CasX | 980 | TTC | Burstein et al., Nature. 2017; 542 (7640): 237-241. |
| CasY | 1200 | TA | Burstein et al., Nature. 2017; 542 (7640): 237-241. |
| Cas12h1 | 870 | RTR | Yan et al., Science. 2019; 363 (6422): 88-91. |
| Cas12i1 | 1093 | TTN | Yan et al., Science. 2019; 363 (6422): 88-91. |
| Cas12c1 | unknown | TG | Yan et al., Science. 2019; 363 (6422): 88-91. |
| Cas12c2 | unknown | TN | Yan et al., Science. 2019; 363 (6422): 88-91. |
| eSpCas9 | 1423 | NGG | Chen et al., Nature. 2017; 550 (7676): 407-410. |
| Cas9-HF1 | 1367 | NGG | Chen et al., Nature. 2017; 550 (7676): 407-410. |
| HypaCas9 | 1404 | NGG | Chen et al., Nature. 2017; 550 (7676): 407-410. |
| dCas9-Fok1 | 1623 | NGG | U.S. Pat. No. 9,322,037 |
| Sniper-Cas9 | 1389 | NGG | Lee et al., Nat Commun. 2018; 9 (1): 3048. |
| xCas9 | 1786 | NGG, NG, | Wang et al., Plant Biotechnol J. 2018; pbi. 13053. |
| | | GAA, | |
| | | GAT | |
| AaCas12b | 1129 | TTN | Teng et al. Cell Discov. 2018; 4: 63. |
| evoCas9 | 1423 | NGG | Casini et al., Nat Biotechnol. 2018; 36 (3): 265-271. |
| SpCas9-NG | 1423 | NG | Nishimasu et al., Science. 2018; 361 (6408): 1259-1262. |
| VRQR | 1368 | NGA | Li et al., The CRISPR Journal, 2018; 01: 01 |
| VRER | 1372 | NGCG | Kleinstiver et al., Nature. 2016; 529 (7587): 490-5. |
| NmeCas9 | 1082 | NNNNGATT | Amrani et al., Genome Biol. 2018; 19 (1): 214. |
| CjCas9 | 984 | NNNNRYAC | Kim et al., Nat Commun. 2017; 8: 14500. |
| BhCas12b | 1108 | ATTN | Strecker et al., Nat Commun. 2019 Jan. 22; 10 (1): 212. |
| BhCas12b | 1108 | ATTN | Strecker et al., Nat Commun. 2019 Jan. 22; 10 (1): 212. |
| V4 | | | |
| CasΦ | 700-800 | TBN | Pausch et al., Science 2020; 369 (6501): 333-337. |
| | | (where B is | |
| | | G, T, or C) | |

Additional suitable RNA-guided nucleases, e.g., Cas9 and Cas12 nucleases, will be apparent to the skilled artisan in view of the present disclosure, and the disclosure is not limited by the exemplary suitable nucleases provided herein. In some embodiments, a suitable nuclease is a Cas9 or Cpf1 (Cas12a) nuclease. In some embodiments, the disclosure also embraces nuclease variants, e.g., Cas9 or Cpf1 nuclease variants. In some embodiments, a nuclease is a nuclease variant, which refers to a nuclease comprising an amino acid sequence characterized by one or more amino acid substitutions, deletions, or additions as compared to the wild type amino acid sequence of the nuclease. In some embodiments, a suitable nuclease and/or nuclease variant may also include purification tags (e.g., polyhistidine tags) and/or signaling peptides, e.g., comprising or consisting of a nuclear localization signal sequence. Some non-limiting examples of suitable nucleases and nuclease variants are described in more detail elsewhere herein and also include those described in PCT application PCT/US2019/22374, filed Mar. 14, 2019, and entitled “Systems and Methods for the Treatment of Hemoglobinopathies,” the entire contents of which are incorporated herein by reference. In some embodiments, the RNA-guided nuclease is an Acidaminococcus sp. Cpf1 variant (AsCpf1 variant). In some embodiments, suitable Cpf1 nuclease variants, including suitable AsCpf1 variants will be known or apparent to those of ordinary skill in the art based on the present disclosure, and include, but are not limited to, the Cpf1 variants disclosed herein or otherwise known in the art. For example, in some embodiments, the RNA-guided nuclease is aAcidaminococcus sp. Cpf1 RR variant (AsCpf1-RR). In another embodiment, the RNA-guided nuclease is a Cpf1 RVR variant. For example, suitable Cpf1 variants include those having an M537R substitution, an H800A substitution, and/or an F870L substitution, or any combination thereof (numbering scheme according to AsCpf1 wild-type sequence).

The term “subject” as used herein means a human or non-human animal. In some embodiments a human subject can be any age (e.g., a fetus, infant, child, young adult, or adult). In some embodiments a human subject may be at risk of or suffer from a disease, or may be in need of alteration of a gene or a combination of specific genes. Alternatively, in some embodiments, a subject may be a non-human animal, which may include, but is not limited to, a mammal. In some embodiments, a non-human animal is a non-human primate, a rodent (e.g., a mouse, rat, hamster, guinea pig, etc.), a rabbit, a dog, a cat, and so on. In certain embodiments of this disclosure, the non-human animal subject is livestock, e.g., avow, a horse, a sheep, a goat, etc. In certain embodiments, the non-human animal subject is poultry, e.g., a chicken, a turkey, a duck, etc.

The terms “treatment,” “treat,” and “treating,” as used herein refer to a clinical intervention aimed to reverse, alleviate, delay the onset of, or inhibit the progress, ameliorate, reduce severity of, prevent or delay the recurrence of a disease, disorder, or condition or one or more symptoms thereof, and/or improve one or more symptoms of a disease, disorder, or condition as described herein. In some embodiments, a condition includes an injury. In some embodiments, an injury may be acute or chronic (e.g., tissue damage from an underlying disease or disorder that causes, e.g., secondary damage such as tissue injury). In some embodiments, treatment, e.g., in the form of a modified NK cell or a population of modified NK cells as described herein, may be administered to a subject after one or more symptoms have developed and/or after a disease has been diagnosed. Treatment may be administered in the absence of symptoms, e.g., to prevent or delay onset of a symptom or inhibit onset or progression of a disease. For example, in some embodiments, treatment may be administered to a susceptible individual prior to the onset of symptoms (e.g., in light of genetic or other susceptibility factors). In some embodiments, treatment may also be continued after symptoms have resolved, for example to prevent or delay their recurrence. In some embodiments, treatment results in improvement and/or resolution of one or more symptoms of a disease, disorder or condition.

The term “variant” as used herein refers to an entity such as a polypeptide, polynucleotide or small molecule that shows significant structural identity with a reference entity but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity.

Stem Cells

Methods of the disclosure can be used to culture stem cells. Stem cells are typically cells that have the capacity to produce unaltered daughter cells (self-renewal; cell division produces at least one daughter cell that is identical to the parent cell) and to give rise to specialized cell types (potency). Stem cells include, but are not limited to, embryonic stem (ES) cells, embryonic germ (EG) cells, germline stem (GS) cells, human mesenchymal stem cells (hMSCs), adipose tissue-derived stem cells (ADSCs), multipotent adult progenitor cells (MAPCs), multipotent adult germline stem cells (maGSCs) and unrestricted somatic stem cell (USSCs). Generally, stem cells can divide without limit. After division, the stem cell may remain as a stem cell, become a precursor cell, or proceed to terminal differentiation. A precursor cell is a cell that can generate a fully differentiated functional cell of at least one given cell type. Generally, precursor cells can divide. After division, a precursor cell can remain a precursor cell, or may proceed to terminal differentiation.

Pluripotent stem cells are generally known in the art. The present disclosure provides technologies (e.g., systems, compositions, methods, etc.) related to pluripotent stem cells. In some embodiments, pluripotent stem cells are stem cells that: (a) are capable of inducing teratomas when transplanted in immunodeficient (SCID) mice; (b) are capable of differentiating to cell types of all three germ layers (e.g., can differentiate to ectodermal, mesodermal, and endodermal cell types); and/or (c) express one or more markers of embryonic stem cells (e.g., human embryonic stem cells express Oct 4, alkaline phosphatase, SSEA-3 surface antigen, SSEA-4 surface antigen, nanog, TRA-1-60, TRA-1-81, SOX2, REX1, etc.). In some aspects, human pluripotent stem cells do not show expression of differentiation markers. In some embodiments, ES cells and/or iPSCs cultured using methods of the disclosure maintain their pluripotency (e.g., (a) are capable of inducing teratomas when transplanted in immunodeficient (SCID) mice; (b) are capable of differentiating to cell types of all three germ layers (e.g., can differentiate to ectodermal, mesodermal, and endodermal cell types); and/or (c) express one or more markers of embryonic stem cells).

In some embodiments, ES cells (e.g., human ES cells) can be derived from the inner cell mass of blastocysts or morulae. In some embodiments, ES cells can be isolated from one or more blastomeres of an embryo, e.g., without destroying the remainder of the embryo. In some embodiments, ES cells can be produced by somatic cell nuclear transfer. In some embodiments, ES cells can be derived from fertilization of an egg cell with sperm or DNA, nuclear transfer, parthenogenesis, or by means to generate ES cells, e.g., with homozygosity in the HLA region. In some embodiments, human ES cells can be produced or derived from a zygote, blastomeres, or blastocyst-staged mammalian embryo produced by the fusion of a sperm and egg cell, nuclear transfer, parthenogenesis, or the reprogramming of chromatin and subsequent incorporation of the reprogrammed chromatin into a plasma membrane to produce an embryonic cell. Exemplary human ES cells are known in the art and include, but are not limited to, MAO1, MAO9, ACT-4, No. 3, H1, H7, H9, H14 and ACT30 ES cells. In some embodiments, human ES cells, regardless of their source or the particular method used to produce them, can be identified based on, e.g., (i) the ability to differentiate into cells of all three germ layers, (ii) expression of at least Oct-4 and alkaline phosphatase, and/or (iii) ability to produce teratomas when transplanted into immunocompromised animals. In some embodiments, ES cells have been serially passaged as cell lines.

iPSCs

Induced pluripotent stem cells (iPSC) are a type of pluripotent stem cell artificially derived from a non-pluripotent cell, such as an adult somatic cell (e.g., a fibroblast cell or other suitable somatic cell), by inducing expression of certain genes. iPSCs can be derived from any organism, such as a mammal. In some embodiments, iPSCs are produced from mice, rats, rabbits, guinea pigs, goats, pigs, cows, non-human primates or humans. iPSCs are similar to ES cells in many respects, such as the expression of certain stem cell genes and proteins, chromatin methylation patterns, doubling time, embryoid body formation, teratoma formation, viable chimera formation, potency and/or differentiability. Various suitable methods for producing iPSCs are known in the art. In some embodiments, iPSCs can be derived by transfection of certain stem cell-associated genes (such asOct-3/4 (Pouf51) and Sox2) into non-pluripotent cells, such as adult fibroblasts. Transfection can be achieved through viral vectors, such as retroviruses, lentiviruses, or adenoviruses. Additional suitable reprogramming methods include the use of vectors that do not integrate into the genome of the host cell, e.g., episomal vectors, or the delivery of reprogramming factors directly via encoding RNA or as proteins has also been described. For example, cells can be transfected with Oct3/4, Sox2, Klf4, and/or c-Myc using a retroviral system or with OCT4, SOX2, NANOG, and/or LIN28 using a lentiviral system. After 3-4 weeks, small numbers of transfected cells begin to become morphologically and biochemically similar to pluripotent stem cells, and can be isolated through morphological selection, doubling time, or through a reporter gene and antibiotic selection. In one example, iPSCs from adult human cells are generated by the method described by Yu et al. (Science 318(5854):1224 (2007)) or Takahashi et al. (Cell 131:861-72 (2007)). In some embodiments, iPSCs are generated by a commercial source. In some embodiments, iPSCs are generated by a vendor. In some embodiments, iPSCs are generated by a contract research organization. Numerous suitable methods for reprogramming are known to those of skill in the art, and the present disclosure is not limited in this respect.

Genetically Engineered Stem Cells

In some embodiments, a stem cell (e.g., iPSC) described herein is genetically engineered to introduce a disruption in one or more targets described herein. For example, in some embodiments, a stem cell (e.g., iPSC) can be genetically engineered to knockout all or a portion of one or more target gene, introduce a frameshift in one or more target genes, and/or cause a truncation of an encoded gene product (e.g., by introducing a premature stop codon). In some embodiments, a stem cell (e.g., iPSC) can be genetically engineered to knockout all or a portion of a target gene using a gene-editing system, e.g., as described herein. In some such embodiments, a gene-editing system may be or comprise a CRISPR system, a zinc finger nuclease system, a TALEN, and/or a meganuclease.

TGF Signaling

In certain embodiments, the disclosure provides a genetically engineered stem cell, and/or progeny cell, comprising a disruption in TGF signaling, e.g., TGF beta signaling. This is useful, for example, in circumstances where it is desirable to generate a differentiated cell from pluripotent stem cell, wherein TGF signaling, e.g., TGF beta signaling is disrupted in the differentiated cell.

For example, TGF beta signaling inhibits or decreases the survival and/or activity of some differentiated cell types that are useful for therapeutic applications, e.g., TGF beta signaling is a negative regulator of natural killer cells, which can be used in immunotherapeutic applications. In some embodiments, it is desirable to generate a clinically effective number of natural killer cells comprising a genetic modification that disrupts TGF beta signaling, thus avoiding the negative effect of TGF beta on the clinical effectiveness of such cells. It is advantageous, in some embodiments, to source such NK cells from a pluripotent stem cell, instead, for example, from mature NK cells obtained from a donor. Modifying the stem cell instead of the differentiated cell has, among others, the advantage of allowing for clonal derivation, characterization, and/or expansion of a specific genotype, e.g., a specific stem cell clone harboring a specific genetic modification (e.g., a targeted disruption of TGFβRII in the absence of any undesired (e.g., off-target) modifications). In some embodiments, the stem cell, e.g., the human iPSC, is genetically engineered not to express one or more TGFβ receptor, e.g., TGFβRII, or to express a dominant negative variant of a TGFβ receptor, e.g., a dominant negative TGFβRII variant. Exemplary sequences of TGFβRII are set forth in KR710923.1, NM 001024847.2, and NM 003242.5. An exemplary dominant negative TGFβRII is disclosed in Immunity. 2000 February; 12(2):171-81.

Additional Loss-of-Function Modifications

In certain embodiments, the disclosure provides a genetically engineered stem cell, and/or progeny cell, that additionally or alternatively comprises a disruption in interleukin signaling, e.g., IL-15 signaling. IL-15 is a cytokine with structural similarity to Interleukin-2 (IL-2), which binds to and signals through a complex composed of IL-2/IL-15 receptor beta chain (CD122) and the common gamma chain (gamma-C, CD132). Exemplary sequences of IL-15 are provided in NG 029605.2. Disruption of IL-15 signaling may be useful, for example, in circumstances where it is desirable to generate a differentiated cell from a pluripotent stem cell, but with certain signaling pathways (e.g., IL-15) disrupted in the differentiated cell. IL-15 signaling can inhibit or decrease survival and/or activity of some types of differentiated cells, such as cells that may be useful for therapeutic applications. For example, IL-15 signaling is a negative regulator of natural killer (NK) cells. CISH (encoded by the CISH gene) is downstream of the IL-15 receptor and can act as a negative regulator of IL-15 signaling in NK cells. As used herein, the term “CISH” refers to the Cytokine Inducible SH2 Containing Protein (see, e.g., Delconte et al., Nat Immunol. 2016 July; 17(7):816-24; exemplary sequences for CISH are set forth as NG 023194.1). In some embodiments, disruption of CISH regulation may increase activation of Jak/STAT pathways, leading to increased survival, proliferation and/or effector functions of NK cells. Thus, in some embodiments, genetically engineered NK cells (e.g., iNK cells, e.g., generated from genetically engineered hiPSCs comprising a disruption of CISH regulation) exhibit greater responsiveness to IL-15-mediated signaling than non-genetically engineered NK cells. In some such embodiments, genetically engineered NK cells exhibit greater effector function relative to non-genetically engineered NK cells.

In some embodiments, a genetically engineered stem cell and/or progeny cell, additionally or alternatively, comprises a disruption and/or loss of function in one or more of B2M, NKG2A, PD1, TIGIT, ADORA2a, CIITA, HLA class II histocompatibility antigen alpha chain genes, HLA class II histocompatibility antigen beta chain genes, CD32B, or TRAC.

As used herein, the term “B2M” ((32 microglobulin) refers to a serum protein found in association with the major histocompatibility complex (MHC) class I heavy chain on the surface of nearly all nucleated cells. Exemplary sequences for B2M are set forth as NG 012920.2.

As used herein, the term “NKG2A” (natural killer group 2A) refers to a protein belonging to the killer cell lectin-like receptor family, also called NKG2 family, which is a group of transmembrane proteins preferentially expressed in NK cells. This family of proteins is characterized by the type II membrane orientation and the presence of a C-type lectin domain. See, e.g., Kamiya-T et al., J Clin Invest 2019 https://doi.org/10.1172/JCI123955. Exemplary sequences for NKG2A are set forth as AF461812.1.

As used herein, the term “PD1” (Programmed cell death protein 1), also known CD279 (cluster of differentiation 279), refers to a protein found on the surface of cells that has a role in regulating the immune system's response to the cells of the human body by down-regulating the immune system and promoting self-tolerance by suppressing T cell inflammatory activity. PD1 is an immune checkpoint and guards against autoimmunity. Exemplary sequences for PD1 are set forth as NM_005018.3.

As used herein, the term “TIGIT” (T cell immunoreceptor with Ig and ITIM domains) refers to a member of the PVR (poliovirus receptor) family of immunoglobulin proteins. The product of this gene is expressed on several classes of T cells including follicular B helper T cells (TFH). Exemplary sequences for TIGIT are set forth in NM 173799.4.

As used herein, the term “ADORA2A” refers to the adenosine A2a receptor, a member of the guanine nucleotide-binding protein (G protein)-coupled receptor (GPCR) superfamily, which is subdivided into classes and subtypes. This protein, an adenosine receptor of A2A subtype, uses adenosine as the preferred endogenous agonist and preferentially interacts with the G(s) and G(olf) family of G proteins to increase intracellular cAMP levels. Exemplary sequences of ADORA2a are provided in NG 052804.1.

As used herein, the term “CIITA” refers to the protein located in the nucleus that acts as a positive regulator of class II major histocompatibility complex gene transcription, and is referred to as the “master control factor” for the expression of these genes. The protein also binds GTP and uses GTP binding to facilitate its own transport into the nucleus. Mutations in this gene have been associated with bare lymphocyte syndrome type II (also known as hereditary MHC class II deficiency or HLA class II-deficient combined immunodeficiency), increased susceptibility to rheumatoid arthritis, multiple sclerosis, and possibly myocardial infarction. See, e.g., Chang et al., J Exp Med 180:1367-1374; and Chang et al., Immunity. 1996 February; 4(2):167-78, the entire contents of each of which are incorporated by reference herein. An exemplary sequence of CIITA is set forth as NG 009628.1.

In some embodiments, two or more HLA class II histocompatibility antigen alpha chain genes and/or two or more HLA class II histocompatibility antigen beta chain genes are disrupted, e.g., knocked out, e.g., by genomic editing. For example, in some embodiments, two or more HLA class II histocompatibility antigen alpha chain genes selected from HLA-DQA1, HLA-DRA, HLA-DPA1, HLA-DMA, HLA-DQA2, and HLA-DOA are disrupted, e.g., knocked out. For another example, in some embodiments, two or more HLA class II histocompatibility antigen beta chain genes selected from HLA-DMB, HLA-DOB, HLA-DPB1, HLA-DQB1, HLA-DQB3, HLA-DQB2, HLA-DRB1, HLA-DRB3, HLA-DRB4, and HLA-DRB5 are disrupted, e.g., knocked out. See, e.g., Crivello et al., J Immunol January 2019, ji1800257; DOI: https://doi.org/10.4049/jimmunol.1800257, the entire contents of which are incorporated herein by reference.

As used herein, the term “CD32B” (cluster of differentiation 32B) refers to a low affinity immunoglobulin gamma Fc region receptor II-b protein that, in humans, is encoded by the FCGR2B gene. See, e.g., Rankin-C T et al., Blood 2006 108(7):2384-91, the entire contents of which are incorporated herein by reference.

As used herein, the term “TRAC” refers to the T-cell receptor alpha subunit (constant), encoded by the TRAC locus.

Gain-of-Function Modifications

In some embodiments, a genetically engineered stem cell and/or progeny cell, additionally or alternatively, comprises a genetic modification that leads to expression of one or more of a CAR; a non-naturally occurring variant of FcγRIII (CD16); interleukin 15 (IL-15); an IL-15 receptor (IL-15R) agonist, or a constitutively active variant of an IL-15 receptor; interleukin 12 (IL-12); an IL-12 receptor (IL-12R) agonist, or a constitutively active variant of an IL-12 receptor; human leukocyte antigen G (HLA-G); human leukocyte antigen E (HLA-E); or leukocyte surface antigen cluster of differentiation CD47 (CD47).

As used herein, the term “chimeric antigen receptor” or “CAR” refers to a receptor protein that has been modified to give cells expressing the CAR the new ability to target a specific protein. Within the context of the disclosure, an cell modified to comprise a CAR may be used for immunotherapy to target and destroy cells associated with a disease or disorder, e.g., cancer cells.

CARs of interest include, but are not limited to, a CAR targeting mesothelin, EGFR, HER2 and/or MICA/B. To date, mesothelin-targeted CAR T-cell therapy has shown early evidence of efficacy in a phase I clinical trial of subjects having mesothelioma, non-small cell lung cancer, and breast cancer (NCT02414269). Similarly, CARs targeting EGFR, HER2 and MICA/B have shown promise in early studies (see, e.g., Li et al. (2018), Cell Death & Disease, 9(177); Han et al. (2018) Am. J. Cancer Res., 8(1):106-119; and Demoulin 2017) Future Oncology, 13(8); the entire contents of each of which are expressly incorporated herein by reference in their entireties).

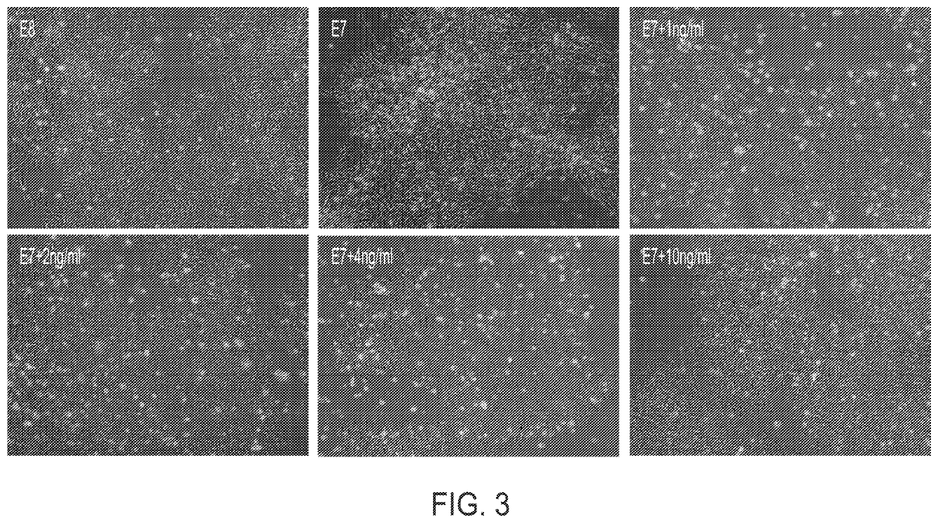

CARs are well-known to those of ordinary skill in the art and include those described in, for example: WO13/063419 (mesothelin), WO15/164594 (EGFR), WO13/063419 (HER2), WO16/154585 (MICA and MICB), the entire contents of each of which are expressly incorporated herein by reference in their entireties. Any suitable CAR, NK-CAR, or other binder that targets a cell, e.g., an NK cell, to a target cell, e.g., a cell associated with a disease or disorder, may be expressed in the modified NK cells provided herein. Exemplary CARs, and binders, include, but are not limited to, CARs and binders that bind BCMA, CD19, CD22, CD20, CD33, CD123, androgen receptor, PSMA, PSCA, Mucl, HPV viral peptides (i.e., E7), EBV viral peptides, CD70, WT1, CEA, EGFRvIII, IL13Rα2, and GD2, CA125, CD7, EpCAM, Muc16, CD30. Additional suitable CARs and binders for use in the modified NK cells provided herein will be apparent to those of skill in the art based on the present disclosure and the general knowledge in the art. Such additional suitable CARs include those described in FIG. 3 of Davies and Maher, Adoptive T-cell Immunotherapy of Cancer Using Chimeric Antigen Receptor-Grafted T Cells, Archivum Immunologiae et Therapiae Experimentalis 58(3):165-78 (2010), the entire contents of which are incorporated herein by reference.

As used herein, the term “CD16” refers to a receptor (FcγRIII) for the Fc portion of immunoglobulin G, and it is involved in the removal of antigen-antibody complexes from the circulation, as well as other antibody-dependent responses.

As used herein, the term “IL-15/IL15RA” or “Interleukin-15” (IL-15) refers to a cytokine with structural similarity to Interleukin-2 (IL-2). Like IL-2, IL-15 binds to and signals through a complex composed of IL-2/IL-15 receptor beta chain (CD122) and the common gamma chain (gamma-C, CD132). IL-15 is secreted by mononuclear phagocytes (and some other cells) following infection by virus(es). This cytokine induces cell proliferation of natural killer cells; cells of the innate immune system whose principal role is to kill virally infected cells. IL-15 Receptor alpha (IL15RA) specifically binds IL-15 with very high affinity, and is capable of binding IL-15 independently of other subunits. It is suggested that this property allows IL-15 to be produced by one cell, endocytosed by another cell, and then presented to a third party cell. IL15RA is reported to enhance cell proliferation and expression of apoptosis inhibitor BCL2L1/BCL2-XL and BCL2. Exemplary sequences of IL-15 are provided in NG 029605.2, and exemplary sequences of IL-15RA are provided in NM 002189.4. In some embodiments, the IL-15R variant is a constitutively active IL-15R variant. In some embodiments, the constitutively active IL-15R variant is a fusion between IL-15R and an IL-15R agonist, e.g., an IL-15 protein or IL-15R-binding fragment thereof. In some embodiments, the IL-15R agonist is IL-15, or an IL-15R-binding variant thereof. Exemplary suitable IL-15R variants include, without limitation, those described, e.g., in Mortier E et al, 2006; The Journal of Biological Chemistry 2006 281: 1612-1619; or in Bessard-A et al., Mol Cancer Ther. 2009 September; 8(9):2736-45, the entire contents of each of which are incorporated by reference herein.

As used herein, the term “IL-12” refers to interleukin-12, a cytokine that acts on T and natural killer cells. In some embodiments, a genetically engineered stem cell and/or progeny cell comprises a genetic modification that leads to expression of one or more of an interleukin 12 (IL12) pathway agonist, e.g., IL-12, interleukin 12 receptor (IL-12R) or a variant thereof (e.g., a constitutively active variant of IL-12R, e.g., an IL-12R fused to an IL-12R agonist (IL-12RA).

As used herein, the term “HLA-G” refers to the HLA non-classical class I heavy chain paralogues. This class I molecule is a heterodimer consisting of a heavy chain and a light chain (beta-2 microglobulin). The heavy chain is anchored in the membrane. HLA-G is expressed on fetal derived placental cells. HLA-G is a ligand for NK cell inhibitory receptor KIR2DL4, and therefore expression of this HLA by the trophoblast defends it against NK cell-mediated death. See e.g., Favier et al., Tolerogenic Function of Dimeric Forms of HLA-G Recombinant Proteins: A Comparative Study In Vivo PLOS One 2011, the entire contents of which are incorporated herein by reference. An exemplary sequence of HLA-G is set forth as NG 029039.1.

As used herein, the term “HLA-E” refers to the HLA class I histocompatibility antigen, alpha chain E, also sometimes referred to as MHC class I antigen E. The HLA-E protein in humans is encoded by the HLA-E gene. The human HLA-E is a non-classical MHC class I molecule that is characterized by a limited polymorphism and a lower cell surface expression than its classical paralogues. This class I molecule is a heterodimer consisting of a heavy chain and a light chain (beta-2 microglobulin). The heavy chain is anchored in the membrane. HLA-E binds a restricted subset of peptides derived from the leader peptides of other class I molecules. HLA-E expressing cells escape allogeneic responses and lysis by NK cells. See e.g., Geornalusse-G et al., Nature Biotechnology 2017 35(8), the entire contents of which are incorporated herein by reference. Exemplary sequences of the HLA-E protein are provided in NM_005516.6.

As used herein, the term “CD47,” also sometimes referred to as “integrin associated protein” (IAP), refers to a transmembrane protein that in humans is encoded by the CD47 gene. CD47 belongs to the immunoglobulin superfamily, partners with membrane integrins, and also binds the ligands thrombospondin-1 (TSP-1) and signal-regulatory protein alpha (SIRPα). CD47 acts as a signal to macrophages that allows CD47-expressing cells to escape macrophage attack. See, e.g., Deuse-T, et al., Nature Biotechnology 2019 37: 252-258, the entire contents of which are incorporated herein by reference.

Generation of iNK Cells

In some embodiments, the present disclosure provides methods of generating iNK cells (e.g., genetically modified iNK cells) that are derived from stem cells described herein.

In some embodiments, genetic modifications (e.g., genomic edits) present in an iNK cell of the present disclosure can be made at any stage during the reprogramming process from donor cell to iPSC, during the iPSC stage, and/or at any stage of the process of differentiating the iPSC to an iNK state, e.g., at an intermediary state, such as, for example, an iPSC-derived HSC state, or even up to or at the final iNK cell state.

For example, one or more genomic edits present in an edited iNK cell of the present disclosure may be made at one or more different cell stages (e.g., reprogramming from donor to iPSC, differentiation of iPSC to iNK). In some embodiments, one or more genomic edits present in modified genetically modified iNK cell provided herein is made before reprogramming a donor cell to an iPSC state. In some embodiments, all edits present in a genetically modified iNK cell provided herein are made at the same time, in close temporal proximity, and/or at the same cell stage of the reprogramming/differentiation process, e.g., at the donor cell stage, during the reprogramming process, at the iPSC stage, or during the differentiation process, e.g., from iPSC to iNK. In some embodiments, two or more edits present in a genetically modified iNK cell provided herein are made at different times and/or at different cell stages of the reprogramming/differentiation process from donor cell to iPSC to iNK. For example, in some embodiments, a first edit is made at the donor cell stage and a second (different) edit is made at the iPSC stage. In some embodiments, a first edit is made at the reprogramming stage (e.g., donor to iPSC) and a second (different) edit is made at the iPSC stage.

A variety of cell types can be used as a donor cell that can be subjected to reprogramming, differentiation, and/or genomic editing strategies described herein. For example, the donor cell can be a pluripotent stem cell or a differentiated cell, e.g., a somatic cell, such as, for example, a fibroblast or a T lymphocyte. In some embodiments, donor cells are manipulated (e.g., subjected to reprogramming, differentiation, and/or genomic editing) to generate iNK cells described herein.

A donor cell can be from any suitable organism. For example, in some embodiments, the donor cell is a mammalian cell, e.g., a human cell or a non-human primate cell. In some embodiments, the donor cell is a somatic cell. In some embodiments, the donor cell is a stem cell or progenitor cell. In certain embodiments, the donor cell is not or was not part of a human embryo and its derivation does not involve destruction of a human embryo.

In some embodiments, an edited iNK cell is derived from an iPSC, which in turn is derived from a somatic donor cell. Any suitable somatic cell can be used in the generation of iPSCs, and in turn, the generation of iNK cells. Suitable strategies for deriving iPSCs from various somatic donor cell types have been described and are known in the art. In some embodiments, a somatic donor cell is a fibroblast cell. In some embodiments, a somatic donor cell is a mature T cell.

For example, in some embodiments, a somatic donor cell, from which an iPSC, and subsequently an iNK cell is derived, is a developmentally mature T cell (a T cell that has undergone thymic selection). One hallmark of developmentally mature T cells is a rearranged T cell receptor locus. During T cell maturation, the TCR locus undergoes V(D)J rearrangements to generate complete V-domain exons. These rearrangements are retained throughout reprogramming of a T cells to an iPSC, and throughout differentiation of the resulting iPSC to a somatic cell.

In certain embodiments, a somatic donor cell is a CD8⁺ T cell, a CD8⁺naïve T cell, a CD4⁺ central memory T cell, a CD8⁺ central memory T cell, a CD4⁺ effector memory T cell, a CD4⁺ effector memory T cell, a CD4⁺ T cell, a CD4⁺ stem cell memory T cell, a CD8+ stem cell memory T cell, a CD4+ helper T cell, a regulatory T cell, a cytotoxic T cell, a natural killer T cell, a CD4+naïve T cell, a TH17 CD4⁺ T cell, a TH1 CD4⁺ T cell, a TH2 CD4⁺ T cell, a TH9 CD4⁺ T cell, a CD4⁺ Foxp3⁺ T cell, a CD4⁺ CD25⁺ CD127⁻ T cell, or a CD4⁺ CD25⁺ CD127⁻ Foxp3⁺ T cell.

T cells can be advantageous for the generation of iPSCs. For example, T cells can be edited with relative ease, e.g., by CRISPR-based methods or other gene-editing methods. Additionally, the rearranged TCR locus allows for genetic tracking of individual cells and their daughter cells. For example, if the reprogramming, expansion, culture, and/or differentiation strategies involved in the generation of NK cells a clonal expansion of a single cell, the rearranged TCR locus can be used as a genetic marker unambiguously identifying a cell and its daughter cells. This, in turn, allows for the characterization of a cell population as truly clonal, or for the identification of mixed populations, or contaminating cells in a clonal population. Another potential advantage of using T cells in generating iNK cells carrying multiple edits is that certain karyotypic aberrations associated with chromosomal translocations are selected against in T cell culture. Such aberrations can pose a concern when editing cells by CRISPR technology, and in particular when generating cells carrying multiple edits. Using T cell derived iPSCs as a starting point for the derivation of therapeutic lymphocytes can allow for the expression of a pre-screened TCR in the lymphocytes, e.g., via selecting the T cells for binding activity against a specific antigen, e.g., a tumor antigen, reprogramming the selected T cells to iPSCs, and then deriving lymphocytes from these iPSCs that express the TCR (e.g., T cells). This strategy can allow for activating the TCR in other cell types, e.g., by genetic or epigenetic strategies. Additionally, T cells retain at least part of their “epigenetic memory” throughout the reprogramming process, and thus subsequent differentiation of the same or a closely related cell type, such as iNK cells can be more efficient and/or result in higher quality cell populations as compared to approaches using non-related cells, such as fibroblasts, as a starting point for iNK derivation.

In some embodiments, a donor cell being manipulated, e.g., a cell being reprogrammed and/or undergoing genomic editing, is one or more of a long-term hematopoietic stem cell, a short term hematopoietic stem cell, a multipotent progenitor cell, a lineage restricted progenitor cell, a lymphoid progenitor cell, a myeloid progenitor cell, a common myeloid progenitor cell, an erythroid progenitor cell, a megakaryocyte erythroid progenitor cell, a retinal cell, a photoreceptor cell, a rod cell, a cone cell, a retinal pigmented epithelium cell, a trabecular meshwork cell, a cochlear hair cell, an outer hair cell, an inner hair cell, a pulmonary epithelial cell, a bronchial epithelial cell, an alveolar epithelial cell, a pulmonary epithelial progenitor cell, a striated muscle cell, a cardiac muscle cell, a muscle satellite cell, a neuron, a neuronal stem cell, a mesenchymal stem cell, an induced pluripotent stem (iPS) cell, an embryonic stem cell, a fibroblast, a monocyte-derived macrophage or dendritic cell, a megakaryocyte, a neutrophil, an eosinophil, a basophil, a mast cell, a reticulocyte, a B cell, e.g., a progenitor B cell, a Pre B cell, a Pro B cell, a memory B cell, a plasma B cell, a gastrointestinal epithelial cell, a biliary epithelial cell, a pancreatic ductal epithelial cell, an intestinal stem cell, a hepatocyte, a liver stellate cell, a Kupffer cell, an osteoblast, an osteoclast, an adipocyte, a preadipocyte, a pancreatic islet cell (e.g., a beta cell, an alpha cell, a delta cell), a pancreatic exocrine cell, a Schwann cell, or an oligodendrocyte.

In some embodiments, a donor cell is one or more of a circulating blood cell, e.g., a reticulocyte, megakaryocyte erythroid progenitor (MEP) cell, myeloid progenitor cell (CMP/GMP), lymphoid progenitor (LP) cell, hematopoietic stem/progenitor cell (HSC), or endothelial cell (EC). In some embodiments, a donor cell is one or more of a bone marrow cell (e.g., a reticulocyte, an erythroid cell (e.g., erythroblast), an MEP cell, myeloid progenitor cell (CMP/GMP), LP cell, erythroid progenitor (EP) cell, HSC, multipotent progenitor (MPP) cell, endothelial cell (EC), hemogenic endothelial (HE) cell, or mesenchymal stem cell). In some embodiments, a donor cell is one or more of a myeloid progenitor cell (e.g., a common myeloid progenitor (CMP) cell or granulocyte macrophage progenitor (GMP) cell). In some embodiments, a donor cell is one or more of a lymphoid progenitor cell, e.g., a common lymphoid progenitor (CLP) cell. In some embodiments, a donor cell is one or more of an erythroid progenitor cell (e.g., an MEP cell). In some embodiments, a donor cell is one or more of a hematopoietic stem/progenitor cell (e.g., a long term HSC (LT-HSC), short term HSC (ST-HSC), MPP cell, or lineage restricted progenitor (LRP) cell). In certain embodiments, the donor cell is a CD34⁺ cell, CD34+CD90⁺ cell, CD34⁺ CD38⁻ cell, CD34⁺ CD90⁺ CD49f⁺ CD38⁻ CD45RA⁻ cell, CD105⁺ cell, CD31⁺, or CD133⁺ cell, or a CD34⁺ CD90⁺ CD133⁺ cell. In some embodiments, a donor cell is one or more of an umbilical cord blood CD34⁺ HSPC, umbilical cord venous endothelial cell, umbilical cord arterial endothelial cell, amniotic fluid CD34⁺ cell, amniotic fluid endothelial cell, placental endothelial cell, or placental hematopoietic CD34⁺ cell. In some embodiments, a donor cell is one or more of a mobilized peripheral blood hematopoietic CD34⁺ cell (after the patient is treated with a mobilization agent, e.g., G-CSF or Plerixafor). In some embodiments, a donor cell is a peripheral blood endothelial cell. In some embodiments, a donor cell is a peripheral blood natural killer cell.

In some embodiments, a donor cell is a dividing cell. In some embodiments, a donor cell is a non-dividing cell.

In some embodiments, a genetically modified (e.g., edited) iNK cell resulting from one or more methods and/or strategies described herein, are administered to a subject in need thereof, e.g., in the context of an immuno-oncology therapeutic approach. In some embodiments, donor cells, or any cells of any stage of the reprogramming, differentiating, and/or editing strategies provided herein, can be maintained in culture or stored (e.g., frozen in liquid nitrogen) using any suitable method known in the art, e.g., for subsequent characterization or administration to a subject in need thereof.

Genome Editing Systems

Genome editing systems of the present disclosure may be used, for example, to edit stem cells. In some embodiments, genome editing systems of the present disclosure include at least two components adapted from naturally occurring CRISPR systems: a guide RNA (gRNA) and an RNA-guided nuclease. These two components form a complex that is capable of associating with a specific nucleic acid sequence and editing the DNA in or around that nucleic acid sequence, for instance by making one or more of a single-strand break (an SSB or nick), a double-strand break (a DSB) and/or a point mutation.

Naturally occurring CRISPR systems are organized evolutionarily into two classes and five types (Makarova et al. Nat Rev Microbiol. 2011 June; 9(6): 467-477 (“Makarova”)), and while genome editing systems of the present disclosure may adapt components of any type or class of naturally occurring CRISPR system, the embodiments presented herein are generally adapted from Class 2, and type II or V CRISPR systems. Class 2 systems, which encompass types II and V, are characterized by relatively large, multidomain RNA-guided nuclease proteins (e.g., Cas9 or Cpf1) and one or more guide RNAs (e.g., a crRNA and, optionally, a tracrRNA) that form ribonucleoprotein (RNP) complexes that associate with (i.e., target) and cleave specific loci complementary to a targeting (or spacer) sequence of the crRNA. Genome editing systems according to the present disclosure similarly target and edit cellular DNA sequences, but differ significantly from CRISPR systems occurring in nature. For example, the unimolecular guide RNAs described herein do not occur in nature, and both guide RNAs and RNA-guided nucleases according to this disclosure may incorporate any number of non-naturally occurring modifications.

Genome editing systems can be implemented (e.g. administered or delivered to a cell or a subject) in a variety of ways, and different implementations may be suitable for distinct applications. For instance, a genome editing system is implemented, in certain embodiments, as a protein/RNA complex (a ribonucleoprotein, or RNP), which can be included in a pharmaceutical composition that optionally includes a pharmaceutically acceptable carrier and/or an encapsulating agent, such as a lipid or polymer micro- or nanoparticle, micelle, liposome, etc. In certain embodiments, a genome editing system is implemented as one or more nucleic acids encoding the RNA-guided nuclease and guide RNA components described above (optionally with one or more additional components); in certain embodiments, the genome editing system is implemented as one or more vectors comprising such nucleic acids, for instance a viral vector such as an adeno-associated virus; and in certain embodiments, the genome editing system is implemented as a combination of any of the foregoing. Additional or modified implementations that operate according to the principles set forth herein will be apparent to the skilled artisan and are within the scope of this disclosure.

It should be noted that the genome editing systems of the present disclosure can be targeted to a single specific nucleotide sequence, or may be targeted to—and capable of editing in parallel—two or more specific nucleotide sequences through the use of two or more guide RNAs. The use of multiple gRNAs is referred to as “multiplexing” throughout this disclosure, and can be employed to target multiple, unrelated target sequences of interest, or to form multiple SSBs or DSBs within a single target domain and, in some cases, to generate specific edits within such target domain. For example, International Patent Publication No. WO 2015/138510 by Maeder et al. (“Maeder”) describes a genome editing system for correcting a point mutation (C.2991+1655A to G) in the human CEP290 gene that results in the creation of a cryptic splice site, which in turn reduces or eliminates the function of the gene. The genome editing system of Maeder utilizes two guide RNAs targeted to sequences on either side of (i.e., flanking) the point mutation, and forms DSBs that flank the mutation. This, in turn, promotes deletion of the intervening sequence, including the mutation, thereby eliminating the cryptic splice site and restoring normal gene function.

As another example, WO 2016/073990 by Cotta-Ramusino, et al. (“Cotta-Ramusino”) describes a genome editing system that utilizes two gRNAs in combination with a Cas9 nickase (a Cas9 that makes a single strand nick such as S. pyogenes D10A), an arrangement termed a “dual-nickase system.” The dual-nickase system of Cotta-Ramusino is configured to make two nicks on opposite strands of a sequence of interest that are offset by one or more nucleotides, which nicks combine to create a double strand break having an overhang (5′ in the case of Cotta-Ramusino, though 3′ overhangs are also possible). The overhang, in turn, can facilitate homology directed repair events in some circumstances. And, as another example, WO 2015/070083 by Palestrant et al. (“Palestrant”) describes a gRNA targeted to a nucleotide sequence encoding Cas9 (referred to as a “governing RNA”), which can be included in a genome editing system comprising one or more additional gRNAs to permit transient expression of a Cas9 that might otherwise be constitutively expressed, for example in some virally transduced cells. These multiplexing applications are intended to be exemplary, rather than limiting, and the skilled artisan will appreciate that other applications of multiplexing are generally compatible with the genome editing systems described here.

Genome editing systems can, in some instances, form double strand breaks that are repaired by cellular DNA double-strand break mechanisms such as NHEJ or HDR. These mechanisms are described throughout the literature, for example by Davis & Maizels, PNAS, 111(10):E924-932, Mar. 11, 2014 (“Davis”) (describing Alt-HDR); Frit et al. DNA Repair 17(2014) 81-97 (“Frit”) (describing Alt-NHEJ); and Iyama and Wilson III, DNA Repair (Amst.) 2013-August; 12(8): 620-636 (“Iyama”) (describing canonical HDR and NHEJ pathways generally).

Where genome editing systems operate by forming DSBs, such systems optionally include one or more components that promote or facilitate a particular mode of double-strand break repair or a particular repair outcome. For instance, Cotta-Ramusino also describes genome editing systems in which a single stranded oligonucleotide “donor template” is added; the donor template is incorporated into a target region of cellular DNA that is cleaved by the genome editing system, and can result in a change in the target sequence.

In certain embodiments, genome editing systems modify a target sequence, or modify expression of a target gene in or near the target sequence, without causing single- or double-strand breaks. For example, a genome editing system may include an RNA-guided nuclease fused to a functional domain that acts on DNA, thereby modifying the target sequence or its expression. As one example, an RNA-guided nuclease can be connected to (e.g., fused to) a cytidine deaminase functional domain, and may operate by generating targeted C-to-A substitutions. Exemplary nuclease/deaminase fusions are described in Komor et al. Nature 533,420-424 (19 May 2016) (“Komor”). Alternatively, a genome editing system may utilize a cleavage-inactivated (i.e., a “dead”) nuclease, such as a dead Cas9 (dCas9), and may operate by forming stable complexes on one or more targeted regions of cellular DNA, thereby interfering with functions involving the targeted region(s) including, without limitation, mRNA transcription, chromatin remodeling, etc.

Guide RNA (gRNA) Molecules

Guide RNAs (gRNAs) of the present disclosure may be unimolecular (comprising a single RNA molecule, and referred to alternatively as chimeric), or modular (comprising more than one, and typically two, separate RNA molecules, such as a crRNA and a tracrRNA, which are usually associated with one another, for instance by duplexing). gRNAs and their component parts are described throughout the literature, for instance in Briner et al. (Molecular Cell 56(2), 333-339, Oct. 23, 2014 (“Briner”)), and in Cotta-Ramusino.

In bacteria and archaea, type II CRISPR systems generally comprise an RNA-guided nuclease protein such as Cas9, a CRISPR RNA (crRNA) that includes a 5′ region that is complementary to a foreign sequence, and a trans-activating crRNA (tracrRNA) that includes a 5′ region that is complementary to, and forms a duplex with, a 3′ region of the crRNA. While not intending to be bound by any theory, it is thought that this duplex facilitates the formation of—and is necessary for the activity of—the Cas9/gRNA complex. As type II CRISPR systems were adapted for use in gene editing, it was discovered that the crRNA and tracrRNA could be joined into a single unimolecular or chimeric guide RNA, in one non-limiting example, by means of a four nucleotide (e.g., GAAA) “tetraloop” or “linker” sequence bridging complementary regions of the crRNA (at its 3′ end) and the tracrRNA (at its 5′ end). (Mali et al. Science. 2013 Feb. 15; 339(6121): 823-826 (“Mali”); Jiang et al. Nat Biotechnol. 2013 March; 31(3): 233-239 (“Jiang”); and Jinek et al., 2012 Science August 17; 337(6096): 816-821 (“Jinek 2012”)).

Guide RNAs, whether unimolecular or modular, include a “targeting domain” that is fully or partially complementary to a target domain within a target sequence, such as a DNA sequence in the genome of a cell where editing is desired. Targeting domains are referred to by various names in the literature, including without limitation “guide sequences” (Hsu et al., Nat Biotechnol. 2013 September; 31(9): 827-832, (“Hsu”)), “complementarity regions” (Cotta-Ramusino), “spacers” (Briner) and generically as “crRNAs” (Jiang). Irrespective of the names they are given, targeting domains are typically 10-30 nucleotides in length, and in certain embodiments are 16-24 nucleotides in length (for instance, 16, 17, 18, 19, 20, 21, 22, 23 or 24 nucleotides in length), and are at or near the 5′ terminus of in the case of a Cas9 gRNA, and at or near the 3′ terminus in the case of a Cpf1 gRNA.

In addition to the targeting domains, gRNAs typically (but not necessarily, as discussed below) include a plurality of domains that may influence the formation or activity of gRNA/Cas9 complexes. For instance, as mentioned above, the duplexed structure formed by first and secondary complementarity domains of a gRNA (also referred to as a repeat:anti-repeat duplex) interacts with the recognition (REC) lobe of Cas9 and can mediate the formation of Cas9/gRNA complexes. (Nishimasu et al., Cell 156, 935-949, Feb. 27, 2014 (“Nishimasu 2014”) and Nishimasu et al., Cell 162, 1113-1126, Aug. 27, 2015 (“Nishimasu 2015”)). It should be noted that the first and/or second complementarity domains may contain one or more poly-A tracts, which can be recognized by RNA polymerases as a termination signal. The sequence of the first and second complementarity domains are, therefore, optionally modified to eliminate these tracts and promote the complete in vitro transcription of gRNAs, for instance through the use of A-G swaps as described in Briner, or A-U swaps. These and other similar modifications to the first and second complementarity domains are within the scope of the present disclosure.

Along with the first and second complementarity domains, Cas9 gRNAs typically include two or more additional duplexed regions that are involved in nuclease activity in vivo but not necessarily in vitro. (Nishimasu 2015). A first stem-loop one near the 3′ portion of the second complementarity domain is referred to variously as the “proximal domain,” (Cotta-Ramusino) “stem loop 1” (Nishimasu 2014 and 2015) and the “nexus” (Briner). One or more additional stem loop structures are generally present near the 3′ end of the gRNA, with the number varying by species: s. pyogenes gRNAs typically include two 3′ stem loops (for a total of four stem loop structures including the repeat:anti-repeat duplex), while S. aureus and other species have only one (for a total of three stem loop structures). A description of conserved stem loop structures (and gRNA structures more generally) organized by species is provided in Briner.

While the foregoing description has focused on gRNAs for use with Cas9, it should be appreciated that other RNA-guided nucleases have been (or may in the future be) discovered or invented which utilize gRNAs that differ in some ways from those described to this point. For instance, Cpf1 (“CRISPR from Prevotella and Franciscella 1”) is a recently discovered RNA-guided nuclease that does not require a tracrRNA to function. (Zetsche et al., 2015, Cell 163, 759-771 Oct. 22, 2015 (“Zetsche I”)). A gRNA for use in a Cpf1 genome editing system generally includes a targeting domain and a complementarily domain (alternately referred to as a “handle”). It should also be noted that, in gRNAs for use with Cpf1, the targeting domain is usually present at or near the 3′ end, rather than the 5′ end as described above in connection with Cas9 gRNAs (the handle is at or near the 5′ end of a Cpf1 gRNA).

Those of skill in the art will appreciate, however, that although structural differences may exist between gRNAs from different prokaryotic species, or between Cpf1 and Cas9 gRNAs, the principles by which gRNAs operate are generally consistent. Because of this consistency of operation, gRNAs can be defined, in broad terms, by their targeting domain sequences, and skilled artisans will appreciate that a given targeting domain sequence can be incorporated in any suitable gRNA, including a unimolecular or chimeric gRNA, or a gRNA that includes one or more chemical modifications and/or sequential modifications (substitutions, additional nucleotides, truncations, etc.). Thus, for economy of presentation in this disclosure, gRNAs may be described solely in terms of their targeting domain sequences.

More generally, skilled artisans will appreciate that some aspects of the present disclosure relate to systems, methods and compositions that can be implemented using multiple RNA-guided nucleases. For this reason, unless otherwise specified, the term gRNA should be understood to encompass any suitable gRNA that can be used with any RNA-guided nuclease, and not only those gRNAs that are compatible with a particular species of Cas9 or Cpf1. By way of illustration, the term gRNA can, in certain embodiments, include a gRNA for use with any RNA-guided nuclease occurring in a Class 2 CRISPR system, such as a type II or type V or CRISPR system, or an RNA-guided nuclease derived or adapted therefrom.

gRNA Design

Methods for selection and validation of target sequences as well as off-target analyses have been described previously, e.g., in Mali; Hsu; Fu et al., 2014 Nat Biotechnol 32(3): 279-84, Heigwer et al., 2014 Nat methods 11(2):122-3; Bae et al. (2014) Bioinformatics 30(10): 1473-5; and Xiao A et al. (2014) Bioinformatics 30(8): 1180-1182. As a non-limiting example, gRNA design may involve the use of a software tool to optimize the choice of potential target sequences corresponding to a user's target sequence, e.g., to minimize total off-target activity across the genome. While off-target activity is not limited to cleavage, the cleavage efficiency at each off-target sequence can be predicted, e.g., using an experimentally-derived weighting scheme. These and other guide selection methods are described in detail in Maeder and Cotta-Ramusino.

For example, methods for selection and validation of target sequences as well as off-target analyses can be performed using cas-offinder (Bae S, Park J, Kim J-S. Cas-OFFinder: a fast and versatile algorithm that searches for potential off-target sites of Cas9 RNA-guided endonucleases. Bioinformatics. 2014; 30:1473-5). Cas-offinder is a tool that can quickly identify all sequences in a genome that have up to a specified number of mismatches to a guide sequence.

As another example, methods for scoring how likely a given sequence is to be an off-target (e.g., once candidate target sequences are identified) can be performed. An exemplary score includes a Cutting Frequency Determination (CFD) score, as described by Doench J G, Fusi N, Sullender M, Hegde M, Vaimberg E W, Donovan K F, et al. Optimized sgRNA design to maximize activity and minimize off-target effects of CRISPR-Cas9. Nat Biotechnol. 2016; 34:184-91.

gRNA Modifications

In certain embodiments, gRNAs as used herein may be modified or unmodified gRNAs. In certain embodiments, a gRNA may include one or more modifications. In certain embodiments, the one or more modifications may include a phosphorothioate linkage modification, a phosphorodithioate (PS2) linkage modification, a 2′-O-methyl modification, or combinations thereof. In certain embodiments, the one or more modifications may be at the 5′ end of the gRNA, at the 3′ end of the gRNA, or combinations thereof.

In certain embodiments, a gRNA modification may comprise one or more phosphorodithioate (PS2) linkage modifications.

In some embodiments, a gRNA used herein includes one or more or a stretch of deoxyribonucleic acid (DNA) bases, also referred to herein as a “DNA extension.” In some embodiments, a gRNA used herein includes a DNA extension at the 5′ end of the gRNA, the 3′ end of the gRNA, or a combination thereof. In certain embodiments, the DNA extension may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 DNA bases long. For example, in certain embodiments, the DNA extension may be 1, 2, 3, 4, 5, 10, 15, 20, or 25 DNA bases long. In certain embodiments, the DNA extension may include one or more DNA bases selected from adenine (A), guanine (G), cytosine (C), or thymine (T). In certain embodiments, the DNA extension includes the same DNA bases. For example, the DNA extension may include a stretch of adenine (A) bases. In certain embodiments, the DNA extension may include a stretch of thymine (T) bases. In certain embodiments, the DNA extension includes a combination of different DNA bases. In certain embodiments, a DNA extension may comprise a sequence set forth in Table 3.

Exemplary suitable 5′ extensions for Cpf1 guide RNAs are provided in Table 3 below:

TABLE 3
Exemplary Cpf1 gRNA 5′ Extensions

| SEQ ID NO: | 5′ extension sequence | 5′ modification |
|---|---|---|
| 1 | rCrUrUrUrU | +5 RNA |
| 2 | rArArGrArCrCrUrUrUrU | +10 RNA |
| 3 | rArUrGrUrGrUrUrUrUrUrGrUrCrArArArArGrArCrCrUrUrUrU | +25 RNA |
| 4 | rArGrGrCrCrArGrCrUrUrGrCrCrGrGrUrUrUrUrUrUrArGrUrCrG | +60 RNA |
| | rUrGrCrUrGrCrUrUrCrArUrGrUrGrUrUrUrUrUrGrUrCrArArArA | |
| | rGrArCrCrUrUrUrU | |
| 5 | CTTTT | +5 DNA |
| 6 | AAGACCTTTT | +10 DNA |
| 7 | ATGTGTTTTTGTCAAAAGACCTTTT | +25 DNA |
| 8 | AGGCCAGCTTGCCGGTTTTTTAGTCGTGCTGCTTCATGTGTTTTTGTCAAAA | +60 DNA |
| | GACCTTTT | |
| 9 | TTTTTGTCAAAAGACCTTTT | +20 DNA |
| 10 | GCTTCATGTGTTTTTGTCAAAAGACCTTTT | +30 DNA |
| 11 | GCCGGTTTTTTAGTCGTGCTGCTTCATGTGTTTTTGTCAAAAGACCTTTT | +50 DNA |
| 12 | TAGTCGTGCTGCTTCATGTGTTTTTGTCAAAAGACCTTTT | +40 DNA |
| 13 | C*C*GAAGTTTTCTTCGGTTTT | +20 DNA + 2xPS |
| 14 | T*T*TTTCCGAAGTTTTCTTCGGTTTT | +25 DNA + 2xPS |
| 15 | A*A*CGCTTTTTCCGAAGTTTTCTTCGGTTTT | +30 DNA + 2xPS |
| 16 | G*C*GTTGTTTTCAACGCTTTTTCCGAAGTTTTCTTCGGTTTT | +41 DNA + 2xPS |
| 17 | G*G*CTTCTTTTGAAGCCTTTTTGCGTTGTTTTCAACGCTTTTTCCGAAGTT | +62 DNA + 2xPS |
| | TTCTTCGGTTTT | |
| 18 | A*T*GTGTTTTTGTCAAAAGACCTTTT | +25 DNA + 2xPS |
| 19 | AAAAAAAAAAAAAAAAAAAAAAAAA | +25 A |
| 20 | TTTTTTTTTTTTTTTTTTTTTTTTT | +25 T |
| 21 | mA*mU*rGrUrGrUrUrUrUrUrGrUrCrArArArArGrArCrCrUrUrUrU | +25 RNA + 2xPS |
| 22 | mA*mA*rArArArArArArArArArArArArArArArArArArArArArArA | Poly A RNA + 2xPS |
| 23 | mU*mU*rUrUrUrUrUrUrUrUrUrUrUrUrUrUrUrUrUrUrUrUrUrUrU | PolyU RNA + 2xPS |

All bases are in upper case
Lowercase “r” represents RNA, 2′-hydroxy; bases not modified by an “r” are DNA All bases are
linked via standard phosphodiester bonds except as noted:
“*” represents phosphorothioate modification
“PS” represents phosphorothioate modification

In certain embodiments, a gRNA used herein includes a DNA extension as well as a chemical modification, e.g., one or more phosphorothioate linkage modifications, one or more phosphorodithioate (PS2) linkage modifications, one or more 2′-O-methyl modifications, or one or more additional suitable chemical gRNA modification disclosed herein, or combinations thereof. In certain embodiments, the one or more modifications may be at the 5′ end of the gRNA, at the 3′ end of the gRNA, or combinations thereof.

Without wishing to be bound by theory, it is contemplated that any DNA extension may be used with any gRNA disclosed herein, so long as it does not hybridize to the target nucleic acid being targeted by the gRNA and it also exhibits an increase in editing at the target nucleic acid site relative to a gRNA which does not include such a DNA extension.

In some embodiments, a gRNA used herein includes one or more or a stretch of ribonucleic acid (RNA) bases, also referred to herein as an “RNA extension.” In some embodiments, a gRNA used herein includes an RNA extension at the 5′ end of the gRNA, the 3′ end of the gRNA, or a combination thereof. In certain embodiments, the RNA extension may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 RNA bases long. For example, in certain embodiments, the RNA extension may be 1, 2, 3, 4, 5, 10, 15, 20, or 25 RNA bases long. In certain embodiments, the RNA extension may include one or more RNA bases selected from adenine (rA), guanine (rG), cytosine (rC), or uracil (rU), in which the “r” represents RNA, 2′-hydroxy. In certain embodiments, the RNA extension includes the same RNA bases. For example, the RNA extension may include a stretch of adenine (rA) bases. In certain embodiments, the RNA extension includes a combination of different RNA bases, In certain embodiments, a gRNA used herein includes an RNA extension as well as one or more phosphorothioate linkage modifications, one or more phosphorodithioate (PS2) linkage modifications, one or more 2′-O-methyl modifications, one or more additional suitable gRNA modification, e.g., chemical modification, disclosed herein, or combinations thereof. In certain embodiments, the one or more modifications may be at the 5′ end of the gRNA, at the 3′ end of the gRNA, or combinations thereof. In certain embodiments, a gRNA including a RNA extension may comprise a sequence set forth herein.

It is contemplated that gRNAs used herein may also include an RNA extension and a DNA extension. In certain embodiments, the RNA extension and DNA extension may both be at the 5′ end of the gRNA, the 3′ end of the gRNA, or a combination thereof. In certain embodiments, the RNA extension is at the 5′ end of the gRNA and the DNA extension is at the 3′ end of the gRNA. In certain embodiments, the RNA extension is at the 3′ end of the gRNA and the DNA extension is at the 5′ end of the gRNA.

In some embodiments, a gRNA which includes a modification, e.g., a DNA extension at the 5′ end and/or a chemical modification as disclosed herein, is complexed with a RNA-guided nuclease, e.g., an AsCpf1 nuclease, to form an RNP, which is then employed to edit a target cell, e.g., a pluripotent stem cell or a daughter cell thereof.

Additional suitable gRNA modifications will be apparent to those of ordinary skill in the art based on the present disclosure. Suitable gRNA modifications include, for example, those described in PCT application PCT/US2018/054027, filed on Oct. 2, 2018, and entitled “MODIFIED CPF1 GUIDE RNA;” in PCT application PCT/US2015/000143, filed on Dec. 3, 2015, and entitled “GUIDE RNA WITH CHEMICAL MODIFICATIONS;” in PCT application PCT/US2016/026028, filed Apr. 5, 2016, and entitled “CHEMICALLY MODIFIED GUIDE RNAS FOR CRISPR/CAS-MEDIATED GENE REGULATION;” and in PCT application PCT/US2016/053344, filed on Sep. 23, 2016, and entitled “NUCLEASE-MEDIATED GENOME EDITING OF PRIMARY CELLS AND ENRICHMENT THEREOF;” the entire contents of each of which are incorporated herein by reference.

Certain exemplary modifications discussed in this section can be included at any position within a gRNA sequence including, without limitation at or near the 5′ end (e.g., within 1-10, 1-5, or 1-2 nucleotides of the 5′ end) and/or at or near the 3′ end (e.g., within 1-10, 1-5, or 1-2 nucleotides of the 3′ end). In some cases, modifications are positioned within functional motifs, such as the repeat-anti-repeat duplex of a Cas9 gRNA, a stem loop structure of a Cas9 or Cpf1 gRNA, and/or a targeting domain of a gRNA.

As one example, the 5′ end of a gRNA can include a eukaryotic mRNA cap structure or cap analog (e.g., a G(5′)ppp(5′)G cap analog, a m7G(5′)ppp(5′)G cap analog, or a 3′-O-Me-m7G(5′)ppp(5′)G anti reverse cap analog (ARCA)), as shown below:

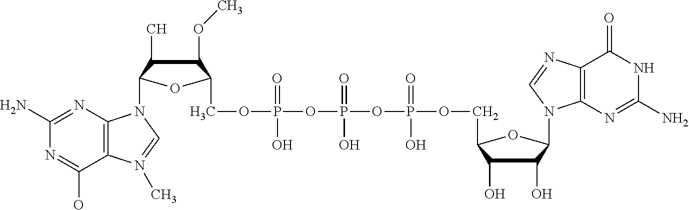

The cap or cap analog can be included during either chemical or enzymatic synthesis of the gRNA.

Along similar lines, the 5′ end of the gRNA can lack a 5′ triphosphate group. For instance, in vitro transcribed gRNAs can be phosphatase-treated (e.g., using calf intestinal alkaline phosphatase) to remove a 5′ triphosphate group.

Another common modification involves the addition, at the 3′ end of a gRNA, of a plurality (e.g., 1-10, 10-20, or 25-200) of adenine (A) residues referred to as a polyA tract. The polyA tract can be added to a gRNA during chemical or enzymatic synthesis, using a polyadenosine polymerase (e.g., E. coli Poly(A)Polymerase).

Guide RNAs can be modified at a 3′ terminal U ribose. For example, the two terminal hydroxyl groups of the U ribose can be oxidized to aldehyde groups and a concomitant opening of the ribose ring to afford a modified nucleoside as shown below:

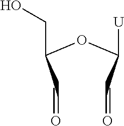

wherein “U” can be an unmodified or modified uridine.

The 3′ terminal U ribose can be modified with a 2′3′ cyclic phosphate as shown below:

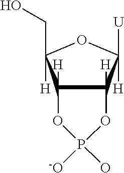

wherein “U” can be an unmodified or modified uridine.

Guide RNAs can contain 3′ nucleotides that can be stabilized against degradation, e.g., by incorporating one or more of the modified nucleotides described herein. In certain embodiments, uridines can be replaced with modified uridines, e.g., 5-(2-amino)propyl uridine, and 5-bromo uridine, or with any of the modified uridines described herein; adenosines and guanosines can be replaced with modified adenosines and guanosines, e.g., with modifications at the 8-position, e.g., 8-bromo guanosine, or with any of the modified adenosines or guanosines described herein.

In certain embodiments, sugar-modified ribonucleotides can be incorporated into a gRNA, e.g., wherein the 2′ OH-group is replaced by a group selected from H, —OR, —R (wherein R can be, e.g., alkyl, cycloalkyl, aryl, aralkyl, heteroaryl or sugar), halo, —SH, —SR (wherein R can be, e.g., alkyl, cycloalkyl, aryl, aralkyl, heteroaryl or sugar), amino (wherein amino can be, e.g., NH₂, alkylamino, dialkylamino, heterocyclyl, arylamino, diarylamino, heteroarylamino, diheteroarylamino, or amino acid); or cyano (—CN). In certain embodiments, the phosphate backbone can be modified as described herein, e.g., with a phosphothioate (PhTx) group. In certain embodiments, one or more of the nucleotides of the gRNA can each independently be a modified or unmodified nucleotide including, but not limited to 2′-sugar modified, such as, 2′-O-methyl, 2′-O-methoxyethyl, or 2′-Fluoro modified including, e.g., 2′-F or 2′-O-methyl, adenosine (A), 2′-F or 2′-O-methyl, cytidine (C), 2′-F or 2′-O-methyl, uridine (U), 2′-F or 2′-O-methyl, thymidine (T), 2′-F or 2′-O-methyl, guanosine (G), 2′-O-methoxyethyl-5-methyluridine (Teo), 2′-O-methoxyethyladenosine (Aeo), 2′-O-methoxyethyl-5-methylcytidine (m5Ceo), and any combinations thereof.

Guide RNAs can also include “locked” nucleic acids (LNA) in which the 2′ OH-group can be connected, e.g., by a C1-6 alkylene or C1-6 heteroalkylene bridge, to the 4′ carbon of the same ribose sugar. Any suitable moiety can be used to provide such bridges, including without limitation methylene, propylene, ether, or amino bridges; O-amino (wherein amino can be, e.g., NH₂, alkylamino, dialkylamino, heterocyclyl, arylamino, diarylamino, heteroarylamino, or diheteroarylamino, ethylenediamine, or polyamino) and aminoalkoxy or O(CH₂)ₙ-amino (wherein amino can be, e.g., NH₂, alkylamino, dialkylamino, heterocyclyl, arylamino, diarylamino, heteroarylamino, or diheteroarylamino, ethylenediamine, or polyamino).

In certain embodiments, a gRNA can include a modified nucleotide which is multicyclic (e.g., tricyclo; and “unlocked” forms, such as glycol nucleic acid (GNA) (e.g., R-GNA or S-GNA, where ribose is replaced by glycol units attached to phosphodiester bonds), or threose nucleic acid (TNA, where ribose is replaced with α-L-threofuranosyl-(3′→2′)).

Generally, gRNAs include the sugar group ribose, which is a 5-membered ring having an oxygen. Exemplary modified gRNAs can include, without limitation, replacement of the oxygen in ribose (e.g., with sulfur (S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). Although the majority of sugar analog alterations are localized to the 2′ position, other sites are amenable to modification, including the 4′ position. In certain embodiments, a gRNA comprises a 4′-S, 4′-Se or a 4′-C-aminomethyl-2′-O-Me modification.

In certain embodiments, deaza nucleotides, e.g., 7-deaza-adenosine, can be incorporated into a gRNA. In certain embodiments, 0- and N-alkylated nucleotides, e.g., N6-methyl adenosine, can be incorporated into a gRNA. In certain embodiments, one or more or all of the nucleotides in a gRNA are deoxynucleotides.

Guide RNAs can also include one or more cross-links between complementary regions of the crRNA (at its 3′ end) and the tracrRNA (at its 5′ end) (e.g., within a “tetraloop” structure and/or positioned in any stem loop structure occurring within a gRNA). A variety of linkers are suitable for use. For example, guide RNAs can include common linking moieties including, without limitation, polyvinylether, polyethylene, polypropylene, polyethylene glycol (PEG), polyvinyl alcohol (PVA), polyglycolide (PGA), polylactide (PLA), polycaprolactone (PCL), and copolymers thereof.

In some embodiments, a bifunctional cross-linker is used to link a 5′ end of a first gRNA fragment and a 3′ end of a second gRNA fragment, and the 3′ or 5′ ends of the gRNA fragments to be linked are modified with functional groups that react with the reactive groups of the cross-linker. In general, these modifications comprise one or more of amine, sulfhydryl, carboxyl, hydroxyl, alkene (e.g., a terminal alkene), azide and/or another suitable functional group. Multifunctional (e.g. bifunctional) cross-linkers are also generally known in the art, and may be either heterofunctional or homofunctional, and may include any suitable functional group, including without limitation isothiocyanate, isocyanate, acyl azide, an NHS ester, sulfonyl chloride, tosyl ester, tresyl ester, aldehyde, amine, epoxide, carbonate (e.g., Bis(p-nitrophenyl) carbonate), aryl halide, alkyl halide, imido ester, carboxylate, alkyl phosphate, anhydride, fluorophenyl ester, HOBt ester, hydroxymethyl phosphine, O-methylisourea, DSC, NHS carbamate, glutaraldehyde, activated double bond, cyclic hemiacetal, NHS carbonate, imidazole carbamate, acyl imidazole, methylpyridinium ether, azlactone, cyanate ester, cyclic imidocarbonate, chlorotriazine, dehydroazepine, 6-sulfo-cytosine derivatives, maleimide, aziridine, TNB thiol, Ellman's reagent, peroxide, vinylsulfone, phenylthioester, diazoalkanes, diazoacetyl, epoxide, diazonium, benzophenone, anthraquinone, diazo derivatives, diazirine derivatives, psoralen derivatives, alkene, phenyl boronic acid, etc. In some embodiments, a first gRNA fragment comprises a first reactive group and the second gRNA fragment comprises a second reactive group. For example, the first and second reactive groups can each comprise an amine moiety, which are crosslinked with a carbonate-containing bifunctional crosslinking reagent to form a urea linkage. In other instances, (a) the first reactive group comprises a bromoacetyl moiety and the second reactive group comprises a sulfhydryl moiety, or (b) the first reactive group comprises a sulfhydryl moiety and the second reactive group comprises a bromoacetyl moiety, which are crosslinked by reacting the bromoacetyl moiety with the sulfhydryl moiety to form a bromoacetyl-thiol linkage. These and other cross-linking chemistries are known in the art, and are summarized in the literature, including by Greg T. Hermanson, Bioconjugate Techniques, 3rd Ed. 2013, published by Academic Press.

Additional suitable gRNA modifications will be apparent to those of ordinary skill in the art based on the present disclosure. Suitable gRNA modifications include, for example, those described in PCT application PCT/US2018/054027, filed on Oct. 2, 2018, and entitled “MODIFIED CPF1 GUIDE RNA;” in PCT application PCT/US2015/000143, filed on Dec. 3, 2015, and entitled “GUIDE RNA WITH CHEMICAL MODIFICATIONS;” in PCT application PCT/US2016/026028, filed Apr. 5, 2016, and entitled “CHEMICALLY MODIFIED GUIDE RNAS FOR CRISPR/CAS-MEDIATED GENE REGULATION;” and in PCT application PCT/US2016/053344, filed on Sep. 23, 2016, and entitled “NUCLEASE-MEDIATED GENOME EDITING OF PRIMARY CELLS AND ENRICHMENT THEREOF;” the entire contents of each of which are incorporated herein by reference.

Exemplary gRNAs

Non-limiting examples of guide RNAs suitable for certain embodiments embraced by the present disclosure are provided herein, for example, in the Tables below. Those of ordinary skill in the art will be able to envision suitable guide RNA sequences for a specific nuclease, e.g., a Cas9 or Cpf-1 nuclease, from the disclosure of the targeting domain sequence, either as a DNA or RNA sequence. For example, a guide RNA comprising a targeting sequence consisting of RNA nucleotides would include the RNA sequence corresponding to the targeting domain sequence provided as a DNA sequence, and this contain uracil instead of thymidine nucleotides. For example, a guide RNA comprising a targeting domain sequence consisting of RNA nucleotides, and described by the DNA sequence TCTGCAGAAATGTTCCCCGT (SEQ ID NO: 24) would have a targeting domain of the corresponding RNA sequence UCUGCAGAAAUGUUCCCCGU (SEQ ID NO: 25). As will be apparent to the skilled artisan, such a targeting sequence would be linked to a suitable guide RNA scaffold, e.g., a crRNA scaffold sequence or a chimeric crRNA/tracrRNA scaffold sequence. Suitable gRNA scaffold sequences are known to those of ordinary skill in the art. For AsCpf1, for example, a suitable scaffold sequence comprises the sequence UAAUUUCUACUCUUGUAGAU (SEQ ID NO: 26) added to the 5′-terminus of the targeting domain. In the example above, this would result in a Cpf1 guide RNA of the sequence UAAUUUCUACUCUUGUAGAUUCUGCAGAAAUGUUCCCCGU (SEQ ID NO: 27). Those of skill in the art would further understand how to modify such a guide RNA, e.g., by adding a DNA extension (e.g., in the example above, adding a 25-mer DNA extension as described herein would result, for example, in a guide RNA of the sequence ATGTGTTTTTGTCAAAAGACCTTTTrUrArArUrUrUrCrUrArCrUrCrUrUrGrUrArGrArU rUrCrUrGrCrArGrArArArUrGrUrUrCrCrCrCrGrU) (SEQ ID NO: 28). It will be understood that the exemplary targeting sequences provided herein are not limiting, and additional suitable sequences, e.g., variants of the specific sequences disclosed herein, will be apparent to the skilled artisan based on the present disclosure in view of the general knowledge in the art.

In some embodiments the gRNA for use in the disclosure is a gRNA targeting TGFβRII (TGFβRII gRNA). In some embodiments, the gRNA targeting TGFβRII is one or more of the gRNAs described in Table 4.

TABLE 4
Exemplary TGFβRII gRNAs

| | gRNA Targeting Domain Sequence | | | |
|---|---|---|---|---|
| Name | Sequence (DNA) | Length | Enzyme | SEQ ID NO: |
| TGFBR24326 | CAGGACGATGTGCAGCGGCC | 20 | AsCpf1 RR | 29 |
| TGFBR24327 | ACCGCACGTTCAGAAGTCGG | 20 | AsCpf1 RR | 30 |
| TGFBR24328 | ACAACTGTGTAAATTTTGTG | 20 | AsCpf1 RR | 31 |
| TGFBR24329 | CAACTGTGTAAATTTTGTGA | 20 | AsCpf1 RR | 32 |
| TGFBR24330 | ACCTGTGACAACCAGAAATC | 20 | AsCpf1 RR | 33 |
| TGFBR24331 | CCTGTGACAACCAGAAATCC | 20 | AsCpf1 RR | 34 |
| TGFBR24332 | TGTGGCTTCTCACAGATGGA | 20 | AsCpf1 RR | 35 |
| TGFBR24333 | TCTGTGAGAAGCCACAGGAA | 20 | AsCpf1 RR | 36 |
| TGFBR24334 | AAGCTCCCCTACCATGACTT | 20 | AsCpf1 RR | 37 |
| TGFBR24335 | GAATAAAGTCATGGTAGGGG | 20 | AsCpf1 RR | 38 |
| TGFBR24336 | AGAATAAAGTCATGGTAGGG | 20 | AsCpf1 RR | 39 |
| TGFBR24337 | CTACCATGACTTTATTCTGG | 20 | AsCpf1 RR | 40 |
| TGFBR24338 | TACCATGACTTTATTCTGGA | 20 | AsCpf1 RR | 41 |
| TGFBR24339 | TAATGCACTTTGGAGAAGCA | 20 | AsCpf1 RR | 42 |
| TGFBR24340 | TTCATAATGCACTTTGGAGA | 20 | AsCpf1 RR | 43 |
| TGFBR24341 | AAGTGCATTATGAAGGAAAA | 20 | AsCpf1 RR | 44 |
| TGFBR24342 | TGTGTTCCTGTAGCTCTGAT | 20 | AsCpf1 RR | 45 |
| TGFBR24343 | TGTAGCTCTGATGAGTGCAA | 20 | AsCpf1 RR | 46 |
| TGFBR24344 | AGTGACAGGCATCAGCCTCC | 20 | AsCpf1 RR | 47 |
| TGFBR24345 | AGTGGTGGCAGGAGGCTGAT | 20 | AsCpf1 RR | 48 |
| TGFBR24346 | AGGTTGAACTCAGCTTCTGC | 20 | AsCpf1 RR | 49 |
| TGFBR24347 | CAGGTTGAACTCAGCTTCTG | 20 | AsCpf1 RR | 50 |
| TGFBR24348 | ACCTGGGAAACCGGCAAGAC | 20 | AsCpf1 RR | 51 |
| TGFBR24349 | CGTCTTGCCGGTTTCCCAGG | 20 | AsCpf1 RR | 52 |
| TGFBR24350 | GCGTCTTGCCGGTTTCCCAG | 20 | AsCpf1 RR | 53 |
| TGFBR24351 | TGAGCTTCCGCGTCTTGCCG | 20 | AsCpf1 RR | 54 |
| TGFBR24352 | GCGAGCACTGTGCCATCATC | 20 | AsCpf1 RR | 55 |
| TGFBR24353 | GGATGATGGCACAGTGCTCG | 20 | AsCpf1 RR | 56 |
| TGFBR24354 | AGGATGATGGCACAGTGCTC | 20 | AsCpf1 RR | 57 |
| TGFBR24355 | CGTGTGCCAACAACATCAAC | 20 | AsCpf1 RR | 58 |
| TGFBR24356 | GCTCAATGGGCAGCAGCTCT | 20 | AsCpf1 RR | 59 |
| TGFBR24357 | ACCAGGGTGTCCAGCTCAAT | 20 | AsCpf1 RR | 60 |
| TGFBR24358 | CACCAGGGTGTCCAGCTCAA | 20 | AsCpf1 RR | 61 |
| TGFBR24359 | CCACCAGGGTGTCCAGCTCA | 20 | AsCpf1 RR | 62 |
| TGFBR24360 | GCTTGGCCTTATAGACCTCA | 20 | AsCpf1 RR | 63 |
| TGFBR24361 | GAGCAGTTTGAGACAGTGGC | 20 | AsCpf1 RR | 64 |
| TGFBR24362 | AGAGGCATACTCCTCATAGG | 20 | AsCpf1 RR | 65 |
| TGFBR24363 | CTATGAGGAGTATGCCTCTT | 20 | AsCpf1 RR | 66 |
| TGFBR24364 | AAGAGGCATACTCCTCATAG | 20 | AsCpf1 RR | 67 |
| TGFBR24365 | TATGAGGAGTATGCCTCTTG | 20 | AsCpf1 RR | 68 |
| TGFBR24366 | GATTGATGTCTGAGAAGATG | 20 | AsCpf1 RR | 69 |
| TGFBR24367 | CTCCTCAGCCGTCAGGAACT | 20 | AsCpf1 RR | 70 |
| TGFBR24368 | GTTCCTGACGGCTGAGGAGC | 20 | AsCpf1 RR | 71 |
| TGFBR24369 | GCTCCTCAGCCGTCAGGAAC | 20 | AsCpf1 RR | 72 |
| TGFBR24370 | TGACGGCTGAGGAGCGGAAG | 20 | AsCpf1 RR | 73 |
| TGFBR24371 | TCTTCCGCTCCTCAGCCGTC | 20 | AsCpf1 RR | 74 |
| TGFBR24372 | AACTCCGTCTTCCGCTCCTC | 20 | AsCpf1 RR | 75 |
| TGFBR24373 | CAACTCCGTCTTCCGCTCCT | 20 | AsCpf1 RR | 76 |
| TGFBR24374 | CCAACTCCGTCTTCCGCTCC | 20 | AsCpf1 RR | 77 |
| TGFBR24375 | ACGCCAAGGGCAACCTACAG | 20 | AsCpf1 RR | 78 |
| TGFBR24376 | CGCCAAGGGCAACCTACAGG | 20 | AsCpf1 RR | 79 |
| TGFBR24377 | AGCTGATGACATGCCGCGTC | 20 | AsCpf1 RR | 80 |
| TGFBR24378 | GGGCGAGGGAGCTGCCCAGC | 20 | AsCpf1 RR | 81 |
| TGFBR24379 | CGGGCGAGGGAGCTGCCCAG | 20 | AsCpf1 RR | 82 |
| TGFBR24380 | CCGGGCGAGGGAGCTGCCCA | 20 | AsCpf1 RR | 83 |
| TGFBR24381 | TCGCCCGGGGGATTGCTCAC | 20 | AsCpf1 RR | 84 |
| TGFBR24382 | ACATGGAGTGTGATCACTGT | 20 | AsCpf1 RR | 85 |
| TGFBR24383 | CAGTGATCACACTCCATGTG | 20 | AsCpf1 RR | 86 |
| TGFBR24384 | TGTGGGAGGCCCAAGATGCC | 20 | AsCpf1 RR | 87 |
| TGFBR24385 | TGTGCACGATGGGCATCTTG | 20 | AsCpf1 RR | 88 |
| TGFBR24386 | CGAGGATATTGGAGCTCTTG | 20 | AsCpf1 RR | 89 |
| TGFBR24387 | ATATCCTCGTGAAGAACGAC | 20 | AsCpf1 RR | 90 |
| TGFBR24388 | GACGCAGGGAAAGCCCAAAG | 20 | AsCpf1 RR | 91 |
| TGFBR24389 | CTGCGTCTGGACCCTACTCT | 20 | AsCpf1 RR | 92 |
| TGFBR24390 | TGCGTCTGGACCCTACTCTG | 20 | AsCpf1 RR | 93 |
| TGFBR24391 | CAGACAGAGTAGGGTCCAGA | 20 | AsCpf1 RR | 94 |
| TGFBR24392 | GCCAGCACGATCCCACCGCA | 20 | AsCpf1 RVR | 95 |
| TGFBR24393 | AAGGAAAAAAAAAAGCCTGG | 20 | AsCpf1 RVR | 96 |
| TGFBR24394 | ACACCAGCAATCCTGACTTG | 20 | AsCpf1 RVR | 97 |
| TGFBR24395 | ACTAGCAACAAGTCAGGATT | 20 | AsCpf1 RVR | 98 |
| TGFBR24396 | GCAACTCCCAGTGGTGGCAG | 20 | AsCpf1 RVR | 99 |
| TGFBR24397 | TGTCATCATCATCTTCTACT | 20 | AsCpf1 RVR | 100 |
| TGFBR24398 | GACCTCAGCAAAGCGACCTT | 20 | AsCpf1 RVR | 101 |
| TGFBR24399 | AGGCCAAGCTGAAGCAGAAC | 20 | AsCpf1 RVR | 102 |
| TGFBR24400 | AGGAGTATGCCTCTTGGAAG | 20 | AsCpf1 RVR | 103 |
| TGFBR24401 | CCTCTTGGAAGACAGAGAAG | 20 | AsCpf1 RVR | 104 |
| TGFBR24402 | TTCTCATGCTTCAGATTGAT | 20 | AsCpf1 RVR | 105 |
| TGFBR24403 | CTCGTGAAGAACGACCTAAC | 20 | AsCpf1 RVR | 106 |
| TGFbR2036 | GGCCGCTGCACATCGTCCTG | 20 | SpyCas9 | 107 |
| TGFbR2037 | GCGGGGTCTGCCATGGGTCG | 20 | SpyCas9 | 108 |
| TGFbR2038 | AGTTGCTCATGCAGGATTTC | 20 | SpyCas9 | 109 |
| TGFbR2039 | CCAGAATAAAGTCATGGTAG | 20 | SpyCas9 | 110 |
| TGFbR2040 | CCCCTACCATGACTTTATTC | 20 | SpyCas9 | 111 |
| TGFbR2041 | AAGTCATGGTAGGGGAGCTT | 20 | SpyCas9 | 112 |
| TGFbR2042 | AGTCATGGTAGGGGAGCTTG | 20 | SpyCas9 | 113 |
| TGFbR2043 | ATTGCACTCATCAGAGCTAC | 20 | SpyCas9 | 114 |
| TGFbR2044 | CCTAGAGTGAAGAGATTCAT | 20 | SpyCas9 | 115 |
| TGFbR2045 | CCAATGAATCTCTTCACTCT | 20 | SpyCas9 | 116 |
| TGFbR2046 | AAAGTCATGGTAGGGGAGCT | 20 | SpyCas9 | 117 |
| TGFbR2047 | GTGAGCAATCCCCCGGGCGA | 20 | SpyCas9 | 118 |
| TGFbR2048 | GTCGTTCTTCACGAGGATAT | 20 | SpyCas9 | 119 |
| TGFbR2049 | GCCGCGTCAGGTACTCCTGT | 20 | SpyCas9 | 120 |
| TGFbR2050 | GACGCGGCATGTCATCAGCT | 20 | SpyCas9 | 121 |
| TGFbR2051 | GCTTCTGCTGCCGGTTAACG | 20 | SpyCas9 | 122 |
| TGFbR2052 | GTGGATGACCTGGCTAACAG | 20 | SpyCas9 | 123 |
| TGFbR2053 | GTGATCACACTCCATGTGGG | 20 | SpyCas9 | 124 |
| TGFbR2054 | GCCCATTGAGCTGGACACCC | 20 | SpyCas9 | 125 |
| TGFbR2055 | GCGGTCATCTTCCAGGATGA | 20 | SpyCas9 | 126 |
| TGFbR2056 | GGGAGCTGCCCAGCTTGCGC | 20 | SpyCas9 | 127 |
| TGFbR2057 | GTTGATGTTGTTGGCACACG | 20 | SpyCas9 | 128 |
| TGFbR2058 | GGCATCTTGGGCCTCCCACA | 20 | SpyCas9 | 129 |
| TGFbR2059 | GCGGCATGTCATCAGCTGGG | 20 | SpyCas9 | 130 |
| TGFbR2060 | GCTCCTCAGCCGTCAGGAAC | 20 | SpyCas9 | 131 |
| TGFbR2061 | GCTGGTGTTATATTCTGATG | 20 | SpyCas9 | 132 |
| TGFbR2062 | CCGACTTCTGAACGTGCGGT | 20 | SpyCas9 | 133 |
| TGFbR2063 | TGCTGGCGATACGCGTCCAC | 20 | SpyCas9 | 134 |
| TGFbR2064 | CCCGACTTCTGAACGTGCGG | 20 | SpyCas9 | 135 |
| TGFbR2065 | CCACCGCACGTTCAGAAGTC | 20 | SpyCas9 | 136 |
| TGFbR2066 | TCACCCGACTTCTGAACGTG | 20 | SpyCas9 | 137 |
| TGFbR2067 | CCCACCGCACGTTCAGAAGT | 20 | SpyCas9 | 138 |
| TGFbR2068 | CGAGCAGCGGGGTCTGCCAT | 20 | SpyCas9 | 139 |
| TGFbR2069 | ACGAGCAGCGGGGTCTGCCA | 20 | SpyCas9 | 140 |
| TGFbR2070 | AGCGGGGTCTGCCATGGGTC | 20 | SpyCas9 | 141 |
| TGFbR2071 | CCTGAGCAGCCCCCGACCCA | 20 | SpyCas9 | 142 |
| TGFbR2072 | CCATGGGTCGGGGGCTGCTC | 20 | SpyCas9 | 143 |
| TGFbR2073 | AACGTGCGGTGGGATCGTGC | 20 | SpyCas9 | 144 |
| TGFbR2074 | GGACGATGTGCAGCGGCCAC | 20 | SpyCas9 | 145 |
| TGFbR2075 | GTCCACAGGACGATGTGCAG | 20 | SpyCas9 | 146 |
| TGFbR2076 | CATGGGTCGGGGGCTGCTCA | 20 | SpyCas9 | 147 |
| TGFbR2077 | CAGCGGGGTCTGCCATGGGT | 20 | SpyCas9 | 148 |
| TGFbR2078 | ATGGGTCGGGGGCTGCTCAG | 20 | SpyCas9 | 149 |
| TGFbR2079 | CGGGGTCTGCCATGGGTCGG | 20 | SpyCas9 | 150 |
| TGFbR2080 | AGGAAGTCTGTGTGGCTGTA | 20 | SpyCas9 | 151 |
| TGFbR2081 | CTCCATCTGTGAGAAGCCAC | 20 | SpyCas9 | 152 |
| TGFbR2082 | ATGATAGTCACTGACAACAA | 20 | SpyCas9 | 153 |
| TGFbR2083 | GATGCTGCAGTTGCTCATGC | 20 | SpyCas9 | 154 |
| TGFbR2084 | ACAGCCACACAGACTTCCTG | 20 | SpyCas9 | 155 |
| TGFbR2085 | GAAGCCACAGGAAGTCTGTG | 20 | SpyCas9 | 156 |
| TGFbR2086 | TTCCTGTGGCTTCTCACAGA | 20 | SpyCas9 | 157 |
| TGFbR2087 | CTGTGGCTTCTCACAGATGG | 20 | SpyCas9 | 158 |
| TGFbR2088 | TCACAAAATTTACACAGTTG | 20 | SpyCas9 | 159 |
| TGFbR2089 | GACAACATCATCTTCTCAGA | 20 | SpyCas9 | 160 |
| TGFbR2090 | TCCAGAATAAAGTCATGGTA | 20 | SpyCas9 | 161 |
| TGFbR2091 | GGTAGGGGAGCTTGGGGTCA | 20 | SpyCas9 | 162 |
| TGFbR2092 | TTCTCCAAAGTGCATTATGA | 20 | SpyCas9 | 163 |
| TGFbR2093 | CATCTTCCAGAATAAAGTCA | 20 | SpyCas9 | 164 |
| TGFbR2094 | CACATGAAGAAAGTCTCACC | 20 | SpyCas9 | 165 |
| TGFbR2095 | TTCCAGAATAAAGTCATGGT | 20 | SpyCas9 | 166 |
| TGFbR2096 | TTTTCCTTCATAATGCACTT | 20 | SpyCas9 | 167 |
| TGFBR24024 | CACAGTTGTGGAAACTTGAC | 20 | AsCpf1 | 168 |
| TGFBR24039 | CCCAACTCCGTCTTCCGCTC | 20 | AsCpf1 | 169 |
| TGFBR24040 | GGCTTTCCCTGCGTCTGGAC | 20 | AsCpf1 | 170 |
| TGFBR24036 | CTGAGGTCTATAAGGCCAAG | 20 | AsCpf1 | 171 |
| TGFBR24026 | TGATGTGAGATTTTCCACCT | 20 | AsCpf1 | 172 |
| TGFBR24038 | CCTATGAGGAGTATGCCTCT | 20 | AsCpf1 | 173 |
| TGFBR24033 | AAGTGACAGGCATCAGCCTC | 20 | AsCpf1 | 174 |
| TGFBR24028 | CCATGACCCCAAGCTCCCCT | 20 | AsCpf1 | 175 |
| TGFBR24031 | CTTCATAATGCACTTTGGAG | 20 | AsCpf1 | 176 |
| TGFBR24032 | TTCATGTGTTCCTGTAGCTC | 20 | AsCpf1 | 177 |
| TGFBR24029 | TTCTGGAAGATGCTGCTTCT | 20 | AsCpf1 | 178 |
| TGFBR24035 | CCCACCAGGGTGTCCAGCTC | 20 | AsCpf1 | 179 |
| TGFBR24037 | AGACAGTGGCAGTCAAGATC | 20 | AsCpf1 | 180 |
| TGFBR24041 | CCTGCGTCTGGACCCTACTC | 20 | AsCpf1 | 181 |
| TGFBR24025 | CACAACTGTGTAAATTTTGT | 20 | AsCpf1 | 182 |
| TGFBR24030 | GAGAAGCAGCATCTTCCAGA | 20 | AsCpf1 | 183 |
| TGFBR24027 | TGGTTGTCACAGGTGGAAAA | 20 | AsCpf1 | 184 |
| TGFBR24034 | CCAGGTTGAACTCAGCTTCT | 20 | AsCpf1 | 185 |
| TGFBR24043 | ATCACAAAATTTACACAGTTG | 21 | SauCas9 | 186 |
| TGFBR24065 | GGCATCAGCCTCCTGCCACCA | 21 | SauCas9 | 187 |
| TGFBR24110 | GTTAGCCAGGTCATCCACAGA | 21 | SauCas9 | 188 |
| TGFBR24099 | GCTGGGCAGCTCCCTCGCCCG | 21 | SauCas9 | 189 |
| TGFBR24064 | CAGGAGGCTGATGCCTGTCAC | 21 | SauCas9 | 190 |
| TGFBR24094 | GAGGAGCGGAAGACGGAGTTG | 21 | SauCas9 | 191 |
| TGFBR24108 | CGTCTGGACCCTACTCTGTCT | 21 | SauCas9 | 192 |
| TGFBR24058 | TTTTTCCTTCATAATGCACTT | 21 | SauCas9 | 193 |
| TGFBR24075 | CCATTGAGCTGGACACCCTGG | 21 | SauCas9 | 194 |
| TGFBR24057 | CTTCTCCAAAGTGCATTATGA | 21 | SauCas9 | 195 |
| TGFBR24103 | GCCCAAGATGCCCATCGTGCA | 21 | SauCas9 | 196 |
| TGFBR24060 | TCATGTGTTCCTGTAGCTCTG | 21 | SauCas9 | 197 |
| TGFBR24048 | GTGATGCTGCAGTTGCTCATG | 21 | SauCas9 | 198 |
| TGFBR24087 | TCTCATGCTTCAGATTGATGT | 21 | SauCas9 | 199 |
| TGFBR24081 | TCCCTATGAGGAGTATGCCTC | 21 | SauCas9 | 200 |
| TGFBR24044 | CATCACAAAATTTACACAGTT | 21 | SauCas9 | 201 |
| TGFBR24077 | ATTGAGCTGGACACCCTGGTG | 21 | SauCas9 | 202 |
| TGFBR24080 | CAGTCAAGATCTTTCCCTATG | 21 | SauCas9 | 203 |
| TGFBR24046 | AGGATTTCTGGTTGTCACAGG | 21 | SauCas9 | 204 |
| TGFBR24101 | TCCACAGTGATCACACTCCAT | 21 | SauCas9 | 205 |
| TGFBR24079 | AGCAGAACACTTCAGAGCAGT | 21 | SauCas9 | 206 |
| TGFBR24072 | CCGGCAAGACGCGGAAGCTCA | 21 | SauCas9 | 207 |
| TGFBR24074 | GATGTCAGAGCGGTCATCTTC | 21 | SauCas9 | 208 |
| TGFBR24062 | TCATTGCACTCATCAGAGCTA | 21 | SauCas9 | 209 |
| TGFBR24054 | CTTCCAGAATAAAGTCATGGT | 21 | SauCas9 | 210 |
| TGFBR24045 | AGATTTTCCACCTGTGACAAC | 21 | SauCas9 | 211 |
| TGFBR24049 | ACTGCAGCATCACCTCCATCT | 21 | SauCas9 | 212 |
| TGFBR24098 | AGCTGGGCAGCTCCCTCGCCC | 21 | SauCas9 | 213 |
| TGFBR24090 | TGACGGCTGAGGAGCGGAAGA | 21 | SauCas9 | 214 |
| TGFBR24076 | CATTGAGCTGGACACCCTGGT | 21 | SauCas9 | 215 |
| TGFBR24078 | AGCAAAGCGACCTTTCCCCAC | 21 | SauCas9 | 216 |
| TGFBR24067 | CGCGTTAACCGGCAGCAGAAG | 21 | SauCas9 | 217 |
| TGFBR24063 | GAAATATGACTAGCAACAAGT | 21 | SauCas9 | 218 |
| TGFBR24107 | AGACAGAGTAGGGTCCAGACG | 21 | SauCas9 | 219 |
| TGFBR24047 | CAGGATTTCTGGTTGTCACAG | 21 | SauCas9 | 220 |
| TGFBR24096 | CTCCTGTAGGTTGCCCTTGGC | 21 | SauCas9 | 221 |
| TGFBR24105 | ACAGAGTAGGGTCCAGACGCA | 21 | SauCas9 | 222 |
| TGFBR24056 | GCTTCTCCAAAGTGCATTATG | 21 | SauCas9 | 223 |
| TGFBR24068 | GCAGCAGAAGCTGAGTTCAAC | 21 | SauCas9 | 224 |
| TGFBR24093 | TGAGGAGCGGAAGACGGAGTT | 21 | SauCas9 | 225 |
| TGFBR24055 | CTTTGGAGAAGCAGCATCTTC | 21 | SauCas9 | 226 |
| TGFBR24053 | CTCCCCTACCATGACTTTATT | 21 | SauCas9 | 227 |
| TGFBR24106 | GACAGAGTAGGGTCCAGACGC | 21 | SauCas9 | 228 |
| TGFBR24092 | CTGAGGAGCGGAAGACGGAGT | 21 | SauCas9 | 229 |
| TGFBR24102 | GGGCATCTTGGGCCTCCCACA | 21 | SauCas9 | 230 |
| TGFBR24082 | CCAAGAGGCATACTCCTCATA | 21 | SauCas9 | 231 |
| TGFBR24051 | AGAATGACGAGAACATAACAC | 21 | SauCas9 | 232 |
| TGFBR24097 | CCTGACGCGGCATGTCATCAG | 21 | SauCas9 | 233 |
| TGFBR24073 | AGCGAGCACTGTGCCATCATC | 21 | SauCas9 | 234 |
| TGFBR24104 | GCAGGTTAGGTCGTTCTTCAC | 21 | SauCas9 | 235 |
| TGFBR24050 | ACCTCCATCTGTGAGAAGCCA | 21 | SauCas9 | 236 |
| TGFBR24052 | TAAAGTCATGGTAGGGGAGCT | 21 | SauCas9 | 237 |
| TGFBR24061 | TCAGAGCTACAGGAACACATG | 21 | SauCas9 | 238 |
| TGFBR24086 | TCTCAGACATCAATCTGAAGC | 21 | SauCas9 | 239 |
| TGFBR24066 | CATCAGCCTCCTGCCACCACT | 21 | SauCas9 | 240 |
| TGFBR24089 | CGCTCCTCAGCCGTCAGGAAC | 21 | SauCas9 | 241 |
| TGFBR24071 | AACCTGGGAAACCGGCAAGAC | 21 | SauCas9 | 242 |
| TGFBR24095 | TCCACGCCAAGGGCAACCTAC | 21 | SauCas9 | 243 |
| TGFBR24100 | GAGGTGAGCAATCCCCCGGGC | 21 | SauCas9 | 244 |
| TGFBR24069 | CAGCAGAAGCTGAGTTCAACC | 21 | SauCas9 | 245 |
| TGFBR24083 | TCCAAGAGGCATACTCCTCAT | 21 | SauCas9 | 246 |
| TGFBR24070 | AGCAGAAGCTGAGTTCAACCT | 21 | SauCas9 | 247 |
| TGFBR24088 | CCAGTTCCTGACGGCTGAGGA | 21 | SauCas9 | 248 |
| TGFBR24085 | AGGAGTATGCCTCTTGGAAGA | 21 | SauCas9 | 249 |
| TGFBR24084 | TTCCAAGAGGCATACTCCTCA | 21 | SauCas9 | 250 |
| TGFBR24042 | CAACTGTGTAAATTTTGTGAT | 21 | SauCas9 | 251 |
| TGFBR24059 | TGAAGGAAAAAAAAAAGCCTG | 21 | SauCas9 | 252 |
| TGFBR24091 | CGTCTTCCGCTCCTCAGCCGT | 21 | SauCas9 | 253 |
| TGFBR24109 | CCAGGTCATCCACAGACAGAG | 21 | SauCas9 | 254 |
| TGFBR2736 | GCCTAGAGTGAAGAGATTCAT | 21 | SpyCas9 | 255 |
| TGFBR2737 | GTTCTCCAAAGTGCATTATGA | 21 | SpyCas9 | 256 |
| TGFBR2738 | GCATCTTCCAGAATAAAGTCA | 21 | SpyCas9 | 257 |
| TGFBR2739 | TGATGTGAGATTTTCCACCTG | 21 | Cas12a | 1172 |

In some embodiments the gRNA for use in the disclosure is a gRNA targeting CISH (CISH gRNA). In some embodiments, the gRNA targeting CISH is one or more of the gRNAs described in Table 5.

TABLE 5
Exemplary CISH gRNAs

| | gRNA Targeting Domain Sequence | | | |
|---|---|---|---|---|
| Name | (DNA) | Length | Enzyme | SEQ ID NO: |
| CISH0873 | CAACCGTCTGGTGGCCGACG | 20 | SpyCas9 | 258 |
| CISHO874 | CAGGATCGGGGCTGTCGCTT | 20 | SpyCas9 | 259 |
| CISH0875 | TCGGGCCTCGCTGGCCGTAA | 20 | SpyCas9 | 260 |
| CISH0876 | GAGGTAGTCGGCCATGCGCC | 20 | SpyCas9 | 261 |
| CISH0877 | CAGGTGTTGTCGGGCCTCGC | 20 | SpyCas9 | 262 |
| CISH0878 | GGAGGTAGTCGGCCATGCGC | 20 | SpyCas9 | 263 |
| CISH0879 | GGCATACTCAATGCGTACAT | 20 | SpyCas9 | 264 |
| CISH0880 | CCGCCTTGTCATCAACCGTC | 20 | SpyCas9 | 265 |
| CISHO881 | AGGATCGGGGCTGTCGCTTC | 20 | SpyCas9 | 266 |
| CISHO882 | CCTTGTCATCAACCGTCTGG | 20 | SpyCas9 | 267 |
| CISHO883 | TACTCAATGCGTACATTGGT | 20 | SpyCas9 | 268 |
| CISHO884 | GGGTTCCATTACGGCCAGCG | 20 | SpyCas9 | 269 |
| CISHO885 | GGCACTGCTTCTGCGTACAA | 20 | SpyCas9 | 270 |
| CISHO886 | GGTTGATGACAAGGCGGCAC | 20 | SpyCas9 | 271 |
| CISHO887 | TGCTGGGGCCTTCCTCGAGG | 20 | SpyCas9 | 272 |
| CISHO888 | TTGCTGGCTGTGGAGCGGAC | 20 | SpyCas9 | 273 |
| CISHO889 | TTCTCCTACCTTCGGGAATC | 20 | SpyCas9 | 274 |
| CISH0890 | GACTGGCTTGGGCAGTTCCA | 20 | SpyCas9 | 275 |
| CISHO891 | CATGCAGCCCTTGCCTGCTG | 20 | SpyCas9 | 276 |
| CISHO892 | AGCAAAGGACGAGGTCTAGA | 20 | SpyCas9 | 277 |
| CISHO893 | GCCTGCTGGGGCCTTCCTCG | 20 | SpyCas9 | 278 |
| CISHO894 | CAGACTCACCAGATTCCCGA | 20 | SpyCas9 | 279 |
| CISHO895 | ACCTCGTCCTTTGCTGGCTG | 20 | SpyCas9 | 280 |
| CISHO896 | CTCACCAGATTCCCGAAGGT | 20 | SpyCas9 | 281 |
| CISH7048 | TACGCAGAAGCAGTGCCCGC | 20 | AsCpf1 | 282 |
| CISH7049 | AGGTGTACAGCAGTGGCTGG | 20 | AsCpf1 | 283 |
| CISH7050 | GGTGTACAGCAGTGGCTGGT | 20 | AsCpf1 | 284 |
| CISH7051 | CGGATGTGGTCAGCCTTGTG | 20 | AsCpf1 | 285 |
| CISH7052 | CACTGACAGCGTGAACAGGT | 20 | AsCpf1 | 286 |
| CISH7053 | ACTGACAGCGTGAACAGGTA | 20 | AsCpf1 | 287 |
| CISH7054 | GCTCACTCTCTGTCTGGGCT | 20 | AsCpf1 | 288 |
| CISH7055 | CTGGCTGTGGAGCGGACTGG | 20 | AsCpf1 | 289 |
| CISH7056 | GCTCTGACTGTACGGGGCAA | 20 | AsCpf1 RR | 290 |
| CISH7057 | AGCTCTGACTGTACGGGGCA | 20 | AsCpf1 RR | 291 |
| CISH7058 | ACAGTACCCCTTCCAGCTCT | 20 | AsCpf1 RR | 292 |
| CISH7059 | CGTCGGCCACCAGACGGTTG | 20 | AsCpf1 RR | 293 |
| CISH7060 | CCAGCCACTGCTGTACACCT | 20 | AsCpf1 RR | 294 |
| CISH7061 | ACCCCGGCCCTGCCTATGCC | 20 | AsCpf1 RR | 295 |
| CISH7062 | GGTATCAGCAGTGCAGGAGG | 20 | AsCpf1 RR | 296 |
| CISH7063 | GATGTGGTCAGCCTTGTGCA | 20 | AsCpf1 RR | 297 |
| CISH7064 | GGATGTGGTCAGCCTTGTGC | 20 | AsCpf1 RR | 298 |
| CISH7065 | GGCCACGCATCCTGGCCTTT | 20 | AsCpf1 RR | 299 |
| CISH7066 | GAAAGGCCAGGATGCGTGGC | 20 | AsCpf1 RR | 300 |
| CISH7067 | ACTGCTTGTCCAGGCCACGC | 20 | AsCpf1 RR | 301 |
| CISH7068 | TCTGGACTCCAACTGCTTGT | 20 | AsCpf1 RR | 302 |
| CISH7069 | GTCTGGACTCCAACTGCTTG | 20 | AsCpf1 RR | 303 |
| CISH7070 | GCTTCCGTCTGGACTCCAAC | 20 | AsCpf1 RR | 304 |
| CISH7071 | GACGGAAGCTGGAGTCGGCA | 20 | AsCpf1 RR | 305 |
| CISH7072 | CGCTGTCAGTGAAAACCACT | 20 | AsCpf1 RR | 306 |
| CISH7073 | CTGACAGCGTGAACAGGTAG | 20 | AsCpf1 RR | 307 |
| CISH7074 | TTACGGCCAGCGAGGCCCGA | 20 | AsCpf1 RR | 308 |
| CISH7075 | ATTACGGCCAGCGAGGCCCG | 20 | AsCpf1 RR | 309 |
| CISH7076 | GGAATCTGGTGAGTCTGAGG | 20 | AsCpf1 RR | 310 |
| CISH7077 | CCCTCAGACTCACCAGATTC | 20 | AsCpf1 RR | 311 |
| CISH7078 | CGAAGGTAGGAGAAGGTCTT | 20 | AsCpf1 RR | 312 |
| CISH7079 | GAAGGTAGGAGAAGGTCTTG | 20 | AsCpf1 RR | 313 |
| CISH7080 | GCACCTTTGGCTCACTCTCT | 20 | AsCpf1 RR | 314 |
| CISH7081 | TCGAGGAGGTGGCAGAGGGT | 20 | AsCpf1 RR | 315 |
| CISH7082 | TGGAACTGCCCAAGCCAGTC | 20 | AsCpf1 RR | 316 |
| CISH7083 | AGGGACGGGGCCCACAGGGG | 20 | AsCpf1 RR | 317 |
| CISH7084 | GGGACGGGGCCCACAGGGGC | 20 | AsCpf1 RR | 318 |
| CISH7085 | CTCCACAGCCAGCAAAGGAC | 20 | AsCpf1 RR | 319 |
| CISH7086 | CAGCCAGCAAAGGACGAGGT | 20 | AsCpf1 RR | 320 |
| CISH7087 | CTGCCTTCTAGACCTCGTCC | 20 | AsCpf1 RR | 321 |
| CISH7088 | CCTAAGGAGGATGCGCCTAG | 20 | AsCpf1 RVR | 322 |
| CISH7089 | TGGCCTCCTGCACTGCTGAT | 20 | AsCpf1 RVR | 323 |
| CISH7090 | AGCAGTGCAGGAGGCCACAT | 20 | AsCpf1 RVR | 324 |
| CISH7091 | CCGACTCCAGCTTCCGTCTG | 20 | AsCpf1 RVR | 325 |
| CISH7092 | GGGGTTCCATTACGGCCAGC | 20 | AsCpf1 RVR | 326 |
| CISH7093 | CACAGCAGATCCTCCTCTGG | 20 | AsCpf1 RVR | 327 |
| CISH7094 | ATTGCCCCGTACAGTCAGAG | 20 | SauCas9 | 328 |
| CISH7095 | CCCGTACAGTCAGAGCTGGA | 20 | SauCas9 | 329 |
| CISH7096 | TGGTGGAGGAGCAGGCAGTG | 20 | SauCas9 | 330 |
| CISH7097 | TCCTTAGGCATAGGCAGGGC | 20 | SauCas9 | 331 |
| CISH7098 | CGGCCCTGCCTATGCCTAAG | 20 | SauCas9 | 332 |
| CISH7099 | TAGGCATAGGCAGGGCCGGG | 20 | SauCas9 | 333 |
| CISH7100 | AGGCAGGGCCGGGGTGGGAG | 20 | SauCas9 | 334 |
| CISH7101 | GCAGGATCGGGGCTGTCGCT | 20 | SauCas9 | 335 |
| CISH7102 | CTGCACAAGGCTGACCACAT | 20 | SauCas9 | 336 |
| CISH7103 | TGCACAAGGCTGACCACATC | 20 | SauCas9 | 337 |
| CISH7104 | CTGACCACATCCGGAAAGGC | 20 | SauCas9 | 338 |
| CISH7105 | GGCCACGCATCCTGGCCTTT | 20 | SauCas9 | 339 |
| CISH7106 | GCGTGGCCTGGACAAGCAGT | 20 | SauCas9 | 340 |
| CISH7107 | GACAAGCAGTTGGAGTCCAG | 20 | SauCas9 | 341 |
| CISH7108 | GTTGGAGTCCAGACGGAAGC | 20 | SauCas9 | 342 |
| CISH7109 | ATGCGTACATTGGTGGGGCC | 20 | SauCas9 | 343 |
| CISH7110 | TGGCCCCACCAATGTACGCA | 20 | SauCas9 | 344 |
| CISH7111 | GCTACCTGTTCACGCTGTCA | 20 | SauCas9 | 345 |
| CISH7112 | TGACAGCGTGAACAGGTAGC | 20 | SauCas9 | 346 |
| CISH7113 | GTCGGGCCTCGCTGGCCGTA | 20 | SauCas9 | 347 |
| CISH7114 | GCACTTGCCTAGGCTGGTAT | 20 | SauCas9 | 348 |
| CISH7115 | GGGAATCTGGTGAGTCTGAG | 20 | SauCas9 | 349 |
| CISH7116 | CTCACCAGATTCCCGAAGGT | 20 | SauCas9 | 350 |
| CISH7117 | CTCCTACCTTCGGGAATCTG | 20 | SauCas9 | 351 |
| CISH7118 | CAAGACCTTCTCCTACCTTC | 20 | SauCas9 | 352 |
| CISH7119 | CCAAGACCTTCTCCTACCTT | 20 | SauCas9 | 353 |
| CISH7120 | GCCAAGACCTTCTCCTACCT | 20 | SauCas9 | 354 |
| CISH7121 | TATGCACAGCAGATCCTCCT | 20 | SauCas9 | 355 |
| CISH7122 | CAAAGGTGCTGGACCCAGAG | 20 | SauCas9 | 356 |
| CISH7123 | GGCTCACTCTCTGTCTGGGC | 20 | SauCas9 | 357 |
| CISH7124 | AGGGTACCCCAGCCCAGACA | 20 | SauCas9 | 358 |
| CISH7125 | AGAGGGTACCCCAGCCCAGA | 20 | SauCas9 | 359 |
| CISH7126 | GTACCCTCTGCCACCTCCTC | 20 | SauCas9 | 360 |
| CISH7127 | CCTTCCTCGAGGAGGTGGCA | 20 | SauCas9 | 361 |
| CISH7128 | ATGACTGGCTTGGGCAGTTC | 20 | SauCas9 | 362 |
| CISH7129 | GGCCCCTGTGGGCCCCGTCC | 20 | SauCas9 | 363 |
| CISH7130 | AGGACGAGGTCTAGAAGGCA | 20 | SauCas9 | 364 |
| CISH7131 | ACTGACAGCGTGAACAGGTAG | 21 | Cas12a | 1173 |

In some embodiments, the gRNA for use in the disclosure is a gRNA targeting B2M (B2M gRNA). In some embodiments, the gRNA targeting B2M is one or more of the gRNAs described in Table 6.

TABLE 6
Exemplary B2M gRNAs

| | gRNA Targeting Domain | | | SEQ ID |
|---|---|---|---|---|
| gRNA name | Target sequence (DNA) | Length | Enzyme | NO: |
| B2M1 | TATAAGTGGAGGCGTCGCGC | 20 | SpyCas9 | 365 |
| B2M2 | GGGCACGCGTTTAATATAAG | 20 | SpyCas9 | 366 |
| B2M3 | ACTCACGCTGGATAGCCTCC | 20 | SpyCas9 | 367 |
| B2M4 | GGCCGAGATGTCTCGCTCCG | 20 | SpyCas9 | 368 |
| B2M5 | CACGCGTTTAATATAAGTGG | 20 | SpyCas9 | 369 |
| B2M6 | AAGTGGAGGCGTCGCGCTGG | 20 | SpyCas9 | 370 |
| B2M7 | GAGTAGCGCGAGCACAGCTA | 20 | SpyCas9 | 371 |
| B2M8 | AGTGGAGGCGTCGCGCTGGC | 20 | SpyCas9 | 372 |
| B2M9 | GCCCGAATGCTGTCAGCTTC | 20 | SpyCas9 | 373 |
| B2M10 | CGCGAGCACAGCTAAGGCCA | 20 | SpyCas9 | 374 |
| B2M11 | CTCGCGCTACTCTCTCTTTC | 20 | SpyCas9 | 375 |
| B2M12 | GGCCACGGAGCGAGACATCT | 20 | SpyCas9 | 376 |
| B2M13 | CGTGAGTAAACCTGAATCTT | 20 | SpyCas9 | 377 |
| B2M14 | AGTCACATGGTTCACACGGC | 20 | SpyCas9 | 378 |
| B2M15 | AAGTCAACTTCAATGTCGGA | 20 | SpyCas9 | 379 |
| B2M16 | CAGTAAGTCAACTTCAATGT | 20 | SpyCas9 | 380 |
| B2M17 | ACCCAGACACATAGCAATTC | 20 | SpyCas9 | 381 |
| B2M18 | GCATACTCATCTTTTTCAGT | 20 | SpyCas9 | 382 |
| B2M19 | ACAGCCCAAGATAGTTAAGT | 20 | SpyCas9 | 383 |
| B2M20 | GGCATACTCATCTTTTTCAG | 20 | SpyCas9 | 384 |
| B2M21 | TTCCTGAAGCTGACAGCATT | 20 | SpyCas9 | 385 |
| B2M22 | TCACGTCATCCAGCAGAGAA | 20 | SpyCas9 | 386 |
| B2M23 | CAGCCCAAGATAGTTAAGTG | 20 | SpyCas9 | 387 |
| B2M-c1 | AAUUCUCUCUCCAUUCUU | 18 | AsCpf1 | 388 |
| B2M-c2 | AAUUCUCUCUCCAUUCUUC | 19 | AsCpf1 | 389 |
| B2M-c3 | AAUUCUCUCUCCAUUCUUCA | 20 | AsCpf1 | 390 |
| B2M-c4 | AAUUCUCUCUCCAUUCUUCAG | 21 | AsCpf1 | 391 |
| B2M-c5 | AAUUCUCUCUCCAUUCUUCAGU | 22 | AsCpf1 | 392 |
| B2M-c6 | AAUUCUCUCUCCAUUCUUCAGUA | 23 | AsCpf1 | 393 |
| B2M-c7 | AAUUCUCUCUCCAUUCUUCAGUAA | 24 | AsCpf1 | 394 |
| B2M-c8 | ACUUUCCAUUCUCUGCUG | 18 | AsCpf1 | 395 |
| B2M-c9 | ACUUUCCAUUCUCUGCUGG | 19 | AsCpf1 | 396 |
| B2M-c10 | ACUUUCCAUUCUCUGCUGGA | 20 | AsCpf1 | 397 |
| B2M-c11 | ACUUUCCAUUCUCUGCUGGAU | 21 | AsCpf1 | 398 |
| B2M-c12 | ACUUUCCAUUCUCUGCUGGAUG | 22 | AsCpf1 | 399 |
| B2M-c13 | ACUUUCCAUUCUCUGCUGGAUGA | 23 | AsCpf1 | 400 |
| B2M-c14 | ACUUUCCAUUCUCUGCUGGAUGAC | 24 | AsCpf1 | 401 |
| B2M-c15 | AGCAAGGACUGGUCUUUC | 18 | AsCpf1 | 402 |
| B2M-c16 | AGCAAGGACUGGUCUUUCU | 19 | AsCpf1 | 403 |
| B2M-c17 | AGCAAGGACUGGUCUUUCUA | 20 | AsCpf1 | 404 |
| B2M-c18 | AGCAAGGACUGGUCUUUCUAU | 21 | AsCpf1 | 405 |
| B2M-c19 | AGCAAGGACUGGUCUUUCUAUC | 22 | AsCpf1 | 406 |
| B2M-c20 | AGCAAGGACUGGUCUUUCUAUCU | 23 | AsCpf1 | 407 |
| B2M-c21 | AGCAAGGACUGGUCUUUCUAUCUC | 24 | AsCpf1 | 408 |
| B2M-c22 | AGUGGGGGUGAAUUCAGU | 18 | AsCpf1 | 409 |
| B2M-c23 | AGUGGGGGUGAAUUCAGUG | 19 | AsCpf1 | 410 |
| B2M-c24 | AGUGGGGGUGAAUUCAGUGU | 20 | AsCpf1 | 411 |
| B2M-c25 | AGUGGGGGUGAAUUCAGUGUA | 21 | AsCpf1 | 412 |
| B2M-c26 | AGUGGGGGUGAAUUCAGUGUAG | 22 | AsCpf1 | 413 |
| B2M-c27 | AGUGGGGGUGAAUUCAGUGUAGU | 23 | AsCpf1 | 414 |
| B2M-c28 | AGUGGGGGUGAAUUCAGUGUAGUA | 24 | AsCpf1 | 415 |
| B2M-c29 | AUCCAUCCGACAUUGAAG | 18 | AsCpf1 | 416 |
| B2M-c30 | AUCCAUCCGACAUUGAAGU | 19 | AsCpf1 | 417 |
| B2M-c31 | AUCCAUCCGACAUUGAAGUU | 20 | AsCpf1 | 418 |
| B2M-c32 | AUCCAUCCGACAUUGAAGUUG | 21 | AsCpf1 | 419 |
| B2M-c33 | AUCCAUCCGACAUUGAAGUUGA | 22 | AsCpf1 | 420 |
| B2M-c34 | AUCCAUCCGACAUUGAAGUUGAC | 23 | AsCpf1 | 421 |
| B2M-c35 | AUCCAUCCGACAUUGAAGUUGACU | 24 | AsCpf1 | 422 |
| B2M-c36 | CAAUUCUCUCUCCAUUCU | 18 | AsCpf1 | 423 |
| B2M-c37 | CAAUUCUCUCUCCAUUCUU | 19 | AsCpf1 | 424 |
| B2M-c38 | CAAUUCUCUCUCCAUUCUUC | 20 | AsCpf1 | 425 |
| B2M-c39 | CAAUUCUCUCUCCAUUCUUCA | 21 | AsCpf1 | 426 |
| B2M-c40 | CAAUUCUCUCUCCAUUCUUCAG | 22 | AsCpf1 | 427 |
| B2M-c41 | CAAUUCUCUCUCCAUUCUUCAGU | 23 | AsCpf1 | 428 |
| B2M-c42 | CAAUUCUCUCUCCAUUCUUCAGUA | 24 | AsCpf1 | 429 |
| B2M-c43 | CAGUGGGGGUGAAUUCAG | 18 | AsCpf1 | 430 |
| B2M-c44 | CAGUGGGGGUGAAUUCAGU | 19 | AsCpf1 | 431 |
| B2M-c45 | CAGUGGGGGUGAAUUCAGUG | 20 | AsCpf1 | 432 |
| B2M-c46 | CAGUGGGGGUGAAUUCAGUGU | 21 | AsCpf1 | 433 |
| B2M-c47 | CAGUGGGGGUGAAUUCAGUGUA | 22 | AsCpf1 | 434 |
| B2M-c48 | CAGUGGGGGUGAAUUCAGUGUAG | 23 | AsCpf1 | 435 |
| B2M-c49 | CAGUGGGGGUGAAUUCAGUGUAGU | 24 | AsCpf1 | 436 |
| B2M-c50 | CAUUCUCUGCUGGAUGAC | 18 | AsCpf1 | 437 |
| B2M-c51 | CAUUCUCUGCUGGAUGACG | 19 | AsCpf1 | 438 |
| B2M-c52 | CAUUCUCUGCUGGAUGACGU | 20 | AsCpf1 | 439 |
| B2M-c53 | CAUUCUCUGCUGGAUGACGUG | 21 | AsCpf1 | 440 |
| B2M-c54 | CAUUCUCUGCUGGAUGACGUGA | 22 | AsCpf1 | 441 |
| B2M-c55 | CAUUCUCUGCUGGAUGACGUGAG | 23 | AsCpf1 | 442 |
| B2M-c56 | CAUUCUCUGCUGGAUGACGUGAGU | 24 | AsCpf1 | 443 |
| B2M-c57 | CCCGAUAUUCCUCAGGUA | 18 | AsCpf1 | 444 |
| B2M-c58 | CCCGAUAUUCCUCAGGUAC | 19 | AsCpf1 | 445 |
| B2M-c59 | CCCGAUAUUCCUCAGGUACU | 20 | AsCpf1 | 446 |
| B2M-c60 | CCCGAUAUUCCUCAGGUACUC | 21 | AsCpf1 | 447 |
| B2M-c61 | CCCGAUAUUCCUCAGGUACUCC | 22 | AsCpf1 | 448 |
| B2M-c62 | CCCGAUAUUCCUCAGGUACUCCA | 23 | AsCpf1 | 449 |
| B2M-c63 | CCCGAUAUUCCUCAGGUACUCCAA | 24 | AsCpf1 | 450 |
| B2M-c64 | CCGAUAUUCCUCAGGUAC | 18 | AsCpf1 | 451 |
| B2M-c65 | CCGAUAUUCCUCAGGUACU | 19 | AsCpf1 | 452 |
| B2M-c66 | CCGAUAUUCCUCAGGUACUC | 20 | AsCpf1 | 453 |
| B2M-c67 | CCGAUAUUCCUCAGGUACUCC | 21 | AsCpf1 | 454 |
| B2M-c68 | CCGAUAUUCCUCAGGUACUCCA | 22 | AsCpf1 | 455 |
| B2M-c69 | CCGAUAUUCCUCAGGUACUCCAA | 23 | AsCpf1 | 456 |
| B2M-c70 | CCGAUAUUCCUCAGGUACUCCAAA | 24 | AsCpf1 | 457 |
| B2M-c71 | CUCACGUCAUCCAGCAGA | 18 | AsCpf1 | 458 |
| B2M-c72 | CUCACGUCAUCCAGCAGAG | 19 | AsCpf1 | 459 |
| B2M-c73 | CUCACGUCAUCCAGCAGAGA | 20 | AsCpf1 | 460 |
| B2M-c74 | CUCACGUCAUCCAGCAGAGAA | 21 | AsCpf1 | 461 |
| B2M-c75 | CUCACGUCAUCCAGCAGAGAAU | 22 | AsCpf1 | 462 |
| B2M-c76 | CUCACGUCAUCCAGCAGAGAAUG | 23 | AsCpf1 | 463 |
| B2M-c77 | CUCACGUCAUCCAGCAGAGAAUGG | 24 | AsCpf1 | 464 |
| B2M-c78 | CUGAAUUGCUAUGUGUCU | 18 | AsCpf1 | 465 |
| B2M-c79 | CUGAAUUGCUAUGUGUCUG | 19 | AsCpf1 | 466 |
| B2M-c80 | CUGAAUUGCUAUGUGUCUGG | 20 | AsCpf1 | 467 |
| B2M-c81 | CUGAAUUGCUAUGUGUCUGGG | 21 | AsCpf1 | 468 |
| B2M-c82 | CUGAAUUGCUAUGUGUCUGGGU | 22 | AsCpf1 | 469 |
| B2M-c83 | CUGAAUUGCUAUGUGUCUGGGUU | 23 | AsCpf1 | 470 |
| B2M-c84 | CUGAAUUGCUAUGUGUCUGGGUUU | 24 | AsCpf1 | 471 |
| B2M-c85 | GAGUACCUGAGGAAUAUC | 18 | AsCpf1 | 472 |
| B2M-c86 | GAGUACCUGAGGAAUAUCG | 19 | AsCpf1 | 473 |
| B2M-c87 | GAGUACCUGAGGAAUAUCGG | 20 | AsCpf1 | 474 |
| B2M-c88 | GAGUACCUGAGGAAUAUCGGG | 21 | AsCpf1 | 475 |
| B2M-c89 | GAGUACCUGAGGAAUAUCGGGA | 22 | AsCpf1 | 476 |
| B2M-c90 | GAGUACCUGAGGAAUAUCGGGAA | 23 | AsCpf1 | 477 |
| B2M-c91 | GAGUACCUGAGGAAUAUCGGGAAA | 24 | AsCpf1 | 478 |
| B2M-c92 | UAUCUCUUGUACUACACU | 18 | AsCpf1 | 479 |
| B2M-c93 | UAUCUCUUGUACUACACUG | 19 | AsCpf1 | 480 |
| B2M-c94 | UAUCUCUUGUACUACACUGA | 20 | AsCpf1 | 481 |
| B2M-c95 | UAUCUCUUGUACUACACUGAA | 21 | AsCpf1 | 482 |
| B2M-c96 | UAUCUCUUGUACUACACUGAAU | 22 | AsCpf1 | 483 |
| B2M-c97 | UAUCUCUUGUACUACACUGAAUU | 23 | AsCpf1 | 484 |
| B2M-c98 | UAUCUCUUGUACUACACUGAAUUC | 24 | AsCpf1 | 485 |
| B2M-c99 | UCAAUUCUCUCUCCAUUC | 18 | AsCpf1 | 486 |
| B2M-c100 | UCAAUUCUCUCUCCAUUCU | 19 | AsCpf1 | 487 |
| B2M-c101 | UCAAUUCUCUCUCCAUUCUU | 20 | AsCpf1 | 488 |
| B2M-c102 | UCAAUUCUCUCUCCAUUCUUC | 21 | AsCpf1 | 489 |
| B2M-c103 | UCAAUUCUCUCUCCAUUCUUCA | 22 | AsCpf1 | 490 |
| B2M-c104 | UCAAUUCUCUCUCCAUUCUUCAG | 23 | AsCpf1 | 491 |
| B2M-c105 | UCAAUUCUCUCUCCAUUCUUCAGU | 24 | AsCpf1 | 492 |
| B2M-c106 | UCACAGCCCAAGAUAGUU | 18 | AsCpf1 | 493 |
| B2M-c107 | UCACAGCCCAAGAUAGUUA | 19 | AsCpf1 | 494 |
| B2M-c108 | UCACAGCCCAAGAUAGUU AA | 20 | AsCpf1 | 495 |
| B2M-c109 | UCACAGCCCAAGAUAGUUAAG | 21 | AsCpf1 | 496 |
| B2M-c110 | UCACAGCCCAAGAUAGUUAAGU | 22 | AsCpf1 | 497 |
| B2M-c111 | UCACAGCCCAAGAUAGUUAAGUG | 23 | AsCpf1 | 498 |
| B2M-c112 | UCACAGCCCAAGAUAGUUAAGUGG | 24 | AsCpf1 | 499 |
| B2M-c113 | UCAGUGGGGGUGAAUUCA | 18 | AsCpf1 | 500 |
| B2M-c114 | UCAGUGGGGGUGAAUUCAG | 19 | AsCpf1 | 501 |
| B2M-c115 | UCAGUGGGGGUGAAUUCAGU | 20 | AsCpf1 | 502 |
| B2M-c116 | UCAGUGGGGGUGAAUUCAGUG | 21 | AsCpf1 | 503 |
| B2M-c117 | UCAGUGGGGGUGAAUUCAGUGU | 22 | AsCpf1 | 504 |
| B2M-c118 | UCAGUGGGGGUGAAUUCAGUGUA | 23 | AsCpf1 | 505 |
| B2M-c119 | UCAGUGGGGGUGAAUUCAGUGUAG | 24 | AsCpf1 | 506 |
| B2M-c120 | UGGCCUGGAGGCUAUCCA | 18 | AsCpf1 | 507 |
| B2M-c121 | UGGCCUGGAGGCUAUCCAG | 19 | AsCpf1 | 508 |
| B2M-c122 | UGGCCUGGAGGCUAUCCAGC | 20 | AsCpf1 | 509 |
| B2M-c123 | UGGCCUGGAGGCUAUCCAGCG | 21 | AsCpf1 | 510 |
| B2M-c124 | UGGCCUGGAGGCUAUCCAGCGU | 22 | AsCpf1 | 511 |
| B2M-c125 | UGGCCUGGAGGCUAUCCAGCGUG | 23 | AsCpf1 | 512 |
| B2M-c126 | UGGCCUGGAGGCUAUCCAGCGUGA | 24 | AsCpf1 | 513 |
| B2M-c127 | AUAGAUCGAGACAUGUAA | 18 | AsCpf1 | 514 |
| B2M-c128 | AUAGAUCGAGACAUGUAAG | 19 | AsCpf1 | 515 |
| B2M-c129 | AUAGAUCGAGACAUGUAAGC | 20 | AsCpf1 | 516 |
| B2M-c130 | AUAGAUCGAGACAUGUAAGCA | 21 | AsCpf1 | 517 |
| B2M-c131 | AUAGAUCGAGACAUGUAAGCAG | 22 | AsCpf1 | 518 |
| B2M-c132 | AUAGAUCGAGACAUGUAAGCAGC | 23 | AsCpf1 | 519 |
| B2M-c133 | AUAGAUCGAGACAUGUAAGCAGCA | 24 | AsCpf1 | 520 |
| B2M-c134 | CAUAGAUCGAGACAUGUA | 18 | AsCpf1 | 521 |
| B2M-c135 | CAUAGAUCGAGACAUGUAA | 19 | AsCpf1 | 522 |
| B2M-c136 | CAUAGAUCGAGACAUGUAAG | 20 | AsCpf1 | 523 |
| B2M-c137 | CAUAGAUCGAGACAUGUAAGC | 21 | AsCpf1 | 524 |
| B2M-c138 | CAUAGAUCGAGACAUGUAAGCA | 22 | AsCpf1 | 525 |
| B2M-c139 | CAUAGAUCGAGACAUGUAAGCAG | 23 | AsCpf1 | 526 |
| B2M-c140 | CAUAGAUCGAGACAUGUAAGCAGC | 24 | AsCpf1 | 527 |
| B2M-c141 | CUCCACUGUCUUUUUCAU | 18 | AsCpf1 | 528 |
| B2M-c142 | CUCCACUGUCUUUUUCAUA | 19 | AsCpf1 | 529 |
| B2M-c143 | CUCCACUGUCUUUUUCAUAG | 20 | AsCpf1 | 530 |
| B2M-c144 | CUCCACUGUCUUUUUCAUAGA | 21 | AsCpf1 | 531 |
| B2M-c145 | CUCCACUGUCUUUUUCAUAGAU | 22 | AsCpf1 | 532 |
| B2M-c146 | CUCCACUGUCUUUUUCAUAGAUC | 23 | AsCpf1 | 533 |
| B2M-c147 | CUCCACUGUCUUUUUCAUAGAUCG | 24 | AsCpf1 | 534 |
| B2M-c148 | UCAUAGAUCGAGACAUGU | 18 | AsCpf1 | 535 |
| B2M-c149 | UCAUAGAUCGAGACAUGUA | 19 | AsCpf1 | 536 |
| B2M-c150 | UCAUAGAUCGAGACAUGUAA | 20 | AsCpf1 | 537 |
| B2M-c151 | UCAUAGAUCGAGACAUGUAAG | 21 | AsCpf1 | 538 |
| B2M-c152 | UCAUAGAUCGAGACAUGUAAGC | 22 | AsCpf1 | 539 |
| B2M-c153 | UCAUAGAUCGAGACAUGUAAGCA | 23 | AsCpf1 | 540 |
| B2M-c154 | UCAUAGAUCGAGACAUGUAAGCAG | 24 | AsCpf1 | 541 |
| B2M-c155 | UCCACUGUCUUUUUCAUA | 18 | AsCpf1 | 542 |
| B2M-c156 | UCCACUGUCUUUUUCAUAG | 19 | AsCpf1 | 543 |
| B2M-c157 | UCCACUGUCUUUUUCAUAGA | 20 | AsCpf1 | 544 |
| B2M-c158 | UCCACUGUCUUUUUCAUAGAU | 21 | AsCpf1 | 545 |
| B2M-c159 | UCCACUGUCUUUUUCAUAGAUC | 22 | AsCpf1 | 546 |
| B2M-c160 | UCCACUGUCUUUUUCAUAGAUCG | 23 | AsCpf1 | 547 |
| B2M-c161 | UCCACUGUCUUUUUCAUAGAUCGA | 24 | AsCpf1 | 548 |
| B2M-c162 | UCUCCACUGUCUUUUUCA | 18 | AsCpf1 | 549 |
| B2M-c163 | UCUCCACUGUCUUUUUCAU | 19 | AsCpf1 | 550 |
| B2M-c164 | UCUCCACUGUCUUUUUCAUA | 20 | AsCpf1 | 551 |
| B2M-c165 | UCUCCACUGUCUUUUUCAUAG | 21 | AsCpf1 | 552 |
| B2M-c166 | UCUCCACUGUCUUUUUCAUAGA | 22 | AsCpf1 | 553 |
| B2M-c167 | UCUCCACUGUCUUUUUCAUAGAU | 23 | AsCpf1 | 554 |
| B2M-c168 | UCUCCACUGUCUUUUUCAUAGAUC | 24 | AsCpf1 | 555 |
| B2M-c169 | UUCUCCACUGUCUUUUUC | 18 | AsCpf1 | 556 |
| B2M-c170 | UUCUCCACUGUCUUUUUCA | 19 | AsCpf1 | 557 |
| B2M-c171 | UUCUCCACUGUCUUUUUCAU | 20 | AsCpf1 | 558 |
| B2M-c172 | UUCUCCACUGUCUUUUUCAUA | 21 | AsCpf1 | 559 |
| B2M-c173 | UUCUCCACUGUCUUUUUCAUAG | 22 | AsCpf1 | 560 |
| B2M-c174 | UUCUCCACUGUCUUUUUCAUAGA | 23 | AsCpf1 | 561 |
| B2M-c175 | UUCUCCACUGUCUUUUUCAUAGAU | 24 | AsCpf1 | 562 |
| B2M-c176 | UUUCUCCACUGUCUUUUU | 18 | AsCpf1 | 563 |
| B2M-c177 | UUUCUCCACUGUCUUUUUC | 19 | AsCpf1 | 564 |
| B2M-c178 | UUUCUCCACUGUCUUUUUCA | 20 | AsCpf1 | 565 |
| B2M-c179 | UUUCUCCACUGUCUUUUUCAU | 21 | AsCpf1 | 566 |
| B2M-c180 | UUUCUCCACUGUCUUUUUCAUA | 22 | AsCpf1 | 567 |
| B2M-c181 | UUUCUCCACUGUCUUUUUCAUAG | 23 | AsCpf1 | 568 |
| B2M-c182 | UUUCUCCACUGUCUUUUUCAUAGA | 24 | AsCpf1 | 569 |
| B2M-c183 | UUUUCUCCACUGUCUUUU | 18 | AsCpf1 | 570 |
| B2M-c184 | UUUUCUCCACUGUCUUUUU | 19 | AsCpf1 | 571 |
| B2M-c185 | UUUUCUCCACUGUCUUUUUC | 20 | AsCpf1 | 572 |
| B2M-c186 | UUUUCUCCACUGUCUUUUUCA | 21 | AsCpf1 | 573 |
| B2M-c187 | UUUUCUCCACUGUCUUUUUCAU | 22 | AsCpf1 | 574 |
| B2M-c188 | UUUUCUCCACUGUCUUUUUCAUA | 23 | AsCpf1 | 575 |
| B2M-c189 | UUUUCUCCACUGUCUUUUUCAUAG | 24 | AsCpf1 | 576 |

In some embodiments, the gRNA for use in the disclosure is a gRNA targeting PD1. gRNAs targeting B2M and PD1 for use in the disclosure are further described in WO2015161276 and WO2017152015 by Welstead et al.; both incorporated in their entirety herein by reference.

In some embodiments, the gRNA for use in the disclosure is a gRNA targeting NKG2A (NKG2A gRNA). In some embodiments, the gRNA targeting NKG2A is one or more of the gRNAs described in Table 7.

TABLE 7
Exemplary NKG2A gRNAs

| | | | | SEQ |
|---|---|---|---|---|
| | gRNA Targeting Domain | | | ID |
| Name | Sequence (DNA) | Length | Enzyme | NO: |
| NKG2A55 | GAGGTAAAGCGTTTGCATTTG | 21 | AsCpf1 | 577 |
| NKG2A56 | CCTCTAAAGCTTATGCTTACA | 21 | AsCpf1 | 578 |
| NKG2A57 | AGTCGATTTACTTGTAGCACT | 21 | AsCpf1 | 579 |
| NKG2A58 | CTTGTAGCACTGCACAGTTAA | 21 | AsCpf1 | 580 |
| NKG2A59 | TCCATTACAGGATAAAAGACT | 21 | AsCpf1 | 581 |
| NKG2A60 | CTCCATTACAGGATAAAAGAC | 21 | AsCpf1 | 582 |
| NKG2A61 | TCTCCATTACAGGATAAAAGA | 21 | AsCpf1 | 583 |
| NKG2A62 | ATCCTGTAATGGAGAAAAATC | 21 | AsCpf1 | 584 |
| NKG2A63 | TCCTGTAATGGAGAAAAATCC | 21 | AsCpf1 | 585 |
| NKG2A136 | AAACATGAGTAAGTTGTTTTG | 21 | AsCpf1 | 586 |
| NKG2A137 | GCTTTCAAACATGAGTAAGTT | 21 | AsCpf1 | 587 |
| NKG2A138 | AAAGCCAAACCATTCATTGTC | 21 | AsCpf1 | 588 |
| NKG2A139 | GTAACAGCAGTCATCATCCAT | 21 | AsCpf1 | 589 |
| NKG2A140 | ACCATCCTCATGGATTGGTGT | 21 | AsCpf1 | 590 |
| NKG2A141 | TGTCCATCATTTCACCATCCT | 21 | AsCpf1 | 591 |
| NKG2A142 | GAAATTTCTGTCCATCATTTC | 21 | AsCpf1 | 592 |
| NKG2A143 | AGAAATTTCTGTCCATCATTT | 21 | AsCpf1 | 593 |
| NKG2A144 | TTTTAGAAATTTCTGTCCATC | 21 | AsCpf1 | 594 |
| NKG2A145 | CTTTTAGAAATTTCTGTCCAT | 21 | AsCpf1 | 595 |
| NKG2A146 | TTTTCTTTTAGAAATTTCTGT | 21 | AsCpf1 | 596 |
| NKG2A147 | TAAAAGAAAAGAAAGAATTTT | 21 | AsCpf1 | 597 |
| NKG2A270 | AAACATTTACATCTTACCATT | 21 | AsCpf1 | 598 |
| NKG2A271 | CATCTTACCATTTCTTCTTCA | 21 | AsCpf1 | 599 |
| NKG2A272 | TATAGATAATGAAGAAGAAAT | 21 | AsCpf1 | 600 |
| NKG2A273 | TTCTTCATTATCTATAGAAAG | 21 | AsCpf1 | 601 |
| NKG2A274 | CTGGCCTGTACTTCGAAGAAC | 21 | AsCpf1 | 602 |
| NKG2A275 | CTTACCAATGTAGTAACAACT | 21 | AsCpf1 | 603 |
| NKG2A276 | GCACGTCATTGTGGCCATTGT | 21 | AsCpf1 | 604 |
| NKG2A277 | TTTAGCACGTCATTGTGGCCA | 21 | AsCpf1 | 605 |
| NKG2A414 | CCATCAGCTCCAGAGAAGCTC | 21 | AsCpf1 | 606 |
| NKG2A415 | TCTCCCTGCAGATTTACCATC | 21 | AsCpf1 | 607 |
| NKG2A437 | AAATGCTTTACCTTTGCAGTG | 21 | AsCpf1 | 608 |
| NKG2A438 | AATGCTTTACCTTTGCAGTGA | 21 | AsCpf1 | 609 |
| NKG2A439 | CCTTTGCAGTGATAGGTTTTG | 21 | AsCpf1 | 610 |
| NKG2A440 | CAGTGATAGGTTTTGTCATTC | 21 | AsCpf1 | 611 |
| NKG2A441 | AAGGGAATGACAAAACCTATC | 21 | AsCpf1 | 612 |
| NKG2A442 | CAAGGGAATGACAAAACCTAT | 21 | AsCpf1 | 613 |
| NKG2A443 | GTCATTCCCTTGAAAATCCTG | 21 | AsCpf1 | 614 |
| NKG2A444 | TCATTCCCTTGAAAATCCTGA | 21 | AsCpf1 | 615 |
| NKG2A445 | TGAAGGTTTAATTCCGCATAG | 21 | AsCpf1 | 616 |
| NKG2A446 | GAAGGTTTAATTCCGCATAGG | 21 | AsCpf1 | 617 |
| NKG2A447 | AAGGTTTAATTCCGCATAGGT | 21 | AsCpf1 | 618 |
| NKG2A448 | ATTCCGCATAGGTTATTTCCT | 21 | AsCpf1 | 619 |
| NKG2A449 | GCAACTGAACAGGAAATAACC | 21 | AsCpf1 | 620 |
| NKG2A450 | AGCAACTGAACAGGAAATAAC | 21 | AsCpf1 | 621 |
| NKG2A451 | CTGTTCAGTTGCTAAAATGGA | 21 | AsCpf1 | 622 |
| NKG2A452 | TATTGCCTTTAGGTTTTCGTT | 21 | AsCpf1 | 623 |
| NKG2A453 | ATTGCCTTTAGGTTTTCGTTG | 21 | AsCpf1 | 624 |
| NKG2A454 | TTGCCTTTAGGTTTTCGTTGC | 21 | AsCpf1 | 625 |
| NKG2A455 | GGTTTTCGTTGCTGCCTCTTT | 21 | AsCpf1 | 626 |
| NKG2A456 | CGTTGCTGCCTCTTTGGGTTT | 21 | AsCpf1 | 627 |
| NKG2A457 | GTTGCTGCCTCTTTGGGTTTG | 21 | AsCpf1 | 628 |
| NKG2A458 | GGTTTGGGGGCAGATTCAGGT | 21 | AsCpf1 | 629 |
| NKG2A459 | GGGGCAGATTCAGGTCTGAGT | 21 | AsCpf1 | 630 |
| NKG2A460 | GCAACTGAACAGGAAATAACC | 21 | Cas12a | 1176 |

In some embodiments, the gRNA for use in the disclosure is a gRNA targeting TIGIT (TIGIT gRNA). In some embodiments, the gRNA targeting TIGIT is one or more of the gRNAs described in Table 8.

TABLE 8
TIGIT gRNAs

| | gRNA Targeting Domain | | | SEQ ID |
|---|---|---|---|---|
| Name | Sequence (DNA) | Length | Enzyme | NO: |
| TIGIT4170 | TCTGCAGAAATGTTCCCCGT | 20 | AsCpf1 | 631 |
| TIGIT4171 | TGCAGAGAAAGGTGGCTCTA | 20 | AsCpf1 | 632 |
| TIGIT4172 | TAATGCTGACTTGGGGTGGC | 20 | AsCpf1 | 633 |
| TIGIT4173 | TAGGACCTCCAGGAAGATTC | 20 | AsCpf1 | 634 |
| TIGIT4174 | TAGTCAACGCGACCACCACG | 20 | AsCpf1 | 635 |
| TIGIT4175 | TCCTGAGGTCACCTTCCACA | 20 | AsCpf1 | 636 |
| TIGIT4176 | TATTGTGCCTGTCATCATTC | 20 | AsCpf1 | 637 |
| TIGIT4177 | TGACAGGCACAATAGAAACAA | 21 | SauCas9 | 638 |
| TIGIT4178 | GACAGGCACAATAGAAACAAC | 21 | SauCas9 | 639 |
| TIGIT4179 | AAACAACGGGGAACATTTCTG | 21 | SauCas9 | 640 |
| TIGIT4180 | ACAACGGGGAACATTTCTGCA | 21 | SauCas9 | 641 |
| TIGIT4181 | TGATAGAGCCACCTTTCTCTG | 21 | SauCas9 | 642 |
| TIGIT4182 | GGGTCACTTGTGCCGTGGTGG | 21 | SauCas9 | 643 |
| TIGIT4183 | GGCACAAGTGACCCAGGTCAA | 21 | SauCas9 | 644 |
| TIGIT4184 | GTCCTGCTGCTCCCAGTTGAC | 21 | SauCas9 | 645 |
| TIGIT4185 | TGGCCATTTGTAATGCTGACT | 21 | SauCas9 | 646 |
| TIGIT4186 | TGGCACATCTCCCCATCCTTC | 21 | SauCas9 | 647 |
| TIGIT4187 | CATCTCCCCATCCTTCAAGGA | 21 | SauCas9 | 648 |
| TIGIT4188 | CCACTCGATCCTTGAAGGATG | 21 | SauCas9 | 649 |
| TIGIT4189 | GGCCACTCGATCCTTGAAGGA | 21 | SauCas9 | 650 |
| TIGIT4190 | CCTGGGGCCACTCGATCCTTG | 21 | SauCas9 | 651 |
| TIGIT4191 | GACTGGAGGGTGAGGCCCAGG | 21 | SauCas9 | 652 |
| TIGIT4192 | ATCGTTCACGGTCAGCGACTG | 21 | SauCas9 | 653 |
| TIGIT4193 | GTCGCTGACCGTGAACGATAC | 21 | SauCas9 | 654 |
| TIGIT4194 | CGCTGACCGTGAACGATACAG | 21 | SauCas9 | 655 |
| TIGIT4195 | GCATCTATCACACCTACCCTG | 21 | SauCas9 | 656 |
| TIGIT4196 | CCTACCCTGATGGGACGTACA | 21 | SauCas9 | 657 |
| TIGIT4197 | TACCCTGATGGGACGTACACT | 21 | SauCas9 | 658 |
| TIGIT4198 | CCCTGATGGGACGTACACTGG | 21 | SauCas9 | 659 |
| TIGIT4199 | TTCTCCCAGTGTACGTCCCAT | 21 | SauCas9 | 660 |
| TIGIT4200 | GGAGAATCTTCCTGGAGGTCC | 21 | SauCas9 | 661 |
| TIGIT4201 | CATGGCTCCAAGCAATGGAAT | 21 | SauCas9 | 662 |
| TIGIT4202 | CGCGGCCATGGCTCCAAGCAA | 21 | SauCas9 | 663 |
| TIGIT4203 | TCGCGGCCATGGCTCCAAGCA | 21 | SauCas9 | 664 |
| TIGIT4204 | CATCGTGGTGGTCGCGTTGAC | 21 | SauCas9 | 665 |
| TIGIT4205 | AAAGCCCTCAGAATCCATTCT | 21 | SauCas9 | 666 |
| TIGIT4206 | CATTCTGTGGAAGGTGACCTC | 21 | SauCas9 | 667 |
| TIGIT4207 | TTCTGTGGAAGGTGACCTCAG | 21 | SauCas9 | 668 |
| TIGIT4208 | CCTGAGGTCACCTTCCACAGA | 21 | SauCas9 | 669 |
| TIGIT4209 | TTCTCCTGAGGTCACCTTCCA | 21 | SauCas9 | 670 |
| TIGIT4210 | AGGAGAAAATCAGCTGGACAG | 21 | SauCas9 | 671 |
| TIGIT4211 | GGAGAAAATCAGCTGGACAGG | 21 | SauCas9 | 672 |
| TIGIT4212 | GCCCCAGTGCTCCCTCACCCC | 21 | SauCas9 | 673 |
| TIGIT4213 | TGGACACAGCTTCCTGGGGGT | 21 | SauCas9 | 674 |
| TIGIT4214 | TCTGCCTGGACACAGCTTCCT | 21 | SauCas9 | 675 |
| TIGIT4215 | AGCTGCACCTGCTGGGCTCTG | 21 | SauCas9 | 676 |
| TIGIT4216 | GCTGGGCTCTGTGGAGAGCAG | 21 | SauCas9 | 677 |
| TIGIT4217 | TGGGCTCTGTGGAGAGCAGCG | 21 | SauCas9 | 678 |
| TIGIT4218 | CTGCATGACTACTTCAATGTC | 21 | SauCas9 | 679 |
| TIGIT4219 | AATGTCCTGAGTTACAGAAGC | 21 | SauCas9 | 680 |
| TIGIT4220 | TGGGTAACTGCAGCTTCTTCA | 21 | SauCas9 | 681 |
| TIGIT4221 | GACAGGCACAATAGAAACAA | 20 | SpyCas9 | 682 |
| TIGIT4222 | ACAGGCACAATAGAAACAAC | 20 | SpyCas9 | 683 |
| TIGIT4223 | CAGGCACAATAGAAACAACG | 20 | SpyCas9 | 684 |
| TIGIT4224 | GGGAACATTTCTGCAGAGAA | 20 | SpyCas9 | 685 |
| TIGIT4225 | AACATTTCTGCAGAGAAAGG | 20 | SpyCas9 | 686 |
| TIGIT4226 | ATGTCACCTCTCCTCCACCA | 20 | SpyCas9 | 687 |
| TIGIT4227 | CTTGTGCCGTGGTGGAGGAG | 20 | SpyCas9 | 688 |
| TIGIT4228 | GGTCACTTGTGCCGTGGTGG | 20 | SpyCas9 | 689 |
| TIGIT4229 | CACCACGGCACAAGTGACCC | 20 | SpyCas9 | 690 |
| TIGIT4230 | CTGGGTCACTTGTGCCGTGG | 20 | SpyCas9 | 691 |
| TIGIT4231 | GACCTGGGTCACTTGTGCCG | 20 | SpyCas9 | 692 |
| TIGIT4232 | CACAAGTGACCCAGGTCAAC | 20 | SpyCas9 | 693 |
| TIGIT4233 | ACAAGTGACCCAGGTCAACT | 20 | SpyCas9 | 694 |
| TIGIT4234 | CCAGGTCAACTGGGAGCAGC | 20 | SpyCas9 | 695 |
| TIGIT4235 | CTGCTGCTCCCAGTTGACCT | 20 | SpyCas9 | 696 |
| TIGIT4236 | CCTGCTGCTCCCAGTTGACC | 20 | SpyCas9 | 697 |
| TIGIT4237 | GGAGCAGCAGGACCAGCTTC | 20 | SpyCas9 | 698 |
| TIGIT4238 | CATTACAAATGGCCAGAAGC | 20 | SpyCas9 | 699 |
| TIGIT4239 | GGCCATTTGTAATGCTGACT | 20 | SpyCas9 | 700 |
| TIGIT4240 | GCCATTTGTAATGCTGACTT | 20 | SpyCas9 | 701 |
| TIGIT4241 | CCATTTGTAATGCTGACTTG | 20 | SpyCas9 | 702 |
| TIGIT4242 | TTTGTAATGCTGACTTGGGG | 20 | SpyCas9 | 703 |
| TIGIT4243 | CCCCAAGTCAGCATTACAAA | 20 | SpyCas9 | 704 |
| TIGIT4244 | GCACATCTCCCCATCCTTCA | 20 | SpyCas9 | 705 |
| TIGIT4245 | CCCATCCTTCAAGGATCGAG | 20 | SpyCas9 | 706 |
| TIGIT4246 | CACTCGATCCTTGAAGGATG | 20 | SpyCas9 | 707 |
| TIGIT4247 | CCACTCGATCCTTGAAGGAT | 20 | SpyCas9 | 708 |
| TIGIT4248 | GCCACTCGATCCTTGAAGGA | 20 | SpyCas9 | 709 |
| TIGIT4249 | TTCAAGGATCGAGTGGCCCC | 20 | SpyCas9 | 710 |
| TIGIT4250 | TGGGGCCACTCGATCCTTGA | 20 | SpyCas9 | 711 |
| TIGIT4251 | GATCGAGTGGCCCCAGGTCC | 20 | SpyCas9 | 712 |
| TIGIT4252 | AGTGGCCCCAGGTCCCGGCC | 20 | SpyCas9 | 713 |
| TIGIT4253 | GTGGCCCCAGGTCCCGGCCT | 20 | SpyCas9 | 714 |
| TIGIT4254 | GAGGCCCAGGCCGGGACCTG | 20 | SpyCas9 | 715 |
| TIGIT4255 | TGAGGCCCAGGCCGGGACCT | 20 | SpyCas9 | 716 |
| TIGIT4256 | GTGAGGCCCAGGCCGGGACC | 20 | SpyCas9 | 717 |
| TIGIT4257 | TGGAGGGTGAGGCCCAGGCC | 20 | SpyCas9 | 718 |
| TIGIT4258 | CTGGAGGGTGAGGCCCAGGC | 20 | SpyCas9 | 719 |
| TIGIT4259 | GCGACTGGAGGGTGAGGCCC | 20 | SpyCas9 | 720 |
| TIGIT4260 | CGGTCAGCGACTGGAGGGTG | 20 | SpyCas9 | 721 |
| TIGIT4261 | GTTCACGGTCAGCGACTGGA | 20 | SpyCas9 | 722 |
| TIGIT4262 | CGTTCACGGTCAGCGACTGG | 20 | SpyCas9 | 723 |
| TIGIT4263 | TATCGTTCACGGTCAGCGAC | 20 | SpyCas9 | 724 |
| TIGIT4264 | TCGCTGACCGTGAACGATAC | 20 | SpyCas9 | 725 |
| TIGIT4265 | CGCTGACCGTGAACGATACA | 20 | SpyCas9 | 726 |
| TIGIT4266 | GCTGACCGTGAACGATACAG | 20 | SpyCas9 | 727 |
| TIGIT4267 | GTACTCCCCTGTATCGTTCA | 20 | SpyCas9 | 728 |
| TIGIT4268 | ATCTATCACACCTACCCTGA | 20 | SpyCas9 | 729 |
| TIGIT4269 | TCTATCACACCTACCCTGAT | 20 | SpyCas9 | 730 |
| TIGIT4270 | TACCCTGATGGGACGTACAC | 20 | SpyCas9 | 731 |
| TIGIT4271 | ACCCTGATGGGACGTACACT | 20 | SpyCas9 | 732 |
| TIGIT4272 | AGTGTACGTCCCATCAGGGT | 20 | SpyCas9 | 733 |
| TIGIT4273 | TCCCAGTGTACGTCCCATCA | 20 | SpyCas9 | 734 |
| TIGIT4274 | CTCCCAGTGTACGTCCCATC | 20 | SpyCas9 | 735 |
| TIGIT4275 | GTACACTGGGAGAATCTTCC | 20 | SpyCas9 | 736 |
| TIGIT4276 | CACTGGGAGAATCTTCCTGG | 20 | SpyCas9 | 737 |
| TIGIT4277 | CTGAGCTTTCTAGGACCTCC | 20 | SpyCas9 | 738 |
| TIGIT4278 | AGGTTCCAGATTCCATTGCT | 20 | SpyCas9 | 739 |
| TIGIT4279 | AAGCAATGGAATCTGGAACC | 20 | SpyCas9 | 740 |
| TIGIT4280 | GATTCCATTGCTTGGAGCCA | 20 | SpyCas9 | 741 |
| TIGIT4281 | TGGCTCCAAGCAATGGAATC | 20 | SpyCas9 | 742 |
| TIGIT4282 | GCGGCCATGGCTCCAAGCAA | 20 | SpyCas9 | 743 |
| TIGIT4283 | TGGAGCCATGGCCGCGACGC | 20 | SpyCas9 | 744 |
| TIGIT4284 | AGCCATGGCCGCGACGCTGG | 20 | SpyCas9 | 745 |
| TIGIT4285 | GACCACCAGCGTCGCGGCCA | 20 | SpyCas9 | 746 |
| TIGIT4286 | GCAGATGACCACCAGCGTCG | 20 | SpyCas9 | 747 |
| TIGIT4287 | CATCTGCACAGCAGTCATCG | 20 | SpyCas9 | 748 |
| TIGIT4288 | CTGCACAGCAGTCATCGTGG | 20 | SpyCas9 | 749 |
| TIGIT4289 | AGCCCTCAGAATCCATTCTG | 20 | SpyCas9 | 750 |
| TIGIT4290 | CTCAGAATCCATTCTGTGGA | 20 | SpyCas9 | 751 |
| TIGIT4291 | TTCCACAGAATGGATTCTGA | 20 | SpyCas9 | 752 |
| TIGIT4292 | CTTCCACAGAATGGATTCTG | 20 | SpyCas9 | 753 |
| TIGIT4293 | ATTCTGTGGAAGGTGACCTC | 20 | SpyCas9 | 754 |
| TIGIT4294 | TGAGGTCACCTTCCACAGAA | 20 | SpyCas9 | 755 |
| TIGIT4295 | GACCTCAGGAGAAAATCAGC | 20 | SpyCas9 | 756 |
| TIGIT4296 | CAGGAGAAAATCAGCTGGAC | 20 | SpyCas9 | 757 |
| TIGIT4297 | GTCCAGCTGATTTTCTCCTG | 20 | SpyCas9 | 758 |
| TIGIT4298 | GAGAAAATCAGCTGGACAGG | 20 | SpyCas9 | 759 |
| TIGIT4299 | AATCAGCTGGACAGGAGGAA | 20 | SpyCas9 | 760 |
| TIGIT4300 | CCCAGTGCTCCCTCACCCCC | 20 | SpyCas9 | 761 |
| TIGIT4301 | CTGGGGGTGAGGGAGCACTG | 20 | SpyCas9 | 762 |
| TIGIT4302 | CCTGGGGGTGAGGGAGCACT | 20 | SpyCas9 | 763 |
| TIGIT4303 | TCCTGGGGGTGAGGGAGCAC | 20 | SpyCas9 | 764 |
| TIGIT4304 | ACACAGCTTCCTGGGGGTGA | 20 | SpyCas9 | 765 |
| TIGIT4305 | GACACAGCTTCCTGGGGGTG | 20 | SpyCas9 | 766 |
| TIGIT4306 | ACCCCCAGGAAGCTGTGTCC | 20 | SpyCas9 | 767 |
| TIGIT4307 | GCCTGGACACAGCTTCCTGG | 20 | SpyCas9 | 768 |
| TIGIT4308 | TGCCTGGACACAGCTTCCTG | 20 | SpyCas9 | 769 |
| TIGIT4309 | CTGCCTGGACACAGCTTCCT | 20 | SpyCas9 | 770 |
| TIGIT4310 | TCTGCCTGGACACAGCTTCC | 20 | SpyCas9 | 771 |
| TIGIT4311 | CAGGCAGAAGCTGCACCTGC | 20 | SpyCas9 | 772 |
| TIGIT4312 | AGGCAGAAGCTGCACCTGCT | 20 | SpyCas9 | 773 |
| TIGIT4313 | CAGCAGGTGCAGCTTCTGCC | 20 | SpyCas9 | 774 |
| TIGIT4314 | GCTGCACCTGCTGGGCTCTG | 20 | SpyCas9 | 775 |
| TIGIT4315 | TGCTCTCCACAGAGCCCAGC | 20 | SpyCas9 | 776 |
| TIGIT4316 | CTGGGCTCTGTGGAGAGCAG | 20 | SpyCas9 | 777 |
| TIGIT4317 | TGGGCTCTGTGGAGAGCAGC | 20 | SpyCas9 | 778 |
| TIGIT4318 | GGGCTCTGTGGAGAGCAGCG | 20 | SpyCas9 | 779 |
| TIGIT4319 | CTGTGGAGAGCAGCGGGGAG | 20 | SpyCas9 | 780 |
| TIGIT4320 | ATTGAAGTAGTCATGCAGCT | 20 | SpyCas9 | 781 |
| TIGIT4321 | TGTCCTGAGTTACAGAAGCC | 20 | SpyCas9 | 782 |
| TIGIT4322 | GTCCTGAGTTACAGAAGCCT | 20 | SpyCas9 | 783 |
| TIGIT4323 | TACCCAGGCTTCTGTAACTC | 20 | SpyCas9 | 784 |
| TIGIT4324 | TGAAGAAGCTGCAGTTACCC | 20 | SpyCas9 | 785 |
| TIGIT4325 | TGCAGCTTCTTCACAGAGAC | 20 | SpyCas9 | 786 |
| TIGIT5053 | GTTGTTTCTATTGTGCCTGT | 20 | AsCpf1 RR | 787 |
| TIGIT5054 | CGTTGTTTCTATTGTGCCTG | 20 | AsCpf1 RR | 788 |
| TIGIT5055 | CCGTTGTTTCTATTGTGCCT | 20 | AsCpf1 RR | 789 |
| TIGIT5056 | CCACGGCACAAGTGACCCAG | 20 | AsCpf1 RR | 790 |
| TIGIT5057 | AGTTGACCTGGGTCACTTGT | 20 | AsCpf1 RR | 791 |
| TIGIT5058 | AAGTCAGCATTACAAATGGC | 20 | AsCpf1 RR | 792 |
| TIGIT5059 | CATCCTTCAAGGATCGAGTG | 20 | AsCpf1 RR | 793 |
| TIGIT5060 | ATCCTTCAAGGATCGAGTGG | 20 | AsCpf1 RR | 794 |
| TIGIT5061 | AGGATCGAGTGGCCCCAGGT | 20 | AsCpf1 RR | 795 |
| TIGIT5062 | AGGTCCCGGCCTGGGCCTCA | 20 | AsCpf1 RR | 796 |
| TIGIT5063 | GGCCTGGGCCTCACCCTCCA | 20 | AsCpf1 RR | 797 |
| TIGIT5064 | CGGTCAGCGACTGGAGGGTG | 20 | AsCpf1 RR | 798 |
| TIGIT5065 | GTCGCTGACCGTGAACGATA | 20 | AsCpf1 RR | 799 |
| TIGIT5066 | TGTATCGTTCACGGTCAGCG | 20 | AsCpf1 RR | 800 |
| TIGIT5067 | CTGTATCGTTCACGGTCAGC | 20 | AsCpf1 RR | 801 |
| TIGIT5068 | ATCAGGGTAGGTGTGATAGA | 20 | AsCpf1 RR | 802 |
| TIGIT5069 | AGTGTACGTCCCATCAGGGT | 20 | AsCpf1 RR | 803 |
| TIGIT5070 | GGAAGATTCTCCCAGTGTAC | 20 | AsCpf1 RR | 804 |
| TIGIT5071 | TGGAGGTCCTAGAAAGCTCA | 20 | AsCpf1 RR | 805 |
| TIGIT5072 | AGCAATGGAATCTGGAACCT | 20 | AsCpf1 RR | 806 |
| TIGIT5073 | AGATTCCATTGCTTGGAGCC | 20 | AsCpf1 RR | 807 |
| TIGIT5074 | GATTCCATTGCTTGGAGCCA | 20 | AsCpf1 RR | 808 |
| TIGIT5075 | ATTGCTTGGAGCCATGGCCG | 20 | AsCpf1 RR | 809 |
| TIGIT5076 | TTGCTTGGAGCCATGGCCGC | 20 | AsCpf1 RR | 810 |
| TIGIT5077 | CAGAATGGATTCTGAGGGCT | 20 | AsCpf1 RR | 811 |
| TIGIT5078 | ACAGAATGGATTCTGAGGGC | 20 | AsCpf1 RR | 812 |
| TIGIT5079 | TTCTGTGGAAGGTGACCTCA | 20 | AsCpf1 RR | 813 |
| TIGIT5080 | GCTGATTTTCTCCTGAGGTC | 20 | AsCpf1 RR | 814 |
| TIGIT5081 | TCCTGTCCAGCTGATTTTCT | 20 | AsCpf1 RR | 815 |
| TIGIT5082 | TTCCTCCTGTCCAGCTGATT | 20 | AsCpf1 RR | 816 |
| TIGIT5083 | TGGGGGTGAGGGAGCACTGG | 20 | AsCpf1 RR | 817 |
| TIGIT5084 | AGTGCTCCCTCACCCCCAGG | 20 | AsCpf1 RR | 818 |
| TIGIT5085 | TCACCCCCAGGAAGCTGTGT | 20 | AsCpf1 RR | 819 |
| TIGIT5086 | CAGGAAGCTGTGTCCAGGCA | 20 | AsCpf1 RR | 820 |
| TIGIT5087 | AGGAAGCTGTGTCCAGGCAG | 20 | AsCpf1 RR | 821 |
| TIGIT5088 | GGCAGAAGCTGCACCTGCTG | 20 | AsCpf1 RR | 822 |
| TIGIT5089 | CAGAGCCCAGCAGGTGCAGC | 20 | AsCpf1 RR | 823 |
| TIGIT5090 | GCTGCTCTCCACAGAGCCCA | 20 | AsCpf1 RR | 824 |
| TIGIT5091 | CGCTGCTCTCCACAGAGCCC | 20 | AsCpf1 RR | 825 |
| TIGIT5092 | ATGTCCTGAGTTACAGAAGC | 20 | AsCpf1 RR | 826 |
| TIGIT5093 | TGCAGAGAAAGGTGGCTCTAT | 21 | Cas12a | 1175 |

In some embodiments the gRNA for use in the disclosure is a gRNA targeting ADORA2a (ADORA2a gRNA). In some embodiments, the gRNA targeting ADORA2a is one or more of the gRNAs described in Table 9.

TABLE 9
ADORA2a gRNAs

| | gRNA Targeting Domain | | | SEQ ID |
|---|---|---|---|---|
| Name | Sequence (DNA) | Length | Enzyme | NO: |
| ADORA2A337 | GAGCACACCCACTGCGATGT | 20 | SpyCas9 | 827 |
| ADORA2A338 | GATGGCCAGGAGACTGAAGA | 20 | SpyCas9 | 828 |
| ADORA2A339 | CTGCTCACCGGAGCGGGATG | 20 | SpyCas9 | 829 |
| ADORA2A340 | GTCTGTGGCCATGCCCATCA | 20 | SpyCas9 | 830 |
| ADORA2A341 | TCACCGGAGCGGGATGCGGA | 20 | SpyCas9 | 831 |
| ADORA2A342 | GTGGCAGGCAGCGCAGAACC | 20 | SpyCas9 | 832 |
| ADORA2A343 | AGCACACCAGCACATTGCCC | 20 | SpyCas9 | 833 |
| ADORA2A344 | CAGGTTGCTGTTGAGCCACA | 20 | SpyCas9 | 834 |
| ADORA2A345 | CTTCATTGCCTGCTTCGTCC | 20 | SpyCas9 | 835 |
| ADORA2A346 | GTACACCGAGGAGCCCATGA | 20 | SpyCas9 | 836 |
| ADORA2A347 | GATGGCAATGTAGCGGTCAA | 20 | SpyCas9 | 837 |
| ADORA2A348 | CTCCTCGGTGTACATCACGG | 20 | SpyCas9 | 838 |
| ADORA2A349 | CGAGGAGCCCATGATGGGCA | 20 | SpyCas9 | 839 |
| ADORA2A350 | GGGCTCCTCGGTGTACATCA | 20 | SpyCas9 | 840 |
| ADORA2A351 | CTTTGTGGTGTCACTGGCGG | 20 | SpyCas9 | 841 |
| ADORA2A352 | CCGCTCCGGTGAGCAGGGCC | 20 | SpyCas9 | 842 |
| ADORA2A353 | GGGTTCTGCGCTGCCTGCCA | 20 | SpyCas9 | 843 |
| ADORA2A354 | GGACGAAGCAGGCAATGAAG | 20 | SpyCas9 | 844 |
| ADORA2A355 | GTGCTGATGGTGATGGCAAA | 20 | SpyCas9 | 845 |
| ADORA2A356 | AGCGCAGAACCCGGTGCTGA | 20 | SpyCas9 | 846 |
| ADORA2A357 | GAGCTCCATCTTCAGTCTCC | 20 | SpyCas9 | 847 |
| ADORA2A358 | TGCTGATGGTGATGGCAAAG | 20 | SpyCas9 | 848 |
| ADORA2A359 | GGCGGCGGCCGACATCGCAG | 20 | SpyCas9 | 849 |
| ADORA2A360 | AATGAAGAGGCAGCCGTGGC | 20 | SpyCas9 | 850 |
| ADORA2A361 | GGGCAATGTGCTGGTGTGCT | 20 | SpyCas9 | 851 |
| ADORA2A362 | CATGCCCATCATGGGCTCCT | 20 | SpyCas9 | 852 |
| ADORA2A363 | AATGTAGCGGTCAATGGCGA | 20 | SpyCas9 | 853 |
| ADORA2A364 | AGTAGTTGGTGACGTTCTGC | 20 | SpyCas9 | 854 |
| ADORA2A365 | AGCGGTCAATGGCGATGGCC | 20 | SpyCas9 | 855 |
| ADORA2A366 | CGCATCCCGCTCCGGTGAGC | 20 | SpyCas9 | 856 |
| ADORA2A367 | GCATCCCGCTCCGGTGAGCA | 20 | SpyCas9 | 857 |
| ADORA2A368 | TGGGCAATGTGCTGGTGTGC | 20 | SpyCas9 | 858 |
| ADORA2A369 | CAACTACTTTGTGGTGTCAC | 20 | SpyCas9 | 859 |
| ADORA2A370 | CGCTCCGGTGAGCAGGGCCG | 20 | SpyCas9 | 860 |
| ADORA2A371 | GATGGTGATGGCAAAGGGGA | 20 | SpyCas9 | 861 |
| ADORA2A372 | GGTGTACATCACGGTGGAGC | 20 | SpyCas9 | 862 |
| ADORA2A373 | GAACGTCACCAACTACTTTG | 20 | SpyCas9 | 863 |
| ADORA2A374 | CAGTGACACCACAAAGTAGT | 20 | SpyCas9 | 864 |
| ADORA2A375 | GGCCATCCTGGGCAATGTGC | 20 | SpyCas9 | 865 |
| ADORA2A376 | CCCGGCCCTGCTCACCGGAG | 20 | SpyCas9 | 866 |
| ADORA2A377 | CACCAGCACATTGCCCAGGA | 20 | SpyCas9 | 867 |
| ADORA2A378 | TTTGCCATCACCATCAGCAC | 20 | SpyCas9 | 868 |
| ADORA2A379 | CTCCACCGTGATGTACACCG | 20 | SpyCas9 | 869 |
| ADORA2A380 | GGAGCTGGCCATTGCTGTGC | 20 | SpyCas9 | 870 |
| ADORA2A381 | CAGGATGGCCAGCACAGCAA | 20 | SpyCas9 | 871 |
| ADORA2A382 | GAACCCGGTGCTGATGGTGA | 20 | SpyCas9 | 872 |
| ADORA2A383 | TGGAGCTCTGCGTGAGGACC | 20 | SpyCas9 | 873 |
| ADORA2A384 | CCCGCTCCGGTGAGCAGGGC | 20 | SpyCas9 | 874 |
| ADORA2A385 | AGGCAATGAAGAGGCAGCCG | 20 | SpyCas9 | 875 |
| ADORA2A386 | CCGGCCCTGCTCACCGGAGC | 20 | SpyCas9 | 876 |
| ADORA2A387 | GCGGCGGCCGACATCGCAGT | 20 | SpyCas9 | 877 |
| ADORA2A388 | GGTGCTGATGGTGATGGCAA | 20 | SpyCas9 | 878 |
| ADORA2A389 | CTACTTTGTGGTGTCACTGG | 20 | SpyCas9 | 879 |
| ADORA2A390 | TACACCGAGGAGCCCATGAT | 20 | SpyCas9 | 880 |
| ADORA2A391 | TCTGTGGCCATGCCCATCAT | 20 | SpyCas9 | 881 |
| ADORA2A392 | ATTGCTGTGCTGGCCATCCT | 20 | SpyCas9 | 882 |
| ADORA2A393 | CGTGAGGACCAGGACGAAGC | 20 | SpyCas9 | 883 |
| ADORA2A394 | TTGCCATCACCATCAGCACC | 20 | SpyCas9 | 884 |
| ADORA2A395 | GGATGCGGATGGCAATGTAG | 20 | SpyCas9 | 885 |
| ADORA2A396 | TTGCCATCCGCATCCCGCTC | 20 | SpyCas9 | 886 |
| ADORA2A397 | TGAAGATGGAGCTCTGCGTG | 20 | SpyCas9 | 887 |
| ADORA2A398 | CATTGCTGTGCTGGCCATCC | 20 | SpyCas9 | 888 |
| ADORA2A399 | TGCTGGTGTGCTGGGCCGTG | 20 | SpyCas9 | 889 |
| ADORA2A820 | GGCTCCTCGGTGTACATCACG | 21 | SauCas9 | 890 |
| ADORA2A821 | GAGCTCTGCGTGAGGACCAGG | 21 | SauCas9 | 891 |
| ADORA2A822 | GATGGAGCTCTGCGTGAGGAC | 21 | SauCas9 | 892 |
| ADORA2A823 | CCAGCACACCAGCACATTGCC | 21 | SauCas9 | 893 |
| ADORA2A824 | AGGACCAGGACGAAGCAGGCA | 21 | SauCas9 | 894 |
| ADORA2A825 | TGCCATCCGCATCCCGCTCCG | 21 | SauCas9 | 895 |
| ADORA2A826 | GTGTGGCTCAACAGCAACCTG | 21 | SauCas9 | 896 |
| ADORA2A827 | AGCTCCACCGTGATGTACACC | 21 | SauCas9 | 897 |
| ADORA2A828 | GTAGCGGTCAATGGCGATGGC | 21 | SauCas9 | 898 |
| ADORA2A829 | CGGTGCTGATGGTGATGGCAA | 21 | SauCas9 | 899 |
| ADORA2A830 | CCCTGCTCACCGGAGCGGGAT | 21 | SauCas9 | 900 |
| ADORA2A831 | GTGACGTTCTGCAGGTTGCTG | 21 | SauCas9 | 901 |
| ADORA2A832 | GCTCCACCGTGATGTACACCG | 21 | SauCas9 | 902 |
| ADORA2A833 | ACTGAAGATGGAGCTCTGCGT | 21 | SauCas9 | 903 |
| ADORA2A834 | CCAGCTCCACCGTGATGTACA | 21 | SauCas9 | 904 |
| ADORA2A835 | CCTTTGCCATCACCATCAGCA | 21 | SauCas9 | 905 |
| ADORA2A836 | CCGGTGCTGATGGTGATGGCA | 21 | SauCas9 | 906 |
| ADORA2A837 | CCTGGGCAATGTGCTGGTGTG | 21 | SauCas9 | 907 |
| ADORA2A838 | AGGCAGCCGTGGCAGGCAGCG | 21 | SauCas9 | 908 |
| ADORA2A839 | GCGATGGCCAGGAGACTGAAG | 21 | SauCas9 | 909 |
| ADORA2A840 | CGATGGCCAGGAGACTGAAGA | 21 | SauCas9 | 910 |
| ADORA2A841 | TCCCGCTCCGGTGAGCAGGGC | 21 | SauCas9 | 911 |
| ADORA2A842 | TGCTTCGTCCTGGTCCTCACG | 21 | SauCas9 | 912 |
| ADORA2A843 | ACCAGGACGAAGCAGGCAATG | 21 | SauCas9 | 913 |
| ADORA2A844 | ATGTACACCGAGGAGCCCATG | 21 | SauCas9 | 914 |
| ADORA2A845 | TCGTCTGTGGCCATGCCCATC | 21 | SauCas9 | 915 |
| ADORA2A846 | TCAATGGCGATGGCCAGGAGA | 21 | SauCas9 | 916 |
| ADORA2A847 | GGTGCTGATGGTGATGGCAAA | 21 | SauCas9 | 917 |
| ADORA2A848 | TAGCGGTCAATGGCGATGGCC | 21 | SauCas9 | 918 |
| ADORA2A849 | TCCGCATCCCGCTCCGGTGAG | 21 | SauCas9 | 919 |
| ADORA2A850 | CTGGCGGCGGCCGACATCGCA | 21 | SauCas9 | 920 |
| ADORA2A851 | GCCATTGCTGTGCTGGCCATC | 21 | SauCas9 | 921 |
| ADORA2A852 | ATCCCGCTCCGGTGAGCAGGG | 21 | SauCas9 | 922 |
| ADORA2A853 | AGACTGAAGATGGAGCTCTGC | 21 | SauCas9 | 923 |
| ADORA2A854 | CCCCGGCCCTGCTCACCGGAG | 21 | SauCas9 | 924 |
| ADORA2A855 | ATGGTGATGGCAAAGGGGATG | 21 | SauCas9 | 925 |
| ADORA2A856 | GCTCCTCGGTGTACATCACGG | 21 | SauCas9 | 926 |
| ADORA2A248 | TGTCGATGGCAATAGCCAAG | 20 | SpyCas9 | 927 |
| ADORA2A249 | AGAAGTTGGTGACGTTCTGC | 20 | SpyCas9 | 928 |
| ADORA2A250 | TTCGCCATCACCATCAGCAC | 20 | SpyCas9 | 929 |
| ADORA2A251 | GAAGAAGAGGCAGCCATGGC | 20 | SpyCas9 | 930 |
| ADORA2A252 | CACAAGCACGTTACCCAGGA | 20 | SpyCas9 | 931 |
| ADORA2A253 | CAACTTCTTCGTGGTATCTC | 20 | SpyCas9 | 932 |
| ADORA2A254 | CAGGATGGCCAGCACAGCAA | 20 | SpyCas9 | 933 |
| ADORA2A255 | AATTCCACTCCGGTGAGCCA | 20 | SpyCas9 | 934 |
| ADORA2A256 | AGCGCAGAAGCCAGTGCTGA | 20 | SpyCas9 | 935 |
| ADORA2A257 | GTGCTGATGGTGATGGCGAA | 20 | SpyCas9 | 936 |
| ADORA2A258 | GGAGCTGGCCATTGCTGTGC | 20 | SpyCas9 | 937 |
| ADORA2A259 | AATAGCCAAGAGGCTGAAGA | 20 | SpyCas9 | 938 |
| ADORA2A260 | CTCCTCGGTGTACATCATGG | 20 | SpyCas9 | 939 |
| ADORA2A261 | GGACAAAGCAGGCGAAGAAG | 20 | SpyCas9 | 940 |
| ADORA2A262 | TCTGGCGGCGGCTGACATCG | 20 | SpyCas9 | 941 |
| ADORA2A263 | TGGGTAACGTGCTTGTGTGC | 20 | SpyCas9 | 942 |
| ADORA2A264 | GATGTACACCGAGGAGCCCA | 20 | SpyCas9 | 943 |
| ADORA2A265 | TAACCCCTGGCTCACCGGAG | 20 | SpyCas9 | 944 |
| ADORA2A266 | TCACCGGAGTGGAATTCGGA | 20 | SpyCas9 | 945 |
| ADORA2A267 | GCGGCGGCTGACATCGCGGT | 20 | SpyCas9 | 946 |
| ADORA2A268 | GATGGTGATGGCGAATGGGA | 20 | SpyCas9 | 947 |
| ADORA2A269 | GGCTTCTGCGCTGCCTGCCA | 20 | SpyCas9 | 948 |
| ADORA2A270 | ATTCCACTCCGGTGAGCCAG | 20 | SpyCas9 | 949 |
| ADORA2A271 | GGTGTACATCATGGTGGAGC | 20 | SpyCas9 | 950 |
| ADORA2A272 | ATTGCTGTGCTGGCCATCCT | 20 | SpyCas9 | 951 |
| ADORA2A273 | CTCCACCATGATGTACACCG | 20 | SpyCas9 | 952 |
| ADORA2A274 | GGCGGCGGCTGACATCGCGG | 20 | SpyCas9 | 953 |
| ADORA2A275 | TACACCGAGGAGCCCATGGC | 20 | SpyCas9 | 954 |
| ADORA2A276 | GGGTAACGTGCTTGTGTGCT | 20 | SpyCas9 | 955 |
| ADORA2A277 | CAGGTTGCTGTTGATCCACA | 20 | SpyCas9 | 956 |
| ADORA2A278 | TGAAGATGGAACTCTGCGTG | 20 | SpyCas9 | 957 |
| ADORA2A279 | GATGGCGATGTATCTGTCGA | 20 | SpyCas9 | 958 |
| ADORA2A280 | CTTCTTCGCCTGCTTTGTCC | 20 | SpyCas9 | 959 |
| ADORA2A281 | AGGCGAAGAAGAGGCAGCCA | 20 | SpyCas9 | 960 |
| ADORA2A282 | TGCTTGTGTGCTGGGCCGTG | 20 | SpyCas9 | 961 |
| ADORA2A283 | GAAGCCAGTGCTGATGGTGA | 20 | SpyCas9 | 962 |
| ADORA2A284 | CGTGAGGACCAGGACAAAGC | 20 | SpyCas9 | 963 |
| ADORA2A285 | TGGAACTCTGCGTGAGGACC | 20 | SpyCas9 | 964 |
| ADORA2A286 | CATTGCTGTGCTGGCCATCC | 20 | SpyCas9 | 965 |
| ADORA2A287 | TTCTCCCGCCATGGGCTCCT | 20 | SpyCas9 | 966 |
| ADORA2A288 | TGGCTCACCGGAGTGGAATT | 20 | SpyCas9 | 967 |
| ADORA2A289 | TGCTGATGGTGATGGCGAAT | 20 | SpyCas9 | 968 |
| ADORA2A290 | CTTCGTGGTATCTCTGGCGG | 20 | SpyCas9 | 969 |
| ADORA2A291 | AGCACACAAGCACGTTACCC | 20 | SpyCas9 | 970 |
| ADORA2A292 | GGGCTCCTCGGTGTACATCA | 20 | SpyCas9 | 971 |
| ADORA2A293 | GTACACCGAGGAGCCCATGG | 20 | SpyCas9 | 972 |
| ADORA2A294 | GAACGTCACCAACTTCTTCG | 20 | SpyCas9 | 973 |
| ADORA2A295 | TCGCCATCCGAATTCCACTC | 20 | SpyCas9 | 974 |
| ADORA2A296 | GAGTTCCATCTTCAGCCTCT | 20 | SpyCas9 | 975 |
| ADORA2A297 | GAATTCCACTCCGGTGAGCC | 20 | SpyCas9 | 976 |
| ADORA2A298 | CAGAGATACCACGAAGAAGT | 20 | SpyCas9 | 977 |
| ADORA2A299 | CTTCTTCGTGGTATCTCTGG | 20 | SpyCas9 | 978 |
| ADORA2A695 | CAGTGCTGATGGTGATGGCGA | 21 | SauCas9 | 979 |
| ADORA2A696 | CGAATTCCACTCCGGTGAGCC | 21 | SauCas9 | 980 |
| ADORA2A697 | CCGAATTCCACTCCGGTGAGC | 21 | SauCas9 | 981 |
| ADORA2A698 | GCTGAAGATGGAACTCTGCGT | 21 | SauCas9 | 982 |
| ADORA2A699 | CGTGCTTGTGTGCTGGGCCGT | 21 | SauCas9 | 983 |
| ADORA2A700 | GTGAGGACCAGGACAAAGCAG | 21 | SauCas9 | 984 |
| ADORA2A701 | TCGATGGCAATAGCCAAGAGG | 21 | SauCas9 | 985 |
| ADORA2A702 | CATCGACAGATACATCGCCAT | 21 | SauCas9 | 986 |
| ADORA2A703 | GTACACCGAGGAGCCCATGGC | 21 | SauCas9 | 987 |
| ADORA2A704 | GCTCCACCATGATGTACACCG | 21 | SauCas9 | 988 |
| ADORA2A705 | AAGCCAGTGCTGATGGTGATG | 21 | SauCas9 | 989 |
| ADORA2A706 | CACCGCGATGTCAGCCGCCGC | 21 | SauCas9 | 990 |
| ADORA2A707 | AGGCTGAAGATGGAACTCTGC | 21 | SauCas9 | 991 |
| ADORA2A708 | GCCGCCGCCAGAGATACCACG | 21 | SauCas9 | 992 |
| ADORA2A709 | AGCTCCACCATGATGTACACC | 21 | SauCas9 | 993 |
| ADORA2A710 | AGGCAGCCATGGCAGGCAGCG | 21 | SauCas9 | 994 |
| ADORA2A711 | CCTGGCTCACCGGAGTGGAAT | 21 | SauCas9 | 995 |
| ADORA2A712 | CCAGCTCCACCATGATGTACA | 21 | SauCas9 | 996 |
| ADORA2A713 | ACCAGGACAAAGCAGGCGAAG | 21 | SauCas9 | 997 |
| ADORA2A714 | CCTGGGTAACGTGCTTGTGTG | 21 | SauCas9 | 998 |
| ADORA2A715 | AGGACCAGGACAAAGCAGGCG | 21 | SauCas9 | 999 |
| ADORA2A716 | TCAGCCGCCGCCAGAGATACC | 21 | SauCas9 | 1000 |
| ADORA2A717 | GGCTCCTCGGTGTACATCATG | 21 | SauCas9 | 1001 |
| ADORA2A718 | CTGGCGGCGGCTGACATCGCG | 21 | SauCas9 | 1002 |
| ADORA2A719 | GATGGAACTCTGCGTGAGGAC | 21 | SauCas9 | 1003 |
| ADORA2A720 | GCTCCTCGGTGTACATCATGG | 21 | SauCas9 | 1004 |
| ADORA2A721 | TGTACACCGAGGAGCCCATGG | 21 | SauCas9 | 1005 |
| ADORA2A722 | GCCATTGCTGTGCTGGCCATC | 21 | SauCas9 | 1006 |
| ADORA2A723 | CAATAGCCAAGAGGCTGAAGA | 21 | SauCas9 | 1007 |
| ADORA2A724 | ATGGTGATGGCGAATGGGATG | 21 | SauCas9 | 1008 |
| ADORA2A725 | ATGTACACCGAGGAGCCCATG | 21 | SauCas9 | 1009 |
| ADORA2A726 | GTGTGGATCAACAGCAACCTG | 21 | SauCas9 | 1010 |
| ADORA2A727 | TGCTTTGTCCTGGTCCTCACG | 21 | SauCas9 | 1011 |
| ADORA2A728 | GTAACCCCTGGCTCACCGGAG | 21 | SauCas9 | 1012 |
| ADORA2A729 | CCAGCACACAAGCACGTTACC | 21 | SauCas9 | 1013 |
| ADORA2A730 | TATCTGTCGATGGCAATAGCC | 21 | SauCas9 | 1014 |
| ADORA2A731 | GCAATAGCCAAGAGGCTGAAG | 21 | SauCas9 | 1015 |
| ADORA2A732 | AGTGCTGATGGTGATGGCGAA | 21 | SauCas9 | 1016 |
| ADORA2A733 | ACACCGAGGAGCCCATGGCGG | 21 | SauCas9 | 1017 |
| ADORA2A734 | CGCCATCCGAATTCCACTCCG | 21 | SauCas9 | 1018 |
| ADORA2A4111 | TGGTGTCACTGGCGGCGGCC | 20 | AsCpf1 | 1019 |
| ADORA2A4112 | CCATCACCATCAGCACCGGG | 20 | AsCpf1 | 1020 |
| ADORA2A4113 | CCATCGGCCTGACTCCCATG | 20 | AsCpf1 | 1021 |
| ADORA2A4114 | GCTGACCGCAGTTGTTCCAA | 20 | AsCpf1 | 1022 |
| ADORA2A4115 | AGGATGTGGTCCCCATGAAC | 20 | AsCpf1 | 1023 |
| ADORA2A4116 | CCTGTGTGCTGGTGCCCCTG | 20 | AsCpf1 | 1024 |
| ADORA2A4117 | CGGATCTTCCTGGCGGCGCG | 20 | AsCpf1 | 1025 |
| ADORA2A4118 | CCCTCTGCTGGCTGCCCCTA | 20 | AsCpf1 | 1026 |
| ADORA2A4119 | TTCTGCCCCGACTGCAGCCA | 20 | AsCpf1 | 1027 |
| ADORA2A4120 | AAGGCAGCTGGCACCAGTGC | 20 | AsCpf1 | 1028 |
| ADORA2A4121 | TAAGGGCATCATTGCCATCTG | 21 | SauCas9 | 1029 |
| ADORA2A4122 | CGGCCTGACTCCCATGCTAGG | 21 | SauCas9 | 1030 |
| ADORA2A4123 | GCAGTTGTTCCAACCTAGCAT | 21 | SauCas9 | 1031 |
| ADORA2A4124 | CCGCAGTTGTTCCAACCTAGC | 21 | SauCas9 | 1032 |
| ADORA2A4125 | CAAGAACCACTCCCAGGGCTG | 21 | SauCas9 | 1033 |
| ADORA2A4126 | CTTGGCCCTCCCCGCAGCCCT | 21 | SauCas9 | 1034 |
| ADORA2A4127 | CACTTGGCCCTCCCCGCAGCC | 21 | SauCas9 | 1035 |
| ADORA2A4128 | GGCCAAGTGGCCTGTCTCTTT | 21 | SauCas9 | 1036 |
| ADORA2A4129 | TTCATGGGGACCACATCCTCA | 21 | SauCas9 | 1037 |
| ADORA2A4130 | TGAAGTACACCATGTAGTTCA | 21 | SauCas9 | 1038 |
| ADORA2A4131 | CTGGTGCCCCTGCTGCTCATG | 21 | SauCas9 | 1039 |
| ADORA2A4132 | GCTCATGCTGGGTGTCTATTT | 21 | SauCas9 | 1040 |
| ADORA2A4133 | CTTCAGCTGTCGTCGCGCCGC | 21 | SauCas9 | 1041 |
| ADORA2A4134 | CGCGACGACAGCTGAAGCAGA | 21 | SauCas9 | 1042 |
| ADORA2A4135 | GATGGAGAGCCAGCCTCTGCC | 21 | SauCas9 | 1043 |
| ADORA2A4136 | GCGTGGCTGCAGTCGGGGCAG | 21 | SauCas9 | 1044 |
| ADORA2A4137 | ACGATGGCCAGGTACATGAGC | 21 | SauCas9 | 1045 |
| ADORA2A4138 | CTCTCCCACACCAATTCGGTT | 21 | SauCas9 | 1046 |
| ADORA2A4139 | GATTCACAACCGAATTGGTGT | 21 | SauCas9 | 1047 |
| ADORA2A4140 | GGGATTCACAACCGAATTGGT | 21 | SauCas9 | 1048 |
| ADORA2A4141 | CGTAGATGAAGGGATTCACAA | 21 | SauCas9 | 1049 |
| ADORA2A4142 | GGATACGGTAGGCGTAGATGA | 21 | SauCas9 | 1050 |
| ADORA2A4143 | TCATCTACGCCTACCGTATCC | 21 | SauCas9 | 1051 |
| ADORA2A4144 | CGGATACGGTAGGCGTAGATG | 21 | SauCas9 | 1052 |
| ADORA2A4145 | GCGGAAGGTCTGGCGGAACTC | 21 | SauCas9 | 1053 |
| ADORA2A4146 | AATGATCTTGCGGAAGGTCTG | 21 | SauCas9 | 1054 |
| ADORA2A4147 | GACGTGGCTGCGAATGATCTT | 21 | SauCas9 | 1055 |
| ADORA2A4148 | TTGCTGCCTCAGGACGTGGCT | 21 | SauCas9 | 1056 |
| ADORA2A4149 | CAAGGCAGCTGGCACCAGTGC | 21 | SauCas9 | 1057 |
| ADORA2A4150 | CGGGCACTGGTGCCAGCTGCC | 21 | SauCas9 | 1058 |
| ADORA2A4151 | CTTGGCAGCTCATGGCAGTGA | 21 | SauCas9 | 1059 |
| ADORA2A4152 | CCGTCTCAACGGCCACCCGCC | 21 | SauCas9 | 1060 |
| ADORA2A4153 | CACACTCCTGGCGGGTGGCCG | 21 | SauCas9 | 1061 |
| ADORA2A4154 | TGCCGTTGGCCCACACTCCTG | 21 | SauCas9 | 1062 |
| ADORA2A4155 | CCATTGGGCCTCCGCTCAGGG | 21 | SauCas9 | 1063 |
| ADORA2A4156 | CATAGCCATTGGGCCTCCGCT | 21 | SauCas9 | 1064 |
| ADORA2A4157 | AATGGCTATGCCCTGGGGCTG | 21 | SauCas9 | 1065 |
| ADORA2A4158 | ATGCCCTGGGGCTGGTGAGTG | 21 | SauCas9 | 1066 |
| ADORA2A4159 | GCCCTGGGGCTGGTGAGTGGA | 21 | SauCas9 | 1067 |
| ADORA2A4160 | TGGTGAGTGGAGGGAGTGCCC | 21 | SauCas9 | 1068 |
| ADORA2A4161 | GAGGGAGTGCCCAAGAGTCCC | 21 | SauCas9 | 1069 |
| ADORA2A4162 | AGGGAGTGCCCAAGAGTCCCA | 21 | SauCas9 | 1070 |
| ADORA2A4163 | GTCTGGGAGGCCCGTGTTCCC | 21 | SauCas9 | 1071 |
| ADORA2A4164 | CATGGCTAAGGAGCTCCACGT | 21 | SauCas9 | 1072 |
| ADORA2A4165 | GAGCTCCTTAGCCATGAGCTC | 21 | SauCas9 | 1073 |
| ADORA2A4166 | GCTCCTTAGCCATGAGCTCAA | 21 | SauCas9 | 1074 |
| ADORA2A4167 | GGCCTAGATGACCCCCTGGCC | 21 | SauCas9 | 1075 |
| ADORA2A4168 | CCCCCTGGCCCAGGATGGAGC | 21 | SauCas9 | 1076 |
| ADORA2A4169 | CTCCTGCTCCATCCTGGGCCA | 21 | SauCas9 | 1077 |
| ADORA2A4416 | CCGTGATGTACACCGAGGAG | 20 | AsCpf1 RR | 1078 |
| ADORA2A4417 | CTTTGCCATCACCATCAGCA | 20 | AsCpf1 RR | 1079 |
| ADORA2A4418 | TTTGCCATCACCATCAGCAC | 20 | AsCpf1 RR | 1080 |
| ADORA2A4419 | TTGCCTGCTTCGTCCTGGTC | 20 | AsCpf1 RR | 1081 |
| ADORA2A4420 | TCCTGGTCCTCACGCAGAGC | 20 | AsCpf1 RR | 1082 |
| ADORA2A4421 | TCTTCAGTCTCCTGGCCATC | 20 | AsCpf1 RR | 1083 |
| ADORA2A4422 | GTCTCCTGGCCATCGCCATT | 20 | AsCpf1 RR | 1084 |
| ADORA2A4423 | ACCTAGCATGGGAGTCAGGC | 20 | AsCpf1 RR | 1085 |
| ADORA2A4424 | AACCTAGCATGGGAGTCAGG | 20 | AsCpf1 RR | 1086 |
| ADORA2A4425 | ATGCTAGGTTGGAACAACTG | 20 | AsCpf1 RR | 1087 |
| ADORA2A4426 | GCAGCCCTGGGAGTGGTTCT | 20 | AsCpf1 RR | 1088 |
| ADORA2A4427 | CGCAGCCCTGGGAGTGGTTC | 20 | AsCpf1 RR | 1089 |
| ADORA2A4428 | AGGGCTGCGGGGAGGGCCAA | 20 | AsCpf1 RR | 1090 |
| ADORA2A4429 | TGGGGACCACATCCTCAAAG | 20 | AsCpf1 RR | 1091 |
| ADORA2A4430 | CATGAACTACATGGTGTACT | 20 | AsCpf1 RR | 1092 |
| ADORA2A4431 | ATGAACTACATGGTGTACTT | 20 | AsCpf1 RR | 1093 |
| ADORA2A4432 | ACTTCTTTGCCTGTGTGCTG | 20 | AsCpf1 RR | 1094 |
| ADORA2A4433 | TGCTGCTCATGCTGGGTGTC | 20 | AsCpf1 RR | 1095 |
| ADORA2A4434 | CAAATAGACACCCAGCATGA | 20 | AsCpf1 RR | 1096 |
| ADORA2A4435 | GCTGTCGTCGCGCCGCCAGG | 20 | AsCpf1 RR | 1097 |
| ADORA2A4436 | TGGCGGCGCGACGACAGCTG | 20 | AsCpf1 RR | 1098 |
| ADORA2A4437 | TCTGCTTCAGCTGTCGTCGC | 20 | AsCpf1 RR | 1099 |
| ADORA2A4438 | GGCAGAGGCTGGCTCTCCAT | 20 | AsCpf1 RR | 1100 |
| ADORA2A4439 | CGGCAGAGGCTGGCTCTCCA | 20 | AsCpf1 RR | 1101 |
| ADORA2A4440 | CCGGCAGAGGCTGGCTCTCC | 20 | AsCpf1 RR | 1102 |
| ADORA2A4441 | CACTGCAGAAGGAGGTCCAT | 20 | AsCpf1 RR | 1103 |
| ADORA2A4442 | TGCTGCCAAGTCACTGGCCA | 20 | AsCpf1 RR | 1104 |
| ADORA2A4443 | ACAATGATGGCCAGTGACTT | 20 | AsCpf1 RR | 1105 |
| ADORA2A4444 | TACACATCATCAACTGCTTC | 20 | AsCpf1 RR | 1106 |
| ADORA2A4445 | CTTTCTTCTGCCCCGACTGC | 20 | AsCpf1 RR | 1107 |
| ADORA2A4446 | GACTGCAGCCACGCCCCTCT | 20 | AsCpf1 RR | 1108 |
| ADORA2A4447 | TCTCTGGCTCATGTACCTGG | 20 | AsCpf1 RR | 1109 |
| ADORA2A4448 | CAACCGAATTGGTGTGGGAG | 20 | AsCpf1 RR | 1110 |
| ADORA2A4449 | ACACCAATTCGGTTGTGAAT | 20 | AsCpf1 RR | 1111 |
| ADORA2A4450 | GTTGTGAATCCCTTCATCTA | 20 | AsCpf1 RR | 1112 |
| ADORA2A4451 | TTCATCTACGCCTACCGTAT | 20 | AsCpf1 RR | 1113 |
| ADORA2A4452 | TCTACGCCTACCGTATCCGC | 20 | AsCpf1 RR | 1114 |
| ADORA2A4453 | CGAGTTCCGCCAGACCTTCC | 20 | AsCpf1 RR | 1115 |
| ADORA2A4454 | GCCAGACCTTCCGCAAGATC | 20 | AsCpf1 RR | 1116 |
| ADORA2A4455 | CCAGACCTTCCGCAAGATCA | 20 | AsCpf1 RR | 1117 |
| ADORA2A4456 | GCAAGATCATTCGCAGCCAC | 20 | AsCpf1 RR | 1118 |
| ADORA2A4457 | CAAGATCATTCGCAGCCACG | 20 | AsCpf1 RR | 1119 |
| ADORA2A4458 | CAGCCACGTCCTGAGGCAGC | 20 | AsCpf1 RR | 1120 |
| ADORA2A4459 | AGGCAGCTGGCACCAGTGCC | 20 | AsCpf1 RR | 1121 |
| ADORA2A4460 | TCACTGCCATGAGCTGCCAA | 20 | AsCpf1 RR | 1122 |
| ADORA2A4461 | TCTCAACGGCCACCCGCCAG | 20 | AsCpf1 RR | 1123 |
| ADORA2A4462 | CTCAGGGTGGGGAGCACTGC | 20 | AsCpf1 RR | 1124 |
| ADORA2A4463 | CACCCTGAGCGGAGGCCCAA | 20 | AsCpf1 RR | 1125 |
| ADORA2A4464 | ACCCTGAGCGGAGGCCCAAT | 20 | AsCpf1 RR | 1126 |
| ADORA2A4465 | AGGGCATAGCCATTGGGCCT | 20 | AsCpf1 RR | 1127 |
| ADORA2A4466 | CTCACCAGCCCCAGGGCATA | 20 | AsCpf1 RR | 1128 |
| ADORA2A4467 | TCCACTCACCAGCCCCAGGG | 20 | AsCpf1 RR | 1129 |
| ADORA2A4468 | TGGGACTCTTGGGCACTCCC | 20 | AsCpf1 RR | 1130 |
| ADORA2A4469 | CTGGGACTCTTGGGCACTCC | 20 | AsCpf1 RR | 1131 |
| ADORA2A4470 | CCTGGGACTCTTGGGCACTC | 20 | AsCpf1 RR | 1132 |
| ADORA2A4471 | AGGGGAACACGGGCCTCCCA | 20 | AsCpf1 RR | 1133 |
| ADORA2A4472 | CGTCTGGGAGGCCCGTGTTC | 20 | AsCpf1 RR | 1134 |
| ADORA2A4473 | AGACGTGGAGCTCCTTAGCC | 20 | AsCpf1 RR | 1135 |
| ADORA2A4474 | TTGAGCTCATGGCTAAGGAG | 20 | AsCpf1 RR | 1136 |
| ADORA2A4475 | CTGGCCTAGATGACCCCCTG | 20 | AsCpf1 RR | 1137 |
| ADORA2A4476 | TGGCCTAGATGACCCCCTGG | 20 | AsCpf1 RR | 1138 |
| ADORA2A4477 | TCCTGGGCCAGGGGGTCATC | 20 | AsCpf1 RR | 1139 |
| ADORA2A4478 | CTGGCCCAGGATGGAGCAGG | 20 | AsCpf1 RR | 1140 |
| ADORA2A4479 | TGGCCCAGGATGGAGCAGGA | 20 | AsCpf1 RR | 1141 |
| ADORA2A4480 | CGCGAGTTCCGCCAGACCTT | 20 | AsCpf1 RVR | 1142 |
| ADORA2A4481 | CCCTGGGGCTGGTGAGTGGA | 20 | AsCpf1 RVR | 1143 |
| ADORA2A4482 | CCATCGGCCTGACTCCCATGC | 21 | Cas12a | 1174 |

It will be understood that the exemplary gRNAs disclosed herein are provided to illustrate non-limiting embodiments embraced by the present disclosure. Additional suitable gRNA sequences will be apparent to the skilled artisan based on the present disclosure, and the disclosure is not limited in this respect.

RNA-Guided Nucleases

RNA-guided nucleases according to the present disclosure include, but are not limited to, naturally-occurring Class 2 CRISPR nucleases such as Cas9, and Cpf1, as well as other nucleases derived or obtained therefrom. In functional terms, RNA-guided nucleases are defined as those nucleases that: (a) interact with (e.g., complex with) a gRNA; and (b) together with the gRNA, associate with, and optionally cleave or modify, a target region of a DNA that includes (i) a sequence complementary to the targeting domain of the gRNA and, optionally, (ii) an additional sequence referred to as a “protospacer adjacent motif,” or “PAM,” which is described in greater detail below. As the following examples will illustrate, RNA-guided nucleases can be defined, in broad terms, by their PAM specificity and cleavage activity, even though variations may exist between individual RNA-guided nucleases that share the same PAM specificity or cleavage activity. Skilled artisans will appreciate that some aspects of the present disclosure relate to systems, methods and compositions that can be implemented using any suitable RNA-guided nuclease having a certain PAM specificity and/or cleavage activity. For this reason, unless otherwise specified, the term RNA-guided nuclease should be understood as a generic term, and not limited to any particular type (e.g., Cas9 vs. Cpf1), species (e.g., S. pyogenes vs. S. aureus) or variation (e.g., full-length vs. truncated or split; naturally-occurring PAM specificity vs. engineered PAM specificity, etc.) of RNA-guided nuclease.

The PAM sequence takes its name from its sequential relationship to the “protospacer” sequence that is complementary to gRNA targeting domains (or “spacers”). Together with protospacer sequences, PAM sequences define target regions or sequences for specific RNA-guided nuclease/gRNA combinations.

Various RNA-guided nucleases may require different sequential relationships between PAMs and protospacers. In general, Cas9s recognize PAM sequences that are 3′ of the protospacer. Cpf1, on the other hand, generally recognizes PAM sequences that are 5′ of the protospacer.

In addition to recognizing specific sequential orientations of PAMs and protospacers, RNA-guided nucleases can also recognize specific PAM sequences. S. aureus Cas9, for instance, recognizes a PAM sequence of NNGRRT or NNGRRV, wherein the N residues are immediately 3′ of the region recognized by the gRNA targeting domain. S. pyogenes Cas9 recognizes NGG PAM sequences. F. novicida Cpf1 recognizes a TTN PAM sequence. PAM sequences have been identified for a variety of RNA-guided nucleases, and a strategy for identifying novel PAM sequences has been described by Shmakov et al., 2015, Molecular Cell 60, 385-397, Nov. 5, 2015. It should also be noted that engineered RNA-guided nucleases can have PAM specificities that differ from the PAM specificities of reference molecules (for instance, in the case of an engineered RNA-guided nuclease, the reference molecule may be the naturally occurring variant from which the RNA-guided nuclease is derived, or the naturally occurring variant having the greatest amino acid sequence homology to the engineered RNA-guided nuclease).

In addition to their PAM specificity, RNA-guided nucleases can be characterized by their DNA cleavage activity: naturally-occurring RNA-guided nucleases typically form DSBs in target nucleic acids, but engineered variants have been produced that generate only SSBs (discussed above) Ran & Hsu, et al., Cell 154(6), 1380-1389, Sep. 12, 2013 (“Ran”)), or that that do not cut at all.

Cas9

Crystal structures have been determined for S. pyogenes Cas9 (Jinek et al., Science 343(6176), 1247997, 2014 (“Jinek 2014”), and for S. aureus Cas9 in complex with a unimolecular guide RNA and a target DNA (Nishimasu 2014; Anders et al., Nature. 2014 Sep. 25; 513(7519):569-73 (“Anders 2014”); and Nishimasu 2015).

A naturally occurring Cas9 protein comprises two lobes: a recognition (REC) lobe and a nuclease (NUC) lobe; each of which comprise particular structural and/or functional domains. The REC lobe comprises an arginine-rich bridge helix (BH) domain, and at least one REC domain (e.g., a REC1 domain and, optionally, a REC2 domain). The REC lobe does not share structural similarity with other known proteins, indicating that it is a unique functional domain. While not wishing to be bound by any theory, mutational analyses suggest specific functional roles for the BH and REC domains: the BH domain appears to play a role in gRNA:DNA recognition, while the REC domain is thought to interact with the repeat:anti-repeat duplex of the gRNA and to mediate the formation of the Cas9/gRNA complex.

The NUC lobe comprises a RuvC domain, an HNH domain, and a PAM-interacting (PI) domain. The RuvC domain shares structural similarity to retroviral integrase superfamily members and cleaves the non-complementary (i.e., bottom) strand of the target nucleic acid. It may be formed from two or more split RuvC motifs (such as RuvC I, RuvCII, and RuvCIII in S. pyogenes and S. aureus). The HNH domain, meanwhile, is structurally similar to HNN endonuclease motifs, and cleaves the complementary (i.e., top) strand of the target nucleic acid. The PI domain, as its name suggests, contributes to PAM specificity.

While certain functions of Cas9 are linked to (but not necessarily fully determined by) the specific domains set forth above, these and other functions may be mediated or influenced by other Cas9 domains, or by multiple domains on either lobe. For instance, in S. pyogenes Cas9, as described in Nishimasu 2014, the repeat:antirepeat duplex of the gRNA falls into a groove between the REC and NUC lobes, and nucleotides in the duplex interact with amino acids in the BH, PI, and REC domains. Some nucleotides in the first stem loop structure also interact with amino acids in multiple domains (PI, BH and REC1), as do some nucleotides in the second and third stem loops (RuvC and PI domains).

Cpf1

The crystal structure of Acidaminococcus sp. Cpf1 in complex with crRNA and a dsDNA target including a TTTN PAM sequence has been solved by Yamano et al. (Cell. 2016 May 5; 165(4): 949-962 (“Yamano”), incorporated by reference herein). Cpf1, like Cas9, has two lobes: a REC (recognition) lobe, and a NUC (nuclease) lobe. The REC lobe includes REC1 and REC2 domains, which lack similarity to any known protein structures. The NUC lobe, meanwhile, includes three RuvC domains (RuvC-I, -II and -III) and a BH domain. However, in contrast to Cas9, the Cpf1 REC lobe lacks an HNH domain, and includes other domains that also lack similarity to known protein structures: a structurally unique PI domain, three Wedge (WED) domains (WED-I, -II and —III), and a nuclease (Nuc) domain.

While Cas9 and Cpf1 share similarities in structure and function, it should be appreciated that certain Cpf1 activities are mediated by structural domains that are not analogous to any Cas9 domains. For instance, cleavage of the complementary strand of the target DNA appears to be mediated by the Nuc domain, which differs sequentially and spatially from the HNH domain of Cas9. Additionally, the non-targeting portion of Cpf1 gRNA (the handle) adopts a pseudoknot structure, rather than a stem loop structure formed by the repeat:antirepeat duplex in Cas9 gRNAs.

Nuclease Variants

The RNA-guided nucleases described herein have activities and properties that can be useful in a variety of applications, but the skilled artisan will appreciate that RNA-guided nucleases can also be modified in certain instances, to alter cleavage activity, PAM specificity, or other structural or functional features.

Turning first to modifications that alter cleavage activity, mutations that reduce or eliminate the activity of domains within the NUC lobe have been described above. Exemplary mutations that may be made in the RuvC domains, in the Cas9 HNH domain, or in the Cpf1 Nuc domain are described in Ran & Hsu, et al., (Cell 154(6), 1380-1389, Sep. 12, 2013), and Yamano, et al. (Cell. 2016 May 5; 165(4): 949-962); as well as in WO 2016/073990 by Cotta-Ramusino, the entire contents of each of which are incorporated herein by reference. In general, mutations that reduce or eliminate activity in one of the two nuclease domains result in RNA-guided nucleases with nickase activity, but it should be noted that the type of nickase activity varies depending on which domain is inactivated. As one example, inactivation of a RuvC domain or of a Cas9 HNH domain results in a nickase.

Modifications of PAM specificity relative to naturally occurring Cas9 reference molecules has been described by Kleinstiver et al. for both S. pyogenes (Kleinstiver et al., Nature. 2015 Jul. 23; 523(7561):481-5); and S. aureus (Kleinstiver et al., Nat Biotechnol. 2015 December; 33(12): 1293-1298). Kleinstiver et al. have also described modifications that improve the targeting fidelity of Cas9 (Nature, 2016 Jan. 28; 529, 490-495). Each of these references is incorporated by reference herein.

RNA-guided nucleases have been split into two or more parts, as described by Zetsche et al. (Nat Biotechnol. 2015 February; 33(2):139-42, incorporated by reference), and by Fine et al. (Sci Rep. 2015 Jul. 1; 5:10777, incorporated by reference).

RNA-guided nucleases can be, in certain embodiments, size-optimized or truncated, for instance via one or more deletions that reduce the size of the nuclease while still retaining gRNA association, target and PAM recognition, and cleavage activities. In certain embodiments, RNA guided nucleases are bound, covalently or non-covalently, to another polypeptide, nucleotide, or other structure, optionally by means of a linker. Exemplary bound nucleases and linkers are described by Guilinger et al., Nature Biotechnology 32, 577-582 (2014), which is incorporated by reference herein.

RNA-guided nucleases also optionally include a tag, such as, but not limited to, a nuclear localization signal, to facilitate movement of RNA-guided nuclease protein into the nucleus. In certain embodiments, the RNA-guided nuclease can incorporate C- and/or N-terminal nuclear localization signals. Nuclear localization sequences are known in the art and are described in Maeder and elsewhere.

The foregoing list of modifications is intended to be exemplary in nature, and the skilled artisan will appreciate, in view of the instant disclosure, that other modifications may be possible or desirable in certain applications. For brevity, therefore, exemplary systems, methods and compositions of the present disclosure are presented with reference to particular RNA-guided nucleases, but it should be understood that the RNA-guided nucleases used may be modified in ways that do not alter their operating principles. Such modifications are within the scope of the present disclosure.

Exemplary suitable nuclease variants include, but are not limited to, AsCpf1 variants comprising an M537R substitution, an H800A substitution, and/or an F870L substitution, or any combination thereof (numbering scheme according to AsCpf1 wild-type sequence). In some embodiments, an ASCpfl variant comprises an M537R substitution, an H800A substitution, and an F870L substitution. Other suitable modifications of the AsCpf1 amino acid sequence are known to those of ordinary skill in the art. Some exemplary sequences of wild-type AsCpf1 and AsCpf1 variants are provided below:

His-AsCpf1-sNLS-sNLS H800A amino acid sequence (SEQ ID NO: 1144):
MGHHHHHHGSTQFEGFTNLYQVSKTLRFELIPQGKTLKHIQEQGFIEEDKARNDHYK
ELKPIIDRIYKTYADQCLQLVQLDWENLSAAIDSYRKEKTEETRNALIEEQATYRNAI
HDYFIGRTDNLTDAINKRHAEIYKGLFKAELFNGKVLKQLGTVTTTEHENALLRSFD
KFTTYFSGFYENRKNVFSAEDISTAIPHRIVQDNFPKFKENCHIFTRLITAVPSLREHFE
NVKKAIGIFVSTSIEEVFSFPFYNQLLTQTQIDLYNQLLGGISREAGTEKIKGLNEVLNL
AIQKNDETAHIIASLPHRFIPLFKQILSDRNTLSFILEEFKSDEEVIQSFCKYKTLLRNEN
VLETAEALFNELNSIDLTHIFISHKKLETISSALCDHWDTLRNALYERRISELTGKITKS
AKEKVQRSLKHEDINLQEIISAAGKELSEAFKQKTSEILSHAHAALDQPLPTTLKKQE
EKEILKSQLDSLLGLYHLLDWFAVDESNEVDPEFSARLTGIKLEMEPSLSFYNKARNY
ATKKPYSVEKFKLNFQMPTLASGWDVNKEKNNGAILFVKNGLYYLGIMPKQKGRY
KALSFEPTEKTSEGFDKMYYDYFPDAAKMIPKCSTQLKAVTAHFQTHTTPILLSNNFI
EPLEITKEIYDLNNPEKEPKKFQTAYAKKTGDQKGYREALCKWIDFTRDFLSKYTKT
TSIDLSSLRPSSQYKDLGEYYAELNPLLYHISFQRIAEKEIMDAVETGKLYLFQIYNKD
FAKGHHGKPNLHTLYWTGLFSPENLAKTSIKLNGQAELFYRPKSRMKRMAARLGEK
MLNKKLKDQKTPIPDTLYQELYDYVNHRLSHDLSDEARALLPNVITKEVSHEIIKDRR
FTSDKFFFHVPITLNYQAANSPSKFNQRVNAYLKEHPETPIIGIDRGERNLIYITVIDST
GKILEQRSLNTIQQFDYQKKLDNREKERVAARQAWSVVGTIKDLKQGYLSQVIHEIV
DLMIHYQAVVVLENLNFGFKSKRTGIAEKAVYQQFEKMLIDKLNCLVLKDYPAEKV
GGVLNPYQLTDQFTSFAKMGTQSGFLFYVPAPYTSKIDPLTGFVDPFVWKTIKNHES
RKHFLEGFDFLHYDVKTGDFILHFKMNRNLSFQRGLPGFMPAWDIVFEKNETQFDA
KGTPFIAGKRIVPVIENHRFTGRYRDLYPANELIALLEEKGIVFRDGSNILPKLLENDD
SHAIDTMVALIRSVLQMRNSNAATGEDYINSPVRDLNGVCFDSRFQNPEWPMDADA
NGAYHIALKGQLLLNHLKESKDLKLQNGISNQDWLAYIQELRNGSPKKKRKVGSPK
KKRKV
Cpf1 variant 1 amino acid sequence (SEQ ID NO: 1145):
MTQFEGFTNLYQVSKTLRFELIPQGKTLKHIQEQGFIEEDKARNDHYKELKPIIDRIYK
TYADQCLQLVQLDWENLSAAIDSYRKEKTEETRNALIEEQATYRNAIHDYFIGRTDN
LTDAINKRHAEIYKGLFKAELFNGKVLKQLGTVTTTEHENALLRSFDKFTTYFSGFYE
NRKNVFSAEDISTAIPHRIVQDNFPKFKENCHIFTRLITAVPSLREHFENVKKAIGIFVS
TSIEEVFSFPFYNQLLTQTQIDLYNQLLGGISREAGTEKIKGLNEVLNLAIQKNDETAH
IIASLPHRFIPLFKQILSDRNTLSFILEEFKSDEEVIQSFCKYKTLLRNENVLETAEALFN
ELNSIDLTHIFISHKKLETISSALCDHWDTLRNALYERRISELTGKITKSAKEKVQRSLK
HEDINLQEIISAAGKELSEAFKQKTSEILSHAHAALDQPLPTTLKKQEEKEILKSQLDS
LLGLYHLLDWFAVDESNEVDPEFSARLTGIKLEMEPSLSFYNKARNYATKKPYSVEK
FKLNFQRPTLASGWDVNKEKNNGAILFVKNGLYYLGIMPKQKGRYKALSFEPTEKT
SEGFDKMYYDYFPDAAKMIPKCSTQLKAVTAHFQTHTTPILLSNNFIEPLEITKEIYDL
NNPEKEPKKFQTAYAKKTGDQKGYREALCKWIDFTRDFLSKYTKTTSIDLSSLRPSS
QYKDLGEYYAELNPLLYHISFQRIAEKEIMDAVETGKLYLFQIYNKDFAKGHHGKPN
LHTLYWTGLFSPENLAKTSIKLNGQAELFYRPKSRMKRMAHRLGEKMLNKKLKDQ
KTPIPDTLYQELYDYVNHRLSHDLSDEARALLPNVITKEVSHEIIKDRRFTSDKFLFHV
PITLNYQAANSPSKFNQRVNAYLKEHPETPIIGIDRGERNLIYITVIDSTGKILEQRSLN
TIQQFDYQKKLDNREKERVAARQAWSVVGTIKDLKQGYLSQVIHEIVDLMIHYQAV
VVLENLNFGFKSKRTGIAEKAVYQQFEKMLIDKLNCLVLKDYPAEKVGGVLNPYQL
TDQFTSFAKMGTQSGFLFYVPAPYTSKIDPLTGFVDPFVWKTIKNHESRKHFLEGFDF
LHYDVKTGDFILHFKMNRNLSFQRGLPGFMPAWDIVFEKNETQFDAKGTPFIAGKRI
VPVIENHRFTGRYRDLYPANELIALLEEKGIVFRDGSNILPKLLENDDSHAIDTMVALI
RSVLQMRNSNAATGEDYINSPVRDLNGVCFDSRFQNPEWPMDADANGAYHIALKG
QLLLNHLKESKDLKLQNGISNQDWLAYIQELRNGRSSDDEATADSQHAAPPKKKRK
VGGSGGSGGSGGSGGSGGSGGSGGSLEHHHHHH
Cpf1 variant 2 amino acid sequence (SEQ ID NO: 1146):
MTQFEGFTNLYQVSKTLRFELIPQGKTLKHIQEQGFIEEDKARNDHYKELKPIIDRIYK
TYADQCLQLVQLDWENLSAAIDSYRKEKTEETRNALIEEQATYRNAIHDYFIGRTDN
LTDAINKRHAEIYKGLFKAELFNGKVLKQLGTVTTTEHENALLRSFDKFTTYFSGFYE
NRKNVFSAEDISTAIPHRIVQDNFPKFKENCHIFTRLITAVPSLREHFENVKKAIGIFVS
TSIEEVFSFPFYNQLLTQTQIDLYNQLLGGISREAGTEKIKGLNEVLNLAIQKNDETAH
IIASLPHRFIPLFKQILSDRNTLSFILEEFKSDEEVIQSFCKYKTLLRNENVLETAEALFN
ELNSIDLTHIFISHKKLETISSALCDHWDTLRNALYERRISELTGKITKSAKEKVQRSLK
HEDINLQEIISAAGKELSEAFKQKTSEILSHAHAALDQPLPTTLKKQEEKEILKSQLDS
LLGLYHLLDWFAVDESNEVDPEFSARLTGIKLEMEPSLSFYNKARNYATKKPYSVEK
FKLNFQMPTLASGWDVNKEKNNGAILFVKNGLYYLGIMPKQKGRYKALSFEPTEKT
SEGFDKMYYDYFPDAAKMIPKCSTQLKAVTAHFQTHTTPILLSNNFIEPLEITKEIYDL
NNPEKEPKKFQTAYAKKTGDQKGYREALCKWIDFTRDFLSKYTKTTSIDLSSLRPSS
QYKDLGEYYAELNPLLYHISFQRIAEKEIMDAVETGKLYLFQIYNKDFAKGHHGKPN
LHTLYWTGLFSPENLAKTSIKLNGQAELFYRPKSRMKRMAHRLGEKMLNKKLKDQ
KTPIPDTLYQELYDYVNHRLSHDLSDEARALLPNVITKEVSHEIIKDRRFTSDKFFFHV
PITLNYQAANSPSKFNQRVNAYLKEHPETPIIGIDRGERNLIYITVIDSTGKILEQRSLN
TIQQFDYQKKLDNREKERVAARQAWSVVGTIKDLKQGYLSQVIHEIVDLMIHYQAV
VVLENLNFGFKSKRTGIAEKAVYQQFEKMLIDKLNCLVLKDYPAEKVGGVLNPYQL
TDQFTSFAKMGTQSGFLFYVPAPYTSKIDPLTGFVDPFVWKTIKNHESRKHFLEGFDF
LHYDVKTGDFILHFKMNRNLSFQRGLPGFMPAWDIVFEKNETQFDAKGTPFIAGKRI
VPVIENHRFTGRYRDLYPANELIALLEEKGIVFRDGSNILPKLLENDDSHAIDTMVALI
RSVLQMRNSNAATGEDYINSPVRDLNGVCFDSRFQNPEWPMDADANGAYHIALKG
QLLLNHLKESKDLKLQNGISNQDWLAYIQELRNGRSSDDEATADSQHAAPPKKKRK
VGGSGGSGGSGGSGGSGGSGGSGGSLEHHHHHH
Cpf1 variant 3 amino acid sequence (SEQ ID NO: 1147):
MTQFEGFTNLYQVSKTLRFELIPQGKTLKHIQEQGFIEEDKARNDHYKELKPIIDRIYK
TYADQCLQLVQLDWENLSAAIDSYRKEKTEETRNALIEEQATYRNAIHDYFIGRTDN
LTDAINKRHAEIYKGLFKAELFNGKVLKQLGTVTTTEHENALLRSFDKFTTYFSGFYE
NRKNVFSAEDISTAIPHRIVQDNFPKFKENCHIFTRLITAVPSLREHFENVKKAIGIFVS
TSIEEVFSFPFYNQLLTQTQIDLYNQLLGGISREAGTEKIKGLNEVLNLAIQKNDETAH
IIASLPHRFIPLFKQILSDRNTLSFILEEFKSDEEVIQSFCKYKTLLRNENVLETAEALFN
ELNSIDLTHIFISHKKLETISSALCDHWDTLRNALYERRISELTGKITKSAKEKVQRSLK
HEDINLQEIISAAGKELSEAFKQKTSEILSHAHAALDQPLPTTLKKQEEKEILKSQLDS
LLGLYHLLDWFAVDESNEVDPEFSARLTGIKLEMEPSLSFYNKARNYATKKPYSVEK
FKLNFQRPTLASGWDVNKEKNNGAILFVKNGLYYLGIMPKQKGRYKALSFEPTEKT
SEGFDKMYYDYFPDAAKMIPKCSTQLKAVTAHFQTHTTPILLSNNFIEPLEITKEIYDL
NNPEKEPKKFQTAYAKKTGDQKGYREALCKWIDFTRDFLSKYTKTTSIDLSSLRPSS
QYKDLGEYYAELNPLLYHISFQRIAEKEIMDAVETGKLYLFQIYNKDFAKGHHGKPN
LHTLYWTGLFSPENLAKTSIKLNGQAELFYRPKSRMKRMAARLGEKMLNKKLKDQ
KTPIPDTLYQELYDYVNHRLSHDLSDEARALLPNVITKEVSHEIIKDRRFTSDKFLFHV
PITLNYQAANSPSKFNQRVNAYLKEHPETPIIGIDRGERNLIYITVIDSTGKILEQRSLN
TIQQFDYQKKLDNREKERVAARQAWSVVGTIKDLKQGYLSQVIHEIVDLMIHYQAV
VVLENLNFGFKSKRTGIAEKAVYQQFEKMLIDKLNCLVLKDYPAEKVGGVLNPYQL
TDQFTSFAKMGTQSGFLFYVPAPYTSKIDPLTGFVDPFVWKTIKNHESRKHFLEGFDF
LHYDVKTGDFILHFKMNRNLSFQRGLPGFMPAWDIVFEKNETQFDAKGTPFIAGKRI
VPVIENHRFTGRYRDLYPANELIALLEEKGIVFRDGSNILPKLLENDDSHAIDTMVALI
RSVLQMRNSNAATGEDYINSPVRDLNGVCFDSRFQNPEWPMDADANGAYHIALKG
QLLLNHLKESKDLKLQNGISNQDWLAYIQELRNGRSSDDEATADSQHAAPPKKKRK
VGGSGGSGGSGGSGGSGGSGGSGGSLEHHHHHH
Cpf1 variant 4 amino acid sequence (SEQ ID NO: 1148):
MTQFEGFTNLYQVSKTLRFELIPQGKTLKHIQEQGFIEEDKARNDHYKELKPIIDRIYK
TYADQCLQLVQLDWENLSAAIDSYRKEKTEETRNALIEEQATYRNAIHDYFIGRTDN
LTDAINKRHAEIYKGLFKAELFNGKVLKQLGTVTTTEHENALLRSFDKFTTYFSGFYE
NRKNVFSAEDISTAIPHRIVQDNFPKFKENCHIFTRLITAVPSLREHFENVKKAIGIFVS
TSIEEVFSFPFYNQLLTQTQIDLYNQLLGGISREAGTEKIKGLNEVLNLAIQKNDETAH
IIASLPHRFIPLFKQILSDRNTLSFILEEFKSDEEVIQSFCKYKTLLRNENVLETAEALFN
ELNSIDLTHIFISHKKLETISSALCDHWDTLRNALYERRISELTGKITKSAKEKVQRSLK
HEDINLQEIISAAGKELSEAFKQKTSEILSHAHAALDQPLPTTLKKQEEKEILKSQLDS
LLGLYHLLDWFAVDESNEVDPEFSARLTGIKLEMEPSLSFYNKARNYATKKPYSVEK
FKLNFQRPTLASGWDVNKEKNNGAILFVKNGLYYLGIMPKQKGRYKALSFEPTEKT
SEGFDKMYYDYFPDAAKMIPKCSTQLKAVTAHFQTHTTPILLSNNFIEPLEITKEIYDL
NNPEKEPKKFQTAYAKKTGDQKGYREALCKWIDFTRDFLSKYTKTTSIDLSSLRPSS
QYKDLGEYYAELNPLLYHISFQRIAEKEIMDAVETGKLYLFQIYNKDFAKGHHGKPN
LHTLYWTGLFSPENLAKTSIKLNGQAELFYRPKSRMKRMAARLGEKMLNKKLKDQ
KTPIPDTLYQELYDYVNHRLSHDLSDEARALLPNVITKEVSHEIIKDRRFTSDKFLFHV
PITLNYQAANSPSKFNQRVNAYLKEHPETPIIGIDRGERNLIYITVIDSTGKILEQRSLN
TIQQFDYQKKLDNREKERVAARQAWSVVGTIKDLKQGYLSQVIHEIVDLMIHYQAV
VVLENLNFGFKSKRTGIAEKAVYQQFEKMLIDKLNCLVLKDYPAEKVGGVLNPYQL
TDQFTSFAKMGTQSGFLFYVPAPYTSKIDPLTGFVDPFVWKTIKNHESRKHFLEGFDF
LHYDVKTGDFILHFKMNRNLSFQRGLPGFMPAWDIVFEKNETQFDAKGTPFIAGKRI
VPVIENHRFTGRYRDLYPANELIALLEEKGIVFRDGSNILPKLLENDDSHAIDTMVALI
RSVLQMRNSNAATGEDYINSPVRDLNGVCFDSRFQNPEWPMDADANGAYHIALKG
QLLLNHLKESKDLKLQNGISNQDWLAYIQELRNGRSSDDEATADSQHAAPPKKKRK
V
Cpf1 variant 5 amino acid sequence (SEQ ID NO: 1149):
MTQFEGFTNLYQVSKTLRFELIPQGKTLKHIQEQGFIEEDKARNDHYKELKPIIDRIYK
TYADQCLQLVQLDWENLSAAIDSYRKEKTEETRNALIEEQATYRNAIHDYFIGRTDN
LTDAINKRHAEIYKGLFKAELFNGKVLKQLGTVTTTEHENALLRSFDKFTTYFSGFYE
NRKNVFSAEDISTAIPHRIVQDNFPKFKENCHIFTRLITAVPSLREHFENVKKAIGIFVS
TSIEEVFSFPFYNQLLTQTQIDLYNQLLGGISREAGTEKIKGLNEVLNLAIQKNDETAH
IIASLPHRFIPLFKQILSDRNTLSFILEEFKSDEEVIQSFCKYKTLLRNENVLETAEALFN
ELNSIDLTHIFISHKKLETISSALCDHWDTLRNALYERRISELTGKITKSAKEKVQRSLK
HEDINLQEIISAAGKELSEAFKQKTSEILSHAHAALDQPLPTTLKKQEEKEILKSQLDS
LLGLYHLLDWFAVDESNEVDPEFSARLTGIKLEMEPSLSFYNKARNYATKKPYSVEK
FKLNFQRPTLASGWDVNKEKNNGAILFVKNGLYYLGIMPKQKGRYKALSFEPTEKT
SEGFDKMYYDYFPDAAKMIPKCSTQLKAVTAHFQTHTTPILLSNNFIEPLEITKEIYDL
NNPEKEPKKFQTAYAKKTGDQKGYREALCKWIDFTRDFLSKYTKTTSIDLSSLRPSS
QYKDLGEYYAELNPLLYHISFQRIAEKEIMDAVETGKLYLFQIYNKDFAKGHHGKPN
LHTLYWTGLFSPENLAKTSIKLNGQAELFYRPKSRMKRMAHRLGEKMLNKKLKDQ
KTPIPDTLYQELYDYVNHRLSHDLSDEARALLPNVITKEVSHEIIKDRRFTSDKFLFHV
PITLNYQAANSPSKFNQRVNAYLKEHPETPIIGIDRGERNLIYITVIDSTGKILEQRSLN
TIQQFDYQKKLDNREKERVAARQAWSVVGTIKDLKQGYLSQVIHEIVDLMIHYQAV
VVLENLNFGFKSKRTGIAEKAVYQQFEKMLIDKLNCLVLKDYPAEKVGGVLNPYQL
TDQFTSFAKMGTQSGFLFYVPAPYTSKIDPLTGFVDPFVWKTIKNHESRKHFLEGFDF
LHYDVKTGDFILHFKMNRNLSFQRGLPGFMPAWDIVFEKNETQFDAKGTPFIAGKRI
VPVIENHRFTGRYRDLYPANELIALLEEKGIVFRDGSNILPKLLENDDSHAIDTMVALI
RSVLQMRNSNAATGEDYINSPVRDLNGVCFDSRFQNPEWPMDADANGAYHIALKG
QLLLNHLKESKDLKLQNGISNQDWLAYIQELRNGRSSDDEATADSQHAAPPKKKRK
V
Cpf1 variant 6 amino acid sequence (SEQ ID NO: 1150):
MTQFEGFTNLYQVSKTLRFELIPQGKTLKHIQEQGFIEEDKARNDHYKELKPIIDRIYK
TYADQCLQLVQLDWENLSAAIDSYRKEKTEETRNALIEEQATYRNAIHDYFIGRTDN
LTDAINKRHAEIYKGLFKAELFNGKVLKQLGTVTTTEHENALLRSFDKFTTYFSGFYE
NRKNVFSAEDISTAIPHRIVQDNFPKFKENCHIFTRLITAVPSLREHFENVKKAIGIFVS
TSIEEVFSFPFYNQLLTQTQIDLYNQLLGGISREAGTEKIKGLNEVLNLAIQKNDETAH
IIASLPHRFIPLFKQILSDRNTLSFILEEFKSDEEVIQSFCKYKTLLRNENVLETAEALFN
ELNSIDLTHIFISHKKLETISSALCDHWDTLRNALYERRISELTGKITKSAKEKVQRSLK
HEDINLQEIISAAGKELSEAFKQKTSEILSHAHAALDQPLPTTLKKQEEKEILKSQLDS
LLGLYHLLDWFAVDESNEVDPEFSARLTGIKLEMEPSLSFYNKARNYATKKPYSVEK
FKLNFQRPTLASGWDVNKEKNNGAILFVKNGLYYLGIMPKQKGRYKALSFEPTEKT
SEGFDKMYYDYFPDAAKMIPKCSTQLKAVTAHFQTHTTPILLSNNFIEPLEITKEIYDL
NNPEKEPKKFQTAYAKKTGDQKGYREALCKWIDFTRDFLSKYTKTTSIDLSSLRPSS
QYKDLGEYYAELNPLLYHISFQRIAEKEIMDAVETGKLYLFQIYNKDFAKGHHGKPN
LHTLYWTGLFSPENLAKTSIKLNGQAELFYRPKSRMKRMAHRLGEKMLNKKLKDQ
KTPIPDTLYQELYDYVNHRLSHDLSDEARALLPNVITKEVSHEIIKDRRFTSDKFLFHV
PITLNYQAANSPSKFNQRVNAYLKEHPETPIIGIDRGERNLIYITVIDSTGKILEQRSLN
TIQQFDYQKKLDNREKERVAARQAWSVVGTIKDLKQGYLSQVIHEIVDLMIHYQAV
VVLENLNFGFKSKRTGIAEKAVYQQFEKMLIDKLNCLVLKDYPAEKVGGVLNPYQL
TDQFTSFAKMGTQSGFLFYVPAPYTSKIDPLTGFVDPFVWKTIKNHESRKHFLEGFDF
LHYDVKTGDFILHFKMNRNLSFQRGLPGFMPAWDIVFEKNETQFDAKGTPFIAGKRI
VPVIENHRFTGRYRDLYPANELIALLEEKGIVFRDGSNILPKLLENDDSHAIDTMVALI
RSVLQMRNSNAATGEDYINSPVRDLNGVCFDSRFQNPEWPMDADANGAYHIALKG
QLLLNHLKESKDLKLQNGISNQDWLAYIQELRNGRSSDDEATADSQHAAPPKKKRK
VGGSGGSGGSGGSGGSGGSGGSGGSLEHHHHHH
Cpf1 variant 7 amino acid sequence (SEQ ID NO: 1151):
MGRDPGKPIPNPLLGLDSTAPKKKRKVGIHGVPAATQFEGFTNLYQVSKTLRFELIPQ
GKTLKHIQEQGFIEEDKARNDHYKELKPIIDRIYKTYADQCLQLVQLDWENLSAAIDS
YRKEKTEETRNALIEEQATYRNAIHDYFIGRTDNLTDAINKRHAEIYKGLFKAELFNG
KVLKQLGTVTTTEHENALLRSFDKFTTYFSGFYENRKNVFSAEDISTAIPHRIVQDNFP
KFKENCHIFTRLITAVPSLREHFENVKKAIGIFVSTSIEEVFSFPFYNQLLTQTQIDLYN
QLLGGISREAGTEKIKGLNEVLNLAIQKNDETAHIIASLPHRFIPLFKQILSDRNTLSFIL
EEFKSDEEVIQSFCKYKTLLRNENVLETAEALFNELNSIDLTHIFISHKKLETISSALCD
HWDTLRNALYERRISELTGKITKSAKEKVQRSLKHEDINLQEIISAAGKELSEAFKQK
TSEILSHAHAALDQPLPTTLKKQEEKEILKSQLDSLLGLYHLLDWFAVDESNEVDPEF
SARLTGIKLEMEPSLSFYNKARNYATKKPYSVEKFKLNFQMPTLASGWDVNKEKNN
GAILFVKNGLYYLGIMPKQKGRYKALSFEPTEKTSEGFDKMYYDYFPDAAKMIPKCS
TQLKAVTAHFQTHTTPILLSNNFIEPLEITKEIYDLNNPEKEPKKFQTAYAKKTGDQK
GYREALCKWIDFTRDFLSKYTKTTSIDLSSLRPSSQYKDLGEYYAELNPLLYHISFQRI
AEKEIMDAVETGKLYLFQIYNKDFAKGHHGKPNLHTLYWTGLFSPENLAKTSIKLNG
QAELFYRPKSRMKRMAHRLGEKMLNKKLKDQKTPIPDTLYQELYDYVNHRLSHDL
SDEARALLPNVITKEVSHEIIKDRRFTSDKFFFHVPITLNYQAANSPSKFNQRVNAYLK
EHPETPIIGIDRGERNLIYITVIDSTGKILEQRSLNTIQQFDYQKKLDNREKERVAARQA
WSVVGTIKDLKQGYLSQVIHEIVDLMIHYQAVVVLENLNFGFKSKRTGIAEKAVYQQ
FEKMLIDKLNCLVLKDYPAEKVGGVLNPYQLTDQFTSFAKMGTQSGFLFYVPAPYTS
KIDPLTGFVDPFVWKTIKNHESRKHFLEGFDFLHYDVKTGDFILHFKMNRNLSFQRG
LPGFMPAWDIVFEKNETQFDAKGTPFIAGKRIVPVIENHRFTGRYRDLYPANELIALL
EEKGIVFRDGSNILPKLLENDDSHAIDTMVALIRSVLQMRNSNAATGEDYINSPVRDL
NGVCFDSRFQNPEWPMDADANGAYHIALKGQLLLNHLKESKDLKLQNGISNQDWL
AYIQELRNPKKKRKVKLAAALEHHHHHH
Exemplary AsCpf1 wild-type amino acid sequence (SEQ ID NO: 1152):
MTQFEGFTNLYQVSKTLRFELIPQGKTLKHIQEQGFIEEDKARNDHYKELKPIIDRIYK
TYADQCLQLVQLDWENLSAAIDSYRKEKTEETRNALIEEQATYRNAIHDYFIGRTDN
LTDAINKRHAEIYKGLFKAELFNGKVLKQLGTVTTTEHENALLRSFDKFTTYFSGFYE
NRKNVFSAEDISTAIPHRIVQDNFPKFKENCHIFTRLITAVPSLREHFENVKKAIGIFVS
TSIEEVFSFPFYNQLLTQTQIDLYNQLLGGISREAGTEKIKGLNEVLNLAIQKNDETAH
IIASLPHRFIPLFKQILSDRNTLSFILEEFKSDEEVIQSFCKYKTLLRNENVLETAEALFN
ELNSIDLTHIFISHKKLETISSALCDHWDTLRNALYERRISELTGKITKSAKEKVQRSLK
HEDINLQEIISAAGKELSEAFKQKTSEILSHAHAALDQPLPTTLKKQEEKEILKSQLDS
LLGLYHLLDWFAVDESNEVDPEFSARLTGIKLEMEPSLSFYNKARNYATKKPYSVEK
FKLNFQMPTLASGWDVNKEKNNGAILFVKNGLYYLGIMPKQKGRYKALSFEPTEKT
SEGFDKMYYDYFPDAAKMIPKCSTQLKAVTAHFQTHTTPILLSNNFIEPLEITKEIYDL
NNPEKEPKKFQTAYAKKTGDQKGYREALCKWIDFTRDFLSKYTKTTSIDLSSLRPSS
QYKDLGEYYAELNPLLYHISFQRIAEKEIMDAVETGKLYLFQIYNKDFAKGHHGKPN
LHTLYWTGLFSPENLAKTSIKLNGQAELFYRPKSRMKRMAHRLGEKMLNKKLKDQ
KTPIPDTLYQELYDYVNHRLSHDLSDEARALLPNVITKEVSHEIIKDRRFTSDKFFFHV
PITLNYQAANSPSKFNQRVNAYLKEHPETPIIGIDRGERNLIYITVIDSTGKILEQRSLN
TIQQFDYQKKLDNREKERVAARQAWSVVGTIKDLKQGYLSQVIHEIVDLMIHYQAV
VVLENLNFGFKSKRTGIAEKAVYQQFEKMLIDKLNCLVLKDYPAEKVGGVLNPYQL
TDQFTSFAKMGTQSGFLFYVPAPYTSKIDPLTGFVDPFVWKTIKNHESRKHFLEGFDF
LHYDVKTGDFILHFKMNRNLSFQRGLPGFMPAWDIVFEKNETQFDAKGTPFIAGKRI
VPVIENHRFTGRYRDLYPANELIALLEEKGIVFRDGSNILPKLLENDDSHAIDTMVALI
RSVLQMRNSNAATGEDYINSPVRDLNGVCFDSRFQNPEWPMDADANGAYHIALKG
QLLLNHLKESKDLKLQNGISNQDWLAYIQELRN

Additional suitable nucleases and nuclease variants will be apparent to the skilled artisan based on the present disclosure in view of the knowledge in the art. Exemplary suitable nucleases may include, but are not limited to, those provided in Table 2 herein.

Nucleic Acids Encoding RNA-Guided Nucleases

Nucleic acids encoding RNA-guided nucleases, e.g., Cas9, Cpf1 or functional fragments thereof, are provided herein. Exemplary nucleic acids encoding RNA-guided nucleases have been described previously (see, e.g., Cong 2013; Wang 2013; Mali 2013; Jinek 2012).

In some cases, a nucleic acid encoding an RNA-guided nuclease can be a synthetic nucleic acid sequence. For example, the synthetic nucleic acid molecule can be chemically modified. In certain embodiments, an mRNA encoding an RNA-guided nuclease will have one or more (e.g., all) of the following properties: it can be capped; polyadenylated; and substituted with 5-methylcytidine and/or pseudouridine.

Synthetic nucleic acid sequences can also be codon optimized, e.g., at least one non-common codon or less-common codon has been replaced by a common codon. For example, the synthetic nucleic acid can direct the synthesis of an optimized messenger mRNA, e.g., optimized for expression in a mammalian expression system, e.g., described herein. Examples of codon optimized Cas9 coding sequences are presented in Cotta-Ramusino.

In addition, or alternatively, a nucleic acid encoding an RNA-guided nuclease may comprise a nuclear localization sequence (NLS). Nuclear localization sequences are known in the art.

As an example, the nucleic acid sequence for Cpf1 variant 4 is set forth below as SEQ ID NO: 1177

ATGACCCAGTTTGAAGGTTTCACCAATCTGTATCAGGTTAGCAAAACCCTGCGTTTTGAACT
GATTCCGCAGGGTAAAACCCTGAAACATATTCAAGAACAGGGCTTCATCGAAGAGGATAAAG
CACGTAACGATCACTACAAAGAACTGAAACCGATTATCGACCGCATCTATAAAACCTATGCA
GATCAGTGTCTGCAGCTGGTTCAGCTGGATTGGGAAAATCTGAGCGCAGCAATTGATAGTTA
TCGCAAAGAAAAAACCGAAGAAACCCGTAATGCACTGATTGAAGAACAGGCAACCTATCGTA
ATGCCATCCATGATTATTTCATTGGTCGTACCGATAATCTGACCGATGCAATTAACAAACGT
CACGCCGAAATCTATAAAGGCCTGTTTAAAGCCGAACTGTTTAATGGCAAAGTTCTGAAACA
GCTGGGCACCGTTACCACCACCGAACATGAAAATGCACTGCTGCGTAGCTTTGATAAATTCA
CCACCTATTTCAGCGGCTTTTATGAGAATCGCAAAAACGTGTTTAGCGCAGAAGATATTAGC
ACCGCAATTCCGCATCGTATTGTGCAGGATAATTTCCCGAAATTCAAAGAGAACTGCCACAT
TTTTACCCGTCTGATTACCGCAGTTCCGAGCCTGCGTGAACATTTTGAAAACGTTAAAAAAG
CCATCGGCATCTTTGTTAGCACCAGCATTGAAGAAGTTTTTAGCTTCCCGTTTTACAATCAG
CTGCTGACCCAGACCCAGATTGATCTGTATAACCAACTGCTGGGTGGTATTAGCCGTGAAGC
AGGCACCGAAAAAATCAAAGGTCTGAATGAAGTGCTGAATCTGGCCATTCAGAAAAATGATG
AAACCGCACATATTATTGCAAGCCTGCCGCATCGTTTTATTCCGCTGTTCAAACAAATTCTG
AGCGATCGTAATACCCTGAGCTTTATTCTGGAAGAATTCAAATCCGATGAAGAGGTGATTCA
GAGCTTTTGCAAATACAAAACGCTGCTGCGCAATGAAAATGTTCTGGAAACTGCCGAAGCAC
TGTTTAACGAACTGAATAGCATTGATCTGACCCACATCTTTATCAGCCACAAAAAACTGGAA
ACCATTTCAAGCGCACTGTGTGATCATTGGGATACCCTGCGTAATGCCCTGTATGAACGTCG
TATTAGCGAACTGACCGGTAAAATTACCAAAAGCGCGAAAGAAAAAGTTCAGCGCAGTCTGA
AACATGAGGATATTAATCTGCAAGAGATTATTAGCGCAGCCGGTAAAGAACTGTCAGAAGCA
TTTAAACAGAAAACCAGCGAAATTCTGTCACATGCACATGCAGCACTGGATCAGCCGCTGCC
GACCACCCTGAAAAAACAAGAAGAAAAAGAAATCCTGAAAAGCCAGCTGGATAGCCTGCTGG
GTCTGTATCATCTGCTGGACTGGTTTGCAGTTGATGAAAGCAATGAAGTTGATCCGGAATTT
AGCGCACGTCTGACCGGCATTAAACTGGAAATGGAACCGAGCCTGAGCTTTTATAACAAAGC
CCGTAATTATGCCACCAAAAAACCGTATAGCGTCGAAAAATTCAAACTGAACTTTCAGCGTC
CGACCCTGGCAAGCGGTTGGGATGTTAATAAAGAAAAAAACAACGGTGCCATCCTGTTCGTG
AAAAATGGCCTGTATTATCTGGGTATTATGCCGAAACAGAAAGGTCGTTATAAAGCGCTGAG
CTTTGAACCGACGGAAAAAACCAGTGAAGGTTTTGATAAAATGTACTACGACTATTTTCCGG
ATGCAGCCAAAATGATTCCGAAATGTAGCACCCAGCTGAAAGCAGTTACCGCACATTTTCAG
ACCCATACCACCCCGATTCTGCTGAGCAATAACTTTATTGAACCGCTGGAAATCACCAAAGA
GATCTACGATCTGAATAACCCGGAAAAAGAGCCGAAAAAATTCCAGACCGCATATGCAAAAA
AAACCGGTGATCAGAAAGGTTATCGTGAAGCGCTGTGTAAATGGATTGATTTCACCCGTGAT
TTTCTGAGCAAATACACCAAAACCACCAGTATCGATCTGAGCAGCCTGCGTCCGAGCAGCCA
GTATAAAGATCTGGGCGAATATTATGCAGAACTGAATCCGCTGCTGTATCATATTAGCTTTC
AGCGTATTGCCGAGAAAGAAATCATGGACGCAGTTGAAACCGGTAAACTGTACCTGTTCCAG
ATCTACAATAAAGATTTTGCCAAAGGCCATCATGGCAAACCGAATCTGCATACCCTGTATTG
GACCGGTCTGTTTAGCCCTGAAAATCTGGCAAAAACCTCGATTAAACTGAATGGTCAGGCGG
AACTGTTTTATCGTCCGAAAAGCCGTATGAAACGTATGGCAGCTCGTCTGGGTGAAAAAATG
CTGAACAAAAAACTGAAAGACCAGAAAACCCCGATCCCGGATACACTGTATCAAGAACTGTA
TGATTATGTGAACCATCGTCTGAGCCATGATCTGAGTGATGAAGCACGTGCCCTGCTGCCGA
ATGTTATTACCAAAGAAGTTAGCCACGAGATCATTAAAGATCGTCGTTTTACCAGCGACAAA
TTCCTGTTTCATGTGCCGATTACCCTGAATTATCAGGCAGCAAATAGCCCGAGCAAATTTAA
CCAGCGTGTTAATGCATATCTGAAAGAACATCCAGAAACGCCGATTATTGGTATTGATCGTG
GTGAACGTAACCTGATTTATATCACCGTTATTGATAGCACCGGCAAAATCCTGGAACAGCGT
AGCCTGAATACCATTCAGCAGTTTGATTACCAGAAAAAACTGGATAATCGCGAGAAAGAACG
TGTTGCAGCACGTCAGGCATGGTCAGTTGTTGGTACAATTAAAGACCTGAAACAGGGTTATC
TGAGCCAGGTTATTCATGAAATTGTGGATCTGATGATTCACTATCAGGCCGTTGTTGTGCTG
GAAAACCTGAATTTTGGCTTTAAAAGCAAACGTACCGGCATTGCAGAAAAAGCAGTTTATCA
GCAGTTCGAGAAAATGCTGATTGACAAACTGAATTGCCTGGTGCTGAAAGATTATCCGGCTG
AAAAAGTTGGTGGTGTTCTGAATCCGTATCAGCTGACCGATCAGTTTACCAGCTTTGCAAAA
ATGGGCACCCAGAGCGGATTTCTGTTTTATGTTCCGGCACCGTATACGAGCAAAATTGATCC
GCTGACCGGTTTTGTTGATCCGTTTGTTTGGAAAACCATCAAAAACCATGAAAGCCGCAAAC
ATTTTCTGGAAGGTTTCGATTTTCTGCATTACGACGTTAAAACGGGTGATTTCATCCTGCAC
TTTAAAATGAATCGCAATCTGAGTTTTCAGCGTGGCCTGCCTGGTTTTATGCCTGCATGGGA
TATTGTGTTTGAGAAAAACGAAACACAGTTCGATGCAAAAGGCACCCCGTTTATTGCAGGTA
AACGTATTGTTCCGGTGATTGAAAATCATCGTTTCACCGGTCGTTATCGCGATCTGTATCCG
GCAAATGAACTGATCGCACTGCTGGAAGAGAAAGGTATTGTTTTTCGTGATGGCTCAAACAT
TCTGCCGAAACTGCTGGAAAATGATGATAGCCATGCAATTGATACCATGGTTGCACTGATTC
GTAGCGTTCTGCAGATGCGTAATAGCAATGCAGCAACCGGTGAAGATTACATTAATAGTCCG
GTTCGTGATCTGAATGGTGTTTGTTTTGATAGCCGTTTTCAGAATCCGGAATGGCCGATGGA
TGCAGATGCAAATGGTGCATATCATATTGCACTGAAAGGACAGCTGCTGCTGAACCACCTGA
AAGAAAGCAAAGATCTGAAACTGCAAAACGGCATTAGCAATCAGGATTGGCTGGCATATATC
CAAGAACTGCGTAACGGTCGTAGCAGTGATGATGAAGCAACCGCAGATAGCCAGCATGCAGC
ACCGCCTAAAAAGAAACGTAAAGTT

Activin

The TGF-β superfamily consists of more than 45 members including activins, inhibins, myostatin, bone morphogenetic proteins (BMPs), growth and differentiation factors (GDFs) and nodal (see, e.g., Morianos et al., Journal of Autoimmunity 104:102314 (2019)). Activins are found either as homodimers or heterodimers of βA or/and βB subunits linked with disulfide bonds. There are three functional isoforms of activins: activin-A (βAβA), activin B (βBβB) and activin AB (βAβB) (Xia et al., J. Endocrinol. 202:1-12 (2009)). The βC and βE subunits are found in mammals and the βB subunit in Xenopus laevis. Transcripts of the βA and βB subunits are detected in nearly every tissue in the human body and exhibit increased expression in the reproductive system, while the βC and βE subunits are predominantly expressed in the liver (Woodruff, Biochem. Pharmacol. 55:953-963 (1998)). Activin-A is a cytokine of approximately 25 kDa and represents the most extensively investigated protein among the family of activins. Activin-A was initially identified as a gonadal protein that induces the biosynthesis and secretion of the follicle-stimulating hormone from the pituitary (Hedger et al., Cytokine Growth Factor Rev. 24:285-295 (2013)). It is highly conserved among vertebrates, reaching up to 95% homology between species. Activin-A regulates fundamental biologic processes, such as, haematopoiesis, embryonic development, stem cell maintenance and pluripotency, tissue repair and fibrosis (Kariyawasam et al., Clin. Exp. Allergy 41:1505-1514 (2011)).

Activin, e.g., Activin A, is well known and commercially available (from, e.g., STEMCELL Technologies Inc., Cambridge, Mass.).

Culture Methods

In general, an ES cell (e.g., an ES cell genetically engineered not to express one or more TGFβ receptor, e.g., TGFβRII) can be cultured to maintain pluripotency by culturing such ES cells in media that contains activin, e.g., a particular, effective level of activin (e.g., during one or more stages of culture).

In some embodiments, ES cells described herein are cultured (e.g., at one or more stages of culture) in a medium that includes activin, e.g., an elevated level of activin, to maintain pluripotency of the cells. In some embodiments, a level of one or more ES markers (e.g., SSEA-3, SSEA-4, TRA-1-60, TRA-1-81, TRA-2-49/6E, ALP, Sox2, E-cadherin, UTF-1, Oct4, Rex1, and/or Nanog) in a sample of cells from the culture is increased relative to the corresponding level(s) in a sample of cells cultured using the same medium that does not include activin, e.g., an elevated level of activin. In some embodiments, the increased level of one or more ES marker is higher than the corresponding level(s) by at least about 10%, 20%, 30%, 40%, 50%, 60%, 70%, 80%, 90%, 100%, 150%, 200%, 250%, 300%, 350%, 400%, 450%, 500%, or more, of the corresponding level.

As used herein, an “elevated level of activin” means a higher concentration of activin than is present in a standard medium, a starting medium, a medium used at one or more stages of culture, and/or in a medium in which ES cells are cultured. In some embodiments, activin is not present in a standard and/or starting medium, a medium used at one or more other stages of culture, and/or in a medium in which ES cells are cultured, and an “elevated level” is any amount of activin. A medium can include an elevated level of activin initially (i.e., at the start of a culture), and/or medium can be supplemented with activin to achieve an elevated level of activin at a particular time or times (e.g., at one or more stages) during culturing.

In some embodiments, an elevated level of activin is an increase of at least about 10%, 20%, 30%, 40%, 50%, 60%, 70%, 80%, 90%, 100%, 150%, 200%, 250%, 300%, 350%, 400%, 450%, 500%, 550%, 600%, 650%, 700%, 750%, 800%, 850%, 900%, 950%, 1000% or more, relative to a level of activin in a standard medium, a starting medium, a medium during one or more stages of culture, and/or in a medium in which ES cells are cultured.

In some embodiments, an elevated level of activin is about 0.5 ng/mL, 1 ng/mL, 2 ng/mL, 3 ng/mL, 4 ng/mL, 5 ng/mL, 10 ng/mL, 15 ng/mL, 20 ng/mL, 25 ng/mL, 30 ng/mL, 35 ng/mL, 40 ng/mL, 45 ng/mL, 50 ng/mL, 60 ng/mL, 70 ng/mL, 80 ng/mL, 90 ng/mL, 100 ng/mL, or more, activin. In some embodiments, an elevated level of activin is about 0.5 ng/mL to about 20 ng/mL activin, about 0.5 ng/mL to about 10 ng/mL activin, about 4 ng/mL to about 10 ng/mL activin.

Cells can be cultured in a variety of cell culture media known in the art, which are modified according to the disclosure to include activin as described herein. Cell culture medium is understood by those of skill in the art to refer to a nutrient solution in which cells, such as animal or mammalian cells, are grown. A cell culture medium generally includes one or more of the following components: an energy source (e.g., a carbohydrate such as glucose); amino acids; vitamins; lipids or free fatty acids; and trace elements, e.g., inorganic compounds or naturally occurring elements in the micromolar range. Cell culture medium can also contain additional components, such as hormones and other growth factors (e.g., insulin, transferrin, epidermal growth factor, serum, and the like); signaling factors (e.g., interleukin 15 (IL-15), transforming growth factor beta (TGF-(3), and the like); salts (e.g., calcium, magnesium and phosphate); buffers (e.g., HEPES); nucleosides and bases (e.g., adenosine, thymidine, hypoxanthine); antibiotics (e.g., gentamycin); and cell protective agents (e.g., a Pluronic polyol (Pluronic F68)).

Media that has been prepared or commercially available can be modified according to the present disclosure for utilization in the methods described herein. Nonlimiting examples of such media include Minimal Essential Medium (MEM, Sigma, St. Louis, Mo.); Ham's F10 Medium (Sigma); Dulbecco's Modified Eagles Medium (DMEM, Sigma); RPM 1-1640 Medium (Sigma); HyClone cell culture medium (HyClone, Logan, Utah); Power CHO2 (Lonza Inc., Allendale, N.J.); and chemically-defined (CD) media, which are formulated for particular cell types. In some embodiments, a culture medium is an E8 medium described in, e.g., Chen et al., Nat. Methods 8:424-429 (2011)). In some embodiments, a cell culture medium includes activin but lacks TGFβ.

Cell culture conditions (including pH, 02, CO2, agitation rate and temperature) suitable for ES cells are those that are known in the art, such as described in Schwartz et al., Methods Mol. Biol. 767:107-123 (2011) and Chen et al., Nat. Methods 8:424-429 (2011).

In some embodiments, cells are cultured in one or more stages, and cells can be cultured in medium having an elevated level of activin in one or more stages. For example, a culture method can include a first stage (e.g., using a medium having a reduced level of or no activin) and a second stage (e.g., using a medium having an elevated level of activin). In some embodiments, a culture method can include a first stage (e.g., using a medium having an elevated level of activin) and a second stage (e.g., using a medium having a reduced level of activin). In some embodiments, a culture method includes more than two stages, e.g., 3, 4, 5, 6, or more stages, and any stage can include medium having an elevated level of activin or a reduced level of activin. The length of culture is not limiting. For example, a culture method can be 5, 6, 7, 8, 9, 10, 11, 12, 13, 14, 15, 16, 17, 18, 19, 20, or more days. In some embodiments, a culture method includes at least two stages. For example, a first stage can include culturing cells in medium having a reduced level of activin (e.g., for about 1, 2, 3, 4, 5, 6, 7, 8, 9, 10, or more days), and a second stage can include culturing cells in medium having an elevated level of activin (e.g., for about 1, 2, 3, 4, 5, 6, 7, 8, 9, 10, or more days). In some embodiments, a first stage can include culturing cells in medium having an elevated level of activin (e.g., for about 1, 2, 3, 4, 5, 6, 7, 8, 9, 10, or more days), and a second stage can include culturing cells in medium having a reduced level of activin (e.g., for about 1, 2, 3, 4, 5, 6, 7, 8, 9, 10, or more days).

In particular methods, levels of one or more ES marker (e.g., SSEA-3, SSEA-4, TRA-1-60, TRA-1-81, TRA-2-49/6E, ALP, Sox2, E-cadherin, UTF-1, Oct4, Rex1, and/or Nanog) expressed in a sample of cells from a cell culture are monitored during one or more times (e.g., one or more stages) of cell culture, thereby allowing adjustment (e.g., increasing or decreasing the amount of activin in the culture) stopping the culture, and/or harvesting the cells from the culture.

Methods of Characterization

Methods of characterizing cells including characterizing cellular phenotype are known to those of skill in the art. In some embodiments, one or more such methods may include, but not be limited to, for example, morphological analyses and flow cytometry. Cellular lineage and identity markers are known to those of skill in the art. One or more such markers may be combined with one or more characterization methods to determine a composition of a cell population or phenotypic identity of one or more cells. For example, in some embodiments, cells of a particular population will be characterized using flow cytometry. In some such embodiments, a sample of a population of cells will be evaluated for presence and proportion of one or more cell surface markers and/or one or more intracellular markers. As will be understood by those of skill in the art, such cell surface markers may be representative of different lineages. For example, pluripotent cells may be identified by one or more of any number of markers known to be associated with such cells, such as, for example, CD34. Further, in some embodiments, cells may be identified by markers that indicate some degree of differentiation. Such markers will be known to one of skill in the art. For example, in some embodiments, markers of differentiated cells may include those associated with differentiated hematopoietic cells such as, e.g., CD43, CD45 (differentiated hematopoietic cells). In some embodiments, markers of differentiated cells may be associated with NK cell phenotypes such as, e.g., CD56 (also known as neural cell adhesion molecule), NK cell receptor immunoglobulin gamma Fc region receptor III (FcγRIII, cluster of differentiation 16 (CD16), natural killer group-2 member A (NKG2A), natural killer group-2 member D (NKG2D), CD69, a natural cytotoxicity receptor (e.g., NCR1, NCR2, NCR3, NKp30, NKp44, NKp46, and/or CD158b), killer immunoglobulin-like receptor (KIR), and CD94 (also known as killer cell lectin-like receptor subfamily D, member 1 (KLRD1)) etc. In some embodiments, markers may be T cell markers (e.g., CD3, CD4, CD8, etc.).

Methods of Use

A variety of diseases, disorders and/or conditions may be treated through use of technologies provided by the present disclosure. For example, in some embodiments, a disease, disorder and/or condition may be treated by introducing modified cells as described herein (e.g., edited iNK cells) to a subject. Examples of diseases that may be treated include, but not limited to, cancer, e.g., solid tumors, e.g., of the brain, prostate, breast, lung, colon, uterus, skin, liver, bone, pancreas, ovary, testes, bladder, kidney, head, neck, stomach, cervix, rectum, larynx, or esophagus; and hematological malignancies, e.g., acute and chronic leukemias, lymphomas, e.g., B-cell lymphomas including Hodgkin's and non-Hodgkin lymphomas, multiple myeloma and myelodysplastic syndromes.

In some embodiments, the present disclosure provides methods of treating a subject in need thereof by administering to the subject a composition comprising any of the cells described herein. In some embodiments, a therapeutic agent or composition may be administered before, during, or after the onset of a disease, disorder, or condition (including, e.g., an injury).

In particular embodiments, the subject has a disease, disorder, or condition, that can be treated by a cell therapy. In some embodiments, a subject in need of cell therapy is a subject with a disease, disorder and/or condition, whereby a cell therapy, e.g., a therapy in which a composition comprising a cell described herein, is administered to the subject, whereby the cell therapy treats at least one symptom associated with the disease, disorder, and/or condition. In some embodiments, a subject in need of cell therapy includes, but is not limited to, a candidate for bone marrow or stem cell transplant, a subject who has received chemotherapy or irradiation therapy, a subject who has or is at risk of having a hyperproliferative disorder or a cancer, e.g., a hyperproliferative disorder or a cancer of hematopoietic system, a subject having or at risk of developing a tumor, e.g., a solid tumor, and/or a subject who has or is at risk of having a viral infection or a disease associated with a viral infection.

Pharmaceutical Compositions

In some embodiments, the present disclosure provides pharmaceutical compositions comprising one or more genetically modified cells described herein, e.g., an edited iNK cell described herein. In some embodiments, a pharmaceutical composition further comprises a pharmaceutically acceptable excipient. In some embodiments, a pharmaceutical composition comprises isolated pluripotent stem cell-derived hematopoietic lineage cells comprising at least 50%, 60%, 70%, 80%, 90%, 95%, 98%, or 99% T cells, NK cells, NKT cells, CD34+HE cells or HSCs, e.g., genetically modified (e.g., edited) T cells, NK cells, NKT cells, CD34+HE cells or HSCs. In some embodiments, a pharmaceutical composition comprises isolated pluripotent stem cell-derived hematopoietic lineage cells comprising about 95% to about 100% T cells, NK cells, NKT cells, CD34+HE cells or HSCs, e.g., genetically modified (e.g., edited) T cells, NK cells, NKT cells, CD34+HE cells or HSCs.

In some embodiments, a pharmaceutical composition of the present disclosure comprises an isolated population of pluripotent stem cell-derived hematopoietic lineage cells, wherein the isolated population has less than about 0.1%, 0.5%, 1%, 2%, 5%, 10%, 15%, 20%, 25%, or 30% T cells, NK cells, NKT cells, CD34+HE cells or HSCs, e.g., genetically modified (e.g., edited) T cells, NK cells, NKT cells, CD34+HE cells or HSCs. In some embodiments, an isolated population of pluripotent stem cell-derived hematopoietic lineage cells has more than about 0.1%, 0.5%, 1%, 2%, 5%, 10%, 15%, 20%, 25%, or 30% T cells, NK cells, NKT cells, CD34+HE cells or HSCs, e.g., genetically modified (e.g., edited) T cells, NK cells, NKT cells, CD34+HE cells or HSCs. In some embodiments, an isolated population of pluripotent stem cell-derived hematopoietic lineage cells has about 0.1% to about 1%, about 1% to about 3%, about 3% to about 5%, about 10%-about 15%, about 15%-20%, about 20%-25%, about 25%-30%, about 30%-35%, about 35%-40%, about 40%-45%, about 45%-50%, about 60%-70%, about 70%-80%, about 80%-90%, about 90%-95%, or about 95% to about 100% T cells, NK cells, NKT cells, CD34+HE cells or HSCs, e.g., genetically modified (e.g., edited) T cells, NK cells, NKT cells, CD34+HE cells or HSCs.

In some embodiments, an isolated population of pluripotent stem cell-derived hematopoietic lineage cells comprises about 0.1%, about 1%, about 3%, about 5%, about 10%, about 15%, about 20%, about 25%, about 30%, about 35%, about 40%, about 45%, about 50%, about 60%, about 70%, about 80%, about 90%, about 95%, about 98%, about 99%, or about 100% T cells, NK cells, NKT cells, CD34+HE cells or HSCs, e.g., genetically modified (e.g., edited) T cells, NK cells, NKT cells, CD34+HE cells or HSCs.

As one of ordinary skill in the art would understand, both autologous and allogeneic cells can be used in adoptive cell therapies. Autologous cell therapies generally have reduced infection, low probability for GVHD, and rapid immune reconstitution relative to other cell therapies. Allogeneic cell therapies generally have an immune mediated graft-versus-malignancy (GVM) effect, and low rate of relapse relative to other cell therapies. Based on the specific condition(s) of the subject in need of the cell therapy, one of ordinary skill in the art would be able to determine which specific type of therapy(ies) to administer.

In some embodiments, a pharmaceutical composition comprises pluripotent stem cell-derived hematopoietic lineage cells that are allogeneic to a subject. In some embodiments, a pharmaceutical composition comprises pluripotent stem cell-derived hematopoietic lineage cells that are autologous to a subject. For autologous transplantation, the isolated population of pluripotent stem cell-derived hematopoietic lineage cells can be either a complete or partial HLA-match with patient subject. In some embodiments, the pluripotent stem cell-derived hematopoietic lineage cells are not HLA-matched to a subject.

In some embodiments, pluripotent stem cell-derived hematopoietic lineage cells can be administered to a subject without being expanded ex vivo or in vitro prior to administration. In particular embodiments, an isolated population of derived hematopoietic lineage cells is modulated and treated ex vivo using one or more agent to obtain immune cells with improved therapeutic potential. In some embodiments, the modulated population of derived hematopoietic lineage cells can be washed to remove the treatment agent(s), and the improved population can be administered to a subject without further expansion of the population in vitro. In some embodiments, an isolated population of derived hematopoietic lineage cells is expanded prior to modulating the isolated population with one or more agents.

In some embodiments, an isolated population of derived hematopoietic lineage cells can be genetically modified (e.g., by recombinant methods) to express TCR, CAR or other proteins. For genetically engineered derived hematopoietic lineage cells that express recombinant TCR or CAR, whether prior to or after genetic modification of the cells, the cells can be activated and expanded using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Cancers

Any cancer can be treated using a composition described herein. Exemplary therapeutic targets of the present disclosure include cancer cells from the bladder, blood, bone, bone marrow, brain, breast, colon, esophagus, eye, gastrointestine, gum, head, kidney, liver, lung, nasopharynx, neck, ovary, prostate, skin, stomach, testis, tongue, or uterus. In addition, a cancer may specifically be of the following non-limiting histological type: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; B-cell lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the cancer is a breast cancer. In some embodiments, the cancer is colon cancer. In some embodiments, the cancer is gastric cancer. In some embodiments, the cancer is RCC. In another embodiment, the cancer is non-small cell lung cancer (NSCLC).

In some embodiments, solid cancer indications that can be treated with iNK cells (e.g., genetically modified iNK cells, e.g., edited iNK cells) provided herein, either alone or in combination with one or more additional cancer treatment modality, include: bladder cancer, hepatocellular carcinoma, prostate cancer, ovarian/uterine cancer, pancreatic cancer, mesothelioma, melanoma, glioblastoma, HPV-associated and/or HPV-positive cancers such as cervical and HPV+ head and neck cancer, oral cavity cancer, cancer of the pharynx, thyroid cancer, gallbladder cancer, and soft tissue sarcomas. In some embodiments, hematological cancer indications that can be treated with the iNK cells (e.g., genetically modified iNK cells, e.g., edited iNK cells) provided herein, either alone or in combination with one or more additional cancer treatment modalities, include: ALL, CLL, NHL, DLBCL, AML, CML, and multiple myeloma (MM).

Examples of cellular proliferative and/or differentiative disorders of the lung include, but are not limited to, tumors such as bronchogenic carcinoma, including paraneoplastic syndromes, bronchioloalveolar carcinoma, neuroendocrine tumors, such as bronchial carcinoid, miscellaneous tumors, metastatic tumors, and pleural tumors, including solitary fibrous tumors (pleural fibroma) and malignant mesothelioma.

Examples of cellular proliferative and/or differentiative disorders of the breast include, but are not limited to, proliferative breast disease including, e.g., epithelial hyperplasia, sclerosing adenosis, and small duct papillomas; tumors, e.g., stromal tumors such as fibroadenoma, phyllodes tumor, and sarcomas, and epithelial tumors such as large duct papilloma; carcinoma of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma, and miscellaneous malignant neoplasms. Disorders in the male breast include, but are not limited to, gynecomastia and carcinoma.

Examples of cellular proliferative and/or differentiative disorders involving the colon include, but are not limited to, tumors of the colon, such as non-neoplastic polyps, adenomas, familial syndromes, colorectal carcinogenesis, colorectal carcinoma, and carcinoid tumors.

Examples of cancers or neoplastic conditions, in addition to the ones described above, include, but are not limited to, a fibrosarcoma, myosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, gastric cancer, esophageal cancer, rectal cancer, pancreatic cancer, ovarian cancer, prostate cancer, uterine cancer, cancer of the head and neck, skin cancer, brain cancer, squamous cell carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular cancer, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, or Kaposi sarcoma.

Exemplary useful additional cancer treatment modalities include, but are not limited to: chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfanide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Intl. Ed. Engl., 33: 183-186 (1994)); dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®), albumin-engineered nanoparticle formulation of paclitaxel (ABRAXANET™), and doxetaxel (TAXOTERE®); chloranbucil; 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; cyclosporine, sirolimus, rapamycin, rapalogs, ibandronate; topoisomerase inhibitor RFS 2000; difluoromethylornithine (DMFO); retinoids such as retinoic acid; CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU, leucovovin; anti-estrogens and selective estrogen receptor modulators (SERMs), including, for example, tamoxifen (including NOLVADEX® tamoxifen), raloxifene (EVISTA®), droloxifene, 4-hydroxytamoxifen, trioxifene, keoxifene, LY117018, onapristone, and toremifene (FARESTON®); anti-progesterones; estrogen receptor down-regulators (ERDs); estrogen receptor antagonists such as fulvestrant (FASLODEX®); agents that function to suppress or shut down the ovaries, for example, leutinizing hormone-releasing hormone (LHRH) agonists such as leuprolide acetate (LUPRON® and ELIGARD®), goserelin acetate, buserelin acetate and tripterelin; other anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE®), exemestane (AROMASIN®), formestanie, fadrozole, vorozole (RIVISOR®), letrozole (FEMARA®), and anastrozole (ARIMIDEX®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); aptamers, described for example in U.S. Pat. No. 6,344,321, which is herein incorporated by reference in its entirety; anti HGF monoclonal antibodies (e.g., AV299 from Aveo, AMG102, from Amgen); truncated mTOR variants (e.g., CGEN241 from Compugen); protein kinase inhibitors that block mTOR induced pathways (e.g., ARQ197 from Arqule, XL880 from Exelexis, SGX523 from SGX Pharmaceuticals, MP470 from Supergen, PF2341066 from Pfizer); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); lapatinib ditosylate (an ErbB-2 and EGFR dual tyrosine kinase small-molecule inhibitor also known as GW572016); COX-2 inhibitors such as celecoxib (CELEBREX®; 4-(5-(4-methylphenyl)-3-(trifluoromethyl)-1H-pyrazol-1-yl) benzenesulfonamide; and pharmaceutically acceptable salts, acids or derivatives of any of the above.

Other compounds that are effective in treating cancer are known in the art and described herein that are suitable for use with the compositions and methods of the present disclosure as additional cancer treatment modalities are described, for example, in the “Physicians' Desk Reference, 62nd edition. Oradell, N.J.: Medical Economics Co., 2008”, Goodman & Gilman's “The Pharmacological Basis of Therapeutics, Eleventh Edition. McGraw-Hill, 2005”, “Remington: The Science and Practice of Pharmacy, 20th Edition. Baltimore, Md.: Lippincott Williams & Wilkins, 2000.”, and “The Merck Index, Fourteenth Edition. Whitehouse Station, N.J.: Merck Research Laboratories, 2006”, incorporated herein by reference in relevant parts.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of:” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. The contents of database entries, e.g., NCBI nucleotide or protein database entries provided herein, are incorporated herein in their entirety. Where database entries are subject to change over time, the contents as of the filing date of the present application are incorporated herein by reference. Aspects of the embodiments can be modified, if necessary to employ concepts of the various patents, applications and publications to provide yet further embodiments.

The disclosure is further illustrated by the following examples. The examples are provided for illustrative purposes only. They are not to be construed as limiting the scope or content of the disclosure in any way.

### EXAMPLES

### Example 1: Generating Edited iPSC Cells Using Cas12a and Testing Effect of Activin a on Pluripotency

To generate natural killer cells from pluripotent stem cells, a representative induced pluripotent stem cell (iPSC) line was generated and designated “PCS-201”. This line was generated by reprogramming adult male human primary dermal fibroblasts purchased from ATCC (ATCC® PCS-201-012) using a commercially available non-modified RNA reprogramming kit (Stemgent/Reprocell, USA). The reprogramming kit contains non-modified reprogramming mRNAs (OCT4, SOX2, KLF4, cMYC, NANOG, and LIN28) with immune evasion mRNAs (E3, K3, and B18R) and double-stranded microRNAs (miRNAs) from the 302/367 clusters. Fibroblasts were seeded in fibroblast expansion medium (DMEM/F12 with 10% FBS). The next day, media was switched to Nutristem medium and daily overnight transfections were performed for 4 days (day 1 to 4). Primary iPSC colonies appeared on day 7 and were picked on day 10-14. Picked colonies were expanded clonally to achieve a sufficient number of cells to establish a master cell bank. The parental line chosen from this process and used for the subsequent experiments passed standard quality controls, including confirmation of stemness marker expression, normal karyotype and pluripotency.

To generate edited iPSC cells, the PCS-201 (PCS) cells were electroporated with a Cas12a RNP designed to cut at the target gene of interest. Briefly, the cells were treated 24 hours prior to transfection with a ROCK inhibitor (Y27632). On the day of transfection, a single cell solution was generated using accutase and 500,000 PCS iPS cells were resuspended in the appropriate electroporation buffer and Cas12a RNP at a final concentration of 2 μM. When two RNPs were added simultaneously, the total RNP concentration was 4 μM (2+2). This solution was electroporated using a Lonza 4D electroporator system. Following electroporation, the cells were plated in 6-well plates in mTESR media containing CloneR (Stemcell Technologies). The cells were allowed to grow for 3-5 days with daily media changes, and the CloneR was removed from the media by 48 hours post electroporation. To pick single colonies, the expanded cells were plated at a low density in 10 cm plates after resuspending them in a single cell suspension. Rock inhibitor was used to support the cells during single cell plating for 3-5 days post plating depending on the size of the colonies on the plate. After 7-10 days, sufficiently sized colonies with acceptable morphology were picked and plated into 24-well plates. The picked colonies were expanded to sufficient numbers to allow harvesting of genomic DNA for subsequent analysis and for cell line cryopreservation. Editing was confirmed by NGS and selected clones were expanded further and banked. Ultimately, karyotyping, sternness flow, and differentiation assays were performed on a subset of selected clones.

Two target genes of interest were CISH and TGFβRII, both of which were hypothesized to enhance natural killer cell function. As the TGFβ:TGFβRII pathway is believed to be involved in the maintenance of pluripotency, it was hypothesized that a functional deletion of TGFβRII in iPSCs could lead to differentiation and prevent generation of TGFβRII edited iPSCs. Due to the convergence of Activin receptor signaling and TGFβRII signaling in regulating SMAD2/3 and other intracellular molecules, it was hypothesized that Activin A could replace TGFβ in commercially available pluripotent stem cell medias to generate edited lines. To test this hypothesis, the pluripotency of unedited and TGFβRII edited iPSCs grown with Activin A was assessed. Several different culture medias were utilized: “E6” (Essential 6™ Medium, #A1516401, ThermoFisher), which lacks TGFβ, “E7”, which was E6 supplemented with 100 ng/ml of bFGF (Peprotech, #100-18B), “E8” (Essential 8™ Medium, #A1517001, ThermoFisher), and “E7+ActA”, which was E6 supplemented with 100 ng/ml of bFGF and varying concentrations of Activin A (Peprotech #120-14P). Typically, E6 and E7 medias are typically insufficient to maintain the sternness and pluripotency of PSCs over multiple passages in culture.

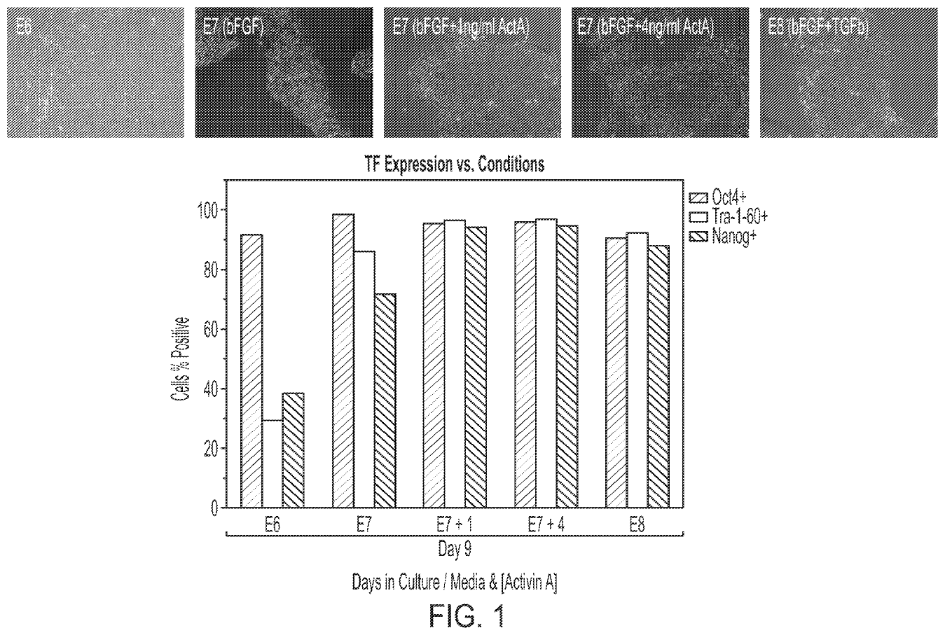

In order to determine whether Activin A could maintain PCS iPSCs in the absence of exogenous TGFβ, unedited PCS iPSCs were plated on a LaminStem™ 521 (Biological Industries) coated 6-well plate and cultured in E6, E7, E8 or E7+ActA (with Activin A at two different concentrations—1 ng/ml and 4 ng/ml). After 2 passages, the cells were assessed for morphology and sternness marker expression. Morphology was assessed using a standard phase contrast setting on an inverted microscope. Colonies with defined edges and non-differentiated cells typical of iPSC colonies, were deemed to be stem like. To confirm the morphological observations, the expression of standard iPS cell sternness markers was measured using intracellular flow cytometry. Briefly, cells were dissociated, stained for extracellular markers, and then fixed overnight and permeabilized using the reagents and standard protocol from the Foxp3/Transcription Factor Staining Buffer Set (eBioscience™). Cells were stained for flow cytometric analysis with anti-human TRA-1-60-R_AF®488 (Biolegend®; Clone TRA-1-60-R), anti-Human Nanog AF®647 (BD Pharmingen™; Clone N31-355), and anti-Oct4 (Oct3) PE (Biolegend®; Clone 3A2A20),. Cells were recorded on a NovoCyte Quanteon Flow Cytometer (Agilent) and analyzed using FlowJo (FlowJo, LLC). As shown in FIG. 1, both 1 ng/mL and 4 ng/ml of Activin A was sufficient to maintain pluripotency with equivalent sternness marker expression to the cells grown in E8. As expected, cells grown in E6 and E7 (which lacked TGFβ) did not maintain sternness gene expression to the same degree as E8, indicating the loss of iPSC sternness in the absence of TGFβ or Activin A. These results suggest that Activin A can supplement iPSC sternness in the absence of TGFβ signaling.

Given the demonstration that Activin A could support iPSC sternness in the absence of TGFβ, TGFβRII knockout (“KO”) iPSCs, CISH KO iPSCs, and TGFβRII/CISH double knockout (“DKO”) iPSC lines were generated. Specifically, iPSCs were edited using an RNP having an engineered Cas12a with three amino acid substitutions (M537R, F870L, and H800A (SEQ ID NO: 1148)) and a gRNA specific for CISH or TGFβRII. To make CISH/TGFβRII DKO iPSCs, iPSCs were treated with an RNP targeting CISH and an RNP targeting TGFβRII simultaneously. The particular guide RNA sequences of Table 10 were used for editing of CISH and TGFβRII. Both guides were generated with a targeting domain consisting of RNA, an AsCpf1 scaffold of the sequence UAAUUUCUACUCUUGUAGAU (SEQ ID NO: 1153) located 5′ of the targeting domain, and a 25-mer DNA extension of the sequence ATGTGTTTTTGTCAAAAGACCTTTT (SEQ ID NO: 1154) at the 5′ terminus of the scaffold sequence.

TABLE 10
Guide RNA sequences

| | gRNA Targeting | Full Length gRNA |
|---|---|---|
| Target | Domain Sequence | Sequence |
| CISH | GGUGUACAGCAGUGGCU | ATGTGTTTTTGTCAAAAGACCTT |
| 7050 | GGU | TTrUrArArUrUrUrCrUrArCr |
| | (SEQ ID NO: 1155) | UrCrUrUrGrUrArGrArUrGrG |
| | | rUrGrUrArCrArGrCrArGrUr |
| | | GrGrCrUrGrGrU (SEQ ID |
| | | NO: 1156) |
| TGFβRII | UGAUGUGAGAUUUUCCA | ATGTGTTTTTGTCAAAAGACCTT |
| 24026 | CCU | TTrUrArArUrUrUrCrUrArCr |
| | (SEQ ID NO: 1157) | UrCrUrUrGrUrArGrArUrUrG |
| | | rArUrGrUrGrArGrArUrUrUr |
| | | UrCrCrArCrCrU (SEQ ID |
| | | NO: 1158) |

The edited clones were generated as described above with a minor modification for the cells treated with TGFβRII RNPs. Briefly, TGFβRII-edited PCS iPSCs and TGFβRII/CISH edited PCS iPSCs were plated after electroporation at the 6-well stage in the mTESR supplemented with 10 ng/ml of Activin A in order to support the generation of edited clones. The cells were cultured with 10 ng/ml of Activin A through the cell colony picking and early expansion stages. Colonies assessed as having the correct single KO (CISH KO or TGFβRII KO) or double KO (CISH/TGFβRII DKO) were picked and expanded (clonal selection).

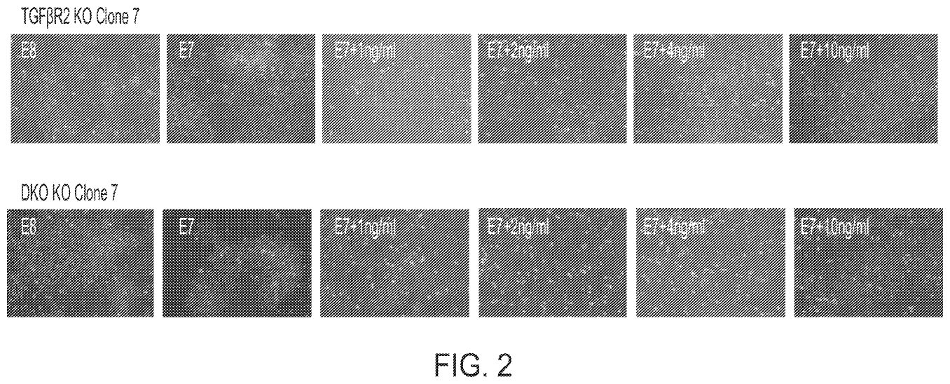

To determine the optimal concentration of Activin A for culturing of TGFβRII KO and TGFβRII/CISH DKO iPSCs, a slightly expanded concentration curve was tested as shown FIG. 2. Similar to the assessment performed previously, the iPSCs were cultured in a Matrigel-treated 6-well plate with concentrations of 1 ng/ml, 2 ng/ml, 4 ng/ml and 10 ng/ml Activin A. As shown in FIG. 2, TGFβRII KO or CISH/TGFβRII DKO cells cultured in E7 medium supplemented with 4 ng/mL Activin A for 19 days (over 5 passages) maintained a wild type morphology. FIG. 3 shows the morphology of TGFβRII KO PCS-201 hiPSC Clone 9.

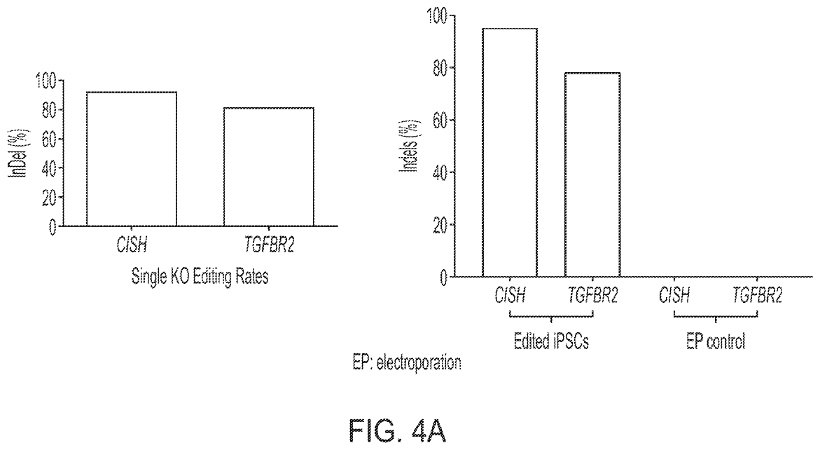

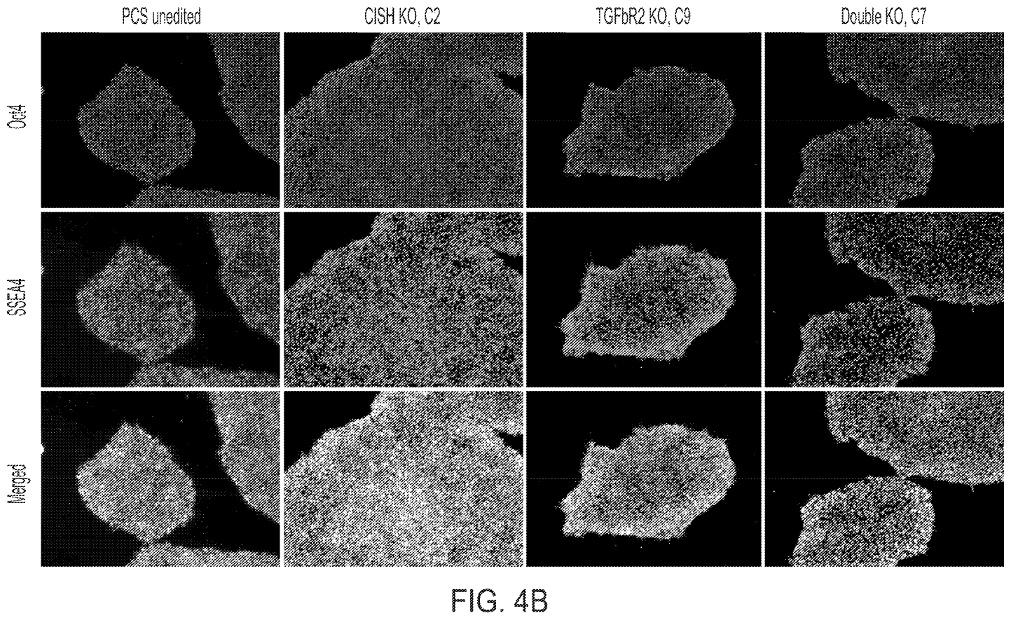

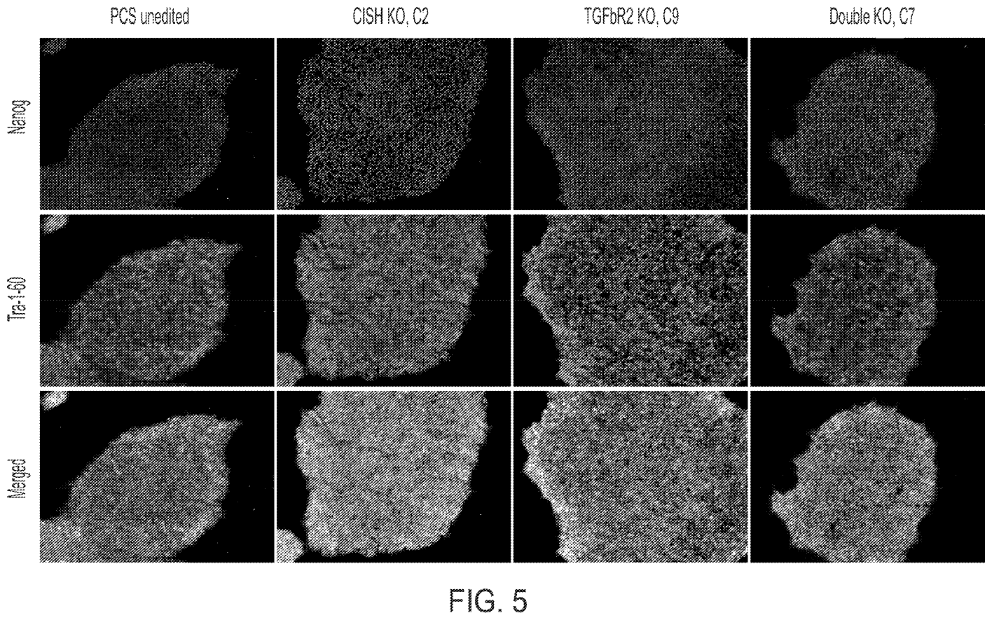

As shown in FIG. 4A, the initial editing efficiency of the iPSCs treated simultaneously with the CISH and TGFβRII RNPs (prior to clonal selection) was high, with 95% of the CISH alleles edited and 78% of the TGFβRII alleles edited. Unedited iPSC controls did not have indels at either loci. iPSCs that were treated with CISH or TGFβRII RNPs individually showed 93% and 82% editing rates prior to clone selection (depicted in FIG. 4A). The KO cell lines (CISH KO iPSCs, TGFβRII KO iPSCs, and CISH/TGFβRII DKO iPSCs) were subsequently assessed for the presence of pluripotency markers Oct4, SSEA4, Nanog, and Tra-1-60 after culturing in the presence of supplemental Activin A. As shown in FIGS. 4B and 5, culturing the KO cell lines in Activin A maintained expression of these pluripotency markers.

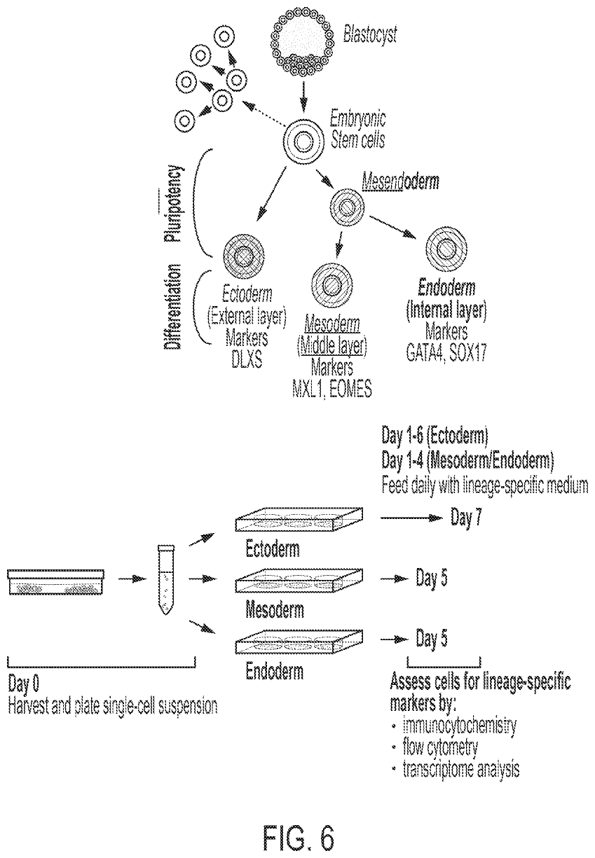

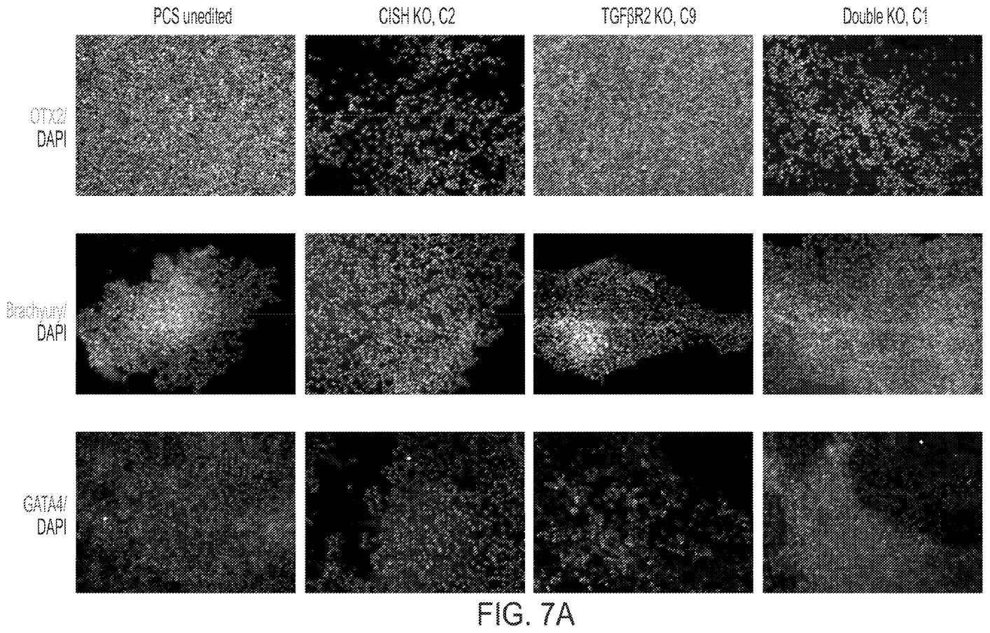

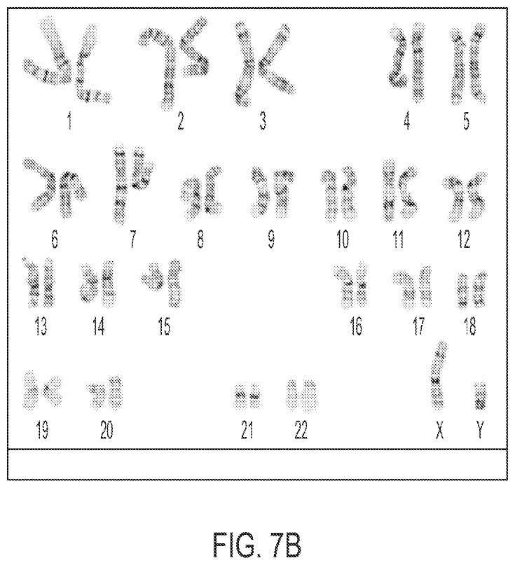

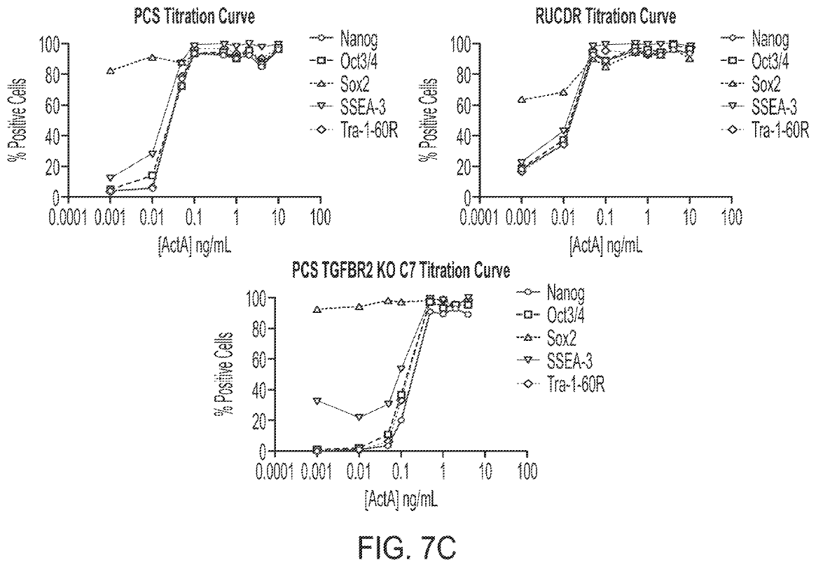

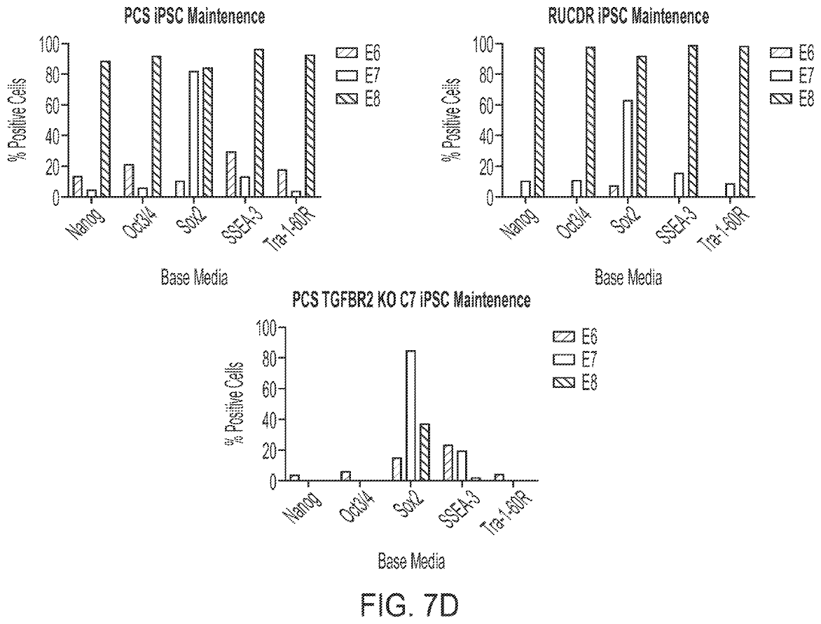

The KO iPSC lines cultured in Activin A were next assessed for their capacity to differentiate using the STEMdiff™ Trilineage Differentiation Kit assay (from STEMCELL Technologies Inc., Vancouver, BC, CA) as depicted schematically in FIG. 6. As shown in FIG. 7A, culturing the single KO (TGFβRII KO iPSCs or CISH KO iPSCs) and DKO (TGFβRII/CISH DKO iPSCs) cell lines in media with supplemental Activin A maintained their ability to differentiate into early progenitors of all 3 germ layers, as shown by expression of ectoderm (OTX2), mesoderm (brachyury), and endoderm (GATA4) markers (FIG. 7A). The unedited PCS control cells were also able to express each of these markers.

The edited iPSCs were next karyotyped to determine whether the Cas12a editing caused large genetic abnormalities, such as translocations. As shown in FIG. 7B, the cells had normal karyotypes with no translocation between the cut sites.

To further support the results described above, an expanded Activin A concentration curve was performed on the unedited parental PSC line, an edited TGFβRII KO iPSC clone (C7), and an additional representative (unedited) cell line designated RUCDR (RUCDR Infinite Biologics group, Piscaway N.J.). At the outset, the iPSCs were seeded at 1e5 cells per well in a 1× LaminStem™ 521 (Biological Industries) coated 12-well plate. Cells were then passaged 10 times over ˜40-50 days using 0.5 mM EDTA in 1×PBS dissociation and Y-27632 (Biological Industries) until wells achieved >75% confluency. Cells were cultured in Essential 6™ Medium (Gibco), TeSR™-E7 ™, and TeSR™-E8™ (StemCell Technologies) for controls and titrated using TeSR™-E7™ supplemented with E. coli-derived recombinant human/murine/rat Activin A (PeproTech) spanning a 4-log concentration dosage (0.001-10 ng/mL). Following 5 and 10 passages, cells were dissociated and then fixed overnight and permeabilized using the reagents and standard protocol from the Foxp3/Transcription Factor Staining Buffer Set (eBioscience™). Cells were stained for flow cytometric analysis with anti-human TRA-1-60-R_AF®488 (Biolegend®; Clone TRA-1-60-R), anti Sox2 PerCP-Cy™5.5 (BD Pharmingen™; Clone 030-678), anti Human Nanog_AF®647 (BD Pharmingen™; Clone N31-355), anti-Oct4 (Oct3) PE (Biolegend®; Clone 3A2A20), and anti-human SSEA-4 PE/Dazzle™ 594 (Biolegend®; Clone MC-813-70). Cells were recorded on a NovoCyte Quanteon Flow Cytometer (Agilent) and analyzed using FlowJo (FlowJo, LLC). FIG. 7C shows the titration curves for the tested iPSC lines. The minimum concentration of Activin A required to maintain each line varied slightly, with the TGFβRII KO iPSCs requiring a higher baseline amount of Activin A as compared to the parental control (0.5 ng/ml vs 0.1 ng/ml). In all 3 cell lines, 4 ng/ml was well above the minimum amount of Activin A necessary to maintain stemness marker expression over an extended culture period. FIG. 7D shows the stemness marker expression in the cells culture with the base medias alone (no Activin A). As expected, the TGFβRII KO iPSCs did not maintain expression, while the two unedited lines were able to maintain stemness marker expression in E8.

### Example 2: Differentiation of Edited CISH KO, TGFβRII KO, and CISH/TGFβRII DKO iPSCs into iNK Cells Exhibiting Enhanced Function

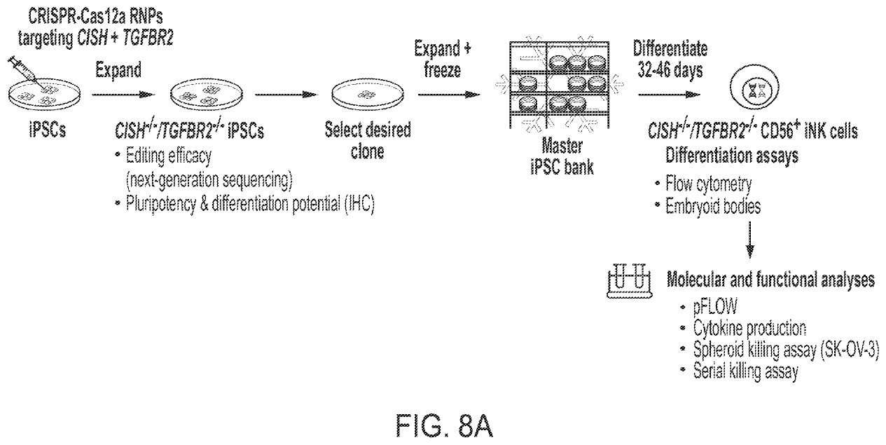

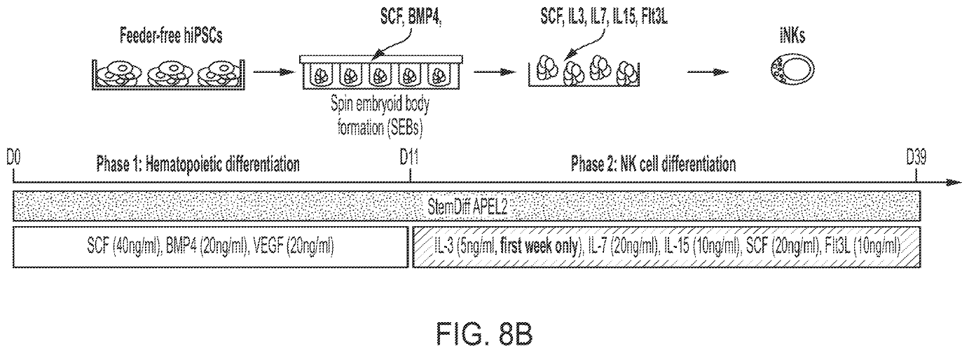

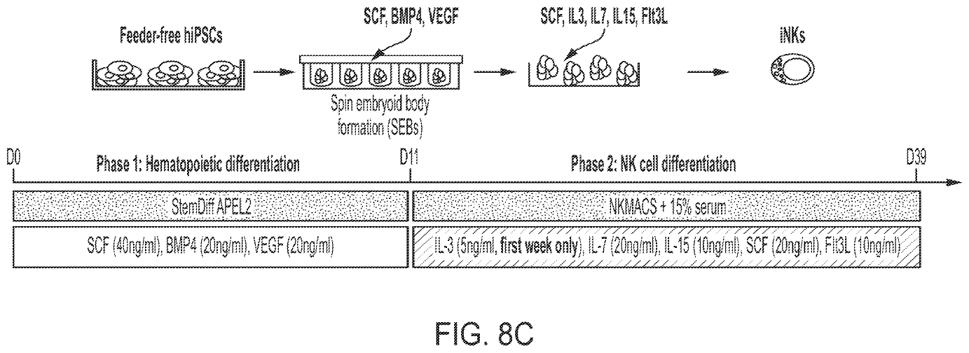

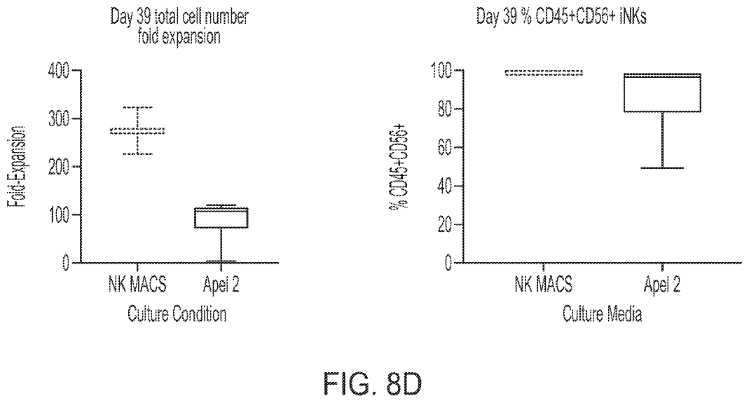

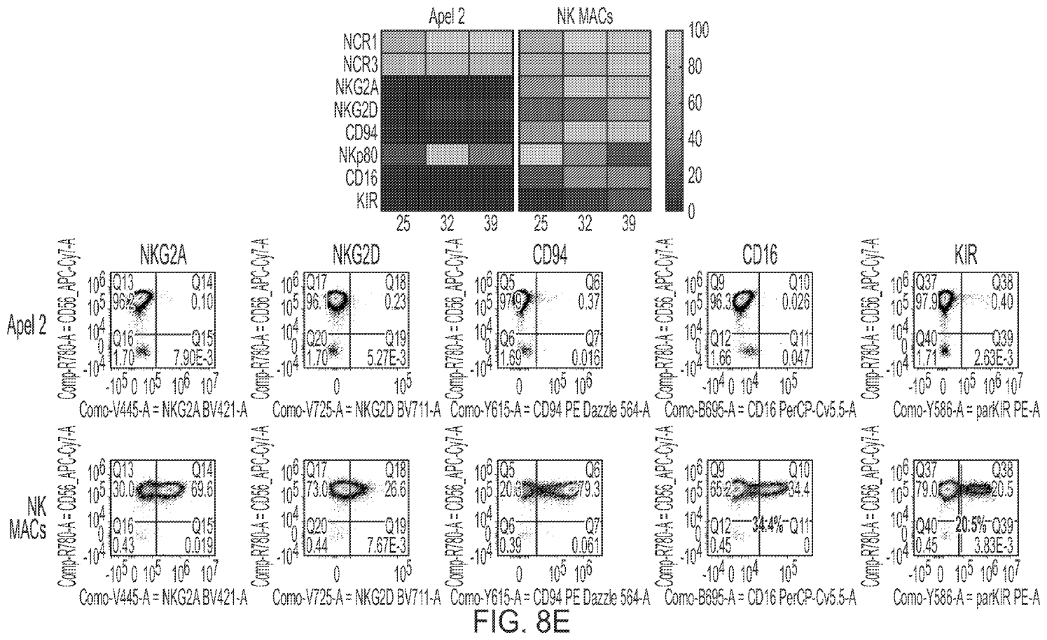

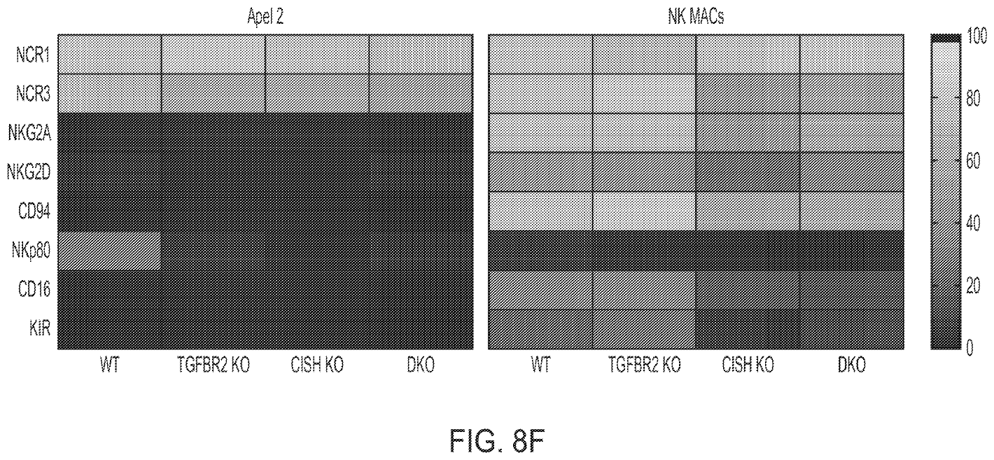

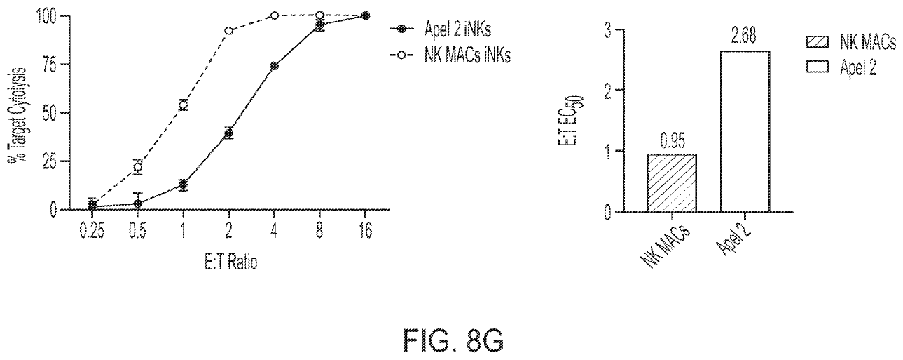

FIG. 8A depicts a schematic of an exemplary workflow for development of a CRISPR-Cas12a-edited iPSC platform for generation of enhanced CD56+ iNK cells. As shown in FIG. 8A, the CISH and TGFβRII genes are targeted in iPSCs via delivery of RNPs to the cells using electroporation to generate CISH/TGFβRII DKO iPSCs. iPSCs with the desired edits at both the CISH and TGFβRII genes can then be selected and expanded to create a master iPSC bank. Edited cells from the iPSC master bank can then be differentiated into CD56+ CISH/TGFβRII DKO iNK cells.

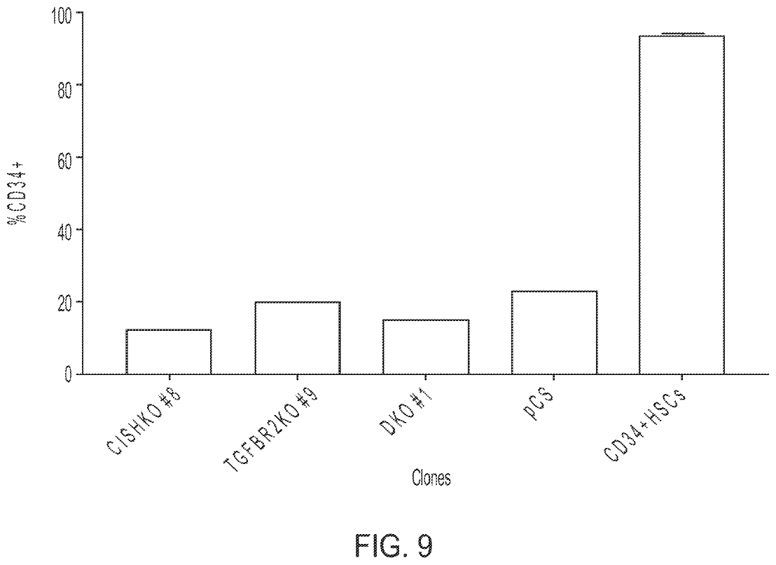

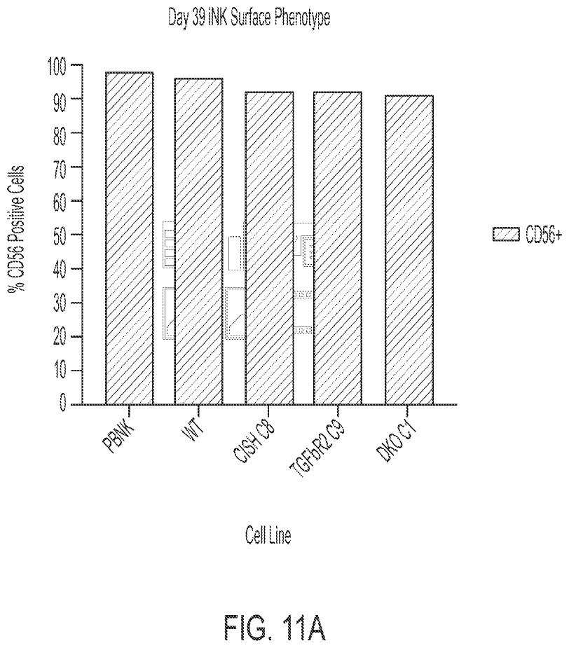

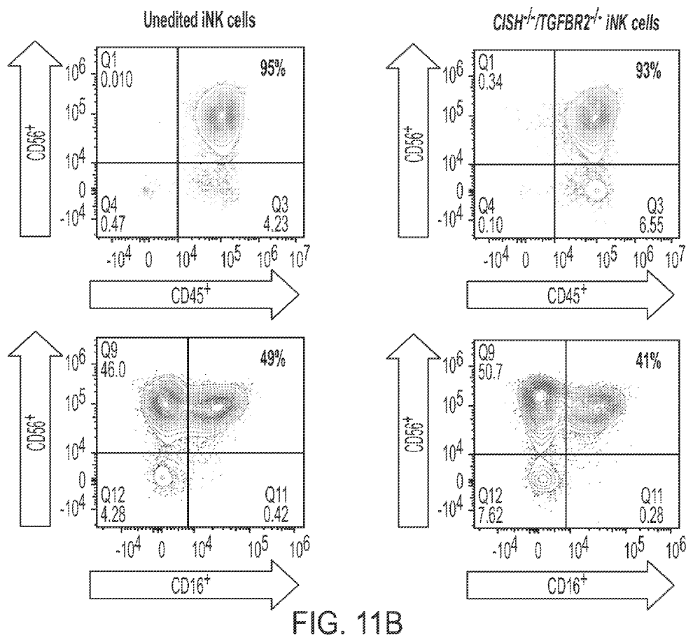

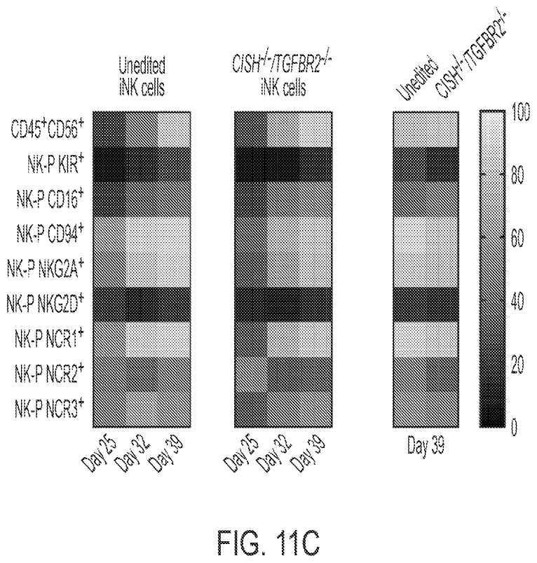

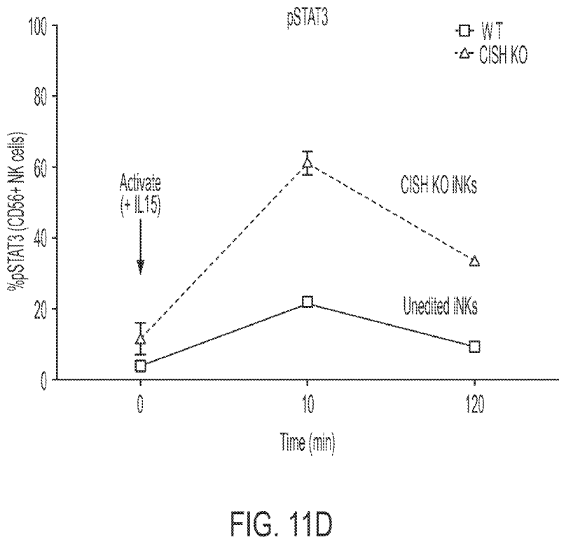

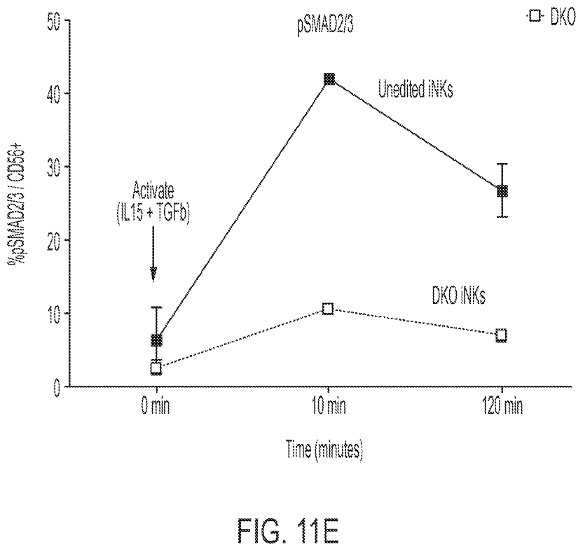

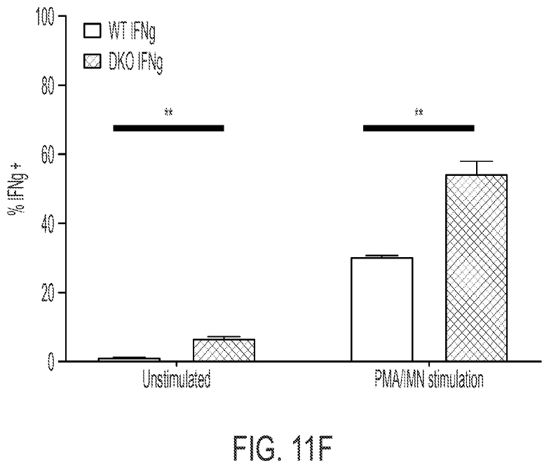

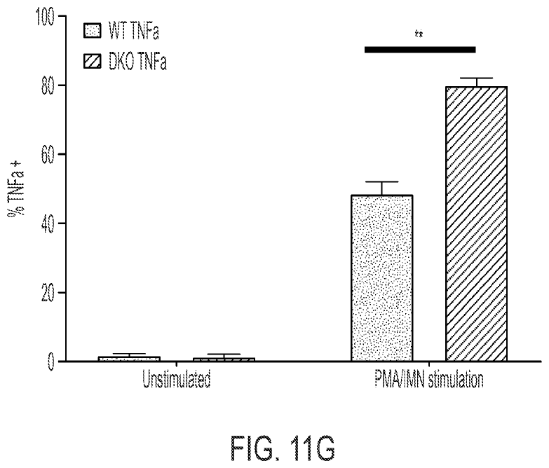

FIGS. 8B and 8C depict two exemplary schematics of the process of differentiating iPSCs into iNK cells. As shown in FIGS. 8B and 8C, edited cells (or unedited control cells) were differentiated using a two-phase process. First, in the “hematopoietic differentiation phase,” hiPSCs (edited and unedited) were cultured in StemDiff™ APEL2™ medium (StemCell Technologies) with SCF (40 ng/mL), BMP4 (20 ng/mL), and VEGF (20 ng/mL) from days 0-10, to produce spin embryoid bodies (SEBs). As shown in FIG. 8B, SEBs were then cultured from days 11-39 in StemDiff™ APEL2™ medium comprising IL-3 (5 ng/mL, only present for the first week of culture), IL-7 (20 ng/mL), IL-15 (10 ng/mL), SCF (20 ng/mL), and Flt3L (10 ng/mL) in an NK cell differentiation phase. CISH KO iPSCs, TGFβRII KO iPSCs, CISH/TGFβRII DKO iPSCs, and unedited wild-type iPSC lines (PCS), were differentiated into iNKs according to the schematic in FIG. 8B, and then characterized to assess whether they exhibited a phenotype congruent with NK cells (see FIGS. 9, 10, and 11A). CISH KO iPSCs, TGFβRII KO iPSCs, CISH/TGFβRII DKO iPSCs, and unedited wild-type iPSC lines, described in FIGS. 11B, 11C, 12B, 12C, and 13 were also differentiated into iNKs utilizing the alternative method shown in FIG. 8C, and then characterized to assess whether they exhibited a phenotype congruent with NK cells (see FIGS. 11B, 11C, 12B, 12C, and 13).

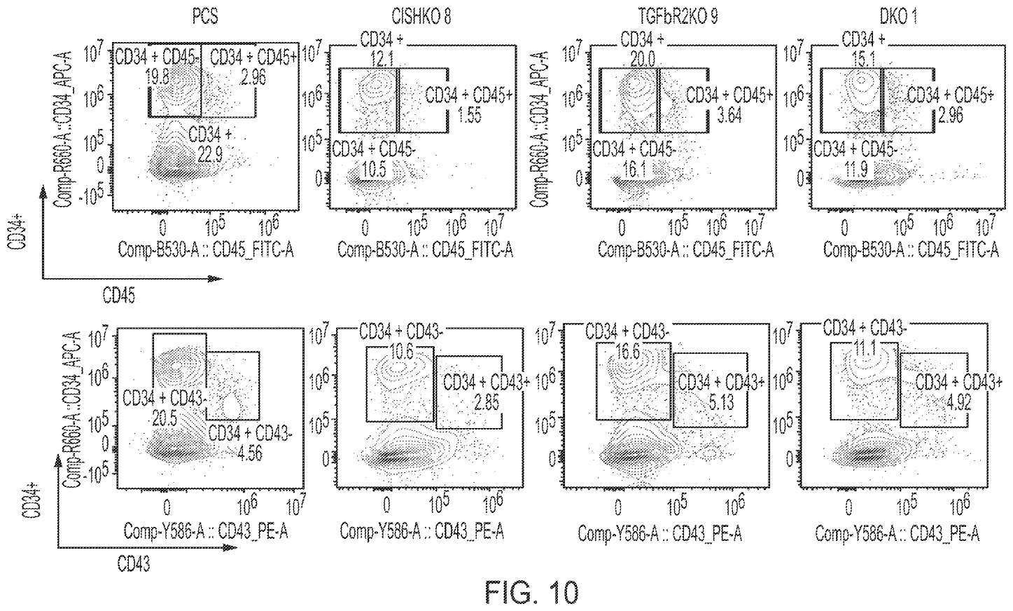

Specifically, the CISH KO iNKs, TGFβRII KO iNKs, CISH/TGFβRII DKO iNKs were assessed for exemplary phenotypic markers of (i) stem cells (CD34); and (ii) hematopoietic cells (CD43 and CD45) by flow cytometry. Briefly, two rows of embryoid bodies from a 96-well plate for each genotype were harvested for staining. Once a single cell solution was generated using Trypsin and mechanical disruption, the cells were stained for the human markers CD34, CD45, CD31, CD43, CD235a and CD41. As shown in FIG. 9, CISH KO iNKs, TGFβRII KO iNKs, CISH/TGFβRII DKO iNKs, and iNKs derived from wild-type parental clones (PCS) exhibited lower levels of CD34 relative to control cells, which were purified CD34+ HSCs. CD34 expression levels were similar across these iNK cell clones indicating that editing of the iPSCs did not affect differentiation to the CD34+ stage. FIG. 10 shows that CISH KO iNKs, TGFβRII KO iNKs, CISH/TGFβRII DKO iNKs, and iNKs derived from wild-type parental clones (PCS) exhibited similar surface expression profiles for CD43 and CD45. Thus, iNKS differentiated from edited and unedited iPSCs exhibited similar levels of markers for stem cells and hematopoietic cells, and both differentiated edited and unedited cells exhibited certain NK cell phenotypes based on marker expression profiles.

CISH KO iNKs, TGFβRII KO iNKs, CISH/TGFβRII DKO iNKs, iNKs derived from wild-type parental clones (WT), and NK cells derived from peripheral blood (PBNKs) were further assayed to determine their surface expression of CD56, a marker for NK cells. Briefly, cells were harvested on day 39 of differentiation, washed and resuspended in a flow staining buffer containing antibodies that recognize human CD56, CD16, NKp80, NKG2A, NKG2D, CD335, CD336, CD337, CD94, CD158. Cells events were recorded on a NovoCyte Quanteon Flow Cytometer (Agilent) and analyzed using FlowJo (FlowJo, LLC). FIG. 11A shows that iNK cells derived from edited iPSCs exhibited similar CD56+ surface expression relative to iNKs derived from unedited iPSC parental clones and PBNK cells (at day 39 in culture). FIG. 11B shows that iNK cells derived from edited iPSCs exhibited similar CD56+ and CD16+ surface expression relative to iNKs derived from unedited iPSC parental clones (at day 39 in culture). FIG. 11C shows that iNK cells derived from edited iPSCs exhibited similar CD56+, CD54+, KIR+, CD16+, CD94+, NKG2A+, NKG2D+, NCR1+, NCR2+, and NCR3+ surface expression relative to iNKs derived from unedited iPSC parental clones and PBNK cells (at day 39 in culture)

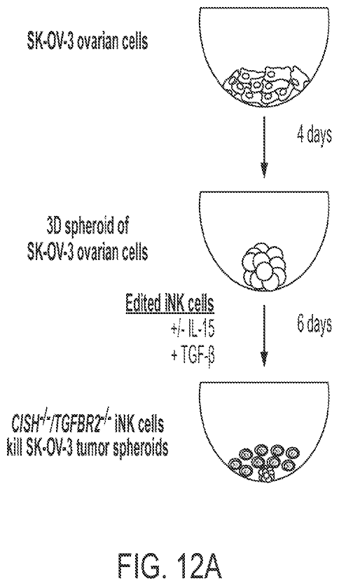

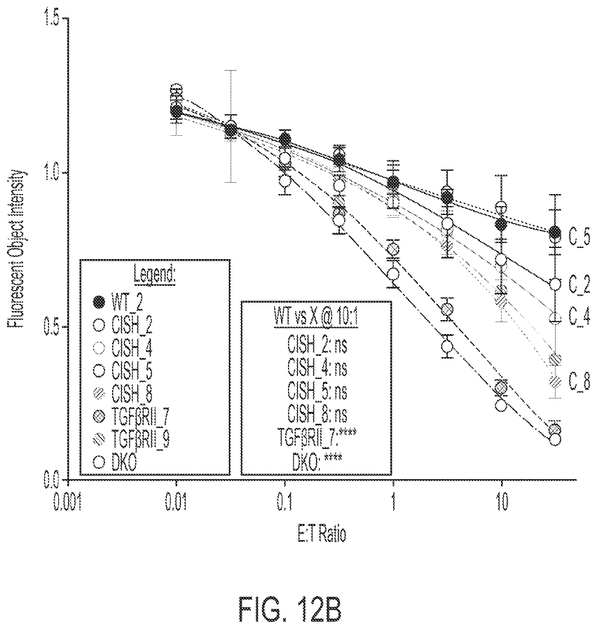

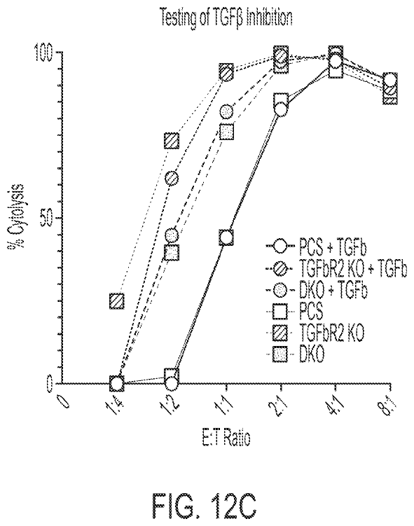

To confirm cell functionality, cells were assessed using a tumor cell cytotoxicity assay on the xCelligence platform. Briefly, tumor targets, SK-OV-3 tumor cells, were plated and grown to an optimal cell density in 96-well xCelligence plates. iNKs were then added to the tumor targets at different E:T ratios (1:4, 1:2, 1:1, 2:1. 4:1 and 8:1) in the presence of TGFβ. FIG. 12C shows that TGFβRII KO and CISH/TGFβRII DKO cells more effectively killed SK-OV-3 cells, as measured by percent cytolysis, relative to unedited iNK cells either in the presence or absence of TGF-β (at E:T ratios of 1:4, 1:2, 1:1, and 2:1).

While iNK cells generated using the alternative method described in FIG. 8B were CD56+ and capable of killing tumor targets in an in vitro cytotoxicity assay, the iNKs did not express many of the canonical markers associated with mature NK cells such as CD16, NKG2A, and KIRs. A K562 feeder cell line is typically used to expand and mature iNKs that are generated by similar differentiation methodologies. After expansion on feeders, the iNKs often express CD16, KIRs and other surface markers indicative of a more mature phenotype. In order to identify a feeder free approach to achieve more mature iNKs with enhanced functionality, an alternative media composition was tested for the stage of differentiation between day 11 and day 39. Instead of culturing cells between day 11 and day 39 in APEL2 (as shown in FIG. 8B), the spin embryoid bodies (SEBs) were cultured in NK MACS® media (MACS Miltenyi Biotec) with 15% human AB serum in the presence of the same cytokines as mentioned above. This protocol is depicted in FIG. 8C. In order to compare the two media compositions, Day 11 SEBs from WT PCS, TGFβRII KO iPSCs, CISH KO iPSCs, and DKO iPSCs were split into two conditions for the second half of the differentiation process, one with APEL2 base and the other with the NKMACS+serum base. At day 39, the cell yield, marker expression, and cytotoxicity levels were assessed. In all cases, the NKMACS+serum condition (depicted in FIG. 8C) outperformed the APEL2 condition (depicted in FIG. 8B). FIG. 8D shows that the NKMACS+serum condition yielded a greater fold expansion at the end of the 39 day process (nearly 300 fold expansion vs 100 fold expansion). When NK marker expression was analyzed by flow cytometry as described above, the iNKs cultured in NKMACS+serum were 34% CD16 positive and exhibited 20% KIR expression while the APEL2 conditions yielded cells that were essentially negative for both markers. This was the case for all genotypes tested. In order to visualize the markers relative to time or condition, flow cytometry data was gated and analyzed in FlowJo and heat maps were constructed (FIGS. 8E and 8F). Samples were first cleaned by gating for live cells (FSC-H vs. LIVE/DEAD™ Fixable Yellow) followed by immune cells (SSC-A vs. FSC-A), singlets (FSC-H vs. FSC-A) and the natural killer cell population (CD56 vs. CD45). The NK population, defined as CD45+56+ cells, was gated and each marker was analyzed along the X-axis in an analysis synonymous to a histogram/count plot (CD16+, CD94+, NKG2A+, NKG2D+, CD335+, CD336+, CD337+, NKp80+, panKIR+). Statistics for the aforementioned markers are visualized with a double-gradient heat map (GraphPad Prism 8) with the key set to the following parameters: black=0, medium intensity 30<x<50, maximum intensity=100. Based on this analysis, the expression kinetics and magnitude across all genotypes were improved by the NKMACS+serum condition. The cells were also assessed in a tumor cell cytotoxicity assay as described previously. The iNKs generated in the NKMACS+serum conditions were capable of killing at a lower E:T ratio than the cells differentiated in APEL2, indicating that the improved NK maturation had a positive impact on the functionality of the cells (FIG. 8G).

Analysis of additional differentiation markers in NKMACS+serum confirmed the presence of CD16 expression. FIG. 11B shows analysis of specific subpopulations (CD45 vs CD56 and CD56 vs CD16) derived from unedited or DKO iPSCs. Additionally, the cell surface marker profile of unedited iNK cells and CISH/TGFβRII DKO iNKs in FIG. 11C confirmed that the NK cell marker profile of the edited iNK cells was similar to that of unedited iNK cells. Taken together, these data show that Cas12a-edited single and double KO iPSC clones differentiate into iNK cells in a similar fashion as unedited iPSC clones, as defined by NK cell markers.

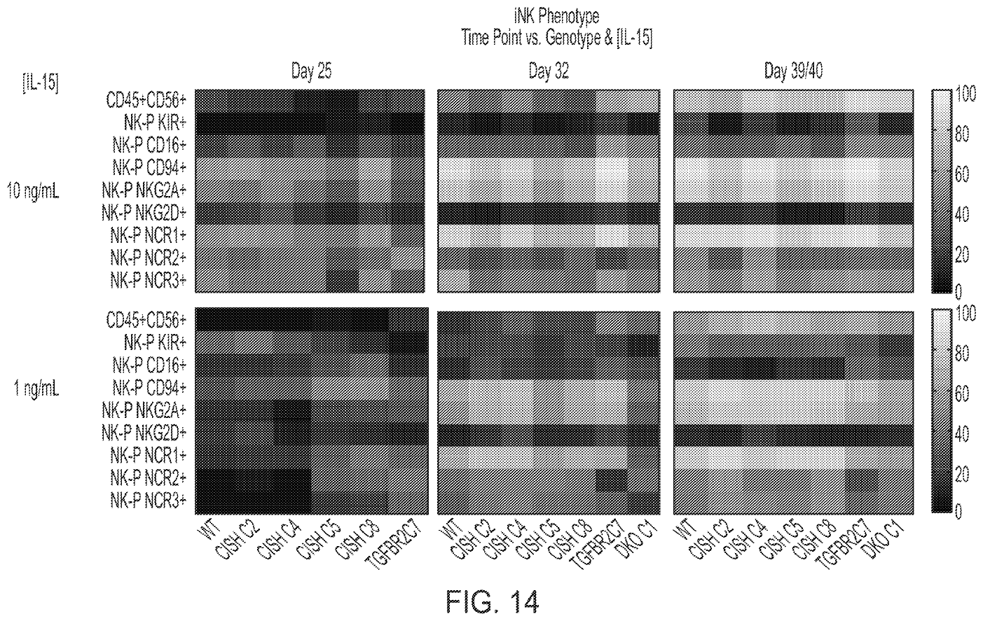

Additionally, certain edited iNK clonal cells (CISH single knockout “CISH_C2, C4, C5, and C8”, TGFβRII single knockout “TGFβRII-C7”, and TGFβRII/CISH double knockout “DKO-C1”), and parental clone iNK cells (“WT”) were cultured in the presence of 1 ng/mL or 10 ng/mL IL-15, and differentiation markers were assessed at day 25, day 32, and day 39 post-hiPSC differentiation. As shown in FIG. 14, surface expression phenotypes (measured as a percentage of the population) culturing in 10 ng/mL IL-15 resulted in a higher proportion of surface expression in the single knockouts, double knockouts, and the parental clonal line.

The edited iNK cells differentiated in NK MACS® medium+serum conditions were assessed for effector function in vitro using a range of molecular and functional analyses. First, a phosphoflow cytometry assay was performed to determine the phosphorylated state of STAT3 (pSTAT3) and SMAD2/3 (pSMAD2/3) in the day 39 iNK cells. CISH KO iNKs exhibited increased pSTAT3 upon IL-15 stimulation (FIG. 11D), and CISH/TGFβRII DKO iNKs exhibited decreased pSMAD2/3 levels upon TGF-β stimulation as compared to unedited iNK cells (FIG. 11E). These data suggest that CISH/TGFβRII DKO iNKs have enhanced sensitivity to IL-15 and resistance to TGF-β mediated immunosuppression. In addition, CISH/TGFβRII DKO iNKs were characterized for IFNγ and TNFα production using a phorbol myristate acetate and Ionomycin (PMA/IMN) stimulation assay. Briefly, cells were treated with 2 ng/ml of PMA and 0.125 μM of Ionomycin along with a protein transport inhibitor for 4 hours. The cells were harvested and stained using a standard intracellular staining protocol. The CISH/TGFβRII DKO iNKs produced significantly higher amounts of IFNγ and TFNa when stimulated with PMA/IMN (FIGS. 11F and 11G), providing evidence of enhanced cytokine production following stimulation relative to unedited control iNKs.

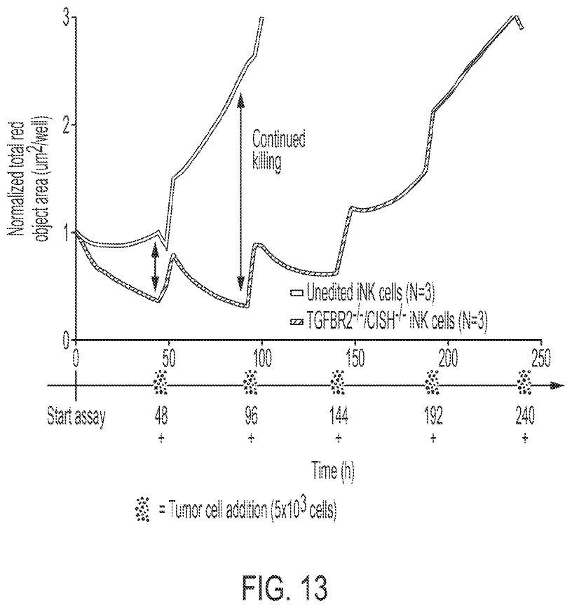

To test iNK tumor cell killing activity, a 3D solid tumor cell killing assay (depicted schematically in FIG. 12A) was utilized. In brief, spheroids were formed by seeding 5,000 NucLight Red labeled SK-OV-3 cells in 96 well ultra-low attachment plates. Spheroids were incubated at 37° C. before addition of effector cells (at different E:T ratios) and 10 ng/mL TGF-β, spheroids were subsequently imaged every 2 hours using the Incucyte S3 system for up to 120 hours. Data shown are normalized to the red object intensity at time of effector addition. Normalization of spheroid curves maintains the same efficacy patterns observed in non-normalized data. Using this assay, the cytotoxicity of iNKs differentiated from four CISH KO iPSC clones, two TGFβRII KO iPSC clones and one CISH/TGFβRII DKO iPSC clone were compared to control iNKs derived from the unedited parental iPSCs. As shown in FIG. 12B, edited iNK cells were capable of reducing the size of SK-OV-3 spheroids more effectively than unedited iNK control cells (averaged data from 6 assays). In particular the CISH/TGFβRII DKO iNK cells reduced the size of SK-OV-3 spheroids to a greater extent than unedited iNK cells at all E:T ratios greater than 0.01, and significantly at E:T ratios of 1 or higher. The TGFβRII KO clone 7 iNKs also exhibited significantly enhanced killing when compared to unedited iNK cells. While a number of single CISH KO clones did not show significant enhancement of killing at the 10:1 E:T ratio, the majority of clones did display a trend towards increased SK-OV-3 spheroid cell killing, with the greatest differential at the highest E:T ratio. To further elucidate the functionality of the edited iNKs, the cells were pushed to kill tumor targets repeatedly over a multiday period, herein described as an in vitro serial killing assay. At day 0 of the assay, 10×10⁶ Nalm6 tumor cells (a B cell leukemia cell line) and 2×10⁵ iNKs were plated in each well of a 96-well plate in the presence of IL-15 (10 ng/ml) and TGF-β (long/ml). At 48 hour intervals, a bolus of 5×10³ Nalm6 tumor cells (a B cell leukemia cell line) was added to re-challenge the iNK population. As shown in FIG. 13, the edited iNK cells (CISH/TGFβRII DKO iNK cells) exhibited continued killing of Nalm6 cells after multiple challenges with Nalm6 tumor cells, whereas unedited iNK cells were limited in their serial killing effect. The data supports the conclusion that the CISH and TGFβRII edits result in prolonged enhancement of cell killing.

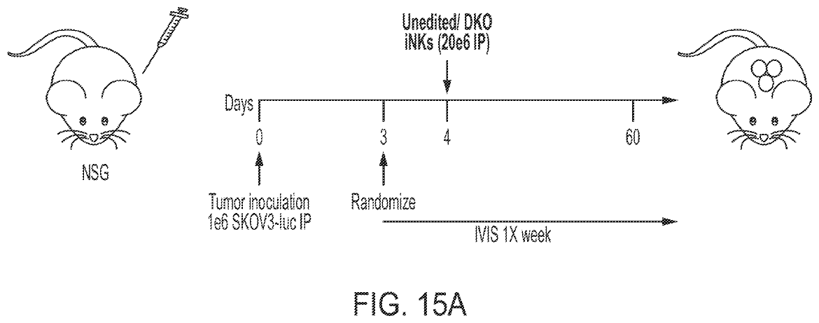

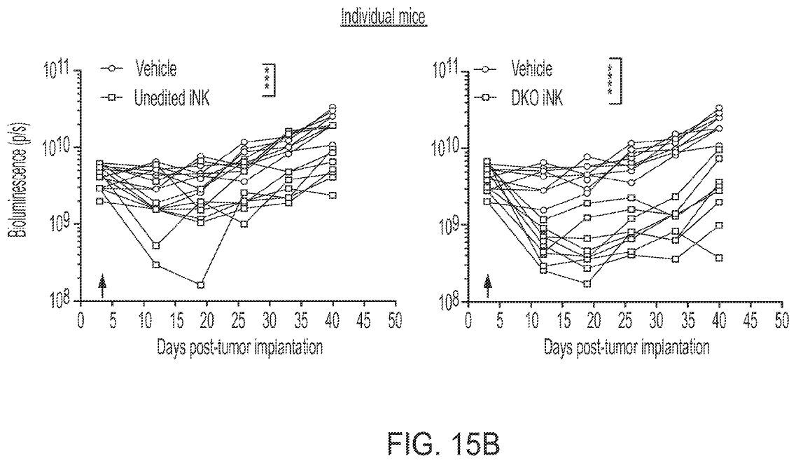

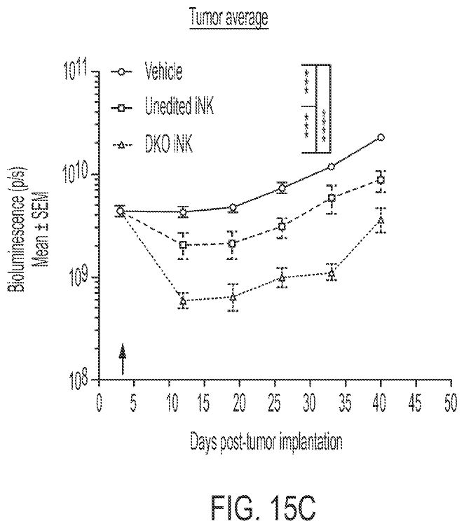

Finally, edited iNK cells (CISH/TGFβRII DKO iNK cells) were assayed for their ability to kill tumor targets in an in vivo model. To this end, an established NOD scid gamma (NSG) xenograft model was utilized in an assay as depicted in FIG. 15A. Briefly, 1×10⁶ SK-OV-3 cells engineered to express luciferase were injected intraperitoneally (IP) at day 0. On day 3, the inoculated mice were imaged using an In vivo imaging system (IVIS) and randomized into 3 groups. The next day (day 4), 20×10⁶ unedited iNKs or CISH/TGFβRII DKO iNKs were administered by IP injection, while a third group was injected with vehicle as a control. Following inoculation of the animals with tumor cells, animals were imaged once a week to measure tumor burden over time. FIG. 15B depicts the bioluminescence of the tumors in the individual mice in the 3 different groups (n=9 in each group), vehicle, unedited iNKs, and CISH/TGFβRII DKO iNKs. The average tumor burden over time for these same animals is depicted in FIG. 15C. A two way anova analysis was performed on the data, and CISH/TGFβRII DKO iNK treated animals had significantly less tumor burden as measured by bioluminescence when compared to animals treated with unedited iNKs (p value: 0.0004). By 10 days post-tumor implantation, mice injected with the CISH/TGFβRII DKO iNKs exhibited a significant reduction in the size of their tumors relative to mice injected with the vehicle controls or the unedited iNKs. The overall reduction in tumor size is seen for several days, and at least until 35 days post-tumor implantation. These data show that the edited DKO iNKs were actively killing tumor cells in this in vivo model.

Overall, these results demonstrate that unedited and CISH/TGFβRII DKO iPSCs can be differentiated into iNK cells exhibiting canonical NK cell markers. Additionally, CISH/TGFβRII DKO iNK cells demonstrated enhanced anti-tumor activity against tumor cell lines derived from both solid and hematological malignancies.

### Example 3: ADORA2A Edited iPSCs Give Rise to Edited iNKs with Enhanced Function

ADORA2A is another target gene of interest, the loss of which is hypothesized to affect NK cell function in a tumor microenvironment (TME). The ADORA2A gene encodes a receptor that responds to adenosine in the TME, resulting in the production of cAMP which functions to drive a number of inhibitory effects on NK cells. We hypothesized that knocking out the function of ADORA2A could enhance iNK cell function. Utilizing a similar approach to the one described in Examples 1 and 2, the PCS iPSC line was edited using a RNP having an engineered Cas12a with three amino acid substitutions (M537R, F870L, and H800A (SEQ ID NO: 1148)) and a gRNA specific to ADORA2A (except that 4 μM RNP was delivered to cells rather than 2 μM RNP). As described in Example 1, the gRNA was generated with a targeting domain consisting of RNA, an AsCpf1 scaffold of the sequence UAAUUUCUACUCUUGUAGAU (SEQ ID NO: 1153) located 5′ of the targeting domain, and a 25-mer DNA extension of the sequence ATGTGTTTTTGTCAAAAGACCTTTT (SEQ ID NO: 1154) at the 5′ terminus of the scaffold sequence. The ADORA2A gRNA sequence is shown in Table 11.

TABLE 11
Guide RNA sequence

| | gRNA Targeting | Full Length gRNA |
|---|---|---|
| Target | Domain Sequence | Sequence |
| ADORA2A | CCAUCGGCCUGACUCCC | ATGTGTTTTTGTCAAAAGACCTT |
| 4113 | AUG | TTrUrArArUrUrUrCrUrArCr |
| | (SEQ ID NO: 1159) | UrCrUrUrGrUrArGrArUrCrC |
| | | rArUrCrGrGrCrCrUrGrArCr |
| | | UrCrCrCrArUrG(SEQ ID |
| | | NO: 1160) |

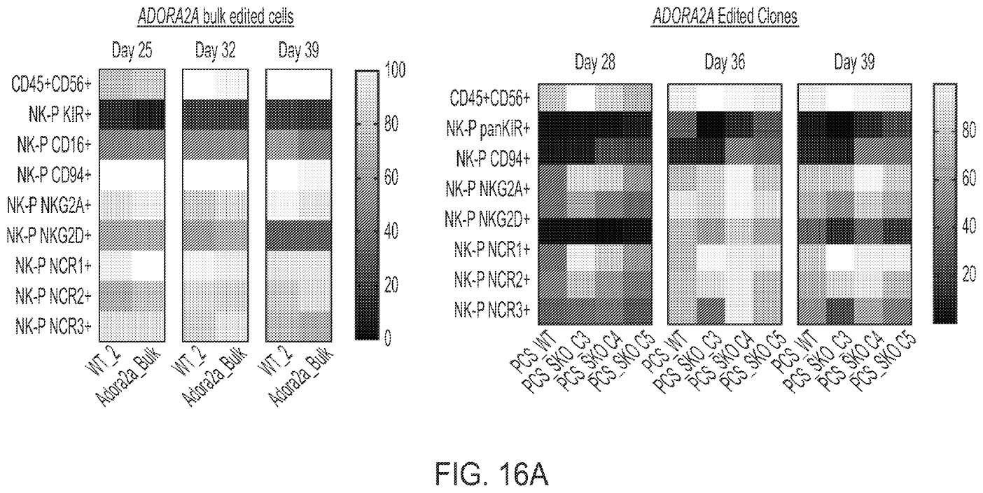

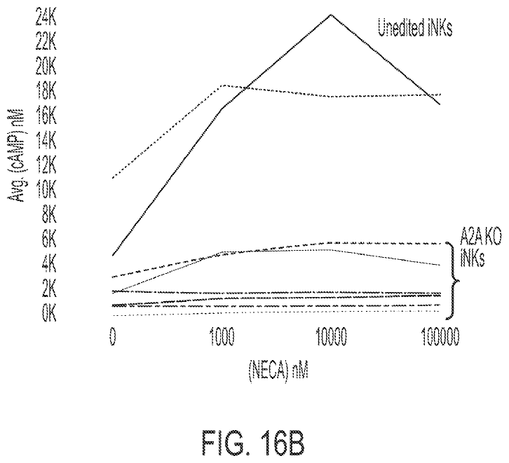

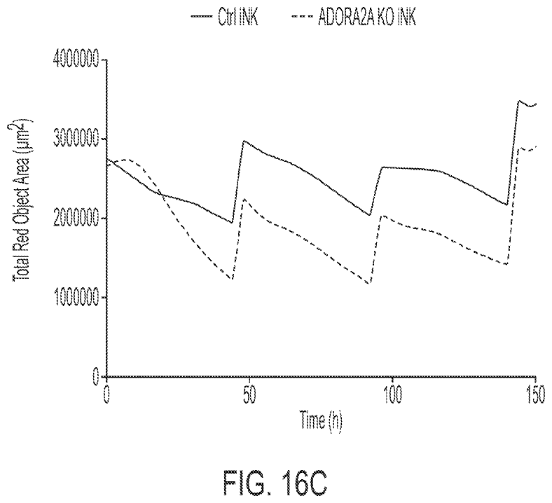

The bulk editing rate by the Cas12a RNP prior to clonal selection was 49% as determined by next-generation sequencing (NGS). Nonetheless, several clones that had both ADORA2A alleles edited were identified, expanded and differentiated. To determine whether an ADORA2A edited iPSC could yield CD45⁺ CD56+ iNKs, both bulk and singled ADORA2A KO clones were differentiated using the NKMACS+serum protocol as described in Example 2 (FIG. 8C). As shown in FIG. 16A, edited iPSCs differentiated to iNKs with similar NK cell marker expression compared to unedited control iPSCs.

To confirm that Cas12a-mediated ADORA2A editing resulted in a functional deletion of the gene, cAMP accumulation in response to treatment with 5′-N-ethylcarboxamide adenosine (“NECA”, a more stable adenosine analog that acts as an ADORA2A agonist) was assessed in both the edited and unedited control iNKs. Edited cells with a functional knockout of ADORA2A would not be expected to accumulate as much cAMP in the cells in response to NECA relative to cells with functional ADORA2A. Briefly, iNK cells were treated with varying concentrations of NECA for 15 minutes. The iNK cells were then lysed, and the cAMP in the lysate was then measured using a CisBio cAMP kit. As shown in FIG. 16B, unedited iNKs had increased levels of cAMP accumulation as the concentration of NECA was increased (n=2). Conversely, the ADORA2A (“A2A KOs”) KO iNKs showed minimal production of cAMP at increasing concentrations of NECA, indicating that the Cas12a-induced edits functionally knocked out ADORA2A function. The bulk iNKs (top two A2A KO iNK lines in FIG. 16B) exhibited slightly higher levels of cAMP than the selected ADORA2A KO clones (lower four A2A KO iNK lines in FIG. 16B), as would be expected from the lower editing rates in the bulk population. Based on this molecular evidence of functional ablation of ADORA2A, the iNKs would be expected to be resistant to the inhibitory effects of adenosine in a tumor microenvironment.

The ADORA2A KO iNKs were also tested in an in vitro NALM6 serial killing assay as described in Example 2, with one main difference: 100 μM of NECA was added in place of TGFβ. The ADORA2A KO iNKs exhibited enhanced serial killing relative to the wild type iNKs in the presence of NECA, indicating that the ADORA2A KO iNKs were resistant to NECA inhibition (FIG. 16C). As a result, the ADORA2A KO iNK cells would be expected to have improved cytotoxicity against tumor cells in the presence of adenosine in the TME relative to unedited iNK cells.

### Example 4: Generation of CISH/TGFβRII/ADORA2A Triple Edited (TKO) iPSCs and the Characterization of Differentiated TKO iNKs

In order to generate CISH, TGFβRII, and ADORA2A triple edited (TKO) iPSCs, two approaches were taken; 1) two step editing in which the CISH/TGFβRII DKO (CR) iPSC clone described in Examples 1 and 2 was edited at the ADORA2A locus via electroporation with an ADORA2A targeting RNP (as described in Example 3), and 2) simultaneous editing of PCS iPS cells with all 3 RNPs, one for each target gene. Both strategies utilized the editing protocol briefly described in Example 1. In the case of simultaneous editing, the total RNP concentration was 8 μM (Cish:2 μM+ TGFβRII:2 μM+ADORA2A:4 μM). Regardless of the approach, cells were plated, expanded and colonies were picked as described above. Using NGS to analyze gDNA harvested from the iPSCs, it was determined that the bulk editing rates were 96.70%, 97.17%, and 90.16% for CISH, TGFβRII and ADORA2A, respectively, when all target genes were edited simultaneously. Picked colonies that had Insertions and/or Deletions (InDels) at all 6 alleles were selected for further analysis.

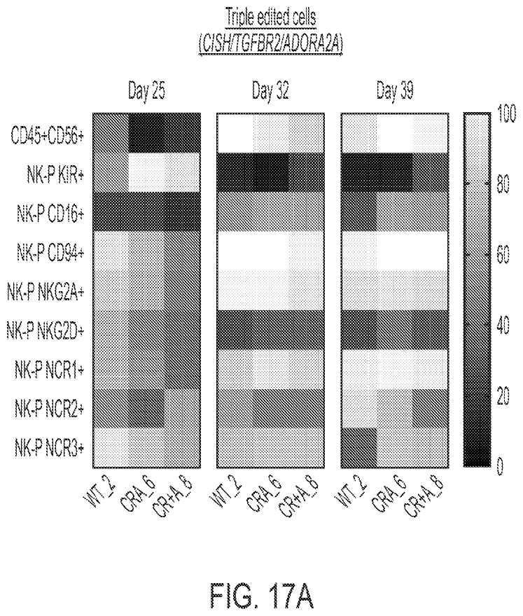

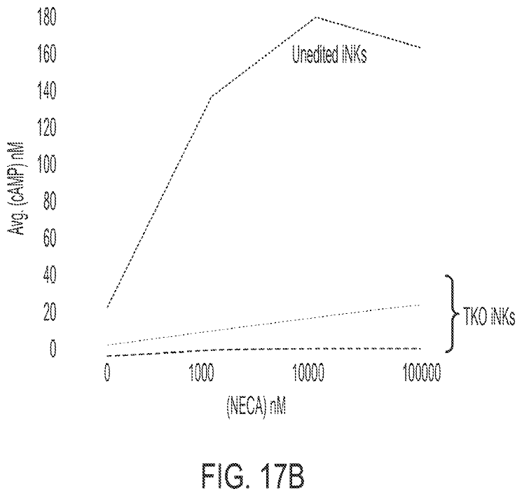

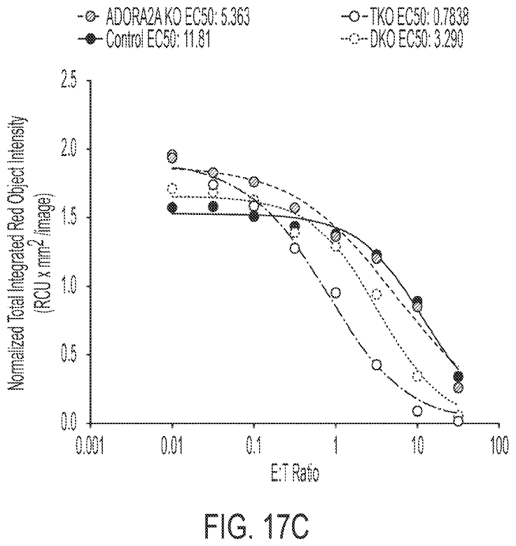

Similar to the analysis described in Example 1, unedited iPSCs and the edited iPSCs were differentiated to iNKs using the NK MACS+Serum condition (described in FIG. 8C) and assessed by flow cytometry at different time points, including at day 25, day 32, and day 39 in culture. As shown in FIG. 17A, analysis of the different NK surface markers revealed no major differences between clones that were generated by the two-step editing method (CR+A 8) or the simultaneous editing method (CRA 6). Both TKO clones (CR+A 8 and CRA 6) showed similar expression profiles to the unedited iNKs (Wt) at each time point. When the TKO iNK cells were analyzed for their responsiveness to NECA (as described in Example 3), both TKO iNKs had little to no cAMP accumulation (FIG. 17B), demonstrating that ADORA2A was functionally knocked out. By contrast, the unedited iNKs demonstrated a NECA dose dependent increase in cAMP (FIG. 17B). These results indicate that the TKO iNKs would be expected to be resistant to the inhibitory effects of adenosine in the TME. Finally, the CISH/TGFβRII/ADORA2A TKO iNKs were assessed alongside CISH/TGFβRII DKO iNKs, ADORA2A single KO (SKO) iNKs, and unedited iNKs in a 3D tumor cell killing assay. This assay was performed as described in Example 2 with IL-15 and TGFβ but without NECA. Interestingly, both the TKO (CRA6) and DKO (CR) iNKs outperformed the unedited iNKs in killing the tumor cells, indicating that both multiplex edited iNKs have enhanced function over unedited control cells (FIG. 17C). These results show that knocking out ADORA2A does not negatively affect the ability of iNKs having CISH and TGFBRII KOs to kill tumor spheroid cells.

### Example 5: Selection of CISH, TGFβRII, ADORA2A, TIGIT, and NKG2A Targeting gRNAs

The cutting efficiency of CISH, TGFBRII, ADORA2A, TIGIT, and NKG2A Cas12a guide RNAs were further tested. Guide RNAs were screened by complexing commercially synthesized gRNAs with Cas12a in vitro and delivering gRNA/Cas12a ribonucleoprotein (RNP) to IPSCs via electroporation. The iPSCs were edited using a RNP having an engineered Cas12a with three amino acid substitutions (M537R, F870L, and H800A (SEQ ID NO: 1148)). The gRNAs were generated with a targeting domain consisting of RNA, an AsCpf1 scaffold of the sequence UAAUUUCUACUCUUGUAGAU (SEQ ID NO: 1153) located 5′ of the targeting domain, and a 25-mer DNA extension of the sequence ATGTGTTTTTGTCAAAAGACCTTTT (SEQ ID NO: 1154) at the 5′ terminus of the scaffold sequence. Table 12 provides the targeting domains of the guide RNAs that were tested for editing activity.

TABLE 12
guide RNA sequences

| Target | gRNA Targeting Domain Sequence |
|---|---|
| TGFβRII | UGAUGUGAGAUUUUCCACCUG |
| | (SEQ ID NO: 1161) |
| CISH | ACUGACAGCGUGAACAGGUAG |
| | (SEQ ID NO: 1162) |
| ADORA2A | CCAUCGGCCUGACUCCCAUGC |
| | (SEQ ID NO: 1163) |
| ADORA2A | CCAUCACCAUCAGCACCGGGU |
| | (SEQ ID NO: 1164) |
| ADORA2A | CCUGUGUGCUGGUGCCCCUGC |
| | (SEQ ID NO: 1165) |
| TIGIT | UGCAGAGAAAGGUGGCUCUAU |
| | (SEQ ID NO: 1166) |
| TIGIT | UCUGCAGAAAUGUUCCCCGUU |
| | (SEQ ID NO: 1167) |
| TIGIT | UAGGACCUCCAGGAAGAUUCU |
| | (SEQ ID NO: 1168) |
| NKG2A | GCAACUGAACAGGAAAUAACC |
| | (SEQ ID NO: 1169) |
| NKG2A | GUUGCUGCCUCUUUGGGUUUG |
| | (SEQ ID NO: 1170) |
| NKG2A | AAGGGAAUGACAAAACCUAUC |
| | (SEQ ID NO: 1171) |

In brief, 100,000 iPSCs/well were transfected with the RNP of interest, cells were incubated at 37° C. for 72 hours, and then harvested for DNA characterization. iPSCs were transfected with gRNA/Cas12a RNPs at various concentrations. The percentage editing events were determined for eight different RNP concentrations ranging from negative control (0 mM), to 8 mM.

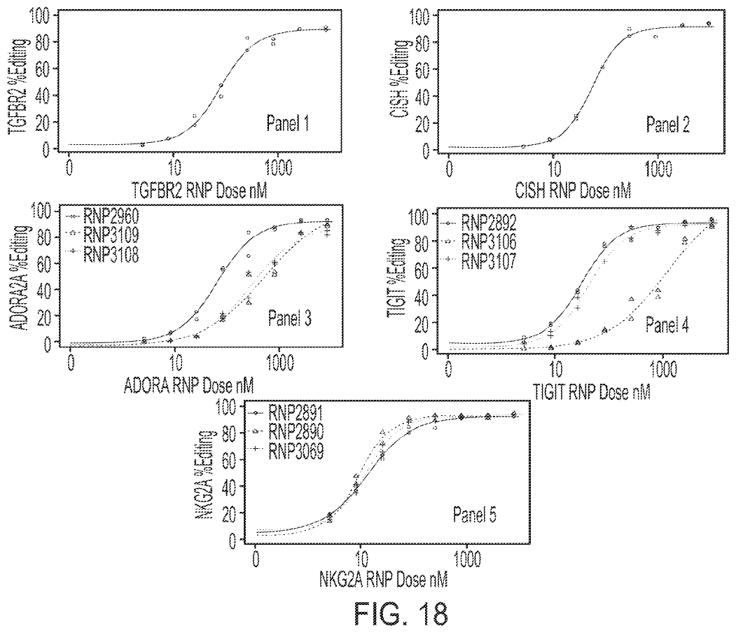

As shown in FIG. 18 panel 1, the TGFβRII gRNA (SEQ ID NO: 1161) exhibited an EC50 of ˜79 nM RNP. As shown in FIG. 18 panel 2, the CISH gRNA (SEQ ID NO: 1162) exhibited an EC50 of ˜50 nM RNP. As shown in FIG. 18 panel 3, an ADORA2A gRNA (SEQ ID NO: 1163) included in RNP2960 exhibited an EC50 of ˜63 nM RNP, while an ADORA2A gRNA (SEQ ID NO: 1164) included in RNP3109, or gRNA (SEQ ID NO: 1165) included in RNP3108 exhibited EC50 values of ˜493 nM and ˜280 nM RNP respectively. As shown in FIG. 18 panel 4, a TIGIT gRNA (SEQ ID NO: 1166) included in RNP2892 exhibited an EC50 of ˜29 nM RNP, while a TIGIT gRNA (SEQ ID NO: 1167) included in RNP3106, or gRNA (SEQ ID NO: 167) included in RNP3107 exhibited EC50 values of ˜1146 nM and ˜40 nM RNP respectively. As shown in FIG. 18 panel 5, a NKG2A gRNA (SEQ ID NO: 1169) included in RNP19142 exhibited an EC50 of —8 nM RNP, while a NKG2A gRNA (SEQ ID NO: 1170) included in RNP3069, or gRNA (SEQ ID NO: 1171) included in RNP2891 exhibited EC50 values of ˜12 nM and ˜13 nM RNP respectively.

### EQUIVALENTS

It is to be understood that while the disclosure has been described in conjunction with the detailed description thereof, the foregoing description is intended to illustrate and not limit the scope of the present disclosure, which is defined by the scope of the appended claims. Other aspects, advantages, and modifications are within the scope of the following claims.

1. A pluripotent human stem cell, wherein the stem cell comprises:
(i) a genomic edit that results in loss of function of Cytokine Inducible SH2 Containing Protein (CISH) and
(ii) a genomic edit that results in a loss of function of an agonist of the TGF beta signaling pathway, or a genomic edit that results in a loss of function of adenosine A2a receptor (ADORA2A).

2. The pluripotent human stem cell of claim 1, wherein the stem cell comprises a genomic edit that results in a loss of function of an agonist of the TGF beta signaling pathway and a genomic edit that results in a loss of function of ADORA2A.

3. The pluripotent human stem cell of claim 1 or 2, wherein the stem cell comprises a genomic edit that results in a loss of function of a TGF beta receptor or a dominant-negative variant of a TGF beta receptor.

4. The pluripotent human stem cell of claim 3, wherein the TGF beta receptor is a TGF beta receptor II (TGFβRID.

5. The pluripotent human stem cell of any one of the preceding claims, wherein the stem cell expresses one or more pluripotency markers selected from the group consisting of SSEA-3, SSEA-4, TRA-1-60, TRA-1-81, TRA-2-49/6E, ALP, Sox2, E-cadherin, UTF-1, Oct4, Rex1, and Nanog.

6. A differentiated cell, wherein the differentiated cell is a daughter cell of the pluripotent human stem cell of any one of the preceding claims.

7. The differentiated cell of claim 6, wherein the differentiated cell is an immune cell.

8. The differentiated cell of claim 6, wherein the differentiated cell is a lymphocyte.

9. The differentiated daughter cell of claim 6, wherein the differentiated cell is a natural killer cell.

10. The differentiated cell of claim 6, wherein the stem cell is a human induced pluripotent stem cell (iPSC), and wherein the differentiated daughter cell is an iNK cell.

11. The differentiated cell of claim 6, wherein the cell:
(a) does not express endogenous CD3, CD4, and/or CD8; and
(b) expresses at least one endogenous gene encoding:
(i) CD56 (NCAM), CD49, CD43, and/or CD45, or any combination thereof;
(ii) NK cell receptor immunoglobulin gamma Fc region receptor III (FcγRIII, cluster of differentiation 16 (CD16));
(iii) natural killer group-2 member D (NKG2D);
(iv) CD69;
(v) a natural cytotoxicity receptor;
or any combination of two or more thereof.

12. The cell of any of the preceding claims, wherein the cell comprises one or more additional genomic edits.

13. The cell of claim 12, wherein the cell:
(1) comprises at least one genomic edit characterized by an exogenous nucleic acid expression construct that comprises a nucleic acid sequence encoding:
(i) a chimeric antigen receptor (CAR);
(ii) a FcγRIII (CD16) or a variant of FcγRIII (CD16);
(iii) interleukin 15 (IL-15);
(iv) an IL-15 receptor (IL-15R) agonist, or a constitutively active variant of an IL-15 receptor;
(v) an IL-12 receptor (IL-12R) agonist, or a constitutively active variant of an IL-12 receptor;
(vi) an IL-12 receptor (IL-12R) agonist, or a constitutively active variant of an IL-12 receptor;
(vii) human leukocyte antigen G (HLA-G);
(viii) human leukocyte antigen E (HLA-E);
(ix) leukocyte surface antigen cluster of differentiation CD47 (CD47);
or any combination of two or more thereof and/or
(2) comprises at least one genomic edit that results in a loss of function of at least one of:
(i) ADORA2A;
(ii) T cell immunoreceptor with Ig and ITIM domains (TIGIT);
(iii) β-2 microglobulin (B2M);
(iv) programmed cell death protein 1 (PD-1);
(v) class II, major histocompatibility complex, transactivator (CIITA);
(vi) natural killer cell receptor NKG2A (natural killer group 2A);
(vii) two or more HLA class II histocompatibility antigen alpha chain genes, and/or two or more HLA class II histocompatibility antigen beta chain genes;
(viii) cluster of differentiation 32B (CD32B, FCGR2B);
(ix) T cell receptor alpha constant (TRAC);
or any combination of two or more thereof.

14. A human induced pluripotent stem cell (iPSC), wherein the iPSC comprises a genomic edit that results in a loss of function of adenosine A2a receptor (ADORA2A).

15. The human iPSC of claim 14, wherein the iPSC comprises a genomic edit that results in a loss of function of an agonist of the TGF beta signaling pathway or a genomic edit that results in loss of function of Cytokine Inducible SH2 Containing Protein (CISH).

16. The human iPSC of claim 15, wherein the iPSC comprises a genomic edit that results in a loss of function of an agonist of the TGF beta signaling pathway and a genomic edit that results in loss of function of CISH.

17. The human iPSC of claim 15 or 16, wherein the iPSC comprises a genomic edit that results in a loss of function of a TGF beta receptor or a dominant-negative variant of a TGF beta receptor.

18. The human iPSC of claim 17, wherein the TGF beta receptor is a TGF beta receptor II (TGFβRII).

19. The human iPSC of any one of claims 14-18, wherein the iPSC expresses one or more pluripotency markers selected from the group consisting of SSEA-3, SSEA-4, TRA-1-60, TRA-1-81, TRA-2-49/6E, ALP, Sox2, E-cadherin, UTF-1, Oct4, Rex1, and Nanog.

20. A differentiated cell, wherein the differentiated cell is a daughter cell of the human iPSC of any one of claims 14-19.

21. The differentiated cell of claim 20, wherein the differentiated cell is an immune cell.

22. The differentiated cell of claim 20, wherein the differentiated cell is a lymphocyte.

23. The differentiated daughter cell of claim 20, wherein the differentiated cell is a natural killer cell.

24. The differentiated cell of claim 20, wherein the differentiated daughter cell is an iNK cell.

25. The differentiated cell of claim 20, wherein the cell:
(a) does not express endogenous CD3, CD4, and/or CD8; and
(b) expresses at least one endogenous gene encoding:
(i) CD56 (NCAM), CD49, CD43, and/or CD45, or any combination thereof;
(ii) NK cell receptor immunoglobulin gamma Fc region receptor III (FcγRIII, cluster of differentiation 16 (CD16));
(iii) natural killer group-2 member D (NKG2D);
(iv) CD69;
(v) a natural cytotoxicity receptor;
or any combination of two or more thereof.

26. The cell of any of claims 14-25, wherein the cell comprises one or more additional genomic edits.

27. The cell of claim 26, wherein the cell:
(1) comprises at least one genomic edit characterized by an exogenous nucleic acid expression construct that comprises a nucleic acid sequence encoding:
(i) a chimeric antigen receptor (CAR);
(ii) a FcγRIII (CD16) or a variant of FcγRIII (CD16);
(iii) interleukin 15 (IL-15);
(iv) an IL-15 receptor (IL-15R) agonist, or a constitutively active variant of an IL-15 receptor;
(v) an IL-12 receptor (IL-12R) agonist, or a constitutively active variant of an IL-12 receptor;
(vi) an IL-12 receptor (IL-12R) agonist, or a constitutively active variant of an IL-12 receptor;
(vii) human leukocyte antigen G (HLA-G);
(viii) human leukocyte antigen E (HLA-E);
(ix) leukocyte surface antigen cluster of differentiation CD47 (CD47);
or any combination of two or more thereof
and/or
(2) comprises at least one genomic edit that results in a loss of function of at least one of:
(i) cytokine inducible SH2 containing protein (CISH);
(ii) T cell immunoreceptor with Ig and ITIM domains (TIGIT);
(iii) β-2 microglobulin (B2M);
(iv) programmed cell death protein 1 (PD-1);
(v) class II, major histocompatibility complex, transactivator (CIITA);
(vi) natural killer cell receptor NKG2A (natural killer group 2A);
(vii) two or more HLA class II histocompatibility antigen alpha chain genes, and/or two or more HLA class II histocompatibility antigen beta chain genes;
(viii) cluster of differentiation 32B (CD32B, FCGR2B);
(ix) T cell receptor alpha constant (TRAC);
or any combination of two or more thereof.

28. The cell of any one of claims 1-27, wherein:
the genomic edit resulting in loss of function of CISH was produced using a guide RNA comprising a targeting domain sequence comprising the nucleotide sequence according to any one of SEQ ID NO: 258-364, 1155, and 1162;
the genomic edit resulting in loss of function of TGFβRII was produced using a guide RNA comprising a targeting domain sequence comprising the nucleotide sequence according to any one of SEQ ID NO: 29-257, 1157, and 1161; and/or
the genomic edit resulting in loss of function of ADORA2A was produced using a guide RNA comprising a targeting domain sequence comprising the nucleotide sequence according to any one of SEQ ID NO: 827-1143, 1159, and 1163.

29. The cell of any one of claims 1-28, wherein:
the genomic edit resulting in loss of function of CISH was produced using a ribonucleoprotein (RNP) complex comprising (i) an RNA-guided nuclease and (ii) a guide RNA comprising a targeting domain sequence comprising the nucleotide sequence according to any one of SEQ ID NO: 258-364, 1155, and 1162;
the genomic edit resulting in loss of function of TGFβRII was produced using a ribonucleoprotein (RNP) complex comprising (i) an RNA-guided nuclease and (ii) a guide RNA comprising a targeting domain sequence comprising the nucleotide sequence according to any one of SEQ ID NO: 29-257, 1157, and 1161; and/or
the genomic edit resulting in loss of function of ADORA2A was produced using a ribonucleoprotein (RNP) complex comprising (i) an RNA-guided nuclease and (ii) a guide RNA comprising a targeting domain sequence comprising the nucleotide sequence according to any one of SEQ ID NO: 827-1143, 1159, and 1163.

30. A method of making the cell of any one of claims 1-29, the method comprising contacting the cell with one or more of:
an RNA-guided nuclease and a guide RNA comprising a targeting domain sequence comprising the nucleotide sequence according to any one of SEQ ID NO: 258-364, 1155, and 1162;
an RNA-guided nuclease and a guide RNA comprising a targeting domain sequence comprising the nucleotide sequence according to any one of SEQ ID NO: 29-257, 1157, and 1161; and/or
an RNA-guided nuclease and a guide RNA comprising a targeting domain sequence comprising the nucleotide sequence according to any one of SEQ ID NO: 827-1143, 1159, and 1163.

31. A method of making the cell of any one of claims 1-30, the method comprising contacting the cell with one or more of:
a ribonucleoprotein (RNP) complex comprising (i) an RNA-guided nuclease and (ii) a guide RNA comprising a targeting domain sequence comprising the nucleotide sequence according to any one of SEQ ID NO: 258-364, 1155, and 1162;
a ribonucleoprotein (RNP) complex comprising (i) an RNA-guided nuclease and (ii) a guide RNA comprising a targeting domain sequence comprising the nucleotide sequence according to any one of SEQ ID NO: 29-257, 1157, and 1161; and/or
a ribonucleoprotein (RNP) complex comprising (i) an RNA-guided nuclease and (ii) a guide RNA comprising a targeting domain sequence comprising the nucleotide sequence according to any one of SEQ ID NO: 827-1143, 1159, and 1163.

32. The method of any one of claims 29-31, wherein the RNA-guided nuclease is a Cas12a variant.

33. The method of claim 32, wherein the Cas12a variant comprises one or more amino acid substitutions selected from M537R, F870L, and H800A.

34. The method of claim 32, wherein the Cas12a variant comprises amino acid substitutions M537R, F870L, and H800A.

35. The method of claim 32, wherein the Cas12a variant comprises the amino acid sequence of SEQ ID NO:1148.

36. The method of any one of claims 30-35, comprising contacting the cell with:
(i) a guide RNA comprising a targeting domain sequence comprising the nucleotide sequence of SEQ ID NO: 1155 or 1162; a guide RNA comprises a targeting domain sequence comprising the nucleotide sequence of SEQ ID NO: 1157 or 1161; and a guide RNA comprises a targeting domain sequence comprising the nucleotide sequence of SEQ ID NO: 1159 or 1163; and
(ii) an RNA-guided nuclease comprising the amino acid sequence of one of SEQ ID NO:1144-1151 (or a portion thereof).

37. A pluripotent human stem cell, wherein the stem cell comprises a disruption in the transforming growth factor beta (TGF beta) signaling pathway.

38. The pluripotent human stem cell of claim 34, wherein the stem cell comprises a genomic edit that results in a loss of function of an agonist of the TGF beta signaling pathway.

39. The pluripotent human stem cell of claim 37 or 38, comprising a loss of function of a TGF beta receptor or a dominant-negative variant of a TGF beta receptor.

40. The pluripotent human stem cell of claim 39, wherein the TGF beta receptor is a TGF beta receptor II (TGFβRII).

41. The pluripotent human stem cell of any one of claims 37-40, further comprising a loss of function of an antagonist of interleukin signaling.

42. The pluripotent human stem cell of any one of claims 37-41, wherein the stem cell further comprises a genomic modification that results in the loss of function of an antagonist of interleukin signaling.

43. The pluripotent human stem cell of claim 41 or 42, wherein the antagonist of interleukin signaling is an antagonist of the IL-15 signaling pathway and/or of the IL-2 signaling pathway.

44. The pluripotent human stem cell of any one of claims 37-43, comprising a loss of function of Cytokine Inducible SH2 Containing Protein (CISH).

45. The pluripotent human stem cell of claim 44, wherein the stem cell comprises a genomic modification that results in the loss of function of CISH.

46. The pluripotent human stem cell of any one of claims 37-45, wherein the stem cell expresses one or more pluripotency markers selected from the group consisting of SSEA-3, SSEA-4, TRA-1-60, TRA-1-81, TRA-2-49/6E, ALP, Sox2, E-cadherin, UTF-1, Oct4, Rex1, and Nanog.

47. A differentiated cell, wherein the differentiated cell is a daughter cell of the pluripotent human stem cell of any one of claims 37-46.

48. The differentiated cell of claim 47, wherein the differentiated cell is an immune cell.

49. The differentiated cell of claim 47, wherein the differentiated cell is a lymphocyte.

50. The differentiated daughter cell of claim 47, wherein the differentiated cell is a natural killer cell.

51. The differentiated cell of claim 47, wherein the stem cell is a human induced pluripotent stem cell (iPSC), and wherein the differentiated daughter cell is an iNK cell.

52. The differentiated cell of claim 47, wherein the cell:
(a) does not express endogenous CD3, CD4, and/or CD8; and
(b) expresses at least one endogenous gene encoding:
(i) CD56 (NCAM), CD49, CD43, and/or CD45, or any combination thereof;
(ii) NK cell receptor immunoglobulin gamma Fc region receptor III (FcγRIII, cluster of differentiation 16 (CD16));
(iii) natural killer group-2 member D (NKG2D);
(iv) CD69;
(v) a natural cytotoxicity receptor;
or any combination of two or more thereof.

53. The cell of any of claims 37-52, wherein the cell comprises one or more additional genomic edits.

54. The cell of claim 53, wherein the cell:
(1) comprises at least one genomic edit characterized by an exogenous nucleic acid expression construct that comprises a nucleic acid sequence encoding:
(i) a chimeric antigen receptor (CAR);
(ii) a FcγRIII (CD16) or a variant of FcγRIII (CD16);
(iii) interleukin 15 (IL-15);
(iv) an IL-15 receptor (IL-15R) agonist, or a constitutively active variant of an IL-15 receptor;
(v) an IL-12 receptor (IL-12R) agonist, or a constitutively active variant of an IL-12 receptor;
(vi) an IL-12 receptor (IL-12R) agonist, or a constitutively active variant of an IL-12 receptor;
(vii) human leukocyte antigen G (HLA-G);
(viii) human leukocyte antigen E (HLA-E);
(ix) leukocyte surface antigen cluster of differentiation CD47 (CD47);
or any combination of two or more thereof;
and/or
(2) comprises at least one genomic edit that results in a loss of function of at least one of:
(i) cytokine inducible SH2 containing protein (CISH);
(ii) adenosine A2a receptor (ADORA2A);
(iii) T cell immunoreceptor with Ig and ITIM domains (TIGIT);
(iv) β-2 microglobulin (B2M);
(v) programmed cell death protein 1 (PD-1);
(vi) class II, major histocompatibility complex, transactivator (CIITA);
(vii) natural killer cell receptor NKG2A (natural killer group 2A);
(viii) two or more HLA class II histocompatibility antigen alpha chain genes, and/or two or more HLA class II histocompatibility antigen beta chain genes;
(ix) cluster of differentiation 32B (CD32B, FCGR2B);
(x) T cell receptor alpha constant (TRAC);
or any combination of two or more thereof.

55. A method of culturing a pluripotent human stem cell, comprising culturing the stem cell in a medium comprising activin.

56. The method of claim 55, wherein the pluripotent human stem cell is an embryonic stem cell or an induced pluripotent stem cell.

57. The method of claim 55 or 56, wherein the pluripotent human stem cell does not express TGFβRII.

58. The method of any one of claims 55-57, wherein the pluripotent human stem cell is genetically engineered not to express TGFβRII.

59. The method of any one of claims 55-57, wherein the pluripotent human stem cell is genetically engineered to knock out a gene encoding TGFβRII.

60. The method of any one of claims 55-59, wherein the activin is activin A.

61. The method of any one of claims 55-60, wherein the medium does not comprise TGFβ.

62. The method of any one of claims 55-61, wherein the culturing is performed for a defined period of time (e.g., 1, 2, 3, 4, 5, 6, 7, 8, 9, 10, 11, 12 days, or more).

63. The method of any one of claims 55-62, wherein at one or more times during or following the culturing step, the pluripotent human stem cell maintains pluripotency (e.g., exhibits one or more pluripotency markers).

64. The method of claim 63, wherein at one or more times during or following the culturing step, the pluripotent human stem cell expresses a detectable level of one or more of SSEA-3, SSEA-4, TRA-1-60, TRA-1-81, TRA-2-49/6E, ALP, Sox2, E-cadherin, UTF-1, Oct4, Rex1, and Nanog.

65. The method of claim 63, wherein at a time during or following the culturing step, the pluripotent human stem cell is differentiated into cells of endoderm, mesoderm, and/or ectoderm lineage.

66. The method of claim 65, wherein the pluripotent human stem cell, or its progeny, is further differentiated into a natural killer (NK) cell.

67. The method of any one of claims 55-66, wherein the pluripotent human stem cell:
(1) comprises at least one genomic edit characterized by an exogenous nucleic acid expression construct that comprises a nucleic acid sequence encoding:
(i) a chimeric antigen receptor (CAR);
(ii) a FcγRIII (CD16) or a variant of FcγRIII (CD16);
(iii) interleukin 15 (IL-15);
(iv) an IL-15 receptor (IL-15R) agonist, or a constitutively active variant of an IL-15 receptor;
(v) an IL-12 receptor (IL-12R) agonist, or a constitutively active variant of an IL-12 receptor;
(vi) an IL-12 receptor (IL-12R) agonist, or a constitutively active variant of an IL-12 receptor;
(vii) human leukocyte antigen G (HLA-G);
(viii) human leukocyte antigen E (HLA-E);
(ix) leukocyte surface antigen cluster of differentiation CD47 (CD47);
or any combination of two or more thereof;
and/or
(2) comprises at least one genomic edit that results in a loss of function of at least one of:
(i) cytokine inducible SH2 containing protein (CISH);
(ii) adenosine A2a receptor (ADORA2A);
(iii) T cell immunoreceptor with Ig and ITIM domains (TIGIT);
(iv) β-2 microglobulin (B2M);
(v) programmed cell death protein 1 (PD-1);
(vi) class II, major histocompatibility complex, transactivator (CIITA);
(vii) natural killer cell receptor NKG2A (natural killer group 2A);
(viii) two or more HLA class II histocompatibility antigen alpha chain genes,
and/or two or more HLA class II histocompatibility antigen beta chain genes;
(ix) cluster of differentiation 32B (CD32B, FCGR2B);
(x) T cell receptor alpha constant (TRAC);
or any combination of two or more thereof.

68. A cell culture comprising (i) a pluripotent human stem cell and (ii) a cell culture medium comprising activin, wherein the pluripotent human stem cell comprises a disruption in the transforming growth factor beta (TGF beta) signaling pathway.

69. The cell culture of claim 68, wherein the stem cell comprises a genomic edit that results in a loss of function of an agonist of the TGF beta signaling pathway.

70. The cell culture of claim 69, wherein the genomic edit is a genomic edit.

71. The cell culture of any one of claims 68-70, wherein the stem cell comprises a loss of function of a TGF beta receptor or a dominant-negative variant of a TGF beta receptor.

72. The cell culture of claim 71, wherein the TGF beta receptor is a TGF beta receptor II (TGFβRII).

73. The cell culture of any one of claims 68-72, wherein the pluripotent human stem cell:
(1) comprises at least one genomic edit characterized by an exogenous nucleic acid expression construct that comprises a nucleic acid sequence encoding:
(i) a chimeric antigen receptor (CAR);
(ii) a FcγRIII (CD16) or a variant of FcγRIII (CD16);
(iii) interleukin 15 (IL-15);
(iv) an IL-15 receptor (IL-15R) agonist, or a constitutively active variant of an IL-15 receptor;
(v) an IL-12 receptor (IL-12R) agonist, or a constitutively active variant of an IL-12 receptor;
(vi) an IL-12 receptor (IL-12R) agonist, or a constitutively active variant of an IL-12 receptor;
(vii) human leukocyte antigen G (HLA-G);
(viii) human leukocyte antigen E (HLA-E);
(ix) leukocyte surface antigen cluster of differentiation CD47 (CD47);
or any combination of two or more thereof;
and/or
(2) comprises at least one genomic edit that results in a loss of function of at least one of:
(i) cytokine inducible SH2 containing protein (CISH);
(ii) adenosine A2a receptor (ADORA2A);
(iii) T cell immunoreceptor with Ig and ITIM domains (TIGIT);
(iv) β-2 microglobulin (B2M);
(v) programmed cell death protein 1 (PD-1);
(vi) class II, major histocompatibility complex, transactivator (CIITA);
(vii) natural killer cell receptor NKG2A (natural killer group 2A);
(viii) two or more HLA class II histocompatibility antigen alpha chain genes,
and/or two or more HLA class II histocompatibility antigen beta chain genes;
(ix) cluster of differentiation 32B (CD32B, FCGR2B);
(x) T cell receptor alpha constant (TRAC);
or any combination of two or more thereof.

74. A method of increasing a level of iNK cell activity comprising:
(i) providing a pluripotent human stem cell comprising a disruption in the transforming growth factor beta (TGF beta) signaling pathway; and
(ii) differentiating the pluripotent human stem cell into an iNK cell, wherein the iNK cell has a higher level of cell activity as compared to an iNK cell not comprising a disruption of the TGF beta signaling pathway.

75. The method of claim 74, wherein the iNK is differentiated from a pluripotent human stem cell cultured in a medium comprising activin.

76. The method of claim 74 or 75, further comprising culturing the pluripotent human stem cell in a medium comprising activin before and/or during the differentiating step.

77. The method of any one of claims 74-76, further comprising disrupting the transforming growth factor beta (TGF beta) signaling pathway in the pluripotent human stem cell.

78. The method of any one of claims 73-77, wherein the stem cell comprises a genomic edit that results in a loss of function of an agonist of the TGF beta signaling pathway.

79. The method of any one of claims 73-78, wherein the stem cell comprises a loss of function of a TGF beta receptor or a dominant-negative variant of a TGF beta receptor.

80. The method of claim 79, wherein the TGF beta receptor is a TGF beta receptor II (TGFβRII).

81. The method of any one of claims 73-80, wherein the pluripotent human stem cell:
(1) comprises at least one genomic edit characterized by an exogenous nucleic acid expression construct that comprises a nucleic acid sequence encoding:
(i) a chimeric antigen receptor (CAR);
(ii) a FcγRIII (CD16) or a variant of FcγRIII (CD16);
(iii) interleukin 15 (IL-15);
(iv) an IL-15 receptor (IL-15R) agonist, or a constitutively active variant of an IL-15 receptor;
(v) an IL-12 receptor (IL-12R) agonist, or a constitutively active variant of an IL-12 receptor;
(vi) an IL-12 receptor (IL-12R) agonist, or a constitutively active variant of an IL-12 receptor;
(vii) human leukocyte antigen G (HLA-G);
(viii) human leukocyte antigen E (HLA-E);
(ix) leukocyte surface antigen cluster of differentiation CD47 (CD47);
or any combination of two or more thereof;
and/or
(2) comprises at least one genomic edit that results in a loss of function of at least one of:
(i) cytokine inducible SH2 containing protein (CISH);
(ii) adenosine A2a receptor (ADORA2A);
(iii) T cell immunoreceptor with Ig and ITIM domains (TIGIT);
(iv) β-2 microglobulin (B2M);
(v) programmed cell death protein 1 (PD-1);
(vi) class II, major histocompatibility complex, transactivator (CIITA);
(vii) natural killer cell receptor NKG2A (natural killer group 2A);
(viii) two or more HLA class II histocompatibility antigen alpha chain genes, and/or two or more HLA class II histocompatibility antigen beta chain genes;
(ix) cluster of differentiation 32B (CD32B, FCGR2B);
(x) T cell receptor alpha constant (TRAC);
or any combination of two or more thereof.

82. A method of treating a subject having or at risk of cancer, the method comprising administering to the subject the cell of any one of claim 6-13, 20-29, or 47-54, thereby treating the cancer in the subject.


SEQUENCE LISTING (1180 sequences)

SEQ ID NO: 1 - RNA, 5 nt, Artificial Sequence
  Description of Artificial Sequence Synthetic oligonucleotide
cuuuu

SEQ ID NO: 2 - RNA, 10 nt, Artificial Sequence
  Description of Artificial Sequence Synthetic oligonucleotide
aagaccuuuu

SEQ ID NO: 3 - RNA, 25 nt, Artificial Sequence
  Description of Artificial Sequence Synthetic oligonucleotide
auguguuuuugucaaaagaccuuuu

SEQ ID NO: 4 - RNA, 60 nt, Artificial Sequence
  Description of Artificial Sequence Synthetic oligonucleotide
aggccagcuugccgguuuuuuagucgugcugcuucauguguuuuugucaaaagaccuuuu

SEQ ID NO: 5 - DNA, 5 bp, Artificial Sequence
  Description of Artificial Sequence Synthetic oligonucleotide
ctttt

SEQ ID NO: 6 - DNA, 10 bp, Artificial Sequence
  Description of Artificial Sequence Synthetic oligonucleotide
aagacctttt

SEQ ID NO: 7 - DNA, 25 bp, Artificial Sequence
  Description of Artificial Sequence Synthetic oligonucleotide
atgtgtttttgtcaaaagacctttt

SEQ ID NO: 8 - DNA, 60 bp, Artificial Sequence
  Description of Artificial Sequence Synthetic oligonucleotide
aggccagcttgccggttttttagtcgtgctgcttcatgtgtttttgtcaaaagacctttt

SEQ ID NO: 9 - DNA, 20 bp, Artificial Sequence
  Description of Artificial Sequence Synthetic oligonucleotide
tttttgtcaaaagacctttt

SEQ ID NO: 10 - DNA, 30 bp, Artificial Sequence
  Description of Artificial Sequence Synthetic oligonucleotide
gcttcatgtgtttttgtcaaaagacctttt

SEQ ID NO: 11 - DNA, 50 bp, Artificial Sequence
  Description of Artificial Sequence Synthetic oligonucleotide
gccggttttttagtcgtgctgcttcatgtgtttttgtcaaaagacctttt

SEQ ID NO: 12 - DNA, 40 bp, Artificial Sequence
  Description of Artificial Sequence Synthetic oligonucleotide
tagtcgtgctgcttcatgtgtttttgtcaaaagacctttt

SEQ ID NO: 13 - DNA, 20 bp, Artificial Sequence
  Description of Artificial Sequence Synthetic oligonucleotide
ccgaagttttcttcggtttt

SEQ ID NO: 14 - DNA, 25 bp, Artificial Sequence
  Description of Artificial Sequence Synthetic oligonucleotide
tttttccgaagttttcttcggtttt

SEQ ID NO: 15 - DNA, 30 bp, Artificial Sequence
  Description of Artificial Sequence Synthetic oligonucleotide
aacgctttttccgaagttttcttcggtttt

SEQ ID NO: 16 - DNA, 41 bp, Artificial Sequence
  Description of Artificial Sequence Synthetic oligonucleotide
gcgttgttttcaacgctttttccgaagttttcttcggtttt

SEQ ID NO: 17 - DNA, 62 bp, Artificial Sequence
  Description of Artificial Sequence Synthetic oligonucleotide
ggcttcttttgaagcctttttgcgttgttttcaacgctttttccgaagttttcttcggtttt

SEQ ID NO: 18 - DNA, 25 bp, Artificial Sequence
  Description of Artificial Sequence Synthetic oligonucleotide
atgtgtttttgtcaaaagacctttt

SEQ ID NO: 19 - RNA, 25 nt, Artificial Sequence
  Description of Artificial Sequence Synthetic oligonucleotide
aaaaaaaaaaaaaaaaaaaaaaaaa

SEQ ID NO: 20 - DNA, 25 bp, Artificial Sequence
  Description of Artificial Sequence Synthetic oligonucleotide
ttttttttttttttttttttttttt

SEQ ID NO: 21 - RNA, 25 nt, Artificial Sequence
  Description of Artificial Sequence Synthetic oligonucleotide
auguguuuuugucaaaagaccuuuu

SEQ ID NO: 22 - RNA, 25 nt, Artificial Sequence
  Description of Artificial Sequence Synthetic oligonucleotide
aaaaaaaaaaaaaaaaaaaaaaaaa

SEQ ID NO: 23 - RNA, 25 nt, Artificial Sequence
  Description of Artificial Sequence Synthetic oligonucleotide
uuuuuuuuuuuuuuuuuuuuuuuuu

SEQ ID NO: 24 - DNA, 20 bp, Artificial Sequence
  Description of Artificial Sequence Synthetic oligonucleotide
tctgcagaaatgttccccgt

SEQ ID NO: 25 - RNA, 20 nt, Artificial Sequence
  Description of Artificial Sequence Synthetic oligonucleotide
ucugcagaaauguuccccgu

SEQ ID NO: 26 - RNA, 20 nt, Artificial Sequence
  Description of Artificial Sequence Synthetic oligonucleotide
uaauuucuacucuuguagau

SEQ ID NO: 27 - RNA, 40 nt, Artificial Sequence
  Description of Artificial Sequence Synthetic oligonucleotide
uaauuucuacucuuguagauucugcagaaauguuccccgu

SEQ ID NO: 28 - DNA, 65 bp, Artificial Sequence
  Description of Artificial Sequence Synthetic oligonucleotide
  Description of Combined DNA/RNA Molecule Synthetic oligonucleotide
atgtgtttttgtcaaaagaccttttuaauuucuacucuuguagauucugcagaaauguuccccgu

SEQ ID NO: 29 - DNA, 20 bp, Artificial Sequence
  Description of Artificial Sequence Synthetic oligonucleotide
caggacgatgtgcagcggcc

SEQ ID NO: 30 - DNA, 20 bp, Artificial Sequence
  Description of Artificial Sequence Synthetic oligonucleotide
accgcacgttcagaagtcgg

SEQ ID NO: 31 - DNA, 20 bp, Artificial Sequence
  Description of Artificial Sequence Synthetic oligonucleotide
acaactgtgtaaattttgtg

SEQ ID NO: 32 - DNA, 20 bp, Artificial Sequence
  Description of Artificial Sequence Synthetic oligonucleotide
caactgtgtaaattttgtga

SEQ ID NO: 33 - DNA, 20 bp, Artificial Sequence
  Description of Artificial Sequence Synthetic oligonucleotide
acctgtgacaaccagaaatc

SEQ ID NO: 34 - DNA, 20 bp, Artificial Sequence
  Description of Artificial Sequence Synthetic oligonucleotide
cctgtgacaaccagaaatcc

SEQ ID NO: 35 - DNA, 20 bp, Artificial Sequence
  Description of Artificial Sequence Synthetic oligonucleotide
tgtggcttctcacagatgga

SEQ ID NO: 36 - DNA, 20 bp, Artificial Sequence
  Description of Artificial Sequence Synthetic oligonucleotide
tctgtgagaagccacaggaa

SEQ ID NO: 37 - DNA, 20 bp, Artificial Sequence
  Description of Artificial Sequence Synthetic oligonucleotide
aagctcccctaccatgactt

SEQ ID NO: 38 - DNA, 20 bp, Artificial Sequence
  Description of Artificial Sequence Synthetic oligonucleotide
gaataaagtcatggtagggg

SEQ ID NO: 39 - DNA, 20 bp, Artificial Sequence
  Description of Artificial Sequence Synthetic oligonucleotide
agaataaagtcatggtaggg

SEQ ID NO: 40 - DNA, 20 bp, Artificial Sequence
  Description of Artificial Sequence Synthetic oligonucleotide
ctaccatgactttattctgg

SEQ ID NO: 41 - DNA, 20 bp, Artificial Sequence
  Description of Artificial Sequence Synthetic oligonucleotide
taccatgactttattctgga

SEQ ID NO: 42 - DNA, 20 bp, Artificial Sequence
  Description of Artificial Sequence Synthetic oligonucleotide
taatgcactttggagaagca

SEQ ID NO: 43 - DNA, 20 bp, Artificial Sequence
  Description of Artificial Sequence Synthetic oligonucleotide
ttcataatgcactttggaga

SEQ ID NO: 44 - DNA, 20 bp, Artificial Sequence
  Description of Artificial Sequence Synthetic oligonucleotide
aagtgcattatgaaggaaaa

SEQ ID NO: 45 - DNA, 20 bp, Artificial Sequence
  Description of Artificial Sequence Synthetic oligonucleotide
tgtgttcctgtagctctgat

SEQ ID NO: 46 - DNA, 20 bp, Artificial Sequence
  Description of Artificial Sequence Synthetic oligonucleotide
tgtagctctgatgagtgcaa

SEQ ID NO: 47 - DNA, 20 bp, Artificial Sequence
  Description of Artificial Sequence Synthetic oligonucleotide
agtgacaggcatcagcctcc

SEQ ID NO: 48 - DNA, 20 bp, Artificial Sequence
  Description of Artificial Sequence Synthetic oligonucleotide
agtggtggcaggaggctgat

SEQ ID NO: 49 - DNA, 20 bp, Artificial Sequence
  Description of Artificial Sequence Synthetic oligonucleotide
aggttgaactcagcttctgc

SEQ ID NO: 50 - DNA, 20 bp, Artificial Sequence
  Description of Artificial Sequence Synthetic oligonucleotide
caggttgaactcagcttctg

SEQ ID NO: 51 - DNA, 20 bp, Artificial Sequence
  Description of Artificial Sequence Synthetic oligonucleotide
acctgggaaaccggcaagac

SEQ ID NO: 52 - DNA, 20 bp, Artificial Sequence
  Description of Artificial Sequence Synthetic oligonucleotide
cgtcttgccggtttcccagg

SEQ ID NO: 53 - DNA, 20 bp, Artificial Sequence
  Description of Artificial Sequence Synthetic oligonucleotide
gcgtcttgccggtttcccag

SEQ ID NO: 54 - DNA, 20 bp, Artificial Sequence
  Description of Artificial Sequence Synthetic oligonucleotide
tgagcttccgcgtcttgccg

SEQ ID NO: 55 - DNA, 20 bp, Artificial Sequence
  Description of Artificial Sequence Synthetic oligonucleotide
gcgagcactgtgccatcatc

SEQ ID NO: 56 - DNA, 20 bp, Artificial Sequence
  Description of Artificial Sequence Synthetic oligonucleotide
ggatgatggcacagtgctcg

SEQ ID NO: 57 - DNA, 20 bp, Artificial Sequence
  Description of Artificial Sequence Synthetic oligonucleotide
aggatgatggcacagtgctc

SEQ ID NO: 58 - DNA, 20 bp, Artificial Sequence
  Description of Artificial Sequence Synthetic oligonucleotide
cgtgtgccaacaacatcaac

SEQ ID NO: 59 - DNA, 20 bp, Artificial Sequence
  Description of Artificial Sequence Synthetic oligonucleotide
gctcaatgggcagcagctct

SEQ ID NO: 60 - DNA, 20 bp, Artificial Sequence
  Description of Artificial Sequence Synthetic oligonucleotide
accagggtgtccagctcaat

SEQ ID NO: 61 - DNA, 20 bp, Artificial Sequence
  Description of Artificial Sequence Synthetic oligonucleotide
caccagggtgtccagctcaa

SEQ ID NO: 62 - DNA, 20 bp, Artificial Sequence
  Description of Artificial Sequence Synthetic oligonucleotide
ccaccagggtgtccagctca

SEQ ID NO: 63 - DNA, 20 bp, Artificial Sequence
  Description of Artificial Sequence Synthetic oligonucleotide
gcttggccttatagacctca

SEQ ID NO: 64 - DNA, 20 bp, Artificial Sequence
  Description of Artificial Sequence Synthetic oligonucleotide
gagcagtttgagacagtggc

SEQ ID NO: 65 - DNA, 20 bp, Artificial Sequence
  Description of Artificial Sequence Synthetic oligonucleotide
agaggcatactcctcatagg

SEQ ID NO: 66 - DNA, 20 bp, Artificial Sequence
  Description of Artificial Sequence Synthetic oligonucleotide
ctatgaggagtatgcctctt

SEQ ID NO: 67 - DNA, 20 bp, Artificial Sequence
  Description of Artificial Sequence Synthetic oligonucleotide
aagaggcatactcctcatag

SEQ ID NO: 68 - DNA, 20 bp, Artificial Sequence
  Description of Artificial Sequence Synthetic oligonucleotide
tatgaggagtatgcctcttg

SEQ ID NO: 69 - DNA, 20 bp, Artificial Sequence
  Description of Artificial Sequence Synthetic oligonucleotide
gattgatgtctgagaagatg

SEQ ID NO: 70 - DNA, 20 bp, Artificial Sequence
  Description of Artificial Sequence Synthetic oligonucleotide
ctcctcagccgtcaggaact

SEQ ID NO: 71 - DNA, 20 bp, Artificial Sequence
  Description of Artificial Sequence Synthetic oligonucleotide
gttcctgacggctgaggagc

SEQ ID NO: 72 - DNA, 20 bp, Artificial Sequence
  Description of Artificial Sequence Synthetic oligonucleotide
gctcctcagccgtcaggaac

SEQ ID NO: 73 - DNA, 20 bp, Artificial Sequence
  Description of Artificial Sequence Synthetic oligonucleotide
tgacggctgaggagcggaag

SEQ ID NO: 74 - DNA, 20 bp, Artificial Sequence
  Description of Artificial Sequence Synthetic oligonucleotide
tcttccgctcctcagccgtc

SEQ ID NO: 75 - DNA, 20 bp, Artificial Sequence
  Description of Artificial Sequence Synthetic oligonucleotide
aactccgtcttccgctcctc

SEQ ID NO: 76 - DNA, 20 bp, Artificial Sequence
  Description of Artificial Sequence Synthetic oligonucleotide
caactccgtcttccgctcct

SEQ ID NO: 77 - DNA, 20 bp, Artificial Sequence
  Description of Artificial Sequence Synthetic oligonucleotide
ccaactccgtcttccgctcc

SEQ ID NO: 78 - DNA, 20 bp, Artificial Sequence
  Description of Artificial Sequence Synthetic oligonucleotide
acgccaagggcaacctacag

SEQ ID NO: 79 - DNA, 20 bp, Artificial Sequence
  Description of Artificial Sequence Synthetic oligonucleotide
cgccaagggcaacctacagg

SEQ ID NO: 80 - DNA, 20 bp, Artificial Sequence
  Description of Artificial Sequence Synthetic oligonucleotide
agctgatgacatgccgcgtc

SEQ ID NO: 81 - DNA, 20 bp, Artificial Sequence
  Description of Artificial Sequence Synthetic oligonucleotide
gggcgagggagctgcccagc

SEQ ID NO: 82 - DNA, 20 bp, Artificial Sequence
  Description of Artificial Sequence Synthetic oligonucleotide
cgggcgagggagctgcccag

SEQ ID NO: 83 - DNA, 20 bp, Artificial Sequence
  Description of Artificial Sequence Synthetic oligonucleotide
ccgggcgagggagctgccca

SEQ ID NO: 84 - DNA, 20 bp, Artificial Sequence
  Description of Artificial Sequence Synthetic oligonucleotide
tcgcccgggggattgctcac

SEQ ID NO: 85 - DNA, 20 bp, Artificial Sequence
  Description of Artificial Sequence Synthetic oligonucleotide
acatggagtgtgatcactgt

SEQ ID NO: 86 - DNA, 20 bp, Artificial Sequence
  Description of Artificial Sequence Synthetic oligonucleotide
cagtgatcacactccatgtg

SEQ ID NO: 87 - DNA, 20 bp, Artificial Sequence
  Description of Artificial Sequence Synthetic oligonucleotide
tgtgggaggcccaagatgcc

SEQ ID NO: 88 - DNA, 20 bp, Artificial Sequence
  Description of Artificial Sequence Synthetic oligonucleotide
tgtgcacgatgggcatcttg

SEQ ID NO: 89 - DNA, 20 bp, Artificial Sequence
  Description of Artificial Sequence Synthetic oligonucleotide
cgaggatattggagctcttg

SEQ ID NO: 90 - DNA, 20 bp, Artificial Sequence
  Description of Artificial Sequence Synthetic oligonucleotide
atatcctcgtgaagaacgac

SEQ ID NO: 91 - DNA, 20 bp, Artificial Sequence
  Description of Artificial Sequence Synthetic oligonucleotide
gacgcagggaaagcccaaag

SEQ ID NO: 92 - DNA, 20 bp, Artificial Sequence
  Description of Artificial Sequence Synthetic oligonucleotide
ctgcgtctggaccctactct

SEQ ID NO: 93 - DNA, 20 bp, Artificial Sequence
  Description of Artificial Sequence Synthetic oligonucleotide
tgcgtctggaccctactctg

SEQ ID NO: 94 - DNA, 20 bp, Artificial Sequence
  Description of Artificial Sequence Synthetic oligonucleotide
cagacagagtagggtccaga

SEQ ID NO: 95 - DNA, 20 bp, Artificial Sequence
  Description of Artificial Sequence Synthetic oligonucleotide
gccagcacgatcccaccgca

SEQ ID NO: 96 - DNA, 20 bp, Artificial Sequence
  Description of Artificial Sequence Synthetic oligonucleotide
aaggaaaaaaaaaagcctgg

SEQ ID NO: 97 - DNA, 20 bp, Artificial Sequence
  Description of Artificial Sequence Synthetic oligonucleotide
acaccagcaatcctgacttg

SEQ ID NO: 98 - DNA, 20 bp, Artificial Sequence
  Description of Artificial Sequence Synthetic oligonucleotide
actagcaacaagtcaggatt

SEQ ID NO: 99 - DNA, 20 bp, Artificial Sequence
  Description of Artificial Sequence Synthetic oligonucleotide
gcaactcccagtggtggcag

SEQ ID NO: 100 - DNA, 20 bp, Artificial Sequence
  Description of Artificial Sequence Synthetic oligonucleotide
tgtcatcatcatcttctact

SEQ ID NO: 101 - DNA, 20 bp, Artificial Sequence
  Description of Artificial Sequence Synthetic oligonucleotide
gacctcagcaaagcgacctt

SEQ ID NO: 102 - DNA, 20 bp, Artificial Sequence
  Description of Artificial Sequence Synthetic oligonucleotide
aggccaagctgaagcagaac

SEQ ID NO: 103 - DNA, 20 bp, Artificial Sequence
  Description of Artificial Sequence Synthetic oligonucleotide
aggagtatgcctcttggaag

SEQ ID NO: 104 - DNA, 20 bp, Artificial Sequence
  Description of Artificial Sequence Synthetic oligonucleotide
cctcttggaagacagagaag

SEQ ID NO: 105 - DNA, 20 bp, Artificial Sequence
  Description of Artificial Sequence Synthetic oligonucleotide
ttctcatgcttcagattgat

SEQ ID NO: 106 - DNA, 20 bp, Artificial Sequence
  Description of Artificial Sequence Synthetic oligonucleotide
ctcgtgaagaacgacctaac

SEQ ID NO: 107 - DNA, 20 bp, Artificial Sequence
  Description of Artificial Sequence Synthetic oligonucleotide
ggccgctgcacatcgtcctg

SEQ ID NO: 108 - DNA, 20 bp, Artificial Sequence
  Description of Artificial Sequence Synthetic oligonucleotide
gcggggtctgccatgggtcg

SEQ ID NO: 109 - DNA, 20 bp, Artificial Sequence
  Description of Artificial Sequence Synthetic oligonucleotide
agttgctcatgcaggatttc

SEQ ID NO: 110 - DNA, 20 bp, Artificial Sequence
  Description of Artificial Sequence Synthetic oligonucleotide
ccagaataaagtcatggtag

SEQ ID NO: 111 - DNA, 20 bp, Artificial Sequence
  Description of Artificial Sequence Synthetic oligonucleotide
cccctaccatgactttattc

SEQ ID NO: 112 - DNA, 20 bp, Artificial Sequence
  Description of Artificial Sequence Synthetic oligonucleotide
aagtcatggtaggggagctt

SEQ ID NO: 113 - DNA, 20 bp, Artificial Sequence
  Description of Artificial Sequence Synthetic oligonucleotide
agtcatggtaggggagcttg

SEQ ID NO: 114 - DNA, 20 bp, Artificial Sequence
  Description of Artificial Sequence Synthetic oligonucleotide
attgcactcatcagagctac

SEQ ID NO: 115 - DNA, 20 bp, Artificial Sequence
  Description of Artificial Sequence Synthetic oligonucleotide
cctagagtgaagagattcat

SEQ ID NO: 116 - DNA, 20 bp, Artificial Sequence
  Description of Artificial Sequence Synthetic oligonucleotide
ccaatgaatctcttcactct

SEQ ID NO: 117 - DNA, 20 bp, Artificial Sequence
  Description of Artificial Sequence Synthetic oligonucleotide
aaagtcatggtaggggagct

SEQ ID NO: 118 - DNA, 20 bp, Artificial Sequence
  Description of Artificial Sequence Synthetic oligonucleotide
gtgagcaatcccccgggcga

SEQ ID NO: 119 - DNA, 20 bp, Artificial Sequence
  Description of Artificial Sequence Synthetic oligonucleotide
gtcgttcttcacgaggatat

SEQ ID NO: 120 - DNA, 20 bp, Artificial Sequence
  Description of Artificial Sequence Synthetic oligonucleotide
gccgcgtcaggtactcctgt

SEQ ID NO: 121 - DNA, 20 bp, Artificial Sequence
  Description of Artificial Sequence Synthetic oligonucleotide
gacgcggcatgtcatcagct

SEQ ID NO: 122 - DNA, 20 bp, Artificial Sequence
  Description of Artificial Sequence Synthetic oligonucleotide
gcttctgctgccggttaacg

SEQ ID NO: 123 - DNA, 20 bp, Artificial Sequence
  Description of Artificial Sequence Synthetic oligonucleotide
gtggatgacctggctaacag

SEQ ID NO: 124 - DNA, 20 bp, Artificial Sequence
  Description of Artificial Sequence Synthetic oligonucleotide
gtgatcacactccatgtggg

SEQ ID NO: 125 - DNA, 20 bp, Artificial Sequence
  Description of Artificial Sequence Synthetic oligonucleotide
gcccattgagctggacaccc

SEQ ID NO: 126 - DNA, 20 bp, Artificial Sequence
  Description of Artificial Sequence Synthetic oligonucleotide
gcggtcatcttccaggatga

SEQ ID NO: 127 - DNA, 20 bp, Artificial Sequence
  Description of Artificial Sequence Synthetic oligonucleotide
gggagctgcccagcttgcgc

SEQ ID NO: 128 - DNA, 20 bp, Artificial Sequence
  Description of Artificial Sequence Synthetic oligonucleotide
gttgatgttgttggcacacg

SEQ ID NO: 129 - DNA, 20 bp, Artificial Sequence
  Description of Artificial Sequence Synthetic oligonucleotide
ggcatcttgggcctcccaca

SEQ ID NO: 130 - DNA, 20 bp, Artificial Sequence
  Description of Artificial Sequence Synthetic oligonucleotide
gcggcatgtcatcagctggg

SEQ ID NO: 131 - DNA, 20 bp, Artificial Sequence
  Description of Artificial Sequence Synthetic oligonucleotide
gctcctcagccgtcaggaac

SEQ ID NO: 132 - DNA, 20 bp, Artificial Sequence
  Description of Artificial Sequence Synthetic oligonucleotide
gctggtgttatattctgatg

SEQ ID NO: 133 - DNA, 20 bp, Artificial Sequence
  Description of Artificial Sequence Synthetic oligonucleotide
ccgacttctgaacgtgcggt

SEQ ID NO: 134 - DNA, 20 bp, Artificial Sequence
  Description of Artificial Sequence Synthetic oligonucleotide
tgctggcgatacgcgtccac

SEQ ID NO: 135 - DNA, 20 bp, Artificial Sequence
  Description of Artificial Sequence Synthetic oligonucleotide
cccgacttctgaacgtgcgg

SEQ ID NO: 136 - DNA, 20 bp, Artificial Sequence
  Description of Artificial Sequence Synthetic oligonucleotide
ccaccgcacgttcagaagtc

SEQ ID NO: 137 - DNA, 20 bp, Artificial Sequence
  Description of Artificial Sequence Synthetic oligonucleotide
tcacccgacttctgaacgtg

SEQ ID NO: 138 - DNA, 20 bp, Artificial Sequence
  Description of Artificial Sequence Synthetic oligonucleotide
cccaccgcacgttcagaagt

SEQ ID NO: 139 - DNA, 20 bp, Artificial Sequence
  Description of Artificial Sequence Synthetic oligonucleotide
cgagcagcggggtctgccat

SEQ ID NO: 140 - DNA, 20 bp, Artificial Sequence
  Description of Artificial Sequence Synthetic oligonucleotide
acgagcagcggggtctgcca

SEQ ID NO: 141 - DNA, 20 bp, Artificial Sequence
  Description of Artificial Sequence Synthetic oligonucleotide
agcggggtctgccatgggtc

SEQ ID NO: 142 - DNA, 20 bp, Artificial Sequence
  Description of Artificial Sequence Synthetic oligonucleotide
cctgagcagcccccgaccca

SEQ ID NO: 143 - DNA, 20 bp, Artificial Sequence
  Description of Artificial Sequence Synthetic oligonucleotide
ccatgggtcgggggctgctc

SEQ ID NO: 144 - DNA, 20 bp, Artificial Sequence
  Description of Artificial Sequence Synthetic oligonucleotide
aacgtgcggtgggatcgtgc

SEQ ID NO: 145 - DNA, 20 bp, Artificial Sequence
  Description of Artificial Sequence Synthetic oligonucleotide
ggacgatgtgcagcggccac

SEQ ID NO: 146 - DNA, 20 bp, Artificial Sequence
  Description of Artificial Sequence Synthetic oligonucleotide
gtccacaggacgatgtgcag

SEQ ID NO: 147 - DNA, 20 bp, Artificial Sequence
  Description of Artificial Sequence Synthetic oligonucleotide
catgggtcgggggctgctca

SEQ ID NO: 148 - DNA, 20 bp, Artificial Sequence
  Description of Artificial Sequence Synthetic oligonucleotide
cagcggggtctgccatgggt

SEQ ID NO: 149 - DNA, 20 bp, Artificial Sequence
  Description of Artificial Sequence Synthetic oligonucleotide
atgggtcgggggctgctcag

SEQ ID NO: 150 - DNA, 20 bp, Artificial Sequence
  Description of Artificial Sequence Synthetic oligonucleotide
cggggtctgccatgggtcgg

SEQ ID NO: 151 - DNA, 20 bp, Artificial Sequence
  Description of Artificial Sequence Synthetic oligonucleotide
aggaagtctgtgtggctgta

SEQ ID NO: 152 - DNA, 20 bp, Artificial Sequence
  Description of Artificial Sequence Synthetic oligonucleotide
ctccatctgtgagaagccac

SEQ ID NO: 153 - DNA, 20 bp, Artificial Sequence
  Description of Artificial Sequence Synthetic oligonucleotide
atgatagtcactgacaacaa

SEQ ID NO: 154 - DNA, 20 bp, Artificial Sequence
  Description of Artificial Sequence Synthetic oligonucleotide
gatgctgcagttgctcatgc

SEQ ID NO: 155 - DNA, 20 bp, Artificial Sequence
  Description of Artificial Sequence Synthetic oligonucleotide
acagccacacagacttcctg

SEQ ID NO: 156 - DNA, 20 bp, Artificial Sequence
  Description of Artificial Sequence Synthetic oligonucleotide
gaagccacaggaagtctgtg

SEQ ID NO: 157 - DNA, 20 bp, Artificial Sequence
  Description of Artificial Sequence Synthetic oligonucleotide
ttcctgtggcttctcacaga

SEQ ID NO: 158 - DNA, 20 bp, Artificial Sequence
  Description of Artificial Sequence Synthetic oligonucleotide
ctgtggcttctcacagatgg

SEQ ID NO: 159 - DNA, 20 bp, Artificial Sequence
  Description of Artificial Sequence Synthetic oligonucleotide
tcacaaaatttacacagttg

SEQ ID NO: 160 - DNA, 20 bp, Artificial Sequence
  Description of Artificial Sequence Synthetic oligonucleotide
gacaacatcatcttctcaga

SEQ ID NO: 161 - DNA, 20 bp, Artificial Sequence
  Description of Artificial Sequence Synthetic oligonucleotide
tccagaataaagtcatggta

SEQ ID NO: 162 - DNA, 20 bp, Artificial Sequence
  Description of Artificial Sequence Synthetic oligonucleotide
ggtaggggagcttggggtca

SEQ ID NO: 163 - DNA, 20 bp, Artificial Sequence
  Description of Artificial Sequence Synthetic oligonucleotide
ttctccaaagtgcattatga

SEQ ID NO: 164 - DNA, 20 bp, Artificial Sequence
  Description of Artificial Sequence Synthetic oligonucleotide
catcttccagaataaagtca

SEQ ID NO: 165 - DNA, 20 bp, Artificial Sequence
  Description of Artificial Sequence Synthetic oligonucleotide
cacatgaagaaagtctcacc

SEQ ID NO: 166 - DNA, 20 bp, Artificial Sequence
  Description of Artificial Sequence Synthetic oligonucleotide
ttccagaataaagtcatggt

SEQ ID NO: 167 - DNA, 20 bp, Artificial Sequence
  Description of Artificial Sequence Synthetic oligonucleotide
ttttccttcataatgcactt

SEQ ID NO: 168 - DNA, 20 bp, Artificial Sequence
  Description of Artificial Sequence Synthetic oligonucleotide
cacagttgtggaaacttgac

SEQ ID NO: 169 - DNA, 20 bp, Artificial Sequence
  Description of Artificial Sequence Synthetic oligonucleotide
cccaactccgtcttccgctc

SEQ ID NO: 170 - DNA, 20 bp, Artificial Sequence
  Description of Artificial Sequence Synthetic oligonucleotide
ggctttccctgcgtctggac

SEQ ID NO: 171 - DNA, 20 bp, Artificial Sequence
  Description of Artificial Sequence Synthetic oligonucleotide
ctgaggtctataaggccaag

SEQ ID NO: 172 - DNA, 20 bp, Artificial Sequence
  Description of Artificial Sequence Synthetic oligonucleotide
tgatgtgagattttccacct

SEQ ID NO: 173 - DNA, 20 bp, Artificial Sequence
  Description of Artificial Sequence Synthetic oligonucleotide
cctatgaggagtatgcctct

SEQ ID NO: 174 - DNA, 20 bp, Artificial Sequence
  Description of Artificial Sequence Synthetic oligonucleotide
aagtgacaggcatcagcctc

SEQ ID NO: 175 - DNA, 20 bp, Artificial Sequence
  Description of Artificial Sequence Synthetic oligonucleotide
ccatgaccccaagctcccct

SEQ ID NO: 176 - DNA, 20 bp, Artificial Sequence
  Description of Artificial Sequence Synthetic oligonucleotide
cttcataatgcactttggag

SEQ ID NO: 177 - DNA, 20 bp, Artificial Sequence
  Description of Artificial Sequence Synthetic oligonucleotide
ttcatgtgttcctgtagctc

SEQ ID NO: 178 - DNA, 20 bp, Artificial Sequence
  Description of Artificial Sequence Synthetic oligonucleotide
ttctggaagatgctgcttct

SEQ ID NO: 179 - DNA, 20 bp, Artificial Sequence
  Description of Artificial Sequence Synthetic oligonucleotide
cccaccagggtgtccagctc

SEQ ID NO: 180 - DNA, 20 bp, Artificial Sequence
  Description of Artificial Sequence Synthetic oligonucleotide
agacagtggcagtcaagatc

SEQ ID NO: 181 - DNA, 20 bp, Artificial Sequence
  Description of Artificial Sequence Synthetic oligonucleotide
cctgcgtctggaccctactc

SEQ ID NO: 182 - DNA, 20 bp, Artificial Sequence
  Description of Artificial Sequence Synthetic oligonucleotide
cacaactgtgtaaattttgt

SEQ ID NO: 183 - DNA, 20 bp, Artificial Sequence
  Description of Artificial Sequence Synthetic oligonucleotide
gagaagcagcatcttccaga

SEQ ID NO: 184 - DNA, 20 bp, Artificial Sequence
  Description of Artificial Sequence Synthetic oligonucleotide
tggttgtcacaggtggaaaa

SEQ ID NO: 185 - DNA, 20 bp, Artificial Sequence
  Description of Artificial Sequence Synthetic oligonucleotide
ccaggttgaactcagcttct

SEQ ID NO: 186 - DNA, 21 bp, Artificial Sequence
  Description of Artificial Sequence Synthetic oligonucleotide
atcacaaaatttacacagttg

SEQ ID NO: 187 - DNA, 21 bp, Artificial Sequence
  Description of Artificial Sequence Synthetic oligonucleotide
ggcatcagcctcctgccacca

SEQ ID NO: 188 - DNA, 21 bp, Artificial Sequence
  Description of Artificial Sequence Synthetic oligonucleotide
gttagccaggtcatccacaga

SEQ ID NO: 189 - DNA, 21 bp, Artificial Sequence
  Description of Artificial Sequence Synthetic oligonucleotide
gctgggcagctccctcgcccg

SEQ ID NO: 190 - DNA, 21 bp, Artificial Sequence
  Description of Artificial Sequence Synthetic oligonucleotide
caggaggctgatgcctgtcac

SEQ ID NO: 191 - DNA, 21 bp, Artificial Sequence
  Description of Artificial Sequence Synthetic oligonucleotide
gaggagcggaagacggagttg

SEQ ID NO: 192 - DNA, 21 bp, Artificial Sequence
  Description of Artificial Sequence Synthetic oligonucleotide
cgtctggaccctactctgtct

SEQ ID NO: 193 - DNA, 21 bp, Artificial Sequence
  Description of Artificial Sequence Synthetic oligonucleotide
tttttccttcataatgcactt

SEQ ID NO: 194 - DNA, 21 bp, Artificial Sequence
  Description of Artificial Sequence Synthetic oligonucleotide
ccattgagctggacaccctgg

SEQ ID NO: 195 - DNA, 21 bp, Artificial Sequence
  Description of Artificial Sequence Synthetic oligonucleotide
cttctccaaagtgcattatga

SEQ ID NO: 196 - DNA, 21 bp, Artificial Sequence
  Description of Artificial Sequence Synthetic oligonucleotide
gcccaagatgcccatcgtgca

SEQ ID NO: 197 - DNA, 21 bp, Artificial Sequence
  Description of Artificial Sequence Synthetic oligonucleotide
tcatgtgttcctgtagctctg

SEQ ID NO: 198 - DNA, 21 bp, Artificial Sequence
  Description of Artificial Sequence Synthetic oligonucleotide
gtgatgctgcagttgctcatg

SEQ ID NO: 199 - DNA, 21 bp, Artificial Sequence
  Description of Artificial Sequence Synthetic oligonucleotide
tctcatgcttcagattgatgt

SEQ ID NO: 200 - DNA, 21 bp, Artificial Sequence
  Description of Artificial Sequence Synthetic oligonucleotide
tccctatgaggagtatgcctc

SEQ ID NO: 201 - DNA, 21 bp, Artificial Sequence
  Description of Artificial Sequence Synthetic oligonucleotide
catcacaaaatttacacagtt

SEQ ID NO: 202 - DNA, 21 bp, Artificial Sequence
  Description of Artificial Sequence Synthetic oligonucleotide
attgagctggacaccctggtg

SEQ ID NO: 203 - DNA, 21 bp, Artificial Sequence
  Description of Artificial Sequence Synthetic oligonucleotide
cagtcaagatctttccctatg

SEQ ID NO: 204 - DNA, 21 bp, Artificial Sequence
  Description of Artificial Sequence Synthetic oligonucleotide
aggatttctggttgtcacagg

SEQ ID NO: 205 - DNA, 21 bp, Artificial Sequence
  Description of Artificial Sequence Synthetic oligonucleotide
tccacagtgatcacactccat

SEQ ID NO: 206 - DNA, 21 bp, Artificial Sequence
  Description of Artificial Sequence Synthetic oligonucleotide
agcagaacacttcagagcagt

SEQ ID NO: 207 - DNA, 21 bp, Artificial Sequence
  Description of Artificial Sequence Synthetic oligonucleotide
ccggcaagacgcggaagctca

SEQ ID NO: 208 - DNA, 21 bp, Artificial Sequence
  Description of Artificial Sequence Synthetic oligonucleotide
gatgtcagagcggtcatcttc

SEQ ID NO: 209 - DNA, 21 bp, Artificial Sequence
  Description of Artificial Sequence Synthetic oligonucleotide
tcattgcactcatcagagcta

SEQ ID NO: 210 - DNA, 21 bp, Artificial Sequence
  Description of Artificial Sequence Synthetic oligonucleotide
cttccagaataaagtcatggt

SEQ ID NO: 211 - DNA, 21 bp, Artificial Sequence
  Description of Artificial Sequence Synthetic oligonucleotide
agattttccacctgtgacaac

SEQ ID NO: 212 - DNA, 21 bp, Artificial Sequence
  Description of Artificial Sequence Synthetic oligonucleotide
actgcagcatcacctccatct

SEQ ID NO: 213 - DNA, 21 bp, Artificial Sequence
  Description of Artificial Sequence Synthetic oligonucleotide
agctgggcagctccctcgccc

SEQ ID NO: 214 - DNA, 21 bp, Artificial Sequence
  Description of Artificial Sequence Synthetic oligonucleotide
tgacggctgaggagcggaaga

SEQ ID NO: 215 - DNA, 21 bp, Artificial Sequence
  Description of Artificial Sequence Synthetic oligonucleotide
cattgagctggacaccctggt

SEQ ID NO: 216 - DNA, 21 bp, Artificial Sequence
  Description of Artificial Sequence Synthetic oligonucleotide
agcaaagcgacctttccccac

SEQ ID NO: 217 - DNA, 21 bp, Artificial Sequence
  Description of Artificial Sequence Synthetic oligonucleotide
cgcgttaaccggcagcagaag

SEQ ID NO: 218 - DNA, 21 bp, Artificial Sequence
  Description of Artificial Sequence Synthetic oligonucleotide
gaaatatgactagcaacaagt

SEQ ID NO: 219 - DNA, 21 bp, Artificial Sequence
  Description of Artificial Sequence Synthetic oligonucleotide
agacagagtagggtccagacg

SEQ ID NO: 220 - DNA, 21 bp, Artificial Sequence
  Description of Artificial Sequence Synthetic oligonucleotide
caggatttctggttgtcacag

SEQ ID NO: 221 - DNA, 21 bp, Artificial Sequence
  Description of Artificial Sequence Synthetic oligonucleotide
ctcctgtaggttgcccttggc

SEQ ID NO: 222 - DNA, 21 bp, Artificial Sequence
  Description of Artificial Sequence Synthetic oligonucleotide
acagagtagggtccagacgca

SEQ ID NO: 223 - DNA, 21 bp, Artificial Sequence
  Description of Artificial Sequence Synthetic oligonucleotide
gcttctccaaagtgcattatg

SEQ ID NO: 224 - DNA, 21 bp, Artificial Sequence
  Description of Artificial Sequence Synthetic oligonucleotide
gcagcagaagctgagttcaac

SEQ ID NO: 225 - DNA, 21 bp, Artificial Sequence
  Description of Artificial Sequence Synthetic oligonucleotide
tgaggagcggaagacggagtt

SEQ ID NO: 226 - DNA, 21 bp, Artificial Sequence
  Description of Artificial Sequence Synthetic oligonucleotide
ctttggagaagcagcatcttc

SEQ ID NO: 227 - DNA, 21 bp, Artificial Sequence
  Description of Artificial Sequence Synthetic oligonucleotide
ctcccctaccatgactttatt

SEQ ID NO: 228 - DNA, 21 bp, Artificial Sequence
  Description of Artificial Sequence Synthetic oligonucleotide
gacagagtagggtccagacgc

SEQ ID NO: 229 - DNA, 21 bp, Artificial Sequence
  Description of Artificial Sequence Synthetic oligonucleotide
ctgaggagcggaagacggagt

SEQ ID NO: 230 - DNA, 21 bp, Artificial Sequence
  Description of Artificial Sequence Synthetic oligonucleotide
gggcatcttgggcctcccaca

SEQ ID NO: 231 - DNA, 21 bp, Artificial Sequence
  Description of Artificial Sequence Synthetic oligonucleotide
ccaagaggcatactcctcata

SEQ ID NO: 232 - DNA, 21 bp, Artificial Sequence
  Description of Artificial Sequence Synthetic oligonucleotide
agaatgacgagaacataacac

SEQ ID NO: 233 - DNA, 21 bp, Artificial Sequence
  Description of Artificial Sequence Synthetic oligonucleotide
cctgacgcggcatgtcatcag

SEQ ID NO: 234 - DNA, 21 bp, Artificial Sequence
  Description of Artificial Sequence Synthetic oligonucleotide
agcgagcactgtgccatcatc

SEQ ID NO: 235 - DNA, 21 bp, Artificial Sequence
  Description of Artificial Sequence Synthetic oligonucleotide
gcaggttaggtcgttcttcac

SEQ ID NO: 236 - DNA, 21 bp, Artificial Sequence
  Description of Artificial Sequence Synthetic oligonucleotide
acctccatctgtgagaagcca

SEQ ID NO: 237 - DNA, 21 bp, Artificial Sequence
  Description of Artificial Sequence Synthetic oligonucleotide
taaagtcatggtaggggagct

SEQ ID NO: 238 - DNA, 21 bp, Artificial Sequence
  Description of Artificial Sequence Synthetic oligonucleotide
tcagagctacaggaacacatg

SEQ ID NO: 239 - DNA, 21 bp, Artificial Sequence
  Description of Artificial Sequence Synthetic oligonucleotide
tctcagacatcaatctgaagc

SEQ ID NO: 240 - DNA, 21 bp, Artificial Sequence
  Description of Artificial Sequence Synthetic oligonucleotide
catcagcctcctgccaccact

SEQ ID NO: 241 - DNA, 21 bp, Artificial Sequence
  Description of Artificial Sequence Synthetic oligonucleotide
cgctcctcagccgtcaggaac

SEQ ID NO: 242 - DNA, 21 bp, Artificial Sequence
  Description of Artificial Sequence Synthetic oligonucleotide
aacctgggaaaccggcaagac

SEQ ID NO: 243 - DNA, 21 bp, Artificial Sequence
  Description of Artificial Sequence Synthetic oligonucleotide
tccacgccaagggcaacctac

SEQ ID NO: 244 - DNA, 21 bp, Artificial Sequence
  Description of Artificial Sequence Synthetic oligonucleotide
gaggtgagcaatcccccgggc

SEQ ID NO: 245 - DNA, 21 bp, Artificial Sequence
  Description of Artificial Sequence Synthetic oligonucleotide
cagcagaagctgagttcaacc

SEQ ID NO: 246 - DNA, 21 bp, Artificial Sequence
  Description of Artificial Sequence Synthetic oligonucleotide
tccaagaggcatactcctcat

SEQ ID NO: 247 - DNA, 21 bp, Artificial Sequence
  Description of Artificial Sequence Synthetic oligonucleotide
agcagaagctgagttcaacct

SEQ ID NO: 248 - DNA, 21 bp, Artificial Sequence
  Description of Artificial Sequence Synthetic oligonucleotide
ccagttcctgacggctgagga

SEQ ID NO: 249 - DNA, 21 bp, Artificial Sequence
  Description of Artificial Sequence Synthetic oligonucleotide
aggagtatgcctcttggaaga

SEQ ID NO: 250 - DNA, 21 bp, Artificial Sequence
  Description of Artificial Sequence Synthetic oligonucleotide
ttccaagaggcatactcctca

SEQ ID NO: 251 - DNA, 21 bp, Artificial Sequence
  Description of Artificial Sequence Synthetic oligonucleotide
caactgtgtaaattttgtgat

SEQ ID NO: 252 - DNA, 21 bp, Artificial Sequence
  Description of Artificial Sequence Synthetic oligonucleotide
tgaaggaaaaaaaaaagcctg

SEQ ID NO: 253 - DNA, 21 bp, Artificial Sequence
  Description of Artificial Sequence Synthetic oligonucleotide
cgtcttccgctcctcagccgt

SEQ ID NO: 254 - DNA, 21 bp, Artificial Sequence
  Description of Artificial Sequence Synthetic oligonucleotide
ccaggtcatccacagacagag

SEQ ID NO: 255 - DNA, 21 bp, Artificial Sequence
  Description of Artificial Sequence Synthetic oligonucleotide
gcctagagtgaagagattcat

SEQ ID NO: 256 - DNA, 21 bp, Artificial Sequence
  Description of Artificial Sequence Synthetic oligonucleotide
gttctccaaagtgcattatga

SEQ ID NO: 257 - DNA, 21 bp, Artificial Sequence
  Description of Artificial Sequence Synthetic oligonucleotide
gcatcttccagaataaagtca

SEQ ID NO: 258 - DNA, 20 bp, Artificial Sequence
  Description of Artificial Sequence Synthetic oligonucleotide
caaccgtctggtggccgacg

SEQ ID NO: 259 - DNA, 20 bp, Artificial Sequence
  Description of Artificial Sequence Synthetic oligonucleotide
caggatcggggctgtcgctt

SEQ ID NO: 260 - DNA, 20 bp, Artificial Sequence
  Description of Artificial Sequence Synthetic oligonucleotide
tcgggcctcgctggccgtaa

SEQ ID NO: 261 - DNA, 20 bp, Artificial Sequence
  Description of Artificial Sequence Synthetic oligonucleotide
gaggtagtcggccatgcgcc

SEQ ID NO: 262 - DNA, 20 bp, Artificial Sequence
  Description of Artificial Sequence Synthetic oligonucleotide
caggtgttgtcgggcctcgc

SEQ ID NO: 263 - DNA, 20 bp, Artificial Sequence
  Description of Artificial Sequence Synthetic oligonucleotide
ggaggtagtcggccatgcgc

SEQ ID NO: 264 - DNA, 20 bp, Artificial Sequence
  Description of Artificial Sequence Synthetic oligonucleotide
ggcatactcaatgcgtacat

SEQ ID NO: 265 - DNA, 20 bp, Artificial Sequence
  Description of Artificial Sequence Synthetic oligonucleotide
ccgccttgtcatcaaccgtc

SEQ ID NO: 266 - DNA, 20 bp, Artificial Sequence
  Description of Artificial Sequence Synthetic oligonucleotide
aggatcggggctgtcgcttc

SEQ ID NO: 267 - DNA, 20 bp, Artificial Sequence
  Description of Artificial Sequence Synthetic oligonucleotide
ccttgtcatcaaccgtctgg

SEQ ID NO: 268 - DNA, 20 bp, Artificial Sequence
  Description of Artificial Sequence Synthetic oligonucleotide
tactcaatgcgtacattggt

SEQ ID NO: 269 - DNA, 20 bp, Artificial Sequence
  Description of Artificial Sequence Synthetic oligonucleotide
gggttccattacggccagcg

SEQ ID NO: 270 - DNA, 20 bp, Artificial Sequence
  Description of Artificial Sequence Synthetic oligonucleotide
ggcactgcttctgcgtacaa

SEQ ID NO: 271 - DNA, 20 bp, Artificial Sequence
  Description of Artificial Sequence Synthetic oligonucleotide
ggttgatgacaaggcggcac

SEQ ID NO: 272 - DNA, 20 bp, Artificial Sequence
  Description of Artificial Sequence Synthetic oligonucleotide
tgctggggccttcctcgagg

SEQ ID NO: 273 - DNA, 20 bp, Artificial Sequence
  Description of Artificial Sequence Synthetic oligonucleotide
ttgctggctgtggagcggac

SEQ ID NO: 274 - DNA, 20 bp, Artificial Sequence
  Description of Artificial Sequence Synthetic oligonucleotide
ttctcctaccttcgggaatc

SEQ ID NO: 275 - DNA, 20 bp, Artificial Sequence
  Description of Artificial Sequence Synthetic oligonucleotide
gactggcttgggcagttcca

SEQ ID NO: 276 - DNA, 20 bp, Artificial Sequence
  Description of Artificial Sequence Synthetic oligonucleotide
catgcagcccttgcctgctg

SEQ ID NO: 277 - DNA, 20 bp, Artificial Sequence
  Description of Artificial Sequence Synthetic oligonucleotide
agcaaaggacgaggtctaga

SEQ ID NO: 278 - DNA, 20 bp, Artificial Sequence
  Description of Artificial Sequence Synthetic oligonucleotide
gcctgctggggccttcctcg

SEQ ID NO: 279 - DNA, 20 bp, Artificial Sequence
  Description of Artificial Sequence Synthetic oligonucleotide
cagactcaccagattcccga

SEQ ID NO: 280 - DNA, 20 bp, Artificial Sequence
  Description of Artificial Sequence Synthetic oligonucleotide
acctcgtcctttgctggctg

SEQ ID NO: 281 - DNA, 20 bp, Artificial Sequence
  Description of Artificial Sequence Synthetic oligonucleotide
ctcaccagattcccgaaggt

SEQ ID NO: 282 - DNA, 20 bp, Artificial Sequence
  Description of Artificial Sequence Synthetic oligonucleotide
tacgcagaagcagtgcccgc

SEQ ID NO: 283 - DNA, 20 bp, Artificial Sequence
  Description of Artificial Sequence Synthetic oligonucleotide
aggtgtacagcagtggctgg

SEQ ID NO: 284 - DNA, 20 bp, Artificial Sequence
  Description of Artificial Sequence Synthetic oligonucleotide
ggtgtacagcagtggctggt

SEQ ID NO: 285 - DNA, 20 bp, Artificial Sequence
  Description of Artificial Sequence Synthetic oligonucleotide
cggatgtggtcagccttgtg

SEQ ID NO: 286 - DNA, 20 bp, Artificial Sequence
  Description of Artificial Sequence Synthetic oligonucleotide
cactgacagcgtgaacaggt

SEQ ID NO: 287 - DNA, 20 bp, Artificial Sequence
  Description of Artificial Sequence Synthetic oligonucleotide
actgacagcgtgaacaggta

SEQ ID NO: 288 - DNA, 20 bp, Artificial Sequence
  Description of Artificial Sequence Synthetic oligonucleotide
gctcactctctgtctgggct

SEQ ID NO: 289 - DNA, 20 bp, Artificial Sequence
  Description of Artificial Sequence Synthetic oligonucleotide
ctggctgtggagcggactgg

SEQ ID NO: 290 - DNA, 20 bp, Artificial Sequence
  Description of Artificial Sequence Synthetic oligonucleotide
gctctgactgtacggggcaa

SEQ ID NO: 291 - DNA, 20 bp, Artificial Sequence
  Description of Artificial Sequence Synthetic oligonucleotide
agctctgactgtacggggca

SEQ ID NO: 292 - DNA, 20 bp, Artificial Sequence
  Description of Artificial Sequence Synthetic oligonucleotide
acagtaccccttccagctct

SEQ ID NO: 293 - DNA, 20 bp, Artificial Sequence
  Description of Artificial Sequence Synthetic oligonucleotide
cgtcggccaccagacggttg

SEQ ID NO: 294 - DNA, 20 bp, Artificial Sequence
  Description of Artificial Sequence Synthetic oligonucleotide
ccagccactgctgtacacct

SEQ ID NO: 295 - DNA, 20 bp, Artificial Sequence
  Description of Artificial Sequence Synthetic oligonucleotide
accccggccctgcctatgcc

SEQ ID NO: 296 - DNA, 20 bp, Artificial Sequence
  Description of Artificial Sequence Synthetic oligonucleotide
ggtatcagcagtgcaggagg

SEQ ID NO: 297 - DNA, 20 bp, Artificial Sequence
  Description of Artificial Sequence Synthetic oligonucleotide
gatgtggtcagccttgtgca

SEQ ID NO: 298 - DNA, 20 bp, Artificial Sequence
  Description of Artificial Sequence Synthetic oligonucleotide
ggatgtggtcagccttgtgc

SEQ ID NO: 299 - DNA, 20 bp, Artificial Sequence
  Description of Artificial Sequence Synthetic oligonucleotide
ggccacgcatcctggccttt

SEQ ID NO: 300 - DNA, 20 bp, Artificial Sequence
  Description of Artificial Sequence Synthetic oligonucleotide
gaaaggccaggatgcgtggc

SEQ ID NO: 301 - DNA, 20 bp, Artificial Sequence
  Description of Artificial Sequence Synthetic oligonucleotide
actgcttgtccaggccacgc

SEQ ID NO: 302 - DNA, 20 bp, Artificial Sequence
  Description of Artificial Sequence Synthetic oligonucleotide
tctggactccaactgcttgt

SEQ ID NO: 303 - DNA, 20 bp, Artificial Sequence
  Description of Artificial Sequence Synthetic oligonucleotide
gtctggactccaactgcttg

SEQ ID NO: 304 - DNA, 20 bp, Artificial Sequence
  Description of Artificial Sequence Synthetic oligonucleotide
gcttccgtctggactccaac

SEQ ID NO: 305 - DNA, 20 bp, Artificial Sequence
  Description of Artificial Sequence Synthetic oligonucleotide
gacggaagctggagtcggca

SEQ ID NO: 306 - DNA, 20 bp, Artificial Sequence
  Description of Artificial Sequence Synthetic oligonucleotide
cgctgtcagtgaaaaccact

SEQ ID NO: 307 - DNA, 20 bp, Artificial Sequence
  Description of Artificial Sequence Synthetic oligonucleotide
ctgacagcgtgaacaggtag

SEQ ID NO: 308 - DNA, 20 bp, Artificial Sequence
  Description of Artificial Sequence Synthetic oligonucleotide
ttacggccagcgaggcccga

SEQ ID NO: 309 - DNA, 20 bp, Artificial Sequence
  Description of Artificial Sequence Synthetic oligonucleotide
attacggccagcgaggcccg

SEQ ID NO: 310 - DNA, 20 bp, Artificial Sequence
  Description of Artificial Sequence Synthetic oligonucleotide
ggaatctggtgagtctgagg

SEQ ID NO: 311 - DNA, 20 bp, Artificial Sequence
  Description of Artificial Sequence Synthetic oligonucleotide
ccctcagactcaccagattc

SEQ ID NO: 312 - DNA, 20 bp, Artificial Sequence
  Description of Artificial Sequence Synthetic oligonucleotide
cgaaggtaggagaaggtctt

SEQ ID NO: 313 - DNA, 20 bp, Artificial Sequence
  Description of Artificial Sequence Synthetic oligonucleotide
gaaggtaggagaaggtcttg

SEQ ID NO: 314 - DNA, 20 bp, Artificial Sequence
  Description of Artificial Sequence Synthetic oligonucleotide
gcacctttggctcactctct

SEQ ID NO: 315 - DNA, 20 bp, Artificial Sequence
  Description of Artificial Sequence Synthetic oligonucleotide
tcgaggaggtggcagagggt

SEQ ID NO: 316 - DNA, 20 bp, Artificial Sequence
  Description of Artificial Sequence Synthetic oligonucleotide
tggaactgcccaagccagtc

SEQ ID NO: 317 - DNA, 20 bp, Artificial Sequence
  Description of Artificial Sequence Synthetic oligonucleotide
agggacggggcccacagggg

SEQ ID NO: 318 - DNA, 20 bp, Artificial Sequence
  Description of Artificial Sequence Synthetic oligonucleotide
gggacggggcccacaggggc

SEQ ID NO: 319 - DNA, 20 bp, Artificial Sequence
  Description of Artificial Sequence Synthetic oligonucleotide
ctccacagccagcaaaggac

SEQ ID NO: 320 - DNA, 20 bp, Artificial Sequence
  Description of Artificial Sequence Synthetic oligonucleotide
cagccagcaaaggacgaggt

SEQ ID NO: 321 - DNA, 20 bp, Artificial Sequence
  Description of Artificial Sequence Synthetic oligonucleotide
ctgccttctagacctcgtcc

SEQ ID NO: 322 - DNA, 20 bp, Artificial Sequence
  Description of Artificial Sequence Synthetic oligonucleotide
cctaaggaggatgcgcctag

SEQ ID NO: 323 - DNA, 20 bp, Artificial Sequence
  Description of Artificial Sequence Synthetic oligonucleotide
tggcctcctgcactgctgat

SEQ ID NO: 324 - DNA, 20 bp, Artificial Sequence
  Description of Artificial Sequence Synthetic oligonucleotide
agcagtgcaggaggccacat

SEQ ID NO: 325 - DNA, 20 bp, Artificial Sequence
  Description of Artificial Sequence Synthetic oligonucleotide
ccgactccagcttccgtctg

SEQ ID NO: 326 - DNA, 20 bp, Artificial Sequence
  Description of Artificial Sequence Synthetic oligonucleotide
ggggttccattacggccagc

SEQ ID NO: 327 - DNA, 20 bp, Artificial Sequence
  Description of Artificial Sequence Synthetic oligonucleotide
cacagcagatcctcctctgg

SEQ ID NO: 328 - DNA, 20 bp, Artificial Sequence
  Description of Artificial Sequence Synthetic oligonucleotide
attgccccgtacagtcagag

SEQ ID NO: 329 - DNA, 20 bp, Artificial Sequence
  Description of Artificial Sequence Synthetic oligonucleotide
cccgtacagtcagagctgga

SEQ ID NO: 330 - DNA, 20 bp, Artificial Sequence
  Description of Artificial Sequence Synthetic oligonucleotide
tggtggaggagcaggcagtg

SEQ ID NO: 331 - DNA, 20 bp, Artificial Sequence
  Description of Artificial Sequence Synthetic oligonucleotide
tccttaggcataggcagggc

SEQ ID NO: 332 - DNA, 20 bp, Artificial Sequence
  Description of Artificial Sequence Synthetic oligonucleotide
cggccctgcctatgcctaag

SEQ ID NO: 333 - DNA, 20 bp, Artificial Sequence
  Description of Artificial Sequence Synthetic oligonucleotide
taggcataggcagggccggg

SEQ ID NO: 334 - DNA, 20 bp, Artificial Sequence
  Description of Artificial Sequence Synthetic oligonucleotide
aggcagggccggggtgggag

SEQ ID NO: 335 - DNA, 20 bp, Artificial Sequence
  Description of Artificial Sequence Synthetic oligonucleotide
gcaggatcggggctgtcgct

SEQ ID NO: 336 - DNA, 20 bp, Artificial Sequence
  Description of Artificial Sequence Synthetic oligonucleotide
ctgcacaaggctgaccacat

SEQ ID NO: 337 - DNA, 20 bp, Artificial Sequence
  Description of Artificial Sequence Synthetic oligonucleotide
tgcacaaggctgaccacatc

SEQ ID NO: 338 - DNA, 20 bp, Artificial Sequence
  Description of Artificial Sequence Synthetic oligonucleotide
ctgaccacatccggaaaggc

SEQ ID NO: 339 - DNA, 20 bp, Artificial Sequence
  Description of Artificial Sequence Synthetic oligonucleotide
ggccacgcatcctggccttt

SEQ ID NO: 340 - DNA, 20 bp, Artificial Sequence
  Description of Artificial Sequence Synthetic oligonucleotide
gcgtggcctggacaagcagt

SEQ ID NO: 341 - DNA, 20 bp, Artificial Sequence
  Description of Artificial Sequence Synthetic oligonucleotide
gacaagcagttggagtccag

SEQ ID NO: 342 - DNA, 20 bp, Artificial Sequence
  Description of Artificial Sequence Synthetic oligonucleotide
gttggagtccagacggaagc

SEQ ID NO: 343 - DNA, 20 bp, Artificial Sequence
  Description of Artificial Sequence Synthetic oligonucleotide
atgcgtacattggtggggcc

SEQ ID NO: 344 - DNA, 20 bp, Artificial Sequence
  Description of Artificial Sequence Synthetic oligonucleotide
tggccccaccaatgtacgca

SEQ ID NO: 345 - DNA, 20 bp, Artificial Sequence
  Description of Artificial Sequence Synthetic oligonucleotide
gctacctgttcacgctgtca

SEQ ID NO: 346 - DNA, 20 bp, Artificial Sequence
  Description of Artificial Sequence Synthetic oligonucleotide
tgacagcgtgaacaggtagc

SEQ ID NO: 347 - DNA, 20 bp, Artificial Sequence
  Description of Artificial Sequence Synthetic oligonucleotide
gtcgggcctcgctggccgta

SEQ ID NO: 348 - DNA, 20 bp, Artificial Sequence
  Description of Artificial Sequence Synthetic oligonucleotide
gcacttgcctaggctggtat

SEQ ID NO: 349 - DNA, 20 bp, Artificial Sequence
  Description of Artificial Sequence Synthetic oligonucleotide
gggaatctggtgagtctgag

SEQ ID NO: 350 - DNA, 20 bp, Artificial Sequence
  Description of Artificial Sequence Synthetic oligonucleotide
ctcaccagattcccgaaggt

SEQ ID NO: 351 - DNA, 20 bp, Artificial Sequence
  Description of Artificial Sequence Synthetic oligonucleotide
ctcctaccttcgggaatctg

SEQ ID NO: 352 - DNA, 20 bp, Artificial Sequence
  Description of Artificial Sequence Synthetic oligonucleotide
caagaccttctcctaccttc

SEQ ID NO: 353 - DNA, 20 bp, Artificial Sequence
  Description of Artificial Sequence Synthetic oligonucleotide
ccaagaccttctcctacctt

SEQ ID NO: 354 - DNA, 20 bp, Artificial Sequence
  Description of Artificial Sequence Synthetic oligonucleotide
gccaagaccttctcctacct

SEQ ID NO: 355 - DNA, 20 bp, Artificial Sequence
  Description of Artificial Sequence Synthetic oligonucleotide
tatgcacagcagatcctcct

SEQ ID NO: 356 - DNA, 20 bp, Artificial Sequence
  Description of Artificial Sequence Synthetic oligonucleotide
caaaggtgctggacccagag

SEQ ID NO: 357 - DNA, 20 bp, Artificial Sequence
  Description of Artificial Sequence Synthetic oligonucleotide
ggctcactctctgtctgggc

SEQ ID NO: 358 - DNA, 20 bp, Artificial Sequence
  Description of Artificial Sequence Synthetic oligonucleotide
agggtaccccagcccagaca

SEQ ID NO: 359 - DNA, 20 bp, Artificial Sequence
  Description of Artificial Sequence Synthetic oligonucleotide
agagggtaccccagcccaga

SEQ ID NO: 360 - DNA, 20 bp, Artificial Sequence
  Description of Artificial Sequence Synthetic oligonucleotide
gtaccctctgccacctcctc

SEQ ID NO: 361 - DNA, 20 bp, Artificial Sequence
  Description of Artificial Sequence Synthetic oligonucleotide
ccttcctcgaggaggtggca

SEQ ID NO: 362 - DNA, 20 bp, Artificial Sequence
  Description of Artificial Sequence Synthetic oligonucleotide
atgactggcttgggcagttc

SEQ ID NO: 363 - DNA, 20 bp, Artificial Sequence
  Description of Artificial Sequence Synthetic oligonucleotide
ggcccctgtgggccccgtcc

SEQ ID NO: 364 - DNA, 20 bp, Artificial Sequence
  Description of Artificial Sequence Synthetic oligonucleotide
aggacgaggtctagaaggca

SEQ ID NO: 365 - DNA, 20 bp, Artificial Sequence
  Description of Artificial Sequence Synthetic oligonucleotide
tataagtggaggcgtcgcgc

SEQ ID NO: 366 - DNA, 20 bp, Artificial Sequence
  Description of Artificial Sequence Synthetic oligonucleotide
gggcacgcgtttaatataag

SEQ ID NO: 367 - DNA, 20 bp, Artificial Sequence
  Description of Artificial Sequence Synthetic oligonucleotide
actcacgctggatagcctcc

SEQ ID NO: 368 - DNA, 20 bp, Artificial Sequence
  Description of Artificial Sequence Synthetic oligonucleotide
ggccgagatgtctcgctccg

SEQ ID NO: 369 - DNA, 20 bp, Artificial Sequence
  Description of Artificial Sequence Synthetic oligonucleotide
cacgcgtttaatataagtgg

SEQ ID NO: 370 - DNA, 20 bp, Artificial Sequence
  Description of Artificial Sequence Synthetic oligonucleotide
aagtggaggcgtcgcgctgg

SEQ ID NO: 371 - DNA, 20 bp, Artificial Sequence
  Description of Artificial Sequence Synthetic oligonucleotide
gagtagcgcgagcacagcta

SEQ ID NO: 372 - DNA, 20 bp, Artificial Sequence
  Description of Artificial Sequence Synthetic oligonucleotide
agtggaggcgtcgcgctggc

SEQ ID NO: 373 - DNA, 20 bp, Artificial Sequence
  Description of Artificial Sequence Synthetic oligonucleotide
gcccgaatgctgtcagcttc

SEQ ID NO: 374 - DNA, 20 bp, Artificial Sequence
  Description of Artificial Sequence Synthetic oligonucleotide
cgcgagcacagctaaggcca

SEQ ID NO: 375 - DNA, 20 bp, Artificial Sequence
  Description of Artificial Sequence Synthetic oligonucleotide
ctcgcgctactctctctttc

SEQ ID NO: 376 - DNA, 20 bp, Artificial Sequence
  Description of Artificial Sequence Synthetic oligonucleotide
ggccacggagcgagacatct

SEQ ID NO: 377 - DNA, 20 bp, Artificial Sequence
  Description of Artificial Sequence Synthetic oligonucleotide
cgtgagtaaacctgaatctt

SEQ ID NO: 378 - DNA, 20 bp, Artificial Sequence
  Description of Artificial Sequence Synthetic oligonucleotide
agtcacatggttcacacggc

SEQ ID NO: 379 - DNA, 20 bp, Artificial Sequence
  Description of Artificial Sequence Synthetic oligonucleotide
aagtcaacttcaatgtcgga

SEQ ID NO: 380 - DNA, 20 bp, Artificial Sequence
  Description of Artificial Sequence Synthetic oligonucleotide
cagtaagtcaacttcaatgt

SEQ ID NO: 381 - DNA, 20 bp, Artificial Sequence
  Description of Artificial Sequence Synthetic oligonucleotide
acccagacacatagcaattc

SEQ ID NO: 382 - DNA, 20 bp, Artificial Sequence
  Description of Artificial Sequence Synthetic oligonucleotide
gcatactcatctttttcagt

SEQ ID NO: 383 - DNA, 20 bp, Artificial Sequence
  Description of Artificial Sequence Synthetic oligonucleotide
acagcccaagatagttaagt

SEQ ID NO: 384 - DNA, 20 bp, Artificial Sequence
  Description of Artificial Sequence Synthetic oligonucleotide
ggcatactcatctttttcag

SEQ ID NO: 385 - DNA, 20 bp, Artificial Sequence
  Description of Artificial Sequence Synthetic oligonucleotide
ttcctgaagctgacagcatt

SEQ ID NO: 386 - DNA, 20 bp, Artificial Sequence
  Description of Artificial Sequence Synthetic oligonucleotide
tcacgtcatccagcagagaa

SEQ ID NO: 387 - DNA, 20 bp, Artificial Sequence
  Description of Artificial Sequence Synthetic oligonucleotide
cagcccaagatagttaagtg

SEQ ID NO: 388 - RNA, 18 nt, Artificial Sequence
  Description of Artificial Sequence Synthetic oligonucleotide
aauucucucuccauucuu

SEQ ID NO: 389 - RNA, 19 nt, Artificial Sequence
  Description of Artificial Sequence Synthetic oligonucleotide
aauucucucuccauucuuc

SEQ ID NO: 390 - RNA, 20 nt, Artificial Sequence
  Description of Artificial Sequence Synthetic oligonucleotide
aauucucucuccauucuuca

SEQ ID NO: 391 - RNA, 21 nt, Artificial Sequence
  Description of Artificial Sequence Synthetic oligonucleotide
aauucucucuccauucuucag

SEQ ID NO: 392 - RNA, 22 nt, Artificial Sequence
  Description of Artificial Sequence Synthetic oligonucleotide
aauucucucuccauucuucagu

SEQ ID NO: 393 - RNA, 23 nt, Artificial Sequence
  Description of Artificial Sequence Synthetic oligonucleotide
aauucucucuccauucuucagua

SEQ ID NO: 394 - RNA, 24 nt, Artificial Sequence
  Description of Artificial Sequence Synthetic oligonucleotide
aauucucucuccauucuucaguaa

SEQ ID NO: 395 - RNA, 18 nt, Artificial Sequence
  Description of Artificial Sequence Synthetic oligonucleotide
acuuuccauucucugcug

SEQ ID NO: 396 - RNA, 19 nt, Artificial Sequence
  Description of Artificial Sequence Synthetic oligonucleotide
acuuuccauucucugcugg

SEQ ID NO: 397 - RNA, 20 nt, Artificial Sequence
  Description of Artificial Sequence Synthetic oligonucleotide
acuuuccauucucugcugga

SEQ ID NO: 398 - RNA, 21 nt, Artificial Sequence
  Description of Artificial Sequence Synthetic oligonucleotide
acuuuccauucucugcuggau

SEQ ID NO: 399 - RNA, 22 nt, Artificial Sequence
  Description of Artificial Sequence Synthetic oligonucleotide
acuuuccauucucugcuggaug

SEQ ID NO: 400 - RNA, 23 nt, Artificial Sequence
  Description of Artificial Sequence Synthetic oligonucleotide
acuuuccauucucugcuggauga

SEQ ID NO: 401 - RNA, 24 nt, Artificial Sequence
  Description of Artificial Sequence Synthetic oligonucleotide
acuuuccauucucugcuggaugac

SEQ ID NO: 402 - RNA, 18 nt, Artificial Sequence
  Description of Artificial Sequence Synthetic oligonucleotide
agcaaggacuggucuuuc

SEQ ID NO: 403 - RNA, 19 nt, Artificial Sequence
  Description of Artificial Sequence Synthetic oligonucleotide
agcaaggacuggucuuucu

SEQ ID NO: 404 - RNA, 20 nt, Artificial Sequence
  Description of Artificial Sequence Synthetic oligonucleotide
agcaaggacuggucuuucua

SEQ ID NO: 405 - RNA, 21 nt, Artificial Sequence
  Description of Artificial Sequence Synthetic oligonucleotide
agcaaggacuggucuuucuau

SEQ ID NO: 406 - RNA, 22 nt, Artificial Sequence
  Description of Artificial Sequence Synthetic oligonucleotide
agcaaggacuggucuuucuauc

SEQ ID NO: 407 - RNA, 23 nt, Artificial Sequence
  Description of Artificial Sequence Synthetic oligonucleotide
agcaaggacuggucuuucuaucu

SEQ ID NO: 408 - RNA, 24 nt, Artificial Sequence
  Description of Artificial Sequence Synthetic oligonucleotide
agcaaggacuggucuuucuaucuc

SEQ ID NO: 409 - RNA, 18 nt, Artificial Sequence
  Description of Artificial Sequence Synthetic oligonucleotide
agugggggugaauucagu

SEQ ID NO: 410 - RNA, 19 nt, Artificial Sequence
  Description of Artificial Sequence Synthetic oligonucleotide
agugggggugaauucagug

SEQ ID NO: 411 - RNA, 20 nt, Artificial Sequence
  Description of Artificial Sequence Synthetic oligonucleotide
agugggggugaauucagugu

SEQ ID NO: 412 - RNA, 21 nt, Artificial Sequence
  Description of Artificial Sequence Synthetic oligonucleotide
agugggggugaauucagugua

SEQ ID NO: 413 - RNA, 22 nt, Artificial Sequence
  Description of Artificial Sequence Synthetic oligonucleotide
agugggggugaauucaguguag

SEQ ID NO: 414 - RNA, 23 nt, Artificial Sequence
  Description of Artificial Sequence Synthetic oligonucleotide
agugggggugaauucaguguagu

SEQ ID NO: 415 - RNA, 24 nt, Artificial Sequence
  Description of Artificial Sequence Synthetic oligonucleotide
agugggggugaauucaguguagua

SEQ ID NO: 416 - RNA, 18 nt, Artificial Sequence
  Description of Artificial Sequence Synthetic oligonucleotide
auccauccgacauugaag

SEQ ID NO: 417 - RNA, 19 nt, Artificial Sequence
  Description of Artificial Sequence Synthetic oligonucleotide
auccauccgacauugaagu

SEQ ID NO: 418 - RNA, 20 nt, Artificial Sequence
  Description of Artificial Sequence Synthetic oligonucleotide
auccauccgacauugaaguu

SEQ ID NO: 419 - RNA, 21 nt, Artificial Sequence
  Description of Artificial Sequence Synthetic oligonucleotide
auccauccgacauugaaguug

SEQ ID NO: 420 - RNA, 22 nt, Artificial Sequence
  Description of Artificial Sequence Synthetic oligonucleotide
auccauccgacauugaaguuga

SEQ ID NO: 421 - RNA, 23 nt, Artificial Sequence
  Description of Artificial Sequence Synthetic oligonucleotide
auccauccgacauugaaguugac

SEQ ID NO: 422 - RNA, 24 nt, Artificial Sequence
  Description of Artificial Sequence Synthetic oligonucleotide
auccauccgacauugaaguugacu

SEQ ID NO: 423 - RNA, 18 nt, Artificial Sequence
  Description of Artificial Sequence Synthetic oligonucleotide
caauucucucuccauucu

SEQ ID NO: 424 - RNA, 19 nt, Artificial Sequence
  Description of Artificial Sequence Synthetic oligonucleotide
caauucucucuccauucuu

SEQ ID NO: 425 - RNA, 20 nt, Artificial Sequence
  Description of Artificial Sequence Synthetic oligonucleotide
caauucucucuccauucuuc

SEQ ID NO: 426 - RNA, 21 nt, Artificial Sequence
  Description of Artificial Sequence Synthetic oligonucleotide
caauucucucuccauucuuca

SEQ ID NO: 427 - RNA, 22 nt, Artificial Sequence
  Description of Artificial Sequence Synthetic oligonucleotide
caauucucucuccauucuucag

SEQ ID NO: 428 - RNA, 23 nt, Artificial Sequence
  Description of Artificial Sequence Synthetic oligonucleotide
caauucucucuccauucuucagu

SEQ ID NO: 429 - RNA, 24 nt, Artificial Sequence
  Description of Artificial Sequence Synthetic oligonucleotide
caauucucucuccauucuucagua

SEQ ID NO: 430 - RNA, 18 nt, Artificial Sequence
  Description of Artificial Sequence Synthetic oligonucleotide
cagugggggugaauucag

SEQ ID NO: 431 - RNA, 19 nt, Artificial Sequence
  Description of Artificial Sequence Synthetic oligonucleotide
cagugggggugaauucagu

SEQ ID NO: 432 - RNA, 20 nt, Artificial Sequence
  Description of Artificial Sequence Synthetic oligonucleotide
cagugggggugaauucagug

SEQ ID NO: 433 - RNA, 21 nt, Artificial Sequence
  Description of Artificial Sequence Synthetic oligonucleotide
cagugggggugaauucagugu

SEQ ID NO: 434 - RNA, 22 nt, Artificial Sequence
  Description of Artificial Sequence Synthetic oligonucleotide
cagugggggugaauucagugua

SEQ ID NO: 435 - RNA, 23 nt, Artificial Sequence
  Description of Artificial Sequence Synthetic oligonucleotide
cagugggggugaauucaguguag

SEQ ID NO: 436 - RNA, 24 nt, Artificial Sequence
  Description of Artificial Sequence Synthetic oligonucleotide
cagugggggugaauucaguguagu

SEQ ID NO: 437 - RNA, 18 nt, Artificial Sequence
  Description of Artificial Sequence Synthetic oligonucleotide
cauucucugcuggaugac

SEQ ID NO: 438 - RNA, 19 nt, Artificial Sequence
  Description of Artificial Sequence Synthetic oligonucleotide
cauucucugcuggaugacg

SEQ ID NO: 439 - RNA, 20 nt, Artificial Sequence
  Description of Artificial Sequence Synthetic oligonucleotide
cauucucugcuggaugacgu

SEQ ID NO: 440 - RNA, 21 nt, Artificial Sequence
  Description of Artificial Sequence Synthetic oligonucleotide
cauucucugcuggaugacgug

SEQ ID NO: 441 - RNA, 22 nt, Artificial Sequence
  Description of Artificial Sequence Synthetic oligonucleotide
cauucucugcuggaugacguga

SEQ ID NO: 442 - RNA, 23 nt, Artificial Sequence
  Description of Artificial Sequence Synthetic oligonucleotide
cauucucugcuggaugacgugag

SEQ ID NO: 443 - RNA, 24 nt, Artificial Sequence
  Description of Artificial Sequence Synthetic oligonucleotide
cauucucugcuggaugacgugagu

SEQ ID NO: 444 - RNA, 18 nt, Artificial Sequence
  Description of Artificial Sequence Synthetic oligonucleotide
cccgauauuccucaggua

SEQ ID NO: 445 - RNA, 19 nt, Artificial Sequence
  Description of Artificial Sequence Synthetic oligonucleotide
cccgauauuccucagguac

SEQ ID NO: 446 - RNA, 20 nt, Artificial Sequence
  Description of Artificial Sequence Synthetic oligonucleotide
cccgauauuccucagguacu

SEQ ID NO: 447 - RNA, 21 nt, Artificial Sequence
  Description of Artificial Sequence Synthetic oligonucleotide
cccgauauuccucagguacuc

SEQ ID NO: 448 - RNA, 22 nt, Artificial Sequence
  Description of Artificial Sequence Synthetic oligonucleotide
cccgauauuccucagguacucc

SEQ ID NO: 449 - RNA, 23 nt, Artificial Sequence
  Description of Artificial Sequence Synthetic oligonucleotide
cccgauauuccucagguacucca

SEQ ID NO: 450 - RNA, 24 nt, Artificial Sequence
  Description of Artificial Sequence Synthetic oligonucleotide
cccgauauuccucagguacuccaa

SEQ ID NO: 451 - RNA, 18 nt, Artificial Sequence
  Description of Artificial Sequence Synthetic oligonucleotide
ccgauauuccucagguac

SEQ ID NO: 452 - RNA, 19 nt, Artificial Sequence
  Description of Artificial Sequence Synthetic oligonucleotide
ccgauauuccucagguacu

SEQ ID NO: 453 - RNA, 20 nt, Artificial Sequence
  Description of Artificial Sequence Synthetic oligonucleotide
ccgauauuccucagguacuc

SEQ ID NO: 454 - RNA, 21 nt, Artificial Sequence
  Description of Artificial Sequence Synthetic oligonucleotide
ccgauauuccucagguacucc

SEQ ID NO: 455 - RNA, 22 nt, Artificial Sequence
  Description of Artificial Sequence Synthetic oligonucleotide
ccgauauuccucagguacucca

SEQ ID NO: 456 - RNA, 23 nt, Artificial Sequence
  Description of Artificial Sequence Synthetic oligonucleotide
ccgauauuccucagguacuccaa

SEQ ID NO: 457 - RNA, 24 nt, Artificial Sequence
  Description of Artificial Sequence Synthetic oligonucleotide
ccgauauuccucagguacuccaaa

SEQ ID NO: 458 - RNA, 18 nt, Artificial Sequence
  Description of Artificial Sequence Synthetic oligonucleotide
cucacgucauccagcaga

SEQ ID NO: 459 - RNA, 19 nt, Artificial Sequence
  Description of Artificial Sequence Synthetic oligonucleotide
cucacgucauccagcagag

SEQ ID NO: 460 - RNA, 20 nt, Artificial Sequence
  Description of Artificial Sequence Synthetic oligonucleotide
cucacgucauccagcagaga

SEQ ID NO: 461 - RNA, 21 nt, Artificial Sequence
  Description of Artificial Sequence Synthetic oligonucleotide
cucacgucauccagcagagaa

SEQ ID NO: 462 - RNA, 22 nt, Artificial Sequence
  Description of Artificial Sequence Synthetic oligonucleotide
cucacgucauccagcagagaau

SEQ ID NO: 463 - RNA, 23 nt, Artificial Sequence
  Description of Artificial Sequence Synthetic oligonucleotide
cucacgucauccagcagagaaug

SEQ ID NO: 464 - RNA, 24 nt, Artificial Sequence
  Description of Artificial Sequence Synthetic oligonucleotide
cucacgucauccagcagagaaugg

SEQ ID NO: 465 - RNA, 18 nt, Artificial Sequence
  Description of Artificial Sequence Synthetic oligonucleotide
cugaauugcuaugugucu

SEQ ID NO: 466 - RNA, 19 nt, Artificial Sequence
  Description of Artificial Sequence Synthetic oligonucleotide
cugaauugcuaugugucug

SEQ ID NO: 467 - RNA, 20 nt, Artificial Sequence
  Description of Artificial Sequence Synthetic oligonucleotide
cugaauugcuaugugucugg

SEQ ID NO: 468 - RNA, 21 nt, Artificial Sequence
  Description of Artificial Sequence Synthetic oligonucleotide
cugaauugcuaugugucuggg

SEQ ID NO: 469 - RNA, 22 nt, Artificial Sequence
  Description of Artificial Sequence Synthetic oligonucleotide
cugaauugcuaugugucugggu

SEQ ID NO: 470 - RNA, 23 nt, Artificial Sequence
  Description of Artificial Sequence Synthetic oligonucleotide
cugaauugcuaugugucuggguu

SEQ ID NO: 471 - RNA, 24 nt, Artificial Sequence
  Description of Artificial Sequence Synthetic oligonucleotide
cugaauugcuaugugucuggguuu

SEQ ID NO: 472 - RNA, 18 nt, Artificial Sequence
  Description of Artificial Sequence Synthetic oligonucleotide
gaguaccugaggaauauc

SEQ ID NO: 473 - RNA, 19 nt, Artificial Sequence
  Description of Artificial Sequence Synthetic oligonucleotide
gaguaccugaggaauaucg

SEQ ID NO: 474 - RNA, 20 nt, Artificial Sequence
  Description of Artificial Sequence Synthetic oligonucleotide
gaguaccugaggaauaucgg

SEQ ID NO: 475 - RNA, 21 nt, Artificial Sequence
  Description of Artificial Sequence Synthetic oligonucleotide
gaguaccugaggaauaucggg

SEQ ID NO: 476 - RNA, 22 nt, Artificial Sequence
  Description of Artificial Sequence Synthetic oligonucleotide
gaguaccugaggaauaucggga

SEQ ID NO: 477 - RNA, 23 nt, Artificial Sequence
  Description of Artificial Sequence Synthetic oligonucleotide
gaguaccugaggaauaucgggaa

SEQ ID NO: 478 - RNA, 24 nt, Artificial Sequence
  Description of Artificial Sequence Synthetic oligonucleotide
gaguaccugaggaauaucgggaaa

SEQ ID NO: 479 - RNA, 18 nt, Artificial Sequence
  Description of Artificial Sequence Synthetic oligonucleotide
uaucucuuguacuacacu

SEQ ID NO: 480 - RNA, 19 nt, Artificial Sequence
  Description of Artificial Sequence Synthetic oligonucleotide
uaucucuuguacuacacug

SEQ ID NO: 481 - RNA, 20 nt, Artificial Sequence
  Description of Artificial Sequence Synthetic oligonucleotide
uaucucuuguacuacacuga

SEQ ID NO: 482 - RNA, 21 nt, Artificial Sequence
  Description of Artificial Sequence Synthetic oligonucleotide
uaucucuuguacuacacugaa

SEQ ID NO: 483 - RNA, 22 nt, Artificial Sequence
  Description of Artificial Sequence Synthetic oligonucleotide
uaucucuuguacuacacugaau

SEQ ID NO: 484 - RNA, 23 nt, Artificial Sequence
  Description of Artificial Sequence Synthetic oligonucleotide
uaucucuuguacuacacugaauu

SEQ ID NO: 485 - RNA, 24 nt, Artificial Sequence
  Description of Artificial Sequence Synthetic oligonucleotide
uaucucuuguacuacacugaauuc

SEQ ID NO: 486 - RNA, 18 nt, Artificial Sequence
  Description of Artificial Sequence Synthetic oligonucleotide
ucaauucucucuccauuc

SEQ ID NO: 487 - RNA, 19 nt, Artificial Sequence
  Description of Artificial Sequence Synthetic oligonucleotide
ucaauucucucuccauucu

SEQ ID NO: 488 - RNA, 20 nt, Artificial Sequence
  Description of Artificial Sequence Synthetic oligonucleotide
ucaauucucucuccauucuu

SEQ ID NO: 489 - RNA, 21 nt, Artificial Sequence
  Description of Artificial Sequence Synthetic oligonucleotide
ucaauucucucuccauucuuc

SEQ ID NO: 490 - RNA, 22 nt, Artificial Sequence
  Description of Artificial Sequence Synthetic oligonucleotide
ucaauucucucuccauucuuca

SEQ ID NO: 491 - RNA, 23 nt, Artificial Sequence
  Description of Artificial Sequence Synthetic oligonucleotide
ucaauucucucuccauucuucag

SEQ ID NO: 492 - RNA, 24 nt, Artificial Sequence
  Description of Artificial Sequence Synthetic oligonucleotide
ucaauucucucuccauucuucagu

SEQ ID NO: 493 - RNA, 18 nt, Artificial Sequence
  Description of Artificial Sequence Synthetic oligonucleotide
ucacagcccaagauaguu

SEQ ID NO: 494 - RNA, 19 nt, Artificial Sequence
  Description of Artificial Sequence Synthetic oligonucleotide
ucacagcccaagauaguua

SEQ ID NO: 495 - RNA, 20 nt, Artificial Sequence
  Description of Artificial Sequence Synthetic oligonucleotide
ucacagcccaagauaguuaa

SEQ ID NO: 496 - RNA, 21 nt, Artificial Sequence
  Description of Artificial Sequence Synthetic oligonucleotide
ucacagcccaagauaguuaag

SEQ ID NO: 497 - RNA, 22 nt, Artificial Sequence
  Description of Artificial Sequence Synthetic oligonucleotide
ucacagcccaagauaguuaagu

SEQ ID NO: 498 - RNA, 23 nt, Artificial Sequence
  Description of Artificial Sequence Synthetic oligonucleotide
ucacagcccaagauaguuaagug

SEQ ID NO: 499 - RNA, 24 nt, Artificial Sequence
  Description of Artificial Sequence Synthetic oligonucleotide
ucacagcccaagauaguuaagugg

SEQ ID NO: 500 - RNA, 18 nt, Artificial Sequence
  Description of Artificial Sequence Synthetic oligonucleotide
ucagugggggugaauuca

SEQ ID NO: 501 - RNA, 19 nt, Artificial Sequence
  Description of Artificial Sequence Synthetic oligonucleotide
ucagugggggugaauucag

SEQ ID NO: 502 - RNA, 20 nt, Artificial Sequence
  Description of Artificial Sequence Synthetic oligonucleotide
ucagugggggugaauucagu

SEQ ID NO: 503 - RNA, 21 nt, Artificial Sequence
  Description of Artificial Sequence Synthetic oligonucleotide
ucagugggggugaauucagug

SEQ ID NO: 504 - RNA, 22 nt, Artificial Sequence
  Description of Artificial Sequence Synthetic oligonucleotide
ucagugggggugaauucagugu

SEQ ID NO: 505 - RNA, 23 nt, Artificial Sequence
  Description of Artificial Sequence Synthetic oligonucleotide
ucagugggggugaauucagugua

SEQ ID NO: 506 - RNA, 24 nt, Artificial Sequence
  Description of Artificial Sequence Synthetic oligonucleotide
ucagugggggugaauucaguguag

SEQ ID NO: 507 - RNA, 18 nt, Artificial Sequence
  Description of Artificial Sequence Synthetic oligonucleotide
uggccuggaggcuaucca

SEQ ID NO: 508 - RNA, 19 nt, Artificial Sequence
  Description of Artificial Sequence Synthetic oligonucleotide
uggccuggaggcuauccag

SEQ ID NO: 509 - RNA, 20 nt, Artificial Sequence
  Description of Artificial Sequence Synthetic oligonucleotide
uggccuggaggcuauccagc

SEQ ID NO: 510 - RNA, 21 nt, Artificial Sequence
  Description of Artificial Sequence Synthetic oligonucleotide
uggccuggaggcuauccagcg

SEQ ID NO: 511 - RNA, 22 nt, Artificial Sequence
  Description of Artificial Sequence Synthetic oligonucleotide
uggccuggaggcuauccagcgu

SEQ ID NO: 512 - RNA, 23 nt, Artificial Sequence
  Description of Artificial Sequence Synthetic oligonucleotide
uggccuggaggcuauccagcgug

SEQ ID NO: 513 - RNA, 24 nt, Artificial Sequence
  Description of Artificial Sequence Synthetic oligonucleotide
uggccuggaggcuauccagcguga

SEQ ID NO: 514 - RNA, 18 nt, Artificial Sequence
  Description of Artificial Sequence Synthetic oligonucleotide
auagaucgagacauguaa

SEQ ID NO: 515 - RNA, 19 nt, Artificial Sequence
  Description of Artificial Sequence Synthetic oligonucleotide
auagaucgagacauguaag

SEQ ID NO: 516 - RNA, 20 nt, Artificial Sequence
  Description of Artificial Sequence Synthetic oligonucleotide
auagaucgagacauguaagc

SEQ ID NO: 517 - RNA, 21 nt, Artificial Sequence
  Description of Artificial Sequence Synthetic oligonucleotide
auagaucgagacauguaagca

SEQ ID NO: 518 - RNA, 22 nt, Artificial Sequence
  Description of Artificial Sequence Synthetic oligonucleotide
auagaucgagacauguaagcag

SEQ ID NO: 519 - RNA, 23 nt, Artificial Sequence
  Description of Artificial Sequence Synthetic oligonucleotide
auagaucgagacauguaagcagc

SEQ ID NO: 520 - RNA, 24 nt, Artificial Sequence
  Description of Artificial Sequence Synthetic oligonucleotide
auagaucgagacauguaagcagca

SEQ ID NO: 521 - RNA, 18 nt, Artificial Sequence
  Description of Artificial Sequence Synthetic oligonucleotide
cauagaucgagacaugua

SEQ ID NO: 522 - RNA, 19 nt, Artificial Sequence
  Description of Artificial Sequence Synthetic oligonucleotide
cauagaucgagacauguaa

SEQ ID NO: 523 - RNA, 20 nt, Artificial Sequence
  Description of Artificial Sequence Synthetic oligonucleotide
cauagaucgagacauguaag

SEQ ID NO: 524 - RNA, 21 nt, Artificial Sequence
  Description of Artificial Sequence Synthetic oligonucleotide
cauagaucgagacauguaagc

SEQ ID NO: 525 - RNA, 22 nt, Artificial Sequence
  Description of Artificial Sequence Synthetic oligonucleotide
cauagaucgagacauguaagca

SEQ ID NO: 526 - RNA, 23 nt, Artificial Sequence
  Description of Artificial Sequence Synthetic oligonucleotide
cauagaucgagacauguaagcag

SEQ ID NO: 527 - RNA, 24 nt, Artificial Sequence
  Description of Artificial Sequence Synthetic oligonucleotide
cauagaucgagacauguaagcagc

SEQ ID NO: 528 - RNA, 18 nt, Artificial Sequence
  Description of Artificial Sequence Synthetic oligonucleotide
cuccacugucuuuuucau

SEQ ID NO: 529 - RNA, 19 nt, Artificial Sequence
  Description of Artificial Sequence Synthetic oligonucleotide
cuccacugucuuuuucaua

SEQ ID NO: 530 - RNA, 20 nt, Artificial Sequence
  Description of Artificial Sequence Synthetic oligonucleotide
cuccacugucuuuuucauag

SEQ ID NO: 531 - RNA, 21 nt, Artificial Sequence
  Description of Artificial Sequence Synthetic oligonucleotide
cuccacugucuuuuucauaga

SEQ ID NO: 532 - RNA, 22 nt, Artificial Sequence
  Description of Artificial Sequence Synthetic oligonucleotide
cuccacugucuuuuucauagau

SEQ ID NO: 533 - RNA, 23 nt, Artificial Sequence
  Description of Artificial Sequence Synthetic oligonucleotide
cuccacugucuuuuucauagauc

SEQ ID NO: 534 - RNA, 24 nt, Artificial Sequence
  Description of Artificial Sequence Synthetic oligonucleotide
cuccacugucuuuuucauagaucg

SEQ ID NO: 535 - RNA, 18 nt, Artificial Sequence
  Description of Artificial Sequence Synthetic oligonucleotide
ucauagaucgagacaugu

SEQ ID NO: 536 - RNA, 19 nt, Artificial Sequence
  Description of Artificial Sequence Synthetic oligonucleotide
ucauagaucgagacaugua

SEQ ID NO: 537 - RNA, 20 nt, Artificial Sequence
  Description of Artificial Sequence Synthetic oligonucleotide
ucauagaucgagacauguaa

SEQ ID NO: 538 - RNA, 21 nt, Artificial Sequence
  Description of Artificial Sequence Synthetic oligonucleotide
ucauagaucgagacauguaag

SEQ ID NO: 539 - RNA, 22 nt, Artificial Sequence
  Description of Artificial Sequence Synthetic oligonucleotide
ucauagaucgagacauguaagc

SEQ ID NO: 540 - RNA, 23 nt, Artificial Sequence
  Description of Artificial Sequence Synthetic oligonucleotide
ucauagaucgagacauguaagca

SEQ ID NO: 541 - RNA, 24 nt, Artificial Sequence
  Description of Artificial Sequence Synthetic oligonucleotide
ucauagaucgagacauguaagcag

SEQ ID NO: 542 - RNA, 18 nt, Artificial Sequence
  Description of Artificial Sequence Synthetic oligonucleotide
uccacugucuuuuucaua

SEQ ID NO: 543 - RNA, 19 nt, Artificial Sequence
  Description of Artificial Sequence Synthetic oligonucleotide
uccacugucuuuuucauag

SEQ ID NO: 544 - RNA, 20 nt, Artificial Sequence
  Description of Artificial Sequence Synthetic oligonucleotide
uccacugucuuuuucauaga

SEQ ID NO: 545 - RNA, 21 nt, Artificial Sequence
  Description of Artificial Sequence Synthetic oligonucleotide
uccacugucuuuuucauagau

SEQ ID NO: 546 - RNA, 22 nt, Artificial Sequence
  Description of Artificial Sequence Synthetic oligonucleotide
uccacugucuuuuucauagauc

SEQ ID NO: 547 - RNA, 23 nt, Artificial Sequence
  Description of Artificial Sequence Synthetic oligonucleotide
uccacugucuuuuucauagaucg

SEQ ID NO: 548 - RNA, 24 nt, Artificial Sequence
  Description of Artificial Sequence Synthetic oligonucleotide
uccacugucuuuuucauagaucga

SEQ ID NO: 549 - RNA, 18 nt, Artificial Sequence
  Description of Artificial Sequence Synthetic oligonucleotide
ucuccacugucuuuuuca

SEQ ID NO: 550 - RNA, 19 nt, Artificial Sequence
  Description of Artificial Sequence Synthetic oligonucleotide
ucuccacugucuuuuucau

SEQ ID NO: 551 - RNA, 20 nt, Artificial Sequence
  Description of Artificial Sequence Synthetic oligonucleotide
ucuccacugucuuuuucaua

SEQ ID NO: 552 - RNA, 21 nt, Artificial Sequence
  Description of Artificial Sequence Synthetic oligonucleotide
ucuccacugucuuuuucauag

SEQ ID NO: 553 - RNA, 22 nt, Artificial Sequence
  Description of Artificial Sequence Synthetic oligonucleotide
ucuccacugucuuuuucauaga

SEQ ID NO: 554 - RNA, 23 nt, Artificial Sequence
  Description of Artificial Sequence Synthetic oligonucleotide
ucuccacugucuuuuucauagau

SEQ ID NO: 555 - RNA, 24 nt, Artificial Sequence
  Description of Artificial Sequence Synthetic oligonucleotide
ucuccacugucuuuuucauagauc

SEQ ID NO: 556 - RNA, 18 nt, Artificial Sequence
  Description of Artificial Sequence Synthetic oligonucleotide
uucuccacugucuuuuuc

SEQ ID NO: 557 - RNA, 19 nt, Artificial Sequence
  Description of Artificial Sequence Synthetic oligonucleotide
uucuccacugucuuuuuca

SEQ ID NO: 558 - RNA, 20 nt, Artificial Sequence
  Description of Artificial Sequence Synthetic oligonucleotide
uucuccacugucuuuuucau

SEQ ID NO: 559 - RNA, 21 nt, Artificial Sequence
  Description of Artificial Sequence Synthetic oligonucleotide
uucuccacugucuuuuucaua

SEQ ID NO: 560 - RNA, 22 nt, Artificial Sequence
  Description of Artificial Sequence Synthetic oligonucleotide
uucuccacugucuuuuucauag

SEQ ID NO: 561 - RNA, 23 nt, Artificial Sequence
  Description of Artificial Sequence Synthetic oligonucleotide
uucuccacugucuuuuucauaga

SEQ ID NO: 562 - RNA, 24 nt, Artificial Sequence
  Description of Artificial Sequence Synthetic oligonucleotide
uucuccacugucuuuuucauagau

SEQ ID NO: 563 - RNA, 18 nt, Artificial Sequence
  Description of Artificial Sequence Synthetic oligonucleotide
uuucuccacugucuuuuu

SEQ ID NO: 564 - RNA, 19 nt, Artificial Sequence
  Description of Artificial Sequence Synthetic oligonucleotide
uuucuccacugucuuuuuc

SEQ ID NO: 565 - RNA, 20 nt, Artificial Sequence
  Description of Artificial Sequence Synthetic oligonucleotide
uuucuccacugucuuuuuca

SEQ ID NO: 566 - RNA, 21 nt, Artificial Sequence
  Description of Artificial Sequence Synthetic oligonucleotide
uuucuccacugucuuuuucau

SEQ ID NO: 567 - RNA, 22 nt, Artificial Sequence
  Description of Artificial Sequence Synthetic oligonucleotide
uuucuccacugucuuuuucaua

SEQ ID NO: 568 - RNA, 23 nt, Artificial Sequence
  Description of Artificial Sequence Synthetic oligonucleotide
uuucuccacugucuuuuucauag

SEQ ID NO: 569 - RNA, 24 nt, Artificial Sequence
  Description of Artificial Sequence Synthetic oligonucleotide
uuucuccacugucuuuuucauaga

SEQ ID NO: 570 - RNA, 18 nt, Artificial Sequence
  Description of Artificial Sequence Synthetic oligonucleotide
uuuucuccacugucuuuu

SEQ ID NO: 571 - RNA, 19 nt, Artificial Sequence
  Description of Artificial Sequence Synthetic oligonucleotide
uuuucuccacugucuuuuu

SEQ ID NO: 572 - RNA, 20 nt, Artificial Sequence
  Description of Artificial Sequence Synthetic oligonucleotide
uuuucuccacugucuuuuuc

SEQ ID NO: 573 - RNA, 21 nt, Artificial Sequence
  Description of Artificial Sequence Synthetic oligonucleotide
uuuucuccacugucuuuuuca

SEQ ID NO: 574 - RNA, 22 nt, Artificial Sequence
  Description of Artificial Sequence Synthetic oligonucleotide
uuuucuccacugucuuuuucau

SEQ ID NO: 575 - RNA, 23 nt, Artificial Sequence
  Description of Artificial Sequence Synthetic oligonucleotide
uuuucuccacugucuuuuucaua

SEQ ID NO: 576 - RNA, 24 nt, Artificial Sequence
  Description of Artificial Sequence Synthetic oligonucleotide
uuuucuccacugucuuuuucauag

SEQ ID NO: 577 - DNA, 21 bp, Artificial Sequence
  Description of Artificial Sequence Synthetic oligonucleotide
gaggtaaagcgtttgcatttg

SEQ ID NO: 578 - DNA, 21 bp, Artificial Sequence
  Description of Artificial Sequence Synthetic oligonucleotide
cctctaaagcttatgcttaca

SEQ ID NO: 579 - DNA, 21 bp, Artificial Sequence
  Description of Artificial Sequence Synthetic oligonucleotide
agtcgatttacttgtagcact

SEQ ID NO: 580 - DNA, 21 bp, Artificial Sequence
  Description of Artificial Sequence Synthetic oligonucleotide
cttgtagcactgcacagttaa

SEQ ID NO: 581 - DNA, 21 bp, Artificial Sequence
  Description of Artificial Sequence Synthetic oligonucleotide
tccattacaggataaaagact

SEQ ID NO: 582 - DNA, 21 bp, Artificial Sequence
  Description of Artificial Sequence Synthetic oligonucleotide
ctccattacaggataaaagac

SEQ ID NO: 583 - DNA, 21 bp, Artificial Sequence
  Description of Artificial Sequence Synthetic oligonucleotide
tctccattacaggataaaaga

SEQ ID NO: 584 - DNA, 21 bp, Artificial Sequence
  Description of Artificial Sequence Synthetic oligonucleotide
atcctgtaatggagaaaaatc

SEQ ID NO: 585 - DNA, 21 bp, Artificial Sequence
  Description of Artificial Sequence Synthetic oligonucleotide
tcctgtaatggagaaaaatcc

SEQ ID NO: 586 - DNA, 21 bp, Artificial Sequence
  Description of Artificial Sequence Synthetic oligonucleotide
aaacatgagtaagttgttttg

SEQ ID NO: 587 - DNA, 21 bp, Artificial Sequence
  Description of Artificial Sequence Synthetic oligonucleotide
gctttcaaacatgagtaagtt

SEQ ID NO: 588 - DNA, 21 bp, Artificial Sequence
  Description of Artificial Sequence Synthetic oligonucleotide
aaagccaaaccattcattgtc

SEQ ID NO: 589 - DNA, 21 bp, Artificial Sequence
  Description of Artificial Sequence Synthetic oligonucleotide
gtaacagcagtcatcatccat

SEQ ID NO: 590 - DNA, 21 bp, Artificial Sequence
  Description of Artificial Sequence Synthetic oligonucleotide
accatcctcatggattggtgt

SEQ ID NO: 591 - DNA, 21 bp, Artificial Sequence
  Description of Artificial Sequence Synthetic oligonucleotide
tgtccatcatttcaccatcct

SEQ ID NO: 592 - DNA, 21 bp, Artificial Sequence
  Description of Artificial Sequence Synthetic oligonucleotide
gaaatttctgtccatcatttc

SEQ ID NO: 593 - DNA, 21 bp, Artificial Sequence
  Description of Artificial Sequence Synthetic oligonucleotide
agaaatttctgtccatcattt

SEQ ID NO: 594 - DNA, 21 bp, Artificial Sequence
  Description of Artificial Sequence Synthetic oligonucleotide
ttttagaaatttctgtccatc

SEQ ID NO: 595 - DNA, 21 bp, Artificial Sequence
  Description of Artificial Sequence Synthetic oligonucleotide
cttttagaaatttctgtccat

SEQ ID NO: 596 - DNA, 21 bp, Artificial Sequence
  Description of Artificial Sequence Synthetic oligonucleotide
ttttcttttagaaatttctgt

SEQ ID NO: 597 - DNA, 21 bp, Artificial Sequence
  Description of Artificial Sequence Synthetic oligonucleotide
taaaagaaaagaaagaatttt

SEQ ID NO: 598 - DNA, 21 bp, Artificial Sequence
  Description of Artificial Sequence Synthetic oligonucleotide
aaacatttacatcttaccatt

SEQ ID NO: 599 - DNA, 21 bp, Artificial Sequence
  Description of Artificial Sequence Synthetic oligonucleotide
catcttaccatttcttcttca

SEQ ID NO: 600 - DNA, 21 bp, Artificial Sequence
  Description of Artificial Sequence Synthetic oligonucleotide
tatagataatgaagaagaaat

SEQ ID NO: 601 - DNA, 21 bp, Artificial Sequence
  Description of Artificial Sequence Synthetic oligonucleotide
ttcttcattatctatagaaag

SEQ ID NO: 602 - DNA, 21 bp, Artificial Sequence
  Description of Artificial Sequence Synthetic oligonucleotide
ctggcctgtacttcgaagaac

SEQ ID NO: 603 - DNA, 21 bp, Artificial Sequence
  Description of Artificial Sequence Synthetic oligonucleotide
cttaccaatgtagtaacaact

SEQ ID NO: 604 - DNA, 21 bp, Artificial Sequence
  Description of Artificial Sequence Synthetic oligonucleotide
gcacgtcattgtggccattgt

SEQ ID NO: 605 - DNA, 21 bp, Artificial Sequence
  Description of Artificial Sequence Synthetic oligonucleotide
tttagcacgtcattgtggcca

SEQ ID NO: 606 - DNA, 21 bp, Artificial Sequence
  Description of Artificial Sequence Synthetic oligonucleotide
ccatcagctccagagaagctc

SEQ ID NO: 607 - DNA, 21 bp, Artificial Sequence
  Description of Artificial Sequence Synthetic oligonucleotide
tctccctgcagatttaccatc

SEQ ID NO: 608 - DNA, 21 bp, Artificial Sequence
  Description of Artificial Sequence Synthetic oligonucleotide
aaatgctttacctttgcagtg

SEQ ID NO: 609 - DNA, 21 bp, Artificial Sequence
  Description of Artificial Sequence Synthetic oligonucleotide
aatgctttacctttgcagtga

SEQ ID NO: 610 - DNA, 21 bp, Artificial Sequence
  Description of Artificial Sequence Synthetic oligonucleotide
cctttgcagtgataggttttg

SEQ ID NO: 611 - DNA, 21 bp, Artificial Sequence
  Description of Artificial Sequence Synthetic oligonucleotide
cagtgataggttttgtcattc

SEQ ID NO: 612 - DNA, 21 bp, Artificial Sequence
  Description of Artificial Sequence Synthetic oligonucleotide
aagggaatgacaaaacctatc

SEQ ID NO: 613 - DNA, 21 bp, Artificial Sequence
  Description of Artificial Sequence Synthetic oligonucleotide
caagggaatgacaaaacctat

SEQ ID NO: 614 - DNA, 21 bp, Artificial Sequence
  Description of Artificial Sequence Synthetic oligonucleotide
gtcattcccttgaaaatcctg

SEQ ID NO: 615 - DNA, 21 bp, Artificial Sequence
  Description of Artificial Sequence Synthetic oligonucleotide
tcattcccttgaaaatcctga

SEQ ID NO: 616 - DNA, 21 bp, Artificial Sequence
  Description of Artificial Sequence Synthetic oligonucleotide
tgaaggtttaattccgcatag

SEQ ID NO: 617 - DNA, 21 bp, Artificial Sequence
  Description of Artificial Sequence Synthetic oligonucleotide
gaaggtttaattccgcatagg

SEQ ID NO: 618 - DNA, 21 bp, Artificial Sequence
  Description of Artificial Sequence Synthetic oligonucleotide
aaggtttaattccgcataggt

SEQ ID NO: 619 - DNA, 21 bp, Artificial Sequence
  Description of Artificial Sequence Synthetic oligonucleotide
attccgcataggttatttcct

SEQ ID NO: 620 - DNA, 21 bp, Artificial Sequence
  Description of Artificial Sequence Synthetic oligonucleotide
gcaactgaacaggaaataacc

SEQ ID NO: 621 - DNA, 21 bp, Artificial Sequence
  Description of Artificial Sequence Synthetic oligonucleotide
agcaactgaacaggaaataac

SEQ ID NO: 622 - DNA, 21 bp, Artificial Sequence
  Description of Artificial Sequence Synthetic oligonucleotide
ctgttcagttgctaaaatgga

SEQ ID NO: 623 - DNA, 21 bp, Artificial Sequence
  Description of Artificial Sequence Synthetic oligonucleotide
tattgcctttaggttttcgtt

SEQ ID NO: 624 - DNA, 21 bp, Artificial Sequence
  Description of Artificial Sequence Synthetic oligonucleotide
attgcctttaggttttcgttg

SEQ ID NO: 625 - DNA, 21 bp, Artificial Sequence
  Description of Artificial Sequence Synthetic oligonucleotide
ttgcctttaggttttcgttgc

SEQ ID NO: 626 - DNA, 21 bp, Artificial Sequence
  Description of Artificial Sequence Synthetic oligonucleotide
ggttttcgttgctgcctcttt

SEQ ID NO: 627 - DNA, 21 bp, Artificial Sequence
  Description of Artificial Sequence Synthetic oligonucleotide
cgttgctgcctctttgggttt

SEQ ID NO: 628 - DNA, 21 bp, Artificial Sequence
  Description of Artificial Sequence Synthetic oligonucleotide
gttgctgcctctttgggtttg

SEQ ID NO: 629 - DNA, 21 bp, Artificial Sequence
  Description of Artificial Sequence Synthetic oligonucleotide
ggtttgggggcagattcaggt

SEQ ID NO: 630 - DNA, 21 bp, Artificial Sequence
  Description of Artificial Sequence Synthetic oligonucleotide
ggggcagattcaggtctgagt

SEQ ID NO: 631 - DNA, 20 bp, Artificial Sequence
  Description of Artificial Sequence Synthetic oligonucleotide
tctgcagaaatgttccccgt

SEQ ID NO: 632 - DNA, 20 bp, Artificial Sequence
  Description of Artificial Sequence Synthetic oligonucleotide
tgcagagaaaggtggctcta

SEQ ID NO: 633 - DNA, 20 bp, Artificial Sequence
  Description of Artificial Sequence Synthetic oligonucleotide
taatgctgacttggggtggc

SEQ ID NO: 634 - DNA, 20 bp, Artificial Sequence
  Description of Artificial Sequence Synthetic oligonucleotide
taggacctccaggaagattc

SEQ ID NO: 635 - DNA, 20 bp, Artificial Sequence
  Description of Artificial Sequence Synthetic oligonucleotide
tagtcaacgcgaccaccacg

SEQ ID NO: 636 - DNA, 20 bp, Artificial Sequence
  Description of Artificial Sequence Synthetic oligonucleotide
tcctgaggtcaccttccaca

SEQ ID NO: 637 - DNA, 20 bp, Artificial Sequence
  Description of Artificial Sequence Synthetic oligonucleotide
tattgtgcctgtcatcattc

SEQ ID NO: 638 - DNA, 21 bp, Artificial Sequence
  Description of Artificial Sequence Synthetic oligonucleotide
tgacaggcacaatagaaacaa

SEQ ID NO: 639 - DNA, 21 bp, Artificial Sequence
  Description of Artificial Sequence Synthetic oligonucleotide
gacaggcacaatagaaacaac

SEQ ID NO: 640 - DNA, 21 bp, Artificial Sequence
  Description of Artificial Sequence Synthetic oligonucleotide
aaacaacggggaacatttctg

SEQ ID NO: 641 - DNA, 21 bp, Artificial Sequence
  Description of Artificial Sequence Synthetic oligonucleotide
acaacggggaacatttctgca

SEQ ID NO: 642 - DNA, 21 bp, Artificial Sequence
  Description of Artificial Sequence Synthetic oligonucleotide
tgatagagccacctttctctg

SEQ ID NO: 643 - DNA, 21 bp, Artificial Sequence
  Description of Artificial Sequence Synthetic oligonucleotide
gggtcacttgtgccgtggtgg

SEQ ID NO: 644 - DNA, 21 bp, Artificial Sequence
  Description of Artificial Sequence Synthetic oligonucleotide
ggcacaagtgacccaggtcaa

SEQ ID NO: 645 - DNA, 21 bp, Artificial Sequence
  Description of Artificial Sequence Synthetic oligonucleotide
gtcctgctgctcccagttgac

SEQ ID NO: 646 - DNA, 21 bp, Artificial Sequence
  Description of Artificial Sequence Synthetic oligonucleotide
tggccatttgtaatgctgact

SEQ ID NO: 647 - DNA, 21 bp, Artificial Sequence
  Description of Artificial Sequence Synthetic oligonucleotide
tggcacatctccccatccttc

SEQ ID NO: 648 - DNA, 21 bp, Artificial Sequence
  Description of Artificial Sequence Synthetic oligonucleotide
catctccccatccttcaagga

SEQ ID NO: 649 - DNA, 21 bp, Artificial Sequence
  Description of Artificial Sequence Synthetic oligonucleotide
ccactcgatccttgaaggatg

SEQ ID NO: 650 - DNA, 21 bp, Artificial Sequence
  Description of Artificial Sequence Synthetic oligonucleotide
ggccactcgatccttgaagga

SEQ ID NO: 651 - DNA, 21 bp, Artificial Sequence
  Description of Artificial Sequence Synthetic oligonucleotide
cctggggccactcgatccttg

SEQ ID NO: 652 - DNA, 21 bp, Artificial Sequence
  Description of Artificial Sequence Synthetic oligonucleotide
gactggagggtgaggcccagg

SEQ ID NO: 653 - DNA, 21 bp, Artificial Sequence
  Description of Artificial Sequence Synthetic oligonucleotide
atcgttcacggtcagcgactg

SEQ ID NO: 654 - DNA, 21 bp, Artificial Sequence
  Description of Artificial Sequence Synthetic oligonucleotide
gtcgctgaccgtgaacgatac

SEQ ID NO: 655 - DNA, 21 bp, Artificial Sequence
  Description of Artificial Sequence Synthetic oligonucleotide
cgctgaccgtgaacgatacag

SEQ ID NO: 656 - DNA, 21 bp, Artificial Sequence
  Description of Artificial Sequence Synthetic oligonucleotide
gcatctatcacacctaccctg

SEQ ID NO: 657 - DNA, 21 bp, Artificial Sequence
  Description of Artificial Sequence Synthetic oligonucleotide
cctaccctgatgggacgtaca

SEQ ID NO: 658 - DNA, 21 bp, Artificial Sequence
  Description of Artificial Sequence Synthetic oligonucleotide
taccctgatgggacgtacact

SEQ ID NO: 659 - DNA, 21 bp, Artificial Sequence
  Description of Artificial Sequence Synthetic oligonucleotide
ccctgatgggacgtacactgg

SEQ ID NO: 660 - DNA, 21 bp, Artificial Sequence
  Description of Artificial Sequence Synthetic oligonucleotide
ttctcccagtgtacgtcccat

SEQ ID NO: 661 - DNA, 21 bp, Artificial Sequence
  Description of Artificial Sequence Synthetic oligonucleotide
ggagaatcttcctggaggtcc

SEQ ID NO: 662 - DNA, 21 bp, Artificial Sequence
  Description of Artificial Sequence Synthetic oligonucleotide
catggctccaagcaatggaat

SEQ ID NO: 663 - DNA, 21 bp, Artificial Sequence
  Description of Artificial Sequence Synthetic oligonucleotide
cgcggccatggctccaagcaa

SEQ ID NO: 664 - DNA, 21 bp, Artificial Sequence
  Description of Artificial Sequence Synthetic oligonucleotide
tcgcggccatggctccaagca

SEQ ID NO: 665 - DNA, 21 bp, Artificial Sequence
  Description of Artificial Sequence Synthetic oligonucleotide
catcgtggtggtcgcgttgac

SEQ ID NO: 666 - DNA, 21 bp, Artificial Sequence
  Description of Artificial Sequence Synthetic oligonucleotide
aaagccctcagaatccattct

SEQ ID NO: 667 - DNA, 21 bp, Artificial Sequence
  Description of Artificial Sequence Synthetic oligonucleotide
cattctgtggaaggtgacctc

SEQ ID NO: 668 - DNA, 21 bp, Artificial Sequence
  Description of Artificial Sequence Synthetic oligonucleotide
ttctgtggaaggtgacctcag

SEQ ID NO: 669 - DNA, 21 bp, Artificial Sequence
  Description of Artificial Sequence Synthetic oligonucleotide
cctgaggtcaccttccacaga

SEQ ID NO: 670 - DNA, 21 bp, Artificial Sequence
  Description of Artificial Sequence Synthetic oligonucleotide
ttctcctgaggtcaccttcca

SEQ ID NO: 671 - DNA, 21 bp, Artificial Sequence
  Description of Artificial Sequence Synthetic oligonucleotide
aggagaaaatcagctggacag

SEQ ID NO: 672 - DNA, 21 bp, Artificial Sequence
  Description of Artificial Sequence Synthetic oligonucleotide
ggagaaaatcagctggacagg

SEQ ID NO: 673 - DNA, 21 bp, Artificial Sequence
  Description of Artificial Sequence Synthetic oligonucleotide
gccccagtgctccctcacccc

SEQ ID NO: 674 - DNA, 21 bp, Artificial Sequence
  Description of Artificial Sequence Synthetic oligonucleotide
tggacacagcttcctgggggt

SEQ ID NO: 675 - DNA, 21 bp, Artificial Sequence
  Description of Artificial Sequence Synthetic oligonucleotide
tctgcctggacacagcttcct

SEQ ID NO: 676 - DNA, 21 bp, Artificial Sequence
  Description of Artificial Sequence Synthetic oligonucleotide
agctgcacctgctgggctctg

SEQ ID NO: 677 - DNA, 21 bp, Artificial Sequence
  Description of Artificial Sequence Synthetic oligonucleotide
gctgggctctgtggagagcag

SEQ ID NO: 678 - DNA, 21 bp, Artificial Sequence
  Description of Artificial Sequence Synthetic oligonucleotide
tgggctctgtggagagcagcg

SEQ ID NO: 679 - DNA, 21 bp, Artificial Sequence
  Description of Artificial Sequence Synthetic oligonucleotide
ctgcatgactacttcaatgtc

SEQ ID NO: 680 - DNA, 21 bp, Artificial Sequence
  Description of Artificial Sequence Synthetic oligonucleotide
aatgtcctgagttacagaagc

SEQ ID NO: 681 - DNA, 21 bp, Artificial Sequence
  Description of Artificial Sequence Synthetic oligonucleotide
tgggtaactgcagcttcttca

SEQ ID NO: 682 - DNA, 20 bp, Artificial Sequence
  Description of Artificial Sequence Synthetic oligonucleotide
gacaggcacaatagaaacaa

SEQ ID NO: 683 - DNA, 20 bp, Artificial Sequence
  Description of Artificial Sequence Synthetic oligonucleotide
acaggcacaatagaaacaac

SEQ ID NO: 684 - DNA, 20 bp, Artificial Sequence
  Description of Artificial Sequence Synthetic oligonucleotide
caggcacaatagaaacaacg

SEQ ID NO: 685 - DNA, 20 bp, Artificial Sequence
  Description of Artificial Sequence Synthetic oligonucleotide
gggaacatttctgcagagaa

SEQ ID NO: 686 - DNA, 20 bp, Artificial Sequence
  Description of Artificial Sequence Synthetic oligonucleotide
aacatttctgcagagaaagg

SEQ ID NO: 687 - DNA, 20 bp, Artificial Sequence
  Description of Artificial Sequence Synthetic oligonucleotide
atgtcacctctcctccacca

SEQ ID NO: 688 - DNA, 20 bp, Artificial Sequence
  Description of Artificial Sequence Synthetic oligonucleotide
cttgtgccgtggtggaggag

SEQ ID NO: 689 - DNA, 20 bp, Artificial Sequence
  Description of Artificial Sequence Synthetic oligonucleotide
ggtcacttgtgccgtggtgg

SEQ ID NO: 690 - DNA, 20 bp, Artificial Sequence
  Description of Artificial Sequence Synthetic oligonucleotide
caccacggcacaagtgaccc

SEQ ID NO: 691 - DNA, 20 bp, Artificial Sequence
  Description of Artificial Sequence Synthetic oligonucleotide
ctgggtcacttgtgccgtgg

SEQ ID NO: 692 - DNA, 20 bp, Artificial Sequence
  Description of Artificial Sequence Synthetic oligonucleotide
gacctgggtcacttgtgccg

SEQ ID NO: 693 - DNA, 20 bp, Artificial Sequence
  Description of Artificial Sequence Synthetic oligonucleotide
cacaagtgacccaggtcaac

SEQ ID NO: 694 - DNA, 20 bp, Artificial Sequence
  Description of Artificial Sequence Synthetic oligonucleotide
acaagtgacccaggtcaact

SEQ ID NO: 695 - DNA, 20 bp, Artificial Sequence
  Description of Artificial Sequence Synthetic oligonucleotide
ccaggtcaactgggagcagc

SEQ ID NO: 696 - DNA, 20 bp, Artificial Sequence
  Description of Artificial Sequence Synthetic oligonucleotide
ctgctgctcccagttgacct

SEQ ID NO: 697 - DNA, 20 bp, Artificial Sequence
  Description of Artificial Sequence Synthetic oligonucleotide
cctgctgctcccagttgacc

SEQ ID NO: 698 - DNA, 20 bp, Artificial Sequence
  Description of Artificial Sequence Synthetic oligonucleotide
ggagcagcaggaccagcttc

SEQ ID NO: 699 - DNA, 20 bp, Artificial Sequence
  Description of Artificial Sequence Synthetic oligonucleotide
cattacaaatggccagaagc

SEQ ID NO: 700 - DNA, 20 bp, Artificial Sequence
  Description of Artificial Sequence Synthetic oligonucleotide
ggccatttgtaatgctgact

SEQ ID NO: 701 - DNA, 20 bp, Artificial Sequence
  Description of Artificial Sequence Synthetic oligonucleotide
gccatttgtaatgctgactt

SEQ ID NO: 702 - DNA, 20 bp, Artificial Sequence
  Description of Artificial Sequence Synthetic oligonucleotide
ccatttgtaatgctgacttg

SEQ ID NO: 703 - DNA, 20 bp, Artificial Sequence
  Description of Artificial Sequence Synthetic oligonucleotide
tttgtaatgctgacttgggg

SEQ ID NO: 704 - DNA, 20 bp, Artificial Sequence
  Description of Artificial Sequence Synthetic oligonucleotide
ccccaagtcagcattacaaa

SEQ ID NO: 705 - DNA, 20 bp, Artificial Sequence
  Description of Artificial Sequence Synthetic oligonucleotide
gcacatctccccatccttca

SEQ ID NO: 706 - DNA, 20 bp, Artificial Sequence
  Description of Artificial Sequence Synthetic oligonucleotide
cccatccttcaaggatcgag

SEQ ID NO: 707 - DNA, 20 bp, Artificial Sequence
  Description of Artificial Sequence Synthetic oligonucleotide
cactcgatccttgaaggatg

SEQ ID NO: 708 - DNA, 20 bp, Artificial Sequence
  Description of Artificial Sequence Synthetic oligonucleotide
ccactcgatccttgaaggat

SEQ ID NO: 709 - DNA, 20 bp, Artificial Sequence
  Description of Artificial Sequence Synthetic oligonucleotide
gccactcgatccttgaagga

SEQ ID NO: 710 - DNA, 20 bp, Artificial Sequence
  Description of Artificial Sequence Synthetic oligonucleotide
ttcaaggatcgagtggcccc

SEQ ID NO: 711 - DNA, 20 bp, Artificial Sequence
  Description of Artificial Sequence Synthetic oligonucleotide
tggggccactcgatccttga

SEQ ID NO: 712 - DNA, 20 bp, Artificial Sequence
  Description of Artificial Sequence Synthetic oligonucleotide
gatcgagtggccccaggtcc

SEQ ID NO: 713 - DNA, 20 bp, Artificial Sequence
  Description of Artificial Sequence Synthetic oligonucleotide
agtggccccaggtcccggcc

SEQ ID NO: 714 - DNA, 20 bp, Artificial Sequence
  Description of Artificial Sequence Synthetic oligonucleotide
gtggccccaggtcccggcct

SEQ ID NO: 715 - DNA, 20 bp, Artificial Sequence
  Description of Artificial Sequence Synthetic oligonucleotide
gaggcccaggccgggacctg

SEQ ID NO: 716 - DNA, 20 bp, Artificial Sequence
  Description of Artificial Sequence Synthetic oligonucleotide
tgaggcccaggccgggacct

SEQ ID NO: 717 - DNA, 20 bp, Artificial Sequence
  Description of Artificial Sequence Synthetic oligonucleotide
gtgaggcccaggccgggacc

SEQ ID NO: 718 - DNA, 20 bp, Artificial Sequence
  Description of Artificial Sequence Synthetic oligonucleotide
tggagggtgaggcccaggcc

SEQ ID NO: 719 - DNA, 20 bp, Artificial Sequence
  Description of Artificial Sequence Synthetic oligonucleotide
ctggagggtgaggcccaggc

SEQ ID NO: 720 - DNA, 20 bp, Artificial Sequence
  Description of Artificial Sequence Synthetic oligonucleotide
gcgactggagggtgaggccc

SEQ ID NO: 721 - DNA, 20 bp, Artificial Sequence
  Description of Artificial Sequence Synthetic oligonucleotide
cggtcagcgactggagggtg

SEQ ID NO: 722 - DNA, 20 bp, Artificial Sequence
  Description of Artificial Sequence Synthetic oligonucleotide
gttcacggtcagcgactgga

SEQ ID NO: 723 - DNA, 20 bp, Artificial Sequence
  Description of Artificial Sequence Synthetic oligonucleotide
cgttcacggtcagcgactgg

SEQ ID NO: 724 - DNA, 20 bp, Artificial Sequence
  Description of Artificial Sequence Synthetic oligonucleotide
tatcgttcacggtcagcgac

SEQ ID NO: 725 - DNA, 20 bp, Artificial Sequence
  Description of Artificial Sequence Synthetic oligonucleotide
tcgctgaccgtgaacgatac

SEQ ID NO: 726 - DNA, 20 bp, Artificial Sequence
  Description of Artificial Sequence Synthetic oligonucleotide
cgctgaccgtgaacgataca

SEQ ID NO: 727 - DNA, 20 bp, Artificial Sequence
  Description of Artificial Sequence Synthetic oligonucleotide
gctgaccgtgaacgatacag

SEQ ID NO: 728 - DNA, 20 bp, Artificial Sequence
  Description of Artificial Sequence Synthetic oligonucleotide
gtactcccctgtatcgttca

SEQ ID NO: 729 - DNA, 20 bp, Artificial Sequence
  Description of Artificial Sequence Synthetic oligonucleotide
atctatcacacctaccctga

SEQ ID NO: 730 - DNA, 20 bp, Artificial Sequence
  Description of Artificial Sequence Synthetic oligonucleotide
tctatcacacctaccctgat

SEQ ID NO: 731 - DNA, 20 bp, Artificial Sequence
  Description of Artificial Sequence Synthetic oligonucleotide
taccctgatgggacgtacac

SEQ ID NO: 732 - DNA, 20 bp, Artificial Sequence
  Description of Artificial Sequence Synthetic oligonucleotide
accctgatgggacgtacact

SEQ ID NO: 733 - DNA, 20 bp, Artificial Sequence
  Description of Artificial Sequence Synthetic oligonucleotide
agtgtacgtcccatcagggt

SEQ ID NO: 734 - DNA, 20 bp, Artificial Sequence
  Description of Artificial Sequence Synthetic oligonucleotide
tcccagtgtacgtcccatca

SEQ ID NO: 735 - DNA, 20 bp, Artificial Sequence
  Description of Artificial Sequence Synthetic oligonucleotide
ctcccagtgtacgtcccatc

SEQ ID NO: 736 - DNA, 20 bp, Artificial Sequence
  Description of Artificial Sequence Synthetic oligonucleotide
gtacactgggagaatcttcc

SEQ ID NO: 737 - DNA, 20 bp, Artificial Sequence
  Description of Artificial Sequence Synthetic oligonucleotide
cactgggagaatcttcctgg

SEQ ID NO: 738 - DNA, 20 bp, Artificial Sequence
  Description of Artificial Sequence Synthetic oligonucleotide
ctgagctttctaggacctcc

SEQ ID NO: 739 - DNA, 20 bp, Artificial Sequence
  Description of Artificial Sequence Synthetic oligonucleotide
aggttccagattccattgct

SEQ ID NO: 740 - DNA, 20 bp, Artificial Sequence
  Description of Artificial Sequence Synthetic oligonucleotide
aagcaatggaatctggaacc

SEQ ID NO: 741 - DNA, 20 bp, Artificial Sequence
  Description of Artificial Sequence Synthetic oligonucleotide
gattccattgcttggagcca

SEQ ID NO: 742 - DNA, 20 bp, Artificial Sequence
  Description of Artificial Sequence Synthetic oligonucleotide
tggctccaagcaatggaatc

SEQ ID NO: 743 - DNA, 20 bp, Artificial Sequence
  Description of Artificial Sequence Synthetic oligonucleotide
gcggccatggctccaagcaa

SEQ ID NO: 744 - DNA, 20 bp, Artificial Sequence
  Description of Artificial Sequence Synthetic oligonucleotide
tggagccatggccgcgacgc

SEQ ID NO: 745 - DNA, 20 bp, Artificial Sequence
  Description of Artificial Sequence Synthetic oligonucleotide
agccatggccgcgacgctgg

SEQ ID NO: 746 - DNA, 20 bp, Artificial Sequence
  Description of Artificial Sequence Synthetic oligonucleotide
gaccaccagcgtcgcggcca

SEQ ID NO: 747 - DNA, 20 bp, Artificial Sequence
  Description of Artificial Sequence Synthetic oligonucleotide
gcagatgaccaccagcgtcg

SEQ ID NO: 748 - DNA, 20 bp, Artificial Sequence
  Description of Artificial Sequence Synthetic oligonucleotide
catctgcacagcagtcatcg

SEQ ID NO: 749 - DNA, 20 bp, Artificial Sequence
  Description of Artificial Sequence Synthetic oligonucleotide
ctgcacagcagtcatcgtgg

SEQ ID NO: 750 - DNA, 20 bp, Artificial Sequence
  Description of Artificial Sequence Synthetic oligonucleotide
agccctcagaatccattctg

SEQ ID NO: 751 - DNA, 20 bp, Artificial Sequence
  Description of Artificial Sequence Synthetic oligonucleotide
ctcagaatccattctgtgga

SEQ ID NO: 752 - DNA, 20 bp, Artificial Sequence
  Description of Artificial Sequence Synthetic oligonucleotide
ttccacagaatggattctga

SEQ ID NO: 753 - DNA, 20 bp, Artificial Sequence
  Description of Artificial Sequence Synthetic oligonucleotide
cttccacagaatggattctg

SEQ ID NO: 754 - DNA, 20 bp, Artificial Sequence
  Description of Artificial Sequence Synthetic oligonucleotide
attctgtggaaggtgacctc

SEQ ID NO: 755 - DNA, 20 bp, Artificial Sequence
  Description of Artificial Sequence Synthetic oligonucleotide
tgaggtcaccttccacagaa

SEQ ID NO: 756 - DNA, 20 bp, Artificial Sequence
  Description of Artificial Sequence Synthetic oligonucleotide
gacctcaggagaaaatcagc

SEQ ID NO: 757 - DNA, 20 bp, Artificial Sequence
  Description of Artificial Sequence Synthetic oligonucleotide
caggagaaaatcagctggac

SEQ ID NO: 758 - DNA, 20 bp, Artificial Sequence
  Description of Artificial Sequence Synthetic oligonucleotide
gtccagctgattttctcctg

SEQ ID NO: 759 - DNA, 20 bp, Artificial Sequence
  Description of Artificial Sequence Synthetic oligonucleotide
gagaaaatcagctggacagg

SEQ ID NO: 760 - DNA, 20 bp, Artificial Sequence
  Description of Artificial Sequence Synthetic oligonucleotide
aatcagctggacaggaggaa

SEQ ID NO: 761 - DNA, 20 bp, Artificial Sequence
  Description of Artificial Sequence Synthetic oligonucleotide
cccagtgctccctcaccccc

SEQ ID NO: 762 - DNA, 20 bp, Artificial Sequence
  Description of Artificial Sequence Synthetic oligonucleotide
ctgggggtgagggagcactg

SEQ ID NO: 763 - DNA, 20 bp, Artificial Sequence
  Description of Artificial Sequence Synthetic oligonucleotide
cctgggggtgagggagcact

SEQ ID NO: 764 - DNA, 20 bp, Artificial Sequence
  Description of Artificial Sequence Synthetic oligonucleotide
tcctgggggtgagggagcac

SEQ ID NO: 765 - DNA, 20 bp, Artificial Sequence
  Description of Artificial Sequence Synthetic oligonucleotide
acacagcttcctgggggtga

SEQ ID NO: 766 - DNA, 20 bp, Artificial Sequence
  Description of Artificial Sequence Synthetic oligonucleotide
gacacagcttcctgggggtg

SEQ ID NO: 767 - DNA, 20 bp, Artificial Sequence
  Description of Artificial Sequence Synthetic oligonucleotide
acccccaggaagctgtgtcc

SEQ ID NO: 768 - DNA, 20 bp, Artificial Sequence
  Description of Artificial Sequence Synthetic oligonucleotide
gcctggacacagcttcctgg

SEQ ID NO: 769 - DNA, 20 bp, Artificial Sequence
  Description of Artificial Sequence Synthetic oligonucleotide
tgcctggacacagcttcctg

SEQ ID NO: 770 - DNA, 20 bp, Artificial Sequence
  Description of Artificial Sequence Synthetic oligonucleotide
ctgcctggacacagcttcct

SEQ ID NO: 771 - DNA, 20 bp, Artificial Sequence
  Description of Artificial Sequence Synthetic oligonucleotide
tctgcctggacacagcttcc

SEQ ID NO: 772 - DNA, 20 bp, Artificial Sequence
  Description of Artificial Sequence Synthetic oligonucleotide
caggcagaagctgcacctgc

SEQ ID NO: 773 - DNA, 20 bp, Artificial Sequence
  Description of Artificial Sequence Synthetic oligonucleotide
aggcagaagctgcacctgct

SEQ ID NO: 774 - DNA, 20 bp, Artificial Sequence
  Description of Artificial Sequence Synthetic oligonucleotide
cagcaggtgcagcttctgcc

SEQ ID NO: 775 - DNA, 20 bp, Artificial Sequence
  Description of Artificial Sequence Synthetic oligonucleotide
gctgcacctgctgggctctg

SEQ ID NO: 776 - DNA, 20 bp, Artificial Sequence
  Description of Artificial Sequence Synthetic oligonucleotide
tgctctccacagagcccagc

SEQ ID NO: 777 - DNA, 20 bp, Artificial Sequence
  Description of Artificial Sequence Synthetic oligonucleotide
ctgggctctgtggagagcag

SEQ ID NO: 778 - DNA, 20 bp, Artificial Sequence
  Description of Artificial Sequence Synthetic oligonucleotide
tgggctctgtggagagcagc

SEQ ID NO: 779 - DNA, 20 bp, Artificial Sequence
  Description of Artificial Sequence Synthetic oligonucleotide
gggctctgtggagagcagcg

SEQ ID NO: 780 - DNA, 20 bp, Artificial Sequence
  Description of Artificial Sequence Synthetic oligonucleotide
ctgtggagagcagcggggag

SEQ ID NO: 781 - DNA, 20 bp, Artificial Sequence
  Description of Artificial Sequence Synthetic oligonucleotide
attgaagtagtcatgcagct

SEQ ID NO: 782 - DNA, 20 bp, Artificial Sequence
  Description of Artificial Sequence Synthetic oligonucleotide
tgtcctgagttacagaagcc

SEQ ID NO: 783 - DNA, 20 bp, Artificial Sequence
  Description of Artificial Sequence Synthetic oligonucleotide
gtcctgagttacagaagcct

SEQ ID NO: 784 - DNA, 20 bp, Artificial Sequence
  Description of Artificial Sequence Synthetic oligonucleotide
tacccaggcttctgtaactc

SEQ ID NO: 785 - DNA, 20 bp, Artificial Sequence
  Description of Artificial Sequence Synthetic oligonucleotide
tgaagaagctgcagttaccc

SEQ ID NO: 786 - DNA, 20 bp, Artificial Sequence
  Description of Artificial Sequence Synthetic oligonucleotide
tgcagcttcttcacagagac

SEQ ID NO: 787 - DNA, 20 bp, Artificial Sequence
  Description of Artificial Sequence Synthetic oligonucleotide
gttgtttctattgtgcctgt

SEQ ID NO: 788 - DNA, 20 bp, Artificial Sequence
  Description of Artificial Sequence Synthetic oligonucleotide
cgttgtttctattgtgcctg

SEQ ID NO: 789 - DNA, 20 bp, Artificial Sequence
  Description of Artificial Sequence Synthetic oligonucleotide
ccgttgtttctattgtgcct

SEQ ID NO: 790 - DNA, 20 bp, Artificial Sequence
  Description of Artificial Sequence Synthetic oligonucleotide
ccacggcacaagtgacccag

SEQ ID NO: 791 - DNA, 20 bp, Artificial Sequence
  Description of Artificial Sequence Synthetic oligonucleotide
agttgacctgggtcacttgt

SEQ ID NO: 792 - DNA, 20 bp, Artificial Sequence
  Description of Artificial Sequence Synthetic oligonucleotide
aagtcagcattacaaatggc

SEQ ID NO: 793 - DNA, 20 bp, Artificial Sequence
  Description of Artificial Sequence Synthetic oligonucleotide
catccttcaaggatcgagtg

SEQ ID NO: 794 - DNA, 20 bp, Artificial Sequence
  Description of Artificial Sequence Synthetic oligonucleotide
atccttcaaggatcgagtgg

SEQ ID NO: 795 - DNA, 20 bp, Artificial Sequence
  Description of Artificial Sequence Synthetic oligonucleotide
aggatcgagtggccccaggt

SEQ ID NO: 796 - DNA, 20 bp, Artificial Sequence
  Description of Artificial Sequence Synthetic oligonucleotide
aggtcccggcctgggcctca

SEQ ID NO: 797 - DNA, 20 bp, Artificial Sequence
  Description of Artificial Sequence Synthetic oligonucleotide
ggcctgggcctcaccctcca

SEQ ID NO: 798 - DNA, 20 bp, Artificial Sequence
  Description of Artificial Sequence Synthetic oligonucleotide
cggtcagcgactggagggtg

SEQ ID NO: 799 - DNA, 20 bp, Artificial Sequence
  Description of Artificial Sequence Synthetic oligonucleotide
gtcgctgaccgtgaacgata

SEQ ID NO: 800 - DNA, 20 bp, Artificial Sequence
  Description of Artificial Sequence Synthetic oligonucleotide
tgtatcgttcacggtcagcg

SEQ ID NO: 801 - DNA, 20 bp, Artificial Sequence
  Description of Artificial Sequence Synthetic oligonucleotide
ctgtatcgttcacggtcagc

SEQ ID NO: 802 - DNA, 20 bp, Artificial Sequence
  Description of Artificial Sequence Synthetic oligonucleotide
atcagggtaggtgtgataga

SEQ ID NO: 803 - DNA, 20 bp, Artificial Sequence
  Description of Artificial Sequence Synthetic oligonucleotide
agtgtacgtcccatcagggt

SEQ ID NO: 804 - DNA, 20 bp, Artificial Sequence
  Description of Artificial Sequence Synthetic oligonucleotide
ggaagattctcccagtgtac

SEQ ID NO: 805 - DNA, 20 bp, Artificial Sequence
  Description of Artificial Sequence Synthetic oligonucleotide
tggaggtcctagaaagctca

SEQ ID NO: 806 - DNA, 20 bp, Artificial Sequence
  Description of Artificial Sequence Synthetic oligonucleotide
agcaatggaatctggaacct

SEQ ID NO: 807 - DNA, 20 bp, Artificial Sequence
  Description of Artificial Sequence Synthetic oligonucleotide
agattccattgcttggagcc

SEQ ID NO: 808 - DNA, 20 bp, Artificial Sequence
  Description of Artificial Sequence Synthetic oligonucleotide
gattccattgcttggagcca

SEQ ID NO: 809 - DNA, 20 bp, Artificial Sequence
  Description of Artificial Sequence Synthetic oligonucleotide
attgcttggagccatggccg

SEQ ID NO: 810 - DNA, 20 bp, Artificial Sequence
  Description of Artificial Sequence Synthetic oligonucleotide
ttgcttggagccatggccgc

SEQ ID NO: 811 - DNA, 20 bp, Artificial Sequence
  Description of Artificial Sequence Synthetic oligonucleotide
cagaatggattctgagggct

SEQ ID NO: 812 - DNA, 20 bp, Artificial Sequence
  Description of Artificial Sequence Synthetic oligonucleotide
acagaatggattctgagggc

SEQ ID NO: 813 - DNA, 20 bp, Artificial Sequence
  Description of Artificial Sequence Synthetic oligonucleotide
ttctgtggaaggtgacctca

SEQ ID NO: 814 - DNA, 20 bp, Artificial Sequence
  Description of Artificial Sequence Synthetic oligonucleotide
gctgattttctcctgaggtc

SEQ ID NO: 815 - DNA, 20 bp, Artificial Sequence
  Description of Artificial Sequence Synthetic oligonucleotide
tcctgtccagctgattttct

SEQ ID NO: 816 - DNA, 20 bp, Artificial Sequence
  Description of Artificial Sequence Synthetic oligonucleotide
ttcctcctgtccagctgatt

SEQ ID NO: 817 - DNA, 20 bp, Artificial Sequence
  Description of Artificial Sequence Synthetic oligonucleotide
tgggggtgagggagcactgg

SEQ ID NO: 818 - DNA, 20 bp, Artificial Sequence
  Description of Artificial Sequence Synthetic oligonucleotide
agtgctccctcacccccagg

SEQ ID NO: 819 - DNA, 20 bp, Artificial Sequence
  Description of Artificial Sequence Synthetic oligonucleotide
tcacccccaggaagctgtgt

SEQ ID NO: 820 - DNA, 20 bp, Artificial Sequence
  Description of Artificial Sequence Synthetic oligonucleotide
caggaagctgtgtccaggca

SEQ ID NO: 821 - DNA, 20 bp, Artificial Sequence
  Description of Artificial Sequence Synthetic oligonucleotide
aggaagctgtgtccaggcag

SEQ ID NO: 822 - DNA, 20 bp, Artificial Sequence
  Description of Artificial Sequence Synthetic oligonucleotide
ggcagaagctgcacctgctg

SEQ ID NO: 823 - DNA, 20 bp, Artificial Sequence
  Description of Artificial Sequence Synthetic oligonucleotide
cagagcccagcaggtgcagc

SEQ ID NO: 824 - DNA, 20 bp, Artificial Sequence
  Description of Artificial Sequence Synthetic oligonucleotide
gctgctctccacagagccca

SEQ ID NO: 825 - DNA, 20 bp, Artificial Sequence
  Description of Artificial Sequence Synthetic oligonucleotide
cgctgctctccacagagccc

SEQ ID NO: 826 - DNA, 20 bp, Artificial Sequence
  Description of Artificial Sequence Synthetic oligonucleotide
atgtcctgagttacagaagc

SEQ ID NO: 827 - DNA, 20 bp, Artificial Sequence
  Description of Artificial Sequence Synthetic oligonucleotide
gagcacacccactgcgatgt

SEQ ID NO: 828 - DNA, 20 bp, Artificial Sequence
  Description of Artificial Sequence Synthetic oligonucleotide
gatggccaggagactgaaga

SEQ ID NO: 829 - DNA, 20 bp, Artificial Sequence
  Description of Artificial Sequence Synthetic oligonucleotide
ctgctcaccggagcgggatg

SEQ ID NO: 830 - DNA, 20 bp, Artificial Sequence
  Description of Artificial Sequence Synthetic oligonucleotide
gtctgtggccatgcccatca

SEQ ID NO: 831 - DNA, 20 bp, Artificial Sequence
  Description of Artificial Sequence Synthetic oligonucleotide
tcaccggagcgggatgcgga

SEQ ID NO: 832 - DNA, 20 bp, Artificial Sequence
  Description of Artificial Sequence Synthetic oligonucleotide
gtggcaggcagcgcagaacc

SEQ ID NO: 833 - DNA, 20 bp, Artificial Sequence
  Description of Artificial Sequence Synthetic oligonucleotide
agcacaccagcacattgccc

SEQ ID NO: 834 - DNA, 20 bp, Artificial Sequence
  Description of Artificial Sequence Synthetic oligonucleotide
caggttgctgttgagccaca

SEQ ID NO: 835 - DNA, 20 bp, Artificial Sequence
  Description of Artificial Sequence Synthetic oligonucleotide
cttcattgcctgcttcgtcc

SEQ ID NO: 836 - DNA, 20 bp, Artificial Sequence
  Description of Artificial Sequence Synthetic oligonucleotide
gtacaccgaggagcccatga

SEQ ID NO: 837 - DNA, 20 bp, Artificial Sequence
  Description of Artificial Sequence Synthetic oligonucleotide
gatggcaatgtagcggtcaa

SEQ ID NO: 838 - DNA, 20 bp, Artificial Sequence
  Description of Artificial Sequence Synthetic oligonucleotide
ctcctcggtgtacatcacgg

SEQ ID NO: 839 - DNA, 20 bp, Artificial Sequence
  Description of Artificial Sequence Synthetic oligonucleotide
cgaggagcccatgatgggca

SEQ ID NO: 840 - DNA, 20 bp, Artificial Sequence
  Description of Artificial Sequence Synthetic oligonucleotide
gggctcctcggtgtacatca

SEQ ID NO: 841 - DNA, 20 bp, Artificial Sequence
  Description of Artificial Sequence Synthetic oligonucleotide
ctttgtggtgtcactggcgg

SEQ ID NO: 842 - DNA, 20 bp, Artificial Sequence
  Description of Artificial Sequence Synthetic oligonucleotide
ccgctccggtgagcagggcc

SEQ ID NO: 843 - DNA, 20 bp, Artificial Sequence
  Description of Artificial Sequence Synthetic oligonucleotide
gggttctgcgctgcctgcca

SEQ ID NO: 844 - DNA, 20 bp, Artificial Sequence
  Description of Artificial Sequence Synthetic oligonucleotide
ggacgaagcaggcaatgaag

SEQ ID NO: 845 - DNA, 20 bp, Artificial Sequence
  Description of Artificial Sequence Synthetic oligonucleotide
gtgctgatggtgatggcaaa

SEQ ID NO: 846 - DNA, 20 bp, Artificial Sequence
  Description of Artificial Sequence Synthetic oligonucleotide
agcgcagaacccggtgctga

SEQ ID NO: 847 - DNA, 20 bp, Artificial Sequence
  Description of Artificial Sequence Synthetic oligonucleotide
gagctccatcttcagtctcc

SEQ ID NO: 848 - DNA, 20 bp, Artificial Sequence
  Description of Artificial Sequence Synthetic oligonucleotide
tgctgatggtgatggcaaag

SEQ ID NO: 849 - DNA, 20 bp, Artificial Sequence
  Description of Artificial Sequence Synthetic oligonucleotide
ggcggcggccgacatcgcag

SEQ ID NO: 850 - DNA, 20 bp, Artificial Sequence
  Description of Artificial Sequence Synthetic oligonucleotide
aatgaagaggcagccgtggc

SEQ ID NO: 851 - DNA, 20 bp, Artificial Sequence
  Description of Artificial Sequence Synthetic oligonucleotide
gggcaatgtgctggtgtgct

SEQ ID NO: 852 - DNA, 20 bp, Artificial Sequence
  Description of Artificial Sequence Synthetic oligonucleotide
catgcccatcatgggctcct

SEQ ID NO: 853 - DNA, 20 bp, Artificial Sequence
  Description of Artificial Sequence Synthetic oligonucleotide
aatgtagcggtcaatggcga

SEQ ID NO: 854 - DNA, 20 bp, Artificial Sequence
  Description of Artificial Sequence Synthetic oligonucleotide
agtagttggtgacgttctgc

SEQ ID NO: 855 - DNA, 20 bp, Artificial Sequence
  Description of Artificial Sequence Synthetic oligonucleotide
agcggtcaatggcgatggcc

SEQ ID NO: 856 - DNA, 20 bp, Artificial Sequence
  Description of Artificial Sequence Synthetic oligonucleotide
cgcatcccgctccggtgagc

SEQ ID NO: 857 - DNA, 20 bp, Artificial Sequence
  Description of Artificial Sequence Synthetic oligonucleotide
gcatcccgctccggtgagca

SEQ ID NO: 858 - DNA, 20 bp, Artificial Sequence
  Description of Artificial Sequence Synthetic oligonucleotide
tgggcaatgtgctggtgtgc

SEQ ID NO: 859 - DNA, 20 bp, Artificial Sequence
  Description of Artificial Sequence Synthetic oligonucleotide
caactactttgtggtgtcac

SEQ ID NO: 860 - DNA, 20 bp, Artificial Sequence
  Description of Artificial Sequence Synthetic oligonucleotide
cgctccggtgagcagggccg

SEQ ID NO: 861 - DNA, 20 bp, Artificial Sequence
  Description of Artificial Sequence Synthetic oligonucleotide
gatggtgatggcaaagggga

SEQ ID NO: 862 - DNA, 20 bp, Artificial Sequence
  Description of Artificial Sequence Synthetic oligonucleotide
ggtgtacatcacggtggagc

SEQ ID NO: 863 - DNA, 20 bp, Artificial Sequence
  Description of Artificial Sequence Synthetic oligonucleotide
gaacgtcaccaactactttg

SEQ ID NO: 864 - DNA, 20 bp, Artificial Sequence
  Description of Artificial Sequence Synthetic oligonucleotide
cagtgacaccacaaagtagt

SEQ ID NO: 865 - DNA, 20 bp, Artificial Sequence
  Description of Artificial Sequence Synthetic oligonucleotide
ggccatcctgggcaatgtgc

SEQ ID NO: 866 - DNA, 20 bp, Artificial Sequence
  Description of Artificial Sequence Synthetic oligonucleotide
cccggccctgctcaccggag

SEQ ID NO: 867 - DNA, 20 bp, Artificial Sequence
  Description of Artificial Sequence Synthetic oligonucleotide
caccagcacattgcccagga

SEQ ID NO: 868 - DNA, 20 bp, Artificial Sequence
  Description of Artificial Sequence Synthetic oligonucleotide
tttgccatcaccatcagcac

SEQ ID NO: 869 - DNA, 20 bp, Artificial Sequence
  Description of Artificial Sequence Synthetic oligonucleotide
ctccaccgtgatgtacaccg

SEQ ID NO: 870 - DNA, 20 bp, Artificial Sequence
  Description of Artificial Sequence Synthetic oligonucleotide
ggagctggccattgctgtgc

SEQ ID NO: 871 - DNA, 20 bp, Artificial Sequence
  Description of Artificial Sequence Synthetic oligonucleotide
caggatggccagcacagcaa

SEQ ID NO: 872 - DNA, 20 bp, Artificial Sequence
  Description of Artificial Sequence Synthetic oligonucleotide
gaacccggtgctgatggtga

SEQ ID NO: 873 - DNA, 20 bp, Artificial Sequence
  Description of Artificial Sequence Synthetic oligonucleotide
tggagctctgcgtgaggacc

SEQ ID NO: 874 - DNA, 20 bp, Artificial Sequence
  Description of Artificial Sequence Synthetic oligonucleotide
cccgctccggtgagcagggc

SEQ ID NO: 875 - DNA, 20 bp, Artificial Sequence
  Description of Artificial Sequence Synthetic oligonucleotide
aggcaatgaagaggcagccg

SEQ ID NO: 876 - DNA, 20 bp, Artificial Sequence
  Description of Artificial Sequence Synthetic oligonucleotide
ccggccctgctcaccggagc

SEQ ID NO: 877 - DNA, 20 bp, Artificial Sequence
  Description of Artificial Sequence Synthetic oligonucleotide
gcggcggccgacatcgcagt

SEQ ID NO: 878 - DNA, 20 bp, Artificial Sequence
  Description of Artificial Sequence Synthetic oligonucleotide
ggtgctgatggtgatggcaa

SEQ ID NO: 879 - DNA, 20 bp, Artificial Sequence
  Description of Artificial Sequence Synthetic oligonucleotide
ctactttgtggtgtcactgg

SEQ ID NO: 880 - DNA, 20 bp, Artificial Sequence
  Description of Artificial Sequence Synthetic oligonucleotide
tacaccgaggagcccatgat

SEQ ID NO: 881 - DNA, 20 bp, Artificial Sequence
  Description of Artificial Sequence Synthetic oligonucleotide
tctgtggccatgcccatcat

SEQ ID NO: 882 - DNA, 20 bp, Artificial Sequence
  Description of Artificial Sequence Synthetic oligonucleotide
attgctgtgctggccatcct

SEQ ID NO: 883 - DNA, 20 bp, Artificial Sequence
  Description of Artificial Sequence Synthetic oligonucleotide
cgtgaggaccaggacgaagc

SEQ ID NO: 884 - DNA, 20 bp, Artificial Sequence
  Description of Artificial Sequence Synthetic oligonucleotide
ttgccatcaccatcagcacc

SEQ ID NO: 885 - DNA, 20 bp, Artificial Sequence
  Description of Artificial Sequence Synthetic oligonucleotide
ggatgcggatggcaatgtag

SEQ ID NO: 886 - DNA, 20 bp, Artificial Sequence
  Description of Artificial Sequence Synthetic oligonucleotide
ttgccatccgcatcccgctc

SEQ ID NO: 887 - DNA, 20 bp, Artificial Sequence
  Description of Artificial Sequence Synthetic oligonucleotide
tgaagatggagctctgcgtg

SEQ ID NO: 888 - DNA, 20 bp, Artificial Sequence
  Description of Artificial Sequence Synthetic oligonucleotide
cattgctgtgctggccatcc

SEQ ID NO: 889 - DNA, 20 bp, Artificial Sequence
  Description of Artificial Sequence Synthetic oligonucleotide
tgctggtgtgctgggccgtg

SEQ ID NO: 890 - DNA, 21 bp, Artificial Sequence
  Description of Artificial Sequence Synthetic oligonucleotide
ggctcctcggtgtacatcacg

SEQ ID NO: 891 - DNA, 21 bp, Artificial Sequence
  Description of Artificial Sequence Synthetic oligonucleotide
gagctctgcgtgaggaccagg

SEQ ID NO: 892 - DNA, 21 bp, Artificial Sequence
  Description of Artificial Sequence Synthetic oligonucleotide
gatggagctctgcgtgaggac

SEQ ID NO: 893 - DNA, 21 bp, Artificial Sequence
  Description of Artificial Sequence Synthetic oligonucleotide
ccagcacaccagcacattgcc

SEQ ID NO: 894 - DNA, 21 bp, Artificial Sequence
  Description of Artificial Sequence Synthetic oligonucleotide
aggaccaggacgaagcaggca

SEQ ID NO: 895 - DNA, 21 bp, Artificial Sequence
  Description of Artificial Sequence Synthetic oligonucleotide
tgccatccgcatcccgctccg

SEQ ID NO: 896 - DNA, 21 bp, Artificial Sequence
  Description of Artificial Sequence Synthetic oligonucleotide
gtgtggctcaacagcaacctg

SEQ ID NO: 897 - DNA, 21 bp, Artificial Sequence
  Description of Artificial Sequence Synthetic oligonucleotide
agctccaccgtgatgtacacc

SEQ ID NO: 898 - DNA, 21 bp, Artificial Sequence
  Description of Artificial Sequence Synthetic oligonucleotide
gtagcggtcaatggcgatggc

SEQ ID NO: 899 - DNA, 21 bp, Artificial Sequence
  Description of Artificial Sequence Synthetic oligonucleotide
cggtgctgatggtgatggcaa

SEQ ID NO: 900 - DNA, 21 bp, Artificial Sequence
  Description of Artificial Sequence Synthetic oligonucleotide
ccctgctcaccggagcgggat

SEQ ID NO: 901 - DNA, 21 bp, Artificial Sequence
  Description of Artificial Sequence Synthetic oligonucleotide
gtgacgttctgcaggttgctg

SEQ ID NO: 902 - DNA, 21 bp, Artificial Sequence
  Description of Artificial Sequence Synthetic oligonucleotide
gctccaccgtgatgtacaccg

SEQ ID NO: 903 - DNA, 21 bp, Artificial Sequence
  Description of Artificial Sequence Synthetic oligonucleotide
actgaagatggagctctgcgt

SEQ ID NO: 904 - DNA, 21 bp, Artificial Sequence
  Description of Artificial Sequence Synthetic oligonucleotide
ccagctccaccgtgatgtaca

SEQ ID NO: 905 - DNA, 21 bp, Artificial Sequence
  Description of Artificial Sequence Synthetic oligonucleotide
cctttgccatcaccatcagca

SEQ ID NO: 906 - DNA, 21 bp, Artificial Sequence
  Description of Artificial Sequence Synthetic oligonucleotide
ccggtgctgatggtgatggca

SEQ ID NO: 907 - DNA, 21 bp, Artificial Sequence
  Description of Artificial Sequence Synthetic oligonucleotide
cctgggcaatgtgctggtgtg

SEQ ID NO: 908 - DNA, 21 bp, Artificial Sequence
  Description of Artificial Sequence Synthetic oligonucleotide
aggcagccgtggcaggcagcg

SEQ ID NO: 909 - DNA, 21 bp, Artificial Sequence
  Description of Artificial Sequence Synthetic oligonucleotide
gcgatggccaggagactgaag

SEQ ID NO: 910 - DNA, 21 bp, Artificial Sequence
  Description of Artificial Sequence Synthetic oligonucleotide
cgatggccaggagactgaaga

SEQ ID NO: 911 - DNA, 21 bp, Artificial Sequence
  Description of Artificial Sequence Synthetic oligonucleotide
tcccgctccggtgagcagggc

SEQ ID NO: 912 - DNA, 21 bp, Artificial Sequence
  Description of Artificial Sequence Synthetic oligonucleotide
tgcttcgtcctggtcctcacg

SEQ ID NO: 913 - DNA, 21 bp, Artificial Sequence
  Description of Artificial Sequence Synthetic oligonucleotide
accaggacgaagcaggcaatg

SEQ ID NO: 914 - DNA, 21 bp, Artificial Sequence
  Description of Artificial Sequence Synthetic oligonucleotide
atgtacaccgaggagcccatg

SEQ ID NO: 915 - DNA, 21 bp, Artificial Sequence
  Description of Artificial Sequence Synthetic oligonucleotide
tcgtctgtggccatgcccatc

SEQ ID NO: 916 - DNA, 21 bp, Artificial Sequence
  Description of Artificial Sequence Synthetic oligonucleotide
tcaatggcgatggccaggaga

SEQ ID NO: 917 - DNA, 21 bp, Artificial Sequence
  Description of Artificial Sequence Synthetic oligonucleotide
ggtgctgatggtgatggcaaa

SEQ ID NO: 918 - DNA, 21 bp, Artificial Sequence
  Description of Artificial Sequence Synthetic oligonucleotide
tagcggtcaatggcgatggcc

SEQ ID NO: 919 - DNA, 21 bp, Artificial Sequence
  Description of Artificial Sequence Synthetic oligonucleotide
tccgcatcccgctccggtgag

SEQ ID NO: 920 - DNA, 21 bp, Artificial Sequence
  Description of Artificial Sequence Synthetic oligonucleotide
ctggcggcggccgacatcgca

SEQ ID NO: 921 - DNA, 21 bp, Artificial Sequence
  Description of Artificial Sequence Synthetic oligonucleotide
gccattgctgtgctggccatc

SEQ ID NO: 922 - DNA, 21 bp, Artificial Sequence
  Description of Artificial Sequence Synthetic oligonucleotide
atcccgctccggtgagcaggg

SEQ ID NO: 923 - DNA, 21 bp, Artificial Sequence
  Description of Artificial Sequence Synthetic oligonucleotide
agactgaagatggagctctgc

SEQ ID NO: 924 - DNA, 21 bp, Artificial Sequence
  Description of Artificial Sequence Synthetic oligonucleotide
ccccggccctgctcaccggag

SEQ ID NO: 925 - DNA, 21 bp, Artificial Sequence
  Description of Artificial Sequence Synthetic oligonucleotide
atggtgatggcaaaggggatg

SEQ ID NO: 926 - DNA, 21 bp, Artificial Sequence
  Description of Artificial Sequence Synthetic oligonucleotide
gctcctcggtgtacatcacgg

SEQ ID NO: 927 - DNA, 20 bp, Artificial Sequence
  Description of Artificial Sequence Synthetic oligonucleotide
tgtcgatggcaatagccaag

SEQ ID NO: 928 - DNA, 20 bp, Artificial Sequence
  Description of Artificial Sequence Synthetic oligonucleotide
agaagttggtgacgttctgc

SEQ ID NO: 929 - DNA, 20 bp, Artificial Sequence
  Description of Artificial Sequence Synthetic oligonucleotide
ttcgccatcaccatcagcac

SEQ ID NO: 930 - DNA, 20 bp, Artificial Sequence
  Description of Artificial Sequence Synthetic oligonucleotide
gaagaagaggcagccatggc

SEQ ID NO: 931 - DNA, 20 bp, Artificial Sequence
  Description of Artificial Sequence Synthetic oligonucleotide
cacaagcacgttacccagga

SEQ ID NO: 932 - DNA, 20 bp, Artificial Sequence
  Description of Artificial Sequence Synthetic oligonucleotide
caacttcttcgtggtatctc

SEQ ID NO: 933 - DNA, 20 bp, Artificial Sequence
  Description of Artificial Sequence Synthetic oligonucleotide
caggatggccagcacagcaa

SEQ ID NO: 934 - DNA, 20 bp, Artificial Sequence
  Description of Artificial Sequence Synthetic oligonucleotide
aattccactccggtgagcca

SEQ ID NO: 935 - DNA, 20 bp, Artificial Sequence
  Description of Artificial Sequence Synthetic oligonucleotide
agcgcagaagccagtgctga

SEQ ID NO: 936 - DNA, 20 bp, Artificial Sequence
  Description of Artificial Sequence Synthetic oligonucleotide
gtgctgatggtgatggcgaa

SEQ ID NO: 937 - DNA, 20 bp, Artificial Sequence
  Description of Artificial Sequence Synthetic oligonucleotide
ggagctggccattgctgtgc

SEQ ID NO: 938 - DNA, 20 bp, Artificial Sequence
  Description of Artificial Sequence Synthetic oligonucleotide
aatagccaagaggctgaaga

SEQ ID NO: 939 - DNA, 20 bp, Artificial Sequence
  Description of Artificial Sequence Synthetic oligonucleotide
ctcctcggtgtacatcatgg

SEQ ID NO: 940 - DNA, 20 bp, Artificial Sequence
  Description of Artificial Sequence Synthetic oligonucleotide
ggacaaagcaggcgaagaag

SEQ ID NO: 941 - DNA, 20 bp, Artificial Sequence
  Description of Artificial Sequence Synthetic oligonucleotide
tctggcggcggctgacatcg

SEQ ID NO: 942 - DNA, 20 bp, Artificial Sequence
  Description of Artificial Sequence Synthetic oligonucleotide
tgggtaacgtgcttgtgtgc

SEQ ID NO: 943 - DNA, 20 bp, Artificial Sequence
  Description of Artificial Sequence Synthetic oligonucleotide
gatgtacaccgaggagccca

SEQ ID NO: 944 - DNA, 20 bp, Artificial Sequence
  Description of Artificial Sequence Synthetic oligonucleotide
taacccctggctcaccggag

SEQ ID NO: 945 - DNA, 20 bp, Artificial Sequence
  Description of Artificial Sequence Synthetic oligonucleotide
tcaccggagtggaattcgga

SEQ ID NO: 946 - DNA, 20 bp, Artificial Sequence
  Description of Artificial Sequence Synthetic oligonucleotide
gcggcggctgacatcgcggt

SEQ ID NO: 947 - DNA, 20 bp, Artificial Sequence
  Description of Artificial Sequence Synthetic oligonucleotide
gatggtgatggcgaatggga

SEQ ID NO: 948 - DNA, 20 bp, Artificial Sequence
  Description of Artificial Sequence Synthetic oligonucleotide
ggcttctgcgctgcctgcca

SEQ ID NO: 949 - DNA, 20 bp, Artificial Sequence
  Description of Artificial Sequence Synthetic oligonucleotide
attccactccggtgagccag

SEQ ID NO: 950 - DNA, 20 bp, Artificial Sequence
  Description of Artificial Sequence Synthetic oligonucleotide
ggtgtacatcatggtggagc

SEQ ID NO: 951 - DNA, 20 bp, Artificial Sequence
  Description of Artificial Sequence Synthetic oligonucleotide
attgctgtgctggccatcct

SEQ ID NO: 952 - DNA, 20 bp, Artificial Sequence
  Description of Artificial Sequence Synthetic oligonucleotide
ctccaccatgatgtacaccg

SEQ ID NO: 953 - DNA, 20 bp, Artificial Sequence
  Description of Artificial Sequence Synthetic oligonucleotide
ggcggcggctgacatcgcgg

SEQ ID NO: 954 - DNA, 20 bp, Artificial Sequence
  Description of Artificial Sequence Synthetic oligonucleotide
tacaccgaggagcccatggc

SEQ ID NO: 955 - DNA, 20 bp, Artificial Sequence
  Description of Artificial Sequence Synthetic oligonucleotide
gggtaacgtgcttgtgtgct

SEQ ID NO: 956 - DNA, 20 bp, Artificial Sequence
  Description of Artificial Sequence Synthetic oligonucleotide
caggttgctgttgatccaca

SEQ ID NO: 957 - DNA, 20 bp, Artificial Sequence
  Description of Artificial Sequence Synthetic oligonucleotide
tgaagatggaactctgcgtg

SEQ ID NO: 958 - DNA, 20 bp, Artificial Sequence
  Description of Artificial Sequence Synthetic oligonucleotide
gatggcgatgtatctgtcga

SEQ ID NO: 959 - DNA, 20 bp, Artificial Sequence
  Description of Artificial Sequence Synthetic oligonucleotide
cttcttcgcctgctttgtcc

SEQ ID NO: 960 - DNA, 20 bp, Artificial Sequence
  Description of Artificial Sequence Synthetic oligonucleotide
aggcgaagaagaggcagcca

SEQ ID NO: 961 - DNA, 20 bp, Artificial Sequence
  Description of Artificial Sequence Synthetic oligonucleotide
tgcttgtgtgctgggccgtg

SEQ ID NO: 962 - DNA, 20 bp, Artificial Sequence
  Description of Artificial Sequence Synthetic oligonucleotide
gaagccagtgctgatggtga

SEQ ID NO: 963 - DNA, 20 bp, Artificial Sequence
  Description of Artificial Sequence Synthetic oligonucleotide
cgtgaggaccaggacaaagc

SEQ ID NO: 964 - DNA, 20 bp, Artificial Sequence
  Description of Artificial Sequence Synthetic oligonucleotide
tggaactctgcgtgaggacc

SEQ ID NO: 965 - DNA, 20 bp, Artificial Sequence
  Description of Artificial Sequence Synthetic oligonucleotide
cattgctgtgctggccatcc

SEQ ID NO: 966 - DNA, 20 bp, Artificial Sequence
  Description of Artificial Sequence Synthetic oligonucleotide
ttctcccgccatgggctcct

SEQ ID NO: 967 - DNA, 20 bp, Artificial Sequence
  Description of Artificial Sequence Synthetic oligonucleotide
tggctcaccggagtggaatt

SEQ ID NO: 968 - DNA, 20 bp, Artificial Sequence
  Description of Artificial Sequence Synthetic oligonucleotide
tgctgatggtgatggcgaat

SEQ ID NO: 969 - DNA, 20 bp, Artificial Sequence
  Description of Artificial Sequence Synthetic oligonucleotide
cttcgtggtatctctggcgg

SEQ ID NO: 970 - DNA, 20 bp, Artificial Sequence
  Description of Artificial Sequence Synthetic oligonucleotide
agcacacaagcacgttaccc

SEQ ID NO: 971 - DNA, 20 bp, Artificial Sequence
  Description of Artificial Sequence Synthetic oligonucleotide
gggctcctcggtgtacatca

SEQ ID NO: 972 - DNA, 20 bp, Artificial Sequence
  Description of Artificial Sequence Synthetic oligonucleotide
gtacaccgaggagcccatgg

SEQ ID NO: 973 - DNA, 20 bp, Artificial Sequence
  Description of Artificial Sequence Synthetic oligonucleotide
gaacgtcaccaacttcttcg

SEQ ID NO: 974 - DNA, 20 bp, Artificial Sequence
  Description of Artificial Sequence Synthetic oligonucleotide
tcgccatccgaattccactc

SEQ ID NO: 975 - DNA, 20 bp, Artificial Sequence
  Description of Artificial Sequence Synthetic oligonucleotide
gagttccatcttcagcctct

SEQ ID NO: 976 - DNA, 20 bp, Artificial Sequence
  Description of Artificial Sequence Synthetic oligonucleotide
gaattccactccggtgagcc

SEQ ID NO: 977 - DNA, 20 bp, Artificial Sequence
  Description of Artificial Sequence Synthetic oligonucleotide
cagagataccacgaagaagt

SEQ ID NO: 978 - DNA, 20 bp, Artificial Sequence
  Description of Artificial Sequence Synthetic oligonucleotide
cttcttcgtggtatctctgg

SEQ ID NO: 979 - DNA, 21 bp, Artificial Sequence
  Description of Artificial Sequence Synthetic oligonucleotide
cagtgctgatggtgatggcga

SEQ ID NO: 980 - DNA, 21 bp, Artificial Sequence
  Description of Artificial Sequence Synthetic oligonucleotide
cgaattccactccggtgagcc

SEQ ID NO: 981 - DNA, 21 bp, Artificial Sequence
  Description of Artificial Sequence Synthetic oligonucleotide
ccgaattccactccggtgagc

SEQ ID NO: 982 - DNA, 21 bp, Artificial Sequence
  Description of Artificial Sequence Synthetic oligonucleotide
gctgaagatggaactctgcgt

SEQ ID NO: 983 - DNA, 21 bp, Artificial Sequence
  Description of Artificial Sequence Synthetic oligonucleotide
cgtgcttgtgtgctgggccgt

SEQ ID NO: 984 - DNA, 21 bp, Artificial Sequence
  Description of Artificial Sequence Synthetic oligonucleotide
gtgaggaccaggacaaagcag

SEQ ID NO: 985 - DNA, 21 bp, Artificial Sequence
  Description of Artificial Sequence Synthetic oligonucleotide
tcgatggcaatagccaagagg

SEQ ID NO: 986 - DNA, 21 bp, Artificial Sequence
  Description of Artificial Sequence Synthetic oligonucleotide
catcgacagatacatcgccat

SEQ ID NO: 987 - DNA, 21 bp, Artificial Sequence
  Description of Artificial Sequence Synthetic oligonucleotide
gtacaccgaggagcccatggc

SEQ ID NO: 988 - DNA, 21 bp, Artificial Sequence
  Description of Artificial Sequence Synthetic oligonucleotide
gctccaccatgatgtacaccg

SEQ ID NO: 989 - DNA, 21 bp, Artificial Sequence
  Description of Artificial Sequence Synthetic oligonucleotide
aagccagtgctgatggtgatg

SEQ ID NO: 990 - DNA, 21 bp, Artificial Sequence
  Description of Artificial Sequence Synthetic oligonucleotide
caccgcgatgtcagccgccgc

SEQ ID NO: 991 - DNA, 21 bp, Artificial Sequence
  Description of Artificial Sequence Synthetic oligonucleotide
aggctgaagatggaactctgc

SEQ ID NO: 992 - DNA, 21 bp, Artificial Sequence
  Description of Artificial Sequence Synthetic oligonucleotide
gccgccgccagagataccacg

SEQ ID NO: 993 - DNA, 21 bp, Artificial Sequence
  Description of Artificial Sequence Synthetic oligonucleotide
agctccaccatgatgtacacc

SEQ ID NO: 994 - DNA, 21 bp, Artificial Sequence
  Description of Artificial Sequence Synthetic oligonucleotide
aggcagccatggcaggcagcg

SEQ ID NO: 995 - DNA, 21 bp, Artificial Sequence
  Description of Artificial Sequence Synthetic oligonucleotide
cctggctcaccggagtggaat

SEQ ID NO: 996 - DNA, 21 bp, Artificial Sequence
  Description of Artificial Sequence Synthetic oligonucleotide
ccagctccaccatgatgtaca

SEQ ID NO: 997 - DNA, 21 bp, Artificial Sequence
  Description of Artificial Sequence Synthetic oligonucleotide
accaggacaaagcaggcgaag

SEQ ID NO: 998 - DNA, 21 bp, Artificial Sequence
  Description of Artificial Sequence Synthetic oligonucleotide
cctgggtaacgtgcttgtgtg

SEQ ID NO: 999 - DNA, 21 bp, Artificial Sequence
  Description of Artificial Sequence Synthetic oligonucleotide
aggaccaggacaaagcaggcg

SEQ ID NO: 1000 - DNA, 21 bp, Artificial Sequence
  Description of Artificial Sequence Synthetic oligonucleotide
tcagccgccgccagagatacc

SEQ ID NO: 1001 - DNA, 21 bp, Artificial Sequence
  Description of Artificial Sequence Synthetic oligonucleotide
ggctcctcggtgtacatcatg

SEQ ID NO: 1002 - DNA, 21 bp, Artificial Sequence
  Description of Artificial Sequence Synthetic oligonucleotide
ctggcggcggctgacatcgcg

SEQ ID NO: 1003 - DNA, 21 bp, Artificial Sequence
  Description of Artificial Sequence Synthetic oligonucleotide
gatggaactctgcgtgaggac

SEQ ID NO: 1004 - DNA, 21 bp, Artificial Sequence
  Description of Artificial Sequence Synthetic oligonucleotide
gctcctcggtgtacatcatgg

SEQ ID NO: 1005 - DNA, 21 bp, Artificial Sequence
  Description of Artificial Sequence Synthetic oligonucleotide
tgtacaccgaggagcccatgg

SEQ ID NO: 1006 - DNA, 21 bp, Artificial Sequence
  Description of Artificial Sequence Synthetic oligonucleotide
gccattgctgtgctggccatc

SEQ ID NO: 1007 - DNA, 21 bp, Artificial Sequence
  Description of Artificial Sequence Synthetic oligonucleotide
caatagccaagaggctgaaga

SEQ ID NO: 1008 - DNA, 21 bp, Artificial Sequence
  Description of Artificial Sequence Synthetic oligonucleotide
atggtgatggcgaatgggatg

SEQ ID NO: 1009 - DNA, 21 bp, Artificial Sequence
  Description of Artificial Sequence Synthetic oligonucleotide
atgtacaccgaggagcccatg

SEQ ID NO: 1010 - DNA, 21 bp, Artificial Sequence
  Description of Artificial Sequence Synthetic oligonucleotide
gtgtggatcaacagcaacctg

SEQ ID NO: 1011 - DNA, 21 bp, Artificial Sequence
  Description of Artificial Sequence Synthetic oligonucleotide
tgctttgtcctggtcctcacg

SEQ ID NO: 1012 - DNA, 21 bp, Artificial Sequence
  Description of Artificial Sequence Synthetic oligonucleotide
gtaacccctggctcaccggag

SEQ ID NO: 1013 - DNA, 21 bp, Artificial Sequence
  Description of Artificial Sequence Synthetic oligonucleotide
ccagcacacaagcacgttacc

SEQ ID NO: 1014 - DNA, 21 bp, Artificial Sequence
  Description of Artificial Sequence Synthetic oligonucleotide
tatctgtcgatggcaatagcc

SEQ ID NO: 1015 - DNA, 21 bp, Artificial Sequence
  Description of Artificial Sequence Synthetic oligonucleotide
gcaatagccaagaggctgaag

SEQ ID NO: 1016 - DNA, 21 bp, Artificial Sequence
  Description of Artificial Sequence Synthetic oligonucleotide
agtgctgatggtgatggcgaa

SEQ ID NO: 1017 - DNA, 21 bp, Artificial Sequence
  Description of Artificial Sequence Synthetic oligonucleotide
acaccgaggagcccatggcgg

SEQ ID NO: 1018 - DNA, 21 bp, Artificial Sequence
  Description of Artificial Sequence Synthetic oligonucleotide
cgccatccgaattccactccg

SEQ ID NO: 1019 - DNA, 20 bp, Artificial Sequence
  Description of Artificial Sequence Synthetic oligonucleotide
tggtgtcactggcggcggcc

SEQ ID NO: 1020 - DNA, 20 bp, Artificial Sequence
  Description of Artificial Sequence Synthetic oligonucleotide
ccatcaccatcagcaccggg

SEQ ID NO: 1021 - DNA, 20 bp, Artificial Sequence
  Description of Artificial Sequence Synthetic oligonucleotide
ccatcggcctgactcccatg

SEQ ID NO: 1022 - DNA, 20 bp, Artificial Sequence
  Description of Artificial Sequence Synthetic oligonucleotide
gctgaccgcagttgttccaa

SEQ ID NO: 1023 - DNA, 20 bp, Artificial Sequence
  Description of Artificial Sequence Synthetic oligonucleotide
aggatgtggtccccatgaac

SEQ ID NO: 1024 - DNA, 20 bp, Artificial Sequence
  Description of Artificial Sequence Synthetic oligonucleotide
cctgtgtgctggtgcccctg

SEQ ID NO: 1025 - DNA, 20 bp, Artificial Sequence
  Description of Artificial Sequence Synthetic oligonucleotide
cggatcttcctggcggcgcg

SEQ ID NO: 1026 - DNA, 20 bp, Artificial Sequence
  Description of Artificial Sequence Synthetic oligonucleotide
ccctctgctggctgccccta

SEQ ID NO: 1027 - DNA, 20 bp, Artificial Sequence
  Description of Artificial Sequence Synthetic oligonucleotide
ttctgccccgactgcagcca

SEQ ID NO: 1028 - DNA, 20 bp, Artificial Sequence
  Description of Artificial Sequence Synthetic oligonucleotide
aaggcagctggcaccagtgc

SEQ ID NO: 1029 - DNA, 21 bp, Artificial Sequence
  Description of Artificial Sequence Synthetic oligonucleotide
taagggcatcattgccatctg

SEQ ID NO: 1030 - DNA, 21 bp, Artificial Sequence
  Description of Artificial Sequence Synthetic oligonucleotide
cggcctgactcccatgctagg

SEQ ID NO: 1031 - DNA, 21 bp, Artificial Sequence
  Description of Artificial Sequence Synthetic oligonucleotide
gcagttgttccaacctagcat

SEQ ID NO: 1032 - DNA, 21 bp, Artificial Sequence
  Description of Artificial Sequence Synthetic oligonucleotide
ccgcagttgttccaacctagc

SEQ ID NO: 1033 - DNA, 21 bp, Artificial Sequence
  Description of Artificial Sequence Synthetic oligonucleotide
caagaaccactcccagggctg

SEQ ID NO: 1034 - DNA, 21 bp, Artificial Sequence
  Description of Artificial Sequence Synthetic oligonucleotide
cttggccctccccgcagccct

SEQ ID NO: 1035 - DNA, 21 bp, Artificial Sequence
  Description of Artificial Sequence Synthetic oligonucleotide
cacttggccctccccgcagcc

SEQ ID NO: 1036 - DNA, 21 bp, Artificial Sequence
  Description of Artificial Sequence Synthetic oligonucleotide
ggccaagtggcctgtctcttt

SEQ ID NO: 1037 - DNA, 21 bp, Artificial Sequence
  Description of Artificial Sequence Synthetic oligonucleotide
ttcatggggaccacatcctca

SEQ ID NO: 1038 - DNA, 21 bp, Artificial Sequence
  Description of Artificial Sequence Synthetic oligonucleotide
tgaagtacaccatgtagttca

SEQ ID NO: 1039 - DNA, 21 bp, Artificial Sequence
  Description of Artificial Sequence Synthetic oligonucleotide
ctggtgcccctgctgctcatg

SEQ ID NO: 1040 - DNA, 21 bp, Artificial Sequence
  Description of Artificial Sequence Synthetic oligonucleotide
gctcatgctgggtgtctattt

SEQ ID NO: 1041 - DNA, 21 bp, Artificial Sequence
  Description of Artificial Sequence Synthetic oligonucleotide
cttcagctgtcgtcgcgccgc

SEQ ID NO: 1042 - DNA, 21 bp, Artificial Sequence
  Description of Artificial Sequence Synthetic oligonucleotide
cgcgacgacagctgaagcaga

SEQ ID NO: 1043 - DNA, 21 bp, Artificial Sequence
  Description of Artificial Sequence Synthetic oligonucleotide
gatggagagccagcctctgcc

SEQ ID NO: 1044 - DNA, 21 bp, Artificial Sequence
  Description of Artificial Sequence Synthetic oligonucleotide
gcgtggctgcagtcggggcag

SEQ ID NO: 1045 - DNA, 21 bp, Artificial Sequence
  Description of Artificial Sequence Synthetic oligonucleotide
acgatggccaggtacatgagc

SEQ ID NO: 1046 - DNA, 21 bp, Artificial Sequence
  Description of Artificial Sequence Synthetic oligonucleotide
ctctcccacaccaattcggtt

SEQ ID NO: 1047 - DNA, 21 bp, Artificial Sequence
  Description of Artificial Sequence Synthetic oligonucleotide
gattcacaaccgaattggtgt

SEQ ID NO: 1048 - DNA, 21 bp, Artificial Sequence
  Description of Artificial Sequence Synthetic oligonucleotide
gggattcacaaccgaattggt

SEQ ID NO: 1049 - DNA, 21 bp, Artificial Sequence
  Description of Artificial Sequence Synthetic oligonucleotide
cgtagatgaagggattcacaa

SEQ ID NO: 1050 - DNA, 21 bp, Artificial Sequence
  Description of Artificial Sequence Synthetic oligonucleotide
ggatacggtaggcgtagatga

SEQ ID NO: 1051 - DNA, 21 bp, Artificial Sequence
  Description of Artificial Sequence Synthetic oligonucleotide
tcatctacgcctaccgtatcc

SEQ ID NO: 1052 - DNA, 21 bp, Artificial Sequence
  Description of Artificial Sequence Synthetic oligonucleotide
cggatacggtaggcgtagatg

SEQ ID NO: 1053 - DNA, 21 bp, Artificial Sequence
  Description of Artificial Sequence Synthetic oligonucleotide
gcggaaggtctggcggaactc

SEQ ID NO: 1054 - DNA, 21 bp, Artificial Sequence
  Description of Artificial Sequence Synthetic oligonucleotide
aatgatcttgcggaaggtctg

SEQ ID NO: 1055 - DNA, 21 bp, Artificial Sequence
  Description of Artificial Sequence Synthetic oligonucleotide
gacgtggctgcgaatgatctt

SEQ ID NO: 1056 - DNA, 21 bp, Artificial Sequence
  Description of Artificial Sequence Synthetic oligonucleotide
ttgctgcctcaggacgtggct

SEQ ID NO: 1057 - DNA, 21 bp, Artificial Sequence
  Description of Artificial Sequence Synthetic oligonucleotide
caaggcagctggcaccagtgc

SEQ ID NO: 1058 - DNA, 21 bp, Artificial Sequence
  Description of Artificial Sequence Synthetic oligonucleotide
cgggcactggtgccagctgcc

SEQ ID NO: 1059 - DNA, 21 bp, Artificial Sequence
  Description of Artificial Sequence Synthetic oligonucleotide
cttggcagctcatggcagtga

SEQ ID NO: 1060 - DNA, 21 bp, Artificial Sequence
  Description of Artificial Sequence Synthetic oligonucleotide
ccgtctcaacggccacccgcc

SEQ ID NO: 1061 - DNA, 21 bp, Artificial Sequence
  Description of Artificial Sequence Synthetic oligonucleotide
cacactcctggcgggtggccg

SEQ ID NO: 1062 - DNA, 21 bp, Artificial Sequence
  Description of Artificial Sequence Synthetic oligonucleotide
tgccgttggcccacactcctg

SEQ ID NO: 1063 - DNA, 21 bp, Artificial Sequence
  Description of Artificial Sequence Synthetic oligonucleotide
ccattgggcctccgctcaggg

SEQ ID NO: 1064 - DNA, 21 bp, Artificial Sequence
  Description of Artificial Sequence Synthetic oligonucleotide
catagccattgggcctccgct

SEQ ID NO: 1065 - DNA, 21 bp, Artificial Sequence
  Description of Artificial Sequence Synthetic oligonucleotide
aatggctatgccctggggctg

SEQ ID NO: 1066 - DNA, 21 bp, Artificial Sequence
  Description of Artificial Sequence Synthetic oligonucleotide
atgccctggggctggtgagtg

SEQ ID NO: 1067 - DNA, 21 bp, Artificial Sequence
  Description of Artificial Sequence Synthetic oligonucleotide
gccctggggctggtgagtgga

SEQ ID NO: 1068 - DNA, 21 bp, Artificial Sequence
  Description of Artificial Sequence Synthetic oligonucleotide
tggtgagtggagggagtgccc

SEQ ID NO: 1069 - DNA, 21 bp, Artificial Sequence
  Description of Artificial Sequence Synthetic oligonucleotide
gagggagtgcccaagagtccc

SEQ ID NO: 1070 - DNA, 21 bp, Artificial Sequence
  Description of Artificial Sequence Synthetic oligonucleotide
agggagtgcccaagagtccca

SEQ ID NO: 1071 - DNA, 21 bp, Artificial Sequence
  Description of Artificial Sequence Synthetic oligonucleotide
gtctgggaggcccgtgttccc

SEQ ID NO: 1072 - DNA, 21 bp, Artificial Sequence
  Description of Artificial Sequence Synthetic oligonucleotide
catggctaaggagctccacgt

SEQ ID NO: 1073 - DNA, 21 bp, Artificial Sequence
  Description of Artificial Sequence Synthetic oligonucleotide
gagctccttagccatgagctc

SEQ ID NO: 1074 - DNA, 21 bp, Artificial Sequence
  Description of Artificial Sequence Synthetic oligonucleotide
gctccttagccatgagctcaa

SEQ ID NO: 1075 - DNA, 21 bp, Artificial Sequence
  Description of Artificial Sequence Synthetic oligonucleotide
ggcctagatgaccccctggcc

SEQ ID NO: 1076 - DNA, 21 bp, Artificial Sequence
  Description of Artificial Sequence Synthetic oligonucleotide
ccccctggcccaggatggagc

SEQ ID NO: 1077 - DNA, 21 bp, Artificial Sequence
  Description of Artificial Sequence Synthetic oligonucleotide
ctcctgctccatcctgggcca

SEQ ID NO: 1078 - DNA, 20 bp, Artificial Sequence
  Description of Artificial Sequence Synthetic oligonucleotide
ccgtgatgtacaccgaggag

SEQ ID NO: 1079 - DNA, 20 bp, Artificial Sequence
  Description of Artificial Sequence Synthetic oligonucleotide
ctttgccatcaccatcagca

SEQ ID NO: 1080 - DNA, 20 bp, Artificial Sequence
  Description of Artificial Sequence Synthetic oligonucleotide
tttgccatcaccatcagcac

SEQ ID NO: 1081 - DNA, 20 bp, Artificial Sequence
  Description of Artificial Sequence Synthetic oligonucleotide
ttgcctgcttcgtcctggtc

SEQ ID NO: 1082 - DNA, 20 bp, Artificial Sequence
  Description of Artificial Sequence Synthetic oligonucleotide
tcctggtcctcacgcagagc

SEQ ID NO: 1083 - DNA, 20 bp, Artificial Sequence
  Description of Artificial Sequence Synthetic oligonucleotide
tcttcagtctcctggccatc

SEQ ID NO: 1084 - DNA, 20 bp, Artificial Sequence
  Description of Artificial Sequence Synthetic oligonucleotide
gtctcctggccatcgccatt

SEQ ID NO: 1085 - DNA, 20 bp, Artificial Sequence
  Description of Artificial Sequence Synthetic oligonucleotide
acctagcatgggagtcaggc

SEQ ID NO: 1086 - DNA, 20 bp, Artificial Sequence
  Description of Artificial Sequence Synthetic oligonucleotide
aacctagcatgggagtcagg

SEQ ID NO: 1087 - DNA, 20 bp, Artificial Sequence
  Description of Artificial Sequence Synthetic oligonucleotide
atgctaggttggaacaactg

SEQ ID NO: 1088 - DNA, 20 bp, Artificial Sequence
  Description of Artificial Sequence Synthetic oligonucleotide
gcagccctgggagtggttct

SEQ ID NO: 1089 - DNA, 20 bp, Artificial Sequence
  Description of Artificial Sequence Synthetic oligonucleotide
cgcagccctgggagtggttc

SEQ ID NO: 1090 - DNA, 20 bp, Artificial Sequence
  Description of Artificial Sequence Synthetic oligonucleotide
agggctgcggggagggccaa

SEQ ID NO: 1091 - DNA, 20 bp, Artificial Sequence
  Description of Artificial Sequence Synthetic oligonucleotide
tggggaccacatcctcaaag

SEQ ID NO: 1092 - DNA, 20 bp, Artificial Sequence
  Description of Artificial Sequence Synthetic oligonucleotide
catgaactacatggtgtact

SEQ ID NO: 1093 - DNA, 20 bp, Artificial Sequence
  Description of Artificial Sequence Synthetic oligonucleotide
atgaactacatggtgtactt

SEQ ID NO: 1094 - DNA, 20 bp, Artificial Sequence
  Description of Artificial Sequence Synthetic oligonucleotide
acttctttgcctgtgtgctg

SEQ ID NO: 1095 - DNA, 20 bp, Artificial Sequence
  Description of Artificial Sequence Synthetic oligonucleotide
tgctgctcatgctgggtgtc

SEQ ID NO: 1096 - DNA, 20 bp, Artificial Sequence
  Description of Artificial Sequence Synthetic oligonucleotide
caaatagacacccagcatga

SEQ ID NO: 1097 - DNA, 20 bp, Artificial Sequence
  Description of Artificial Sequence Synthetic oligonucleotide
gctgtcgtcgcgccgccagg

SEQ ID NO: 1098 - DNA, 20 bp, Artificial Sequence
  Description of Artificial Sequence Synthetic oligonucleotide
tggcggcgcgacgacagctg

SEQ ID NO: 1099 - DNA, 20 bp, Artificial Sequence
  Description of Artificial Sequence Synthetic oligonucleotide
tctgcttcagctgtcgtcgc

SEQ ID NO: 1100 - DNA, 20 bp, Artificial Sequence
  Description of Artificial Sequence Synthetic oligonucleotide
ggcagaggctggctctccat

SEQ ID NO: 1101 - DNA, 20 bp, Artificial Sequence
  Description of Artificial Sequence Synthetic oligonucleotide
cggcagaggctggctctcca

SEQ ID NO: 1102 - DNA, 20 bp, Artificial Sequence
  Description of Artificial Sequence Synthetic oligonucleotide
ccggcagaggctggctctcc

SEQ ID NO: 1103 - DNA, 20 bp, Artificial Sequence
  Description of Artificial Sequence Synthetic oligonucleotide
cactgcagaaggaggtccat

SEQ ID NO: 1104 - DNA, 20 bp, Artificial Sequence
  Description of Artificial Sequence Synthetic oligonucleotide
tgctgccaagtcactggcca

SEQ ID NO: 1105 - DNA, 20 bp, Artificial Sequence
  Description of Artificial Sequence Synthetic oligonucleotide
acaatgatggccagtgactt

SEQ ID NO: 1106 - DNA, 20 bp, Artificial Sequence
  Description of Artificial Sequence Synthetic oligonucleotide
tacacatcatcaactgcttc

SEQ ID NO: 1107 - DNA, 20 bp, Artificial Sequence
  Description of Artificial Sequence Synthetic oligonucleotide
ctttcttctgccccgactgc

SEQ ID NO: 1108 - DNA, 20 bp, Artificial Sequence
  Description of Artificial Sequence Synthetic oligonucleotide
gactgcagccacgcccctct

SEQ ID NO: 1109 - DNA, 20 bp, Artificial Sequence
  Description of Artificial Sequence Synthetic oligonucleotide
tctctggctcatgtacctgg

SEQ ID NO: 1110 - DNA, 20 bp, Artificial Sequence
  Description of Artificial Sequence Synthetic oligonucleotide
caaccgaattggtgtgggag

SEQ ID NO: 1111 - DNA, 20 bp, Artificial Sequence
  Description of Artificial Sequence Synthetic oligonucleotide
acaccaattcggttgtgaat

SEQ ID NO: 1112 - DNA, 20 bp, Artificial Sequence
  Description of Artificial Sequence Synthetic oligonucleotide
gttgtgaatcccttcatcta

SEQ ID NO: 1113 - DNA, 20 bp, Artificial Sequence
  Description of Artificial Sequence Synthetic oligonucleotide
ttcatctacgcctaccgtat

SEQ ID NO: 1114 - DNA, 20 bp, Artificial Sequence
  Description of Artificial Sequence Synthetic oligonucleotide
tctacgcctaccgtatccgc

SEQ ID NO: 1115 - DNA, 20 bp, Artificial Sequence
  Description of Artificial Sequence Synthetic oligonucleotide
cgagttccgccagaccttcc

SEQ ID NO: 1116 - DNA, 20 bp, Artificial Sequence
  Description of Artificial Sequence Synthetic oligonucleotide
gccagaccttccgcaagatc

SEQ ID NO: 1117 - DNA, 20 bp, Artificial Sequence
  Description of Artificial Sequence Synthetic oligonucleotide
ccagaccttccgcaagatca

SEQ ID NO: 1118 - DNA, 20 bp, Artificial Sequence
  Description of Artificial Sequence Synthetic oligonucleotide
gcaagatcattcgcagccac

SEQ ID NO: 1119 - DNA, 20 bp, Artificial Sequence
  Description of Artificial Sequence Synthetic oligonucleotide
caagatcattcgcagccacg

SEQ ID NO: 1120 - DNA, 20 bp, Artificial Sequence
  Description of Artificial Sequence Synthetic oligonucleotide
cagccacgtcctgaggcagc

SEQ ID NO: 1121 - DNA, 20 bp, Artificial Sequence
  Description of Artificial Sequence Synthetic oligonucleotide
aggcagctggcaccagtgcc

SEQ ID NO: 1122 - DNA, 20 bp, Artificial Sequence
  Description of Artificial Sequence Synthetic oligonucleotide
tcactgccatgagctgccaa

SEQ ID NO: 1123 - DNA, 20 bp, Artificial Sequence
  Description of Artificial Sequence Synthetic oligonucleotide
tctcaacggccacccgccag

SEQ ID NO: 1124 - DNA, 20 bp, Artificial Sequence
  Description of Artificial Sequence Synthetic oligonucleotide
ctcagggtggggagcactgc

SEQ ID NO: 1125 - DNA, 20 bp, Artificial Sequence
  Description of Artificial Sequence Synthetic oligonucleotide
caccctgagcggaggcccaa

SEQ ID NO: 1126 - DNA, 20 bp, Artificial Sequence
  Description of Artificial Sequence Synthetic oligonucleotide
accctgagcggaggcccaat

SEQ ID NO: 1127 - DNA, 20 bp, Artificial Sequence
  Description of Artificial Sequence Synthetic oligonucleotide
agggcatagccattgggcct

SEQ ID NO: 1128 - DNA, 20 bp, Artificial Sequence
  Description of Artificial Sequence Synthetic oligonucleotide
ctcaccagccccagggcata

SEQ ID NO: 1129 - DNA, 20 bp, Artificial Sequence
  Description of Artificial Sequence Synthetic oligonucleotide
tccactcaccagccccaggg

SEQ ID NO: 1130 - DNA, 20 bp, Artificial Sequence
  Description of Artificial Sequence Synthetic oligonucleotide
tgggactcttgggcactccc

SEQ ID NO: 1131 - DNA, 20 bp, Artificial Sequence
  Description of Artificial Sequence Synthetic oligonucleotide
ctgggactcttgggcactcc

SEQ ID NO: 1132 - DNA, 20 bp, Artificial Sequence
  Description of Artificial Sequence Synthetic oligonucleotide
cctgggactcttgggcactc

SEQ ID NO: 1133 - DNA, 20 bp, Artificial Sequence
  Description of Artificial Sequence Synthetic oligonucleotide
aggggaacacgggcctccca

SEQ ID NO: 1134 - DNA, 20 bp, Artificial Sequence
  Description of Artificial Sequence Synthetic oligonucleotide
cgtctgggaggcccgtgttc

SEQ ID NO: 1135 - DNA, 20 bp, Artificial Sequence
  Description of Artificial Sequence Synthetic oligonucleotide
agacgtggagctccttagcc

SEQ ID NO: 1136 - DNA, 20 bp, Artificial Sequence
  Description of Artificial Sequence Synthetic oligonucleotide
ttgagctcatggctaaggag

SEQ ID NO: 1137 - DNA, 20 bp, Artificial Sequence
  Description of Artificial Sequence Synthetic oligonucleotide
ctggcctagatgaccccctg

SEQ ID NO: 1138 - DNA, 20 bp, Artificial Sequence
  Description of Artificial Sequence Synthetic oligonucleotide
tggcctagatgaccccctgg

SEQ ID NO: 1139 - DNA, 20 bp, Artificial Sequence
  Description of Artificial Sequence Synthetic oligonucleotide
tcctgggccagggggtcatc

SEQ ID NO: 1140 - DNA, 20 bp, Artificial Sequence
  Description of Artificial Sequence Synthetic oligonucleotide
ctggcccaggatggagcagg

SEQ ID NO: 1141 - DNA, 20 bp, Artificial Sequence
  Description of Artificial Sequence Synthetic oligonucleotide
tggcccaggatggagcagga

SEQ ID NO: 1142 - DNA, 20 bp, Artificial Sequence
  Description of Artificial Sequence Synthetic oligonucleotide
cgcgagttccgccagacctt

SEQ ID NO: 1143 - DNA, 20 bp, Artificial Sequence
  Description of Artificial Sequence Synthetic oligonucleotide
ccctggggctggtgagtgga

SEQ ID NO: 1144 - protein, 1334 aa, Artificial Sequence
  Description of Artificial Sequence Synthetic polypeptide
MGHHHHHHGSTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAARLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGSPKKKRKVGSPKKKRKV

SEQ ID NO: 1145 - protein, 1363 aa, Artificial Sequence
  Description of Artificial Sequence Synthetic polypeptide
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQRPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFLFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGRSSDDEATADSQHAAPPKKKRKVGGSGGSGGSGGSGGSGGSGGSGGSLEHHHHHH

SEQ ID NO: 1146 - protein, 1363 aa, Artificial Sequence
  Description of Artificial Sequence Synthetic polypeptide
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGRSSDDEATADSQHAAPPKKKRKVGGSGGSGGSGGSGGSGGSGGSGGSLEHHHHHH

SEQ ID NO: 1147 - protein, 1363 aa, Artificial Sequence
  Description of Artificial Sequence Synthetic polypeptide
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQRPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAARLGEKMLNKKLKDQKTPIPDTLYQELYDYVNHRLSHDLSDEARALLPNVITKEVSHEIIKDRRFTSDKFLFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGRSSDDEATADSQHAAPPKKKRKVGGSGGSGGSGGSGGSGGSGGSGGSLEHHHHHH

SEQ ID NO: 1148 - protein, 1331 aa, Artificial Sequence
  Description of Artificial Sequence Synthetic polypeptide
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQRPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAARLGEKMLNKKLKDQKTPIPDTLYQELYDYVNHRLSHDLSDEARALLPNVITKEVSHEIIKDRRFTSDKFLFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGRSSDDEATADSQHAAPPKKKRKV

SEQ ID NO: 1149 - protein, 1331 aa, Artificial Sequence
  Description of Artificial Sequence Synthetic polypeptide
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQRPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFLFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGRSSDDEATADSQHAAPPKKKRKV

SEQ ID NO: 1150 - protein, 1363 aa, Artificial Sequence; identical to SEQ ID NO: 1145
  Description of Artificial Sequence Synthetic polypeptide

SEQ ID NO: 1151 - protein, 1361 aa, Artificial Sequence
  Description of Artificial Sequence Synthetic polypeptide
MGRDPGKPIPNPLLGLDSTAPKKKRKVGIHGVPAATQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNPKKKRKVKLAAALEHHHHHH

SEQ ID NO: 1152 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 1153 - RNA, 20 nt, Artificial Sequence
  Description of Artificial Sequence Synthetic oligonucleotide
uaauuucuacucuuguagau

SEQ ID NO: 1154 - DNA, 25 bp, Artificial Sequence
  Description of Artificial Sequence Synthetic oligonucleotide
atgtgtttttgtcaaaagacctttt

SEQ ID NO: 1155 - RNA, 20 nt, Artificial Sequence
  Description of Artificial Sequence Synthetic oligonucleotide
gguguacagcaguggcuggu

SEQ ID NO: 1156 - DNA, 65 bp, Artificial Sequence
  Description of Artificial Sequence Synthetic oligonucleotide
  Description of Combined DNA/RNA Molecule Synthetic oligonucleotide
atgtgtttttgtcaaaagaccttttuaauuucuacucuuguagaugguguacagcaguggcuggu

SEQ ID NO: 1157 - RNA, 20 nt, Artificial Sequence
  Description of Artificial Sequence Synthetic oligonucleotide
ugaugugagauuuuccaccu

SEQ ID NO: 1158 - DNA, 65 bp, Artificial Sequence
  Description of Artificial Sequence Synthetic oligonucleotide
  Description of Combined DNA/RNA Molecule Synthetic oligonucleotide
atgtgtttttgtcaaaagaccttttuaauuucuacucuuguagauugaugugagauuuuccaccu

SEQ ID NO: 1159 - RNA, 20 nt, Artificial Sequence
  Description of Artificial Sequence Synthetic oligonucleotide
ccaucggccugacucccaug

SEQ ID NO: 1160 - DNA, 65 bp, Artificial Sequence
  Description of Artificial Sequence Synthetic oligonucleotide
  Description of Combined DNA/RNA Molecule Synthetic oligonucleotide
atgtgtttttgtcaaaagaccttttuaauuucuacucuuguagauccaucggccugacucccaug

SEQ ID NO: 1161 - RNA, 21 nt, Artificial Sequence
  Description of Artificial Sequence Synthetic oligonucleotide
ugaugugagauuuuccaccug

SEQ ID NO: 1162 - RNA, 21 nt, Artificial Sequence
  Description of Artificial Sequence Synthetic oligonucleotide
acugacagcgugaacagguag

SEQ ID NO: 1163 - RNA, 21 nt, Artificial Sequence
  Description of Artificial Sequence Synthetic oligonucleotide
ccaucggccugacucccaugc

SEQ ID NO: 1164 - RNA, 21 nt, Artificial Sequence
  Description of Artificial Sequence Synthetic oligonucleotide
ccaucaccaucagcaccgggu

SEQ ID NO: 1165 - RNA, 21 nt, Artificial Sequence
  Description of Artificial Sequence Synthetic oligonucleotide
ccugugugcuggugccccugc

SEQ ID NO: 1166 - RNA, 21 nt, Artificial Sequence
  Description of Artificial Sequence Synthetic oligonucleotide
ugcagagaaagguggcucuau

SEQ ID NO: 1167 - RNA, 21 nt, Artificial Sequence
  Description of Artificial Sequence Synthetic oligonucleotide
ucugcagaaauguuccccguu

SEQ ID NO: 1168 - RNA, 21 nt, Artificial Sequence
  Description of Artificial Sequence Synthetic oligonucleotide
uaggaccuccaggaagauucu

SEQ ID NO: 1169 - RNA, 21 nt, Artificial Sequence
  Description of Artificial Sequence Synthetic oligonucleotide
gcaacugaacaggaaauaacc

SEQ ID NO: 1170 - RNA, 21 nt, Artificial Sequence
  Description of Artificial Sequence Synthetic oligonucleotide
guugcugccucuuuggguuug

SEQ ID NO: 1171 - RNA, 21 nt, Artificial Sequence
  Description of Artificial Sequence Synthetic oligonucleotide
aagggaaugacaaaaccuauc

SEQ ID NO: 1172 - DNA, 21 bp, Artificial Sequence
  Description of Artificial Sequence Synthetic oligonucleotide
tgatgtgagattttccacctg

SEQ ID NO: 1173 - DNA, 21 bp, Artificial Sequence
  Description of Artificial Sequence Synthetic oligonucleotide
actgacagcgtgaacaggtag

SEQ ID NO: 1174 - DNA, 21 bp, Artificial Sequence
  Description of Artificial Sequence Synthetic oligonucleotide
ccatcggcctgactcccatgc

SEQ ID NO: 1175 - DNA, 21 bp, Artificial Sequence
  Description of Artificial Sequence Synthetic oligonucleotide
tgcagagaaaggtggctctat

SEQ ID NO: 1176 - DNA, 21 bp, Artificial Sequence
  Description of Artificial Sequence Synthetic oligonucleotide
gcaactgaacaggaaataacc

SEQ ID NO: 1177 - DNA, 3993 bp, Artificial Sequence
  Description of Artificial Sequence Synthetic polynucleotide
atgacccagtttgaaggtttcaccaatctgtatcaggttagcaaaaccctgcgttttgaactgattccgcagggtaaaaccctgaaacatattcaagaacagggcttcatcgaagaggataaagcacgtaacgatcactacaaagaactgaaaccgattatcgaccgcatctataaaacctatgcagatcagtgtctgcagctggttcagctggattgggaaaatctgagcgcagcaattgatagttatcgcaaagaaaaaaccgaagaaacccgtaatgcactgattgaagaacaggcaacctatcgtaatgccatccatgattatttcattggtcgtaccgataatctgaccgatgcaattaacaaacgtcacgccgaaatctataaaggcctgtttaaagccgaactgtttaatggcaaagttctgaaacagctgggcaccgttaccaccaccgaacatgaaaatgcactgctgcgtagctttgataaattcaccacctatttcagcggcttttatgagaatcgcaaaaacgtgtttagcgcagaagatattagcaccgcaattccgcatcgtattgtgcaggataatttcccgaaattcaaagagaactgccacatttttacccgtctgattaccgcagttccgagcctgcgtgaacattttgaaaacgttaaaaaagccatcggcatctttgttagcaccagcattgaagaagtttttagcttcccgttttacaatcagctgctgacccagacccagattgatctgtataaccaactgctgggtggtattagccgtgaagcaggcaccgaaaaaatcaaaggtctgaatgaagtgctgaatctggccattcagaaaaatgatgaaaccgcacatattattgcaagcctgccgcatcgttttattccgctgttcaaacaaattctgagcgatcgtaataccctgagctttattctggaagaattcaaatccgatgaagaggtgattcagagcttttgcaaatacaaaacgctgctgcgcaatgaaaatgttctggaaactgccgaagcactgtttaacgaactgaatagcattgatctgacccacatctttatcagccacaaaaaactggaaaccatttcaagcgcactgtgtgatcattgggataccctgcgtaatgccctgtatgaacgtcgtattagcgaactgaccggtaaaattaccaaaagcgcgaaagaaaaagttcagcgcagtctgaaacatgaggatattaatctgcaagagattattagcgcagccggtaaagaactgtcagaagcatttaaacagaaaaccagcgaaattctgtcacatgcacatgcagcactggatcagccgctgccgaccaccctgaaaaaacaagaagaaaaagaaatcctgaaaagccagctggatagcctgctgggtctgtatcatctgctggactggtttgcagttgatgaaagcaatgaagttgatccggaatttagcgcacgtctgaccggcattaaactggaaatggaaccgagcctgagcttttataacaaagcccgtaattatgccaccaaaaaaccgtatagcgtcgaaaaattcaaactgaactttcagcgtccgaccctggcaagcggttgggatgttaataaagaaaaaaacaacggtgccatcctgttcgtgaaaaatggcctgtattatctgggtattatgccgaaacagaaaggtcgttataaagcgctgagctttgaaccgacggaaaaaaccagtgaaggttttgataaaatgtactacgactattttccggatgcagccaaaatgattccgaaatgtagcacccagctgaaagcagttaccgcacattttcagacccataccaccccgattctgctgagcaataactttattgaaccgctggaaatcaccaaagagatctacgatctgaataacccggaaaaagagccgaaaaaattccagaccgcatatgcaaaaaaaaccggtgatcagaaaggttatcgtgaagcgctgtgtaaatggattgatttcacccgtgattttctgagcaaatacaccaaaaccaccagtatcgatctgagcagcctgcgtccgagcagccagtataaagatctgggcgaatattatgcagaactgaatccgctgctgtatcatattagctttcagcgtattgccgagaaagaaatcatggacgcagttgaaaccggtaaactgtacctgttccagatctacaataaagattttgccaaaggccatcatggcaaaccgaatctgcataccctgtattggaccggtctgtttagccctgaaaatctggcaaaaacctcgattaaactgaatggtcaggcggaactgttttatcgtccgaaaagccgtatgaaacgtatggcagctcgtctgggtgaaaaaatgctgaacaaaaaactgaaagaccagaaaaccccgatcccggatacactgtatcaagaactgtatgattatgtgaaccatcgtctgagccatgatctgagtgatgaagcacgtgccctgctgccgaatgttattaccaaagaagttagccacgagatcattaaagatcgtcgttttaccagcgacaaattcctgtttcatgtgccgattaccctgaattatcaggcagcaaatagcccgagcaaatttaaccagcgtgttaatgcatatctgaaagaacatccagaaacgccgattattggtattgatcgtggtgaacgtaacctgatttatatcaccgttattgatagcaccggcaaaatcctggaacagcgtagcctgaataccattcagcagtttgattaccagaaaaaactggataatcgcgagaaagaacgtgttgcagcacgtcaggcatggtcagttgttggtacaattaaagacctgaaacagggttatctgagccaggttattcatgaaattgtggatctgatgattcactatcaggccgttgttgtgctggaaaacctgaattttggctttaaaagcaaacgtaccggcattgcagaaaaagcagtttatcagcagttcgagaaaatgctgattgacaaactgaattgcctggtgctgaaagattatccggctgaaaaagttggtggtgttctgaatccgtatcagctgaccgatcagtttaccagctttgcaaaaatgggcacccagagcggatttctgttttatgttccggcaccgtatacgagcaaaattgatccgctgaccggttttgttgatccgtttgtttggaaaaccatcaaaaaccatgaaagccgcaaacattttctggaaggtttcgattttctgcattacgacgttaaaacgggtgatttcatcctgcactttaaaatgaatcgcaatctgagttttcagcgtggcctgcctggttttatgcctgcatgggatattgtgtttgagaaaaacgaaacacagttcgatgcaaaaggcaccccgtttattgcaggtaaacgtattgttccggtgattgaaaatcatcgtttcaccggtcgttatcgcgatctgtatccggcaaatgaactgatcgcactgctggaagagaaaggtattgtttttcgtgatggctcaaacattctgccgaaactgctggaaaatgatgatagccatgcaattgataccatggttgcactgattcgtagcgttctgcagatgcgtaatagcaatgcagcaaccggtgaagattacattaatagtccggttcgtgatctgaatggtgtttgttttgatagccgttttcagaatccggaatggccgatggatgcagatgcaaatggtgcatatcatattgcactgaaaggacagctgctgctgaaccacctgaaagaaagcaaagatctgaaactgcaaaacggcattagcaatcaggattggctggcatatatccaagaactgcgtaacggtcgtagcagtgatgatgaagcaaccgcagatagccagcatgcagcaccgcctaaaaagaaacgtaaagtt

SEQ ID NO: 1178 - RNA, 10 nt, Artificial Sequence
  Description of Artificial Sequence Synthetic oligonucleotide
  misc_feature (1)..(10): This sequence may encompass 1-10 nucleotides
aaaaaaaaaa

SEQ ID NO: 1179 - RNA, 20 nt, Artificial Sequence
  Description of Artificial Sequence Synthetic oligonucleotide
  misc_feature (1)..(20): This sequence may encompass 10-20 nucleotides
aaaaaaaaaaaaaaaaaaaa

SEQ ID NO: 1180 - RNA, 200 nt, Artificial Sequence
  Description of Artificial Sequence Synthetic polynucleotide
  misc_feature (1)..(200): This sequence may encompass 25-200 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa